SEQUENCE LISTING (180 sequences)

SEQ ID NO: 1 - protein, 10 aa, Artificial Sequence
  heavy chain CDR1, <Pb-Dotam>
GFSLSTYSMS

SEQ ID NO: 2 - protein, 16 aa, Artificial Sequence
  heavy chain CDR2 <Pb-Dotam>
FIGSRGDTYYASWAKG

SEQ ID NO: 3 - protein, 14 aa, Artificial Sequence
  heavy chain CDR3 <Pb-Dotam>
ERDPYGGGAYPPHL

SEQ ID NO: 4 - protein, 13 aa, Artificial Sequence
  light chain CDR1, <Pb-Dotam>
QSSHSVYSDNDLA

SEQ ID NO: 5 - protein, 7 aa, Artificial Sequence
  light chain CDR2 <Pb-Dotam>
QASKLAS

SEQ ID NO: 6 - protein, 12 aa, Artificial Sequence
  light chain CDR3 <Pb-Dotam>
LGGYDDESDTYG

SEQ ID NO: 7 - protein, 121 aa, Artificial Sequence
  heavy chain variable domain  <Pb-Dotam> PRIT-0213
VTLKESGPVLVKPTETLTLTCTVSGFSLSTYSMSWIRQPPGKALEWLGFIGSRGDTYYASWAKGRLTISKDTSKSQVVLTMTNMDPVDTATYYCARERDPYGGGAYPPHLWGRGTLVTVSS

SEQ ID NO: 8 - protein, 111 aa, Artificial Sequence
  light chain variable domain <Pb-Dotam> PRIT-0213
IQMTQSPSSLSASVGDRVTITCQSSHSVYSDNDLAWYQQKPGKAPKLLIYQASKLASGVPSRFSGSGSGTDFTLTISSLQPEDFATYYCLGGYDDESDTYGFGGGTKVEIK

SEQ ID NO: 9 - protein, 121 aa, Artificial Sequence
  heavy chain variable domain  <Pb-Dotam> PRIT-0214
VQLQQWGAGLLKPSETLSLTCAVYGFSLSTYSMSWIRQPPGKGLEWIGFIGSRGDTYYASWAKGRVTISRDTSKNQVSLKLSSVTAADTAVYYCARERDPYGGGAYPPHLWGRGTLVTVSS

SEQ ID NO: 10 - protein, 111 aa, Artificial Sequence; identical to SEQ ID NO: 8
  light chain variable domain <Pb-Dotam> PRIT-0214

SEQ ID NO: 11 - protein, 10 aa, Artificial Sequence
  heavy chain CDR1 <CEA> T84.66
GFNIKDTYMH

SEQ ID NO: 12 - protein, 17 aa, Artificial Sequence
  heavy chain CDR2  <CEA> T84.66
RIDPANGNSKYVPKFQG

SEQ ID NO: 13 - protein, 12 aa, Artificial Sequence
  heavy chain CDR3  <CEA> T84.66
FGYYVSDYAMAY

SEQ ID NO: 14 - protein, 15 aa, Artificial Sequence
  light chain CDR1  <CEA> T84.66
RAGESVDIFGVGFLH

SEQ ID NO: 15 - protein, 7 aa, Artificial Sequence
  light chain CDR2  <CEA> T84.66
RASNRAT

SEQ ID NO: 16 - protein, 9 aa, Artificial Sequence
  light chain CDR3  <CEA> T84.66
QQTNEDPYT

SEQ ID NO: 17 - protein, 121 aa, Artificial Sequence
  heavy chain variable domain <CEA>  T84.66
QVQLVQSGAEVKKPGSSVKVSCKASGFNIKDTYMHWVRQAPGQGLEWMGRIDPANGNSKYVPKFQGRVTITADTSTSTAYMELSSLRSEDTAVYYCAPFGYYVSDYAMAYWGQGTLVTVSS

SEQ ID NO: 18 - protein, 111 aa, Artificial Sequence
  light chain variable domain <CEA> T84.66
EIVLTQSPATLSLSPGERATLSCRAGESVDIFGVGFLHWYQQKPGQAPRLLIYRASNRATGIPARFSGSGSGTDFTLTISSLEPEDFAVYYCQQTNEDPYTFGQGTKLEIK

SEQ ID NO: 19 - protein, 10 aa, Artificial Sequence
  heavy chain CDR1 <CEA> CH1A1A
GYTFTEFGMN

SEQ ID NO: 20 - protein, 17 aa, Artificial Sequence
  heavy chain CDR2  <CEA> CH1A1A
WINTKTGEATYVEEFKG

SEQ ID NO: 21 - protein, 12 aa, Artificial Sequence
  heavy chain CDR3  <CEA> CH1A1A
WDFAYYVEAMDY

SEQ ID NO: 22 - protein, 11 aa, Artificial Sequence
  light chain CDR1  <CEA> CH1A1A
KASAAVGTYVA

SEQ ID NO: 23 - protein, 7 aa, Artificial Sequence
  light chain CDR2  <CEA> CH1A1A
SASYRKR

SEQ ID NO: 24 - protein, 10 aa, Artificial Sequence
  light chain CDR3  <CEA> CH1A1A
HQYYTYPLFT

SEQ ID NO: 25 - protein, 121 aa, Artificial Sequence
  heavy chain variable domain <CEA>  CH1A1A
QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSS

SEQ ID NO: 26 - protein, 108 aa, Artificial Sequence
  light chain variable domain <CEA>  CH1A1A
DIQMTQSPSSLSASVGDRVTITCKASAAVGTYVAWYQQKPGKAPKLLIYSASYRKRGVPSRFSGSGSGTDFTLTISSLQPEDFATYYCHQYYTYPLFTFGQGTKLEIK

SEQ ID NO: 27 - protein, 450 aa, Artificial Sequence
  Heavy chain <CEA> of P1AD8749 without linker and <DOTAM-VH>′ Same Plasmid as SeqID32, lacking linker and <DOTAM>
QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 28 - protein, 450 aa, Artificial Sequence
  P1AD8749 Heavy chain hole <CEA> CH1A1A
QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 29 - protein, 450 aa, Artificial Sequence; identical to SEQ ID NO: 28
  Heavy chain <CEA> of P1AD8592 without linker and <DOTAM-VL>′ Same Plasmid as SeqID33, lacking linker and <DOTAM>

SEQ ID NO: 30 - protein, 450 aa, Artificial Sequence; identical to SEQ ID NO: 27
  P1AD8592 Heavy chain Knob <CEA>CH1A1A

SEQ ID NO: 31 - protein, 20 aa, Artificial Sequence
  Linker
GGGGSGGGGSGGGGSGGGGS

SEQ ID NO: 32 - protein, 591 aa, Artificial Sequence
  P1AD8749 heavy chain knob <CEA>CH1A1A<Dotam-VH>
QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSVTLKESGPVLVKPTETLTLTCTVSGFSLSTYSMSWIRQPPGKALEWLGFIGSRGDTYYASWAKGRLTISKDTSKSQVVLTMTNMDPVDTATYYCARERDPYGGGAYPPHLWGRGTLVTVSS

SEQ ID NO: 33 - protein, 581 aa, Artificial Sequence
  P1AD8592 heavy chain hole <CEA>CH1A1A<Dotam-VL>
QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSGGGGSGGGGSIQMTQSPSSLSASVGDRVTITCQSSHSVYSDNDLAWYQQKPGKAPKLLIYQASKLASGVPSRFSGSGSGTDFTLTISSLQPEDFATYYCLGGYDDESDTYGFGGGTKVEIK

SEQ ID NO: 34 - protein, 215 aa, Artificial Sequence
  P1AD8749 and P1AD8592 light chain <CEA> CH1A1A
DIQMTQSPSSLSASVGDRVTITCKASAAVGTYVAWYQQKPGKAPKLLIYSASYRKRGVPSRFSGSGSGTDFTLTISSLQPEDFATYYCHQYYTYPLFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - protein, 5 aa, Artificial Sequence
  Heavy chain CDR 1, <C825>
DYGVH

SEQ ID NO: 36 - protein, 16 aa, Artificial Sequence
  Heavy chain CDR 2, <C825>
VIWSGGGTAYNTALIS

SEQ ID NO: 37 - protein, 11 aa, Artificial Sequence
  Heavy chain CDR 3, <C825>
RGSYPYNYFDA

SEQ ID NO: 38 - protein, 14 aa, Artificial Sequence
  Light chain CDR 1, <C825>
GSSTGAVTASNYAN

SEQ ID NO: 39 - protein, 7 aa, Artificial Sequence
  Light chain CDR 2, <C825>
GHNNRPP

SEQ ID NO: 40 - protein, 9 aa, Artificial Sequence
  Light chain CDR 3, <C825>
ALWYSDHWV

SEQ ID NO: 41 - protein, 119 aa, Artificial Sequence
  Heavy chain variable domain <C825>
HVKLQESGPGLVQPSQSLSLTCTVSGFSLTDYGVHWVRQSPGKGLEWLGVIWSGGGTAYNTALISRLNIYRDNSKNQVFLEMNSLQAEDTAMYYCARRGSYPYNYFDAWGQGTTVTVSS

SEQ ID NO: 42 - protein, 109 aa, Artificial Sequence
  Light chain variable domain, <C825>
QAVVIQESALTTPPGETVTLTCGSSTGAVTASNYANWVQEKPDHLFTGLIGGHNNRPPGVPARFSGSLIGDKAALTIAGTQTEDEAIYFCALWYSDHWVIGGGTKLTVL

SEQ ID NO: 43 - protein, 5 aa, Artificial Sequence
  heavy chain CDR1 <CEA> A5B7
DYYMN

SEQ ID NO: 44 - protein, 19 aa, Artificial Sequence
  heavy chain CDR2  <CEA> A5B7
FIGNKANAYTTEYSASVKG

SEQ ID NO: 45 - protein, 10 aa, Artificial Sequence
  heavy chain CDR3  <CEA> A5B7
DRGLRFYFDY

SEQ ID NO: 46 - protein, 10 aa, Artificial Sequence
  light chain CDR1  <CEA> A5B7
RASSSVTYIH

SEQ ID NO: 47 - protein, 7 aa, Artificial Sequence
  light chain CDR2  <CEA> A5B7
ATSNLAS

SEQ ID NO: 48 - protein, 9 aa, Artificial Sequence
  light chain CDR3  <CEA> A5B7
QHWSSKPPT

SEQ ID NO: 49 - protein, 121 aa, Artificial Sequence
  heavy chain variable domain <CEA>  A5B7
EVQLLESGGGLVQPGGSLRLSCAASGFTFTDYYMNWVRQAPGKGLEWLGFIGNKANAYTTEYSASVKGRFTISRDKSKNTLYLQMNSLRAEDTATYYCTRDRGLRFYFDYWGQGTTVTVSS

SEQ ID NO: 50 - protein, 106 aa, Artificial Sequence
  light chain variable domain <CEA>  A5B7
EIVLTQSPATLSLSPGERATLSCRASSSVTYIHWYQQKPGQAPRSWIYATSNLASGIPARFSGSGSGTDFTLTISSLEPEDFAVYYCQHWSSKPPTFGQGTKLEIK

SEQ ID NO: 51 - protein, 450 aa, Artificial Sequence
  P1AE4956 heavy chain hole <CEA>  A5B7
EVQLLESGGGLVQPGGSLRLSCAASGFTFTDYYMNWVRQAPGKGLEWLGFIGNKANAYTTEYSASVKGRFTISRDKSKNTLYLQMNSLRAEDTATYYCTRDRGLRFYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 52 - protein, 591 aa, Artificial Sequence
  P1AE4956 heavy chain knob <CEA> A5B7 <Dotam-VH>
EVQLLESGGGLVQPGGSLRLSCAASGFTFTDYYMNWVRQAPGKGLEWLGFIGNKANAYTTEYSASVKGRFTISRDKSKNTLYLQMNSLRAEDTATYYCTRDRGLRFYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSVTLKESGPVLVKPTETLTLTCTVSGFSLSTYSMSWIRQPPGKALEWLGFIGSRGDTYYASWAKGRLTISKDTSKSQVVLTMTNMDPVDTATYYCARERDPYGGGAYPPHLWGRGTLVTVSS

SEQ ID NO: 53 - protein, 449 aa, Artificial Sequence
  Heavy chain <CEA> of P1AE4956 without linker and DOTAM-VH
EVQLLESGGGLVQPGGSLRLSCAASGFTFTDYYMNWVRQAPGKGLEWLGFIGNKANAYTTEYSASVKGRFTISRDKSKNTLYLQMNSLRAEDTATYYCTRDRGLRFYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 54 - protein, 213 aa, Artificial Sequence
  P1AE4956 light chain <CEA>  A5B7
EIVLTQSPATLSLSPGERATLSCRASSSVTYIHWYQQKPGQAPRSWIYATSNLASGIPARFSGSGSGTDFTLTISSLEPEDFAVYYCQHWSSKPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 55 - protein, 450 aa, Artificial Sequence
  P1AE4957 heavy chain knob <CEA>  A5B7
EVQLLESGGGLVQPGGSLRLSCAASGFTFTDYYMNWVRQAPGKGLEWLGFIGNKANAYTTEYSASVKGRFTISRDKSKNTLYLQMNSLRAEDTATYYCTRDRGLRFYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 56 - protein, 581 aa, Artificial Sequence
  P1AE4957 heavy chain hole <CEA> A5B7 <Dotam-VL>
EVQLLESGGGLVQPGGSLRLSCAASGFTFTDYYMNWVRQAPGKGLEWLGFIGNKANAYTTEYSASVKGRFTISRDKSKNTLYLQMNSLRAEDTATYYCTRDRGLRFYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSGGGGSGGGGSIQMTQSPSSLSASVGDRVTITCQSSHSVYSDNDLAWYQQKPGKAPKLLIYQASKLASGVPSRFSGSGSGTDFTLTISSLQPEDFATYYCLGGYDDESDTYGFGGGTKVEIK

SEQ ID NO: 57 - protein, 449 aa, Artificial Sequence
  Heavy chain <CEA> of P1AE4957 without linker and DOTAM-VL
EVQLLESGGGLVQPGGSLRLSCAASGFTFTDYYMNWVRQAPGKGLEWLGFIGNKANAYTTEYSASVKGRFTISRDKSKNTLYLQMNSLRAEDTATYYCTRDRGLRFYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 58 - protein, 213 aa, Artificial Sequence; identical to SEQ ID NO: 54
  P1AE4957 light chain <CEA>  A5B7

SEQ ID NO: 59 - protein, 10 aa, Artificial Sequence
  heavy chain CDR1 <CEA> 28A9
GGTFSYYAIS

SEQ ID NO: 60 - protein, 17 aa, Artificial Sequence
  heavy chain CDR2 <CEA> 28A9
GILPAFGAANYAQKFQG

SEQ ID NO: 61 - protein, 11 aa, Artificial Sequence
  heavy chain CDR3 <CEA> 28A9
LPPLPGAGLDY

SEQ ID NO: 62 - protein, 11 aa, Artificial Sequence
  light chain CDR1 <CEA> 28A9
RASQSISSWLA

SEQ ID NO: 63 - protein, 7 aa, Artificial Sequence
  light chain CDR2 <CEA> 28A9
DASSLES

SEQ ID NO: 64 - protein, 9 aa, Artificial Sequence
  light chain CDR3 <CEA> 28A9
QQNTQYPMT

SEQ ID NO: 65 - protein, 120 aa, Artificial Sequence
  heavy chain variable domain <CEA>  28A9
QVQLVQSGAEVKKPGSSVKVSCKASGGTFSYYAISWVRQAPGQGLEWMGGILPAFGAANYAQKFQGRVTITADKSTSTAYMELSSLRSEDTAVYYCARLPPLPGAGLDYWGQGTTVTVSS

SEQ ID NO: 66 - protein, 107 aa, Artificial Sequence
  light chain variable domain <CEA>  28A9
DIQMTQSPSTLSASVGDRVTITCRASQSISSWLAWYQQKPGKAPKLLIYDASSLESGVPSRFSGSGSGTEFTLTISSLQPDDFATYYCQQNTQYPMTFGQGTKVEIK

SEQ ID NO: 67 - protein, 10 aa, Artificial Sequence
  heavy chain CDR1 <GPRC5D>
GFTFSKYAMA

SEQ ID NO: 68 - protein, 17 aa, Artificial Sequence
  heavy chain CDR2 <GPRC5D>
SISTGGVNTYYADSVKG

SEQ ID NO: 69 - protein, 8 aa, Artificial Sequence
  heavy chain CDR3 <GPRC5D>
HTGDYFDY

SEQ ID NO: 70 - protein, 15 aa, Artificial Sequence
  light chain CDR1 <GPRC5D>
RASQSVSISGINLMN

SEQ ID NO: 71 - protein, 7 aa, Artificial Sequence
  light chain CDR2 <GPRC5D>
HASILAS

SEQ ID NO: 72 - protein, 9 aa, Artificial Sequence
  light chain CDR3 <GPRC5D>
QQTRESPLT

SEQ ID NO: 73 - protein, 117 aa, Artificial Sequence
  Heavy chain variable domain <GPRC5D>
EVQLLESGGGLVQPGGSLRLSCAASGFTFSKYAMAWVRQAPGKGLEWVASISTGGVNTYYADSVKGRFTISRDNSKNTLYLQMNSLRAEDTAVYYCATHTGDYFDYWGQGTMVTVSS

SEQ ID NO: 74 - protein, 111 aa, Artificial Sequence
  Light chain variable domain <GPRC5D>
EIVLTQSPGTLSLSPGERATLSCRASQSVSISGINLMNWYQQKPGQQPKLLIYHASILASGIPDRFSGSGSGTDFTLTISRLEPEDFAVYYCQQTRESPLTFGQGTRLEIK

SEQ ID NO: 75 - protein, 10 aa, Artificial Sequence
  heavy chain CDR1 <FAP> 4B9
GFTFSSYAMS

SEQ ID NO: 76 - protein, 17 aa, Artificial Sequence
  heavy chain CDR2 <FAP> 4B9
AIIGSGASTYYADSVKG

SEQ ID NO: 77 - protein, 8 aa, Artificial Sequence
  heavy chain CDR3 <FAP> 4B9
GWFGGFNY

SEQ ID NO: 78 - protein, 12 aa, Artificial Sequence
  light chain CDR1 <FAP> 4B9
RASQSVTSSYLA

SEQ ID NO: 79 - protein, 7 aa, Artificial Sequence
  light chain CDR2 <FAP> 4B9
VGSRRAT

SEQ ID NO: 80 - protein, 9 aa, Artificial Sequence
  light chain CDR3 <FAP> 4B9
QQGIMLPPT

SEQ ID NO: 81 - protein, 117 aa, Artificial Sequence
  Heavy chain variable domain <FAP> 4B9
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSS

SEQ ID NO: 82 - protein, 108 aa, Artificial Sequence
  Light chain variable domain <FAP> 4B9
EIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIK

SEQ ID NO: 83 - protein, 450 aa, Artificial Sequence
  P1AF0709 HCknob <CEA> T84.66 (D1AE4688)
QVQLVQSGAEVKKPGSSVKVSCKASGFNIKDTYMHWVRQAPGQGLEWMGRIDPANGNSKYVPKFQGRVTITADTSTSTAYMELSSLRSEDTAVYYCAPFGYYVSDYAMAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 84 - protein, 581 aa, Artificial Sequence
  P1AF0709 HChole <CEA> T84.66 Dotam-VL (D1AA4920)
QVQLVQSGAEVKKPGSSVKVSCKASGFNIKDTYMHWVRQAPGQGLEWMGRIDPANGNSKYVPKFQGRVTITADTSTSTAYMELSSLRSEDTAVYYCAPFGYYVSDYAMAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSGGGGSGGGGSIQMTQSPSSLSASVGDRVTITCQSSHSVYSDNDLAWYQQKPGKAPKLLIYQASKLASGVPSRFSGSGSGTDFTLTISSLQPEDFATYYCLGGYDDESDTYGFGGGTKVEIK

SEQ ID NO: 85 - protein, 450 aa, Artificial Sequence
  P1AF0709 HChole <CEA> T84.66 without linker and DOTAM
QVQLVQSGAEVKKPGSSVKVSCKASGFNIKDTYMHWVRQAPGQGLEWMGRIDPANGNSKYVPKFQGRVTITADTSTSTAYMELSSLRSEDTAVYYCAPFGYYVSDYAMAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 86 - protein, 450 aa, Artificial Sequence; identical to SEQ ID NO: 85
  PIAF0298 HCHole <CEA> T84.66 (D1AE4687)

SEQ ID NO: 87 - protein, 594 aa, Artificial Sequence
  PIAF0298 HCknob <CEA> T84.66 Dotam-VH-AST (D1AE3668)
QVQLVQSGAEVKKPGSSVKVSCKASGFNIKDTYMHWVRQAPGQGLEWMGRIDPANGNSKYVPKFQGRVTITADTSTSTAYMELSSLRSEDTAVYYCAPFGYYVSDYAMAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSVTLKESGPVLVKPTETLTLTCTVSGFSLSTYSMSWIRQPPGKALEWLGFIGSRGDTYYASWAKGRLTISKDTSKSQVVLTMTNMDPVDTATYYCARERDPYGGGAYPPHLWGRGTLVTVSSAST

SEQ ID NO: 88 - protein, 450 aa, Artificial Sequence; identical to SEQ ID NO: 83
  PIAF0298 HCknob <CEA> T84.66 without linker and DOTAM

SEQ ID NO: 89 - protein, 218 aa, Artificial Sequence
  P1AF0709 and PIAF0298 light chain (D1AA4120)
EIVLTQSPATLSLSPGERATLSCRAGESVDIFGVGFLHWYQQKPGQAPRLLIYRASNRATGIPARFSGSGSGTDFTLTISSLEPEDFAVYYCQQTNEDPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 90 - protein, 449 aa, Artificial Sequence
  P1AF710 HCknob <CEA> 28A9 (D1AE4690)
QVQLVQSGAEVKKPGSSVKVSCKASGGTFSYYAISWVRQAPGQGLEWMGGILPAFGAANYAQKFQGRVTITADKSTSTAYMELSSLRSEDTAVYYCARLPPLPGAGLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 91 - protein, 580 aa, Artificial Sequence
  P1AF710 HChole <CEA> 28A9 Dotam-VL (D1AC3172)
QVQLVQSGAEVKKPGSSVKVSCKASGGTFSYYAISWVRQAPGQGLEWMGGILPAFGAANYAQKFQGRVTITADKSTSTAYMELSSLRSEDTAVYYCARLPPLPGAGLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSGGGGSGGGGSIQMTQSPSSLSASVGDRVTITCQSSHSVYSDNDLAWYQQKPGKAPKLLIYQASKLASGVPSRFSGSGSGTDFTLTISSLQPEDFATYYCLGGYDDESDTYGFGGGTKVEIK

SEQ ID NO: 92 - protein, 449 aa, Artificial Sequence
  P1AF710 HChole <CEA> 28A9 without linker or DOTAM
QVQLVQSGAEVKKPGSSVKVSCKASGGTFSYYAISWVRQAPGQGLEWMGGILPAFGAANYAQKFQGRVTITADKSTSTAYMELSSLRSEDTAVYYCARLPPLPGAGLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 93 - protein, 449 aa, Artificial Sequence; identical to SEQ ID NO: 92
  P1AF711 HChole <CEA> 28A9 (D1AE4689)

SEQ ID NO: 94 - protein, 593 aa, Artificial Sequence
  P1AF711 1 HCknob <CEA> 28A9 Dotam-VH-AST (D1AE3671)
QVQLVQSGAEVKKPGSSVKVSCKASGGTFSYYAISWVRQAPGQGLEWMGGILPAFGAANYAQKFQGRVTITADKSTSTAYMELSSLRSEDTAVYYCARLPPLPGAGLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSVTLKESGPVLVKPTETLTLTCTVSGFSLSTYSMSWIRQPPGKALEWLGFIGSRGDTYYASWAKGRLTISKDTSKSQVVLTMTNMDPVDTATYYCARERDPYGGGAYPPHLWGRGTLVTVSSAST

SEQ ID NO: 95 - protein, 449 aa, Artificial Sequence; identical to SEQ ID NO: 90
  P1AF711 HCknob <CEA> 28A9 without linker and DOTAM

SEQ ID NO: 96 - protein, 214 aa, Artificial Sequence
  P1AF710 and P1AF711 light chain (D1AA2299)
DIQMTQSPSTLSASVGDRVTITCRASQSISSWLAWYQQKPGKAPKLLIYDASSLESGVPSRFSGSGSGTEFTLTISSLQPDDFATYYCQQNTQYPMTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 97 - protein, 450 aa, Artificial Sequence; identical to SEQ ID NO: 27
  P1AF0712 HCknob <CEA> CH1A1A (D1AC4023)

SEQ ID NO: 98 - protein, 579 aa, Artificial Sequence
  P1AF0712 HChole <CEA> CH1A1A DOTA-VL (D1AE4684)
QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSGGGGSGGGGSQAVVIQESALTTPPGETVTLTCGSSTGAVTASNYANWVQEKPDHLFTGLIGGHNNRPPGVPARFSGSLIGDKAALTIAGTQTEDEAIYFCALWYSDHWVIGGGTKLTVL

SEQ ID NO: 99 - protein, 450 aa, Artificial Sequence; identical to SEQ ID NO: 28
  P1AF0712 HChole <CEA> without linker or DOTA

SEQ ID NO: 100 - protein, 450 aa, Artificial Sequence; identical to SEQ ID NO: 28
  P1AF0713 HCHole <CEA> CH1A1A (D1AC4022)

SEQ ID NO: 101 - protein, 592 aa, Artificial Sequence
  P1AF0713 HCknob <CEA> CH1A1A DOTA-VH-AST (D1AE3670)
QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSHVKLQESGPGLVQPSQSLSLTCTVSGFSLTDYGVHWVRQSPGKGLEWLGVIWSGGGTAYNTALISRLNIYRDNSKNQVFLEMNSLQAEDTAMYYCARRGSYPYNYFDAWGQGTTVTVSSAST

SEQ ID NO: 102 - protein, 450 aa, Artificial Sequence; identical to SEQ ID NO: 27
  P1AF0713 HCknob <CEA> CH1A1A without linker and DOTA

SEQ ID NO: 103 - protein, 215 aa, Artificial Sequence; identical to SEQ ID NO: 34
  P1AF0712 and P1AF0713 light chain (D1AA3384)

SEQ ID NO: 104 - protein, 447 aa, Artificial Sequence
  P1AF8284 and P1AF8285 HCknob <GPRC5D> (D1AF6517)
EVQLLESGGGLVQPGGSLRLSCAASGFTFSKYAMAWVRQAPGKGLEWVASISTGGVNTYYADSVKGRFTISRDNSKNTLYLQMNSLRAEDTAVYYCATHTGDYFDYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 105 - protein, 359 aa, Artificial Sequence
  P1AF8284  HChole Dotam-VL (D1AG3592)
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSGGGGSSIQMTQSPSSLSASVGDRVTITCQSSHSVYSDNDLAWYQQKPGKAPKLLIYQASKLASGVPSRFSGSGSGTDFTLTISSLQPEDFATYYCLGGYDDESDTYGFGGGTKVEIK

SEQ ID NO: 106 - protein, 369 aa, Artificial Sequence
  P1AF8285 Hhole Dotam-VHA  (D1AG3591)
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSGGGGSVTLKESGPVLVKPTETLTLTCTVSGFSLSTYSMSWIRQPPGKALEWLGFIGSRGDTYYASWAKGRLTISKDTSKSQVVLTMTNMDPVDTATYYCARERDPYGGGAYPPHLWGRGTLVTVSSA

SEQ ID NO: 107 - protein, 218 aa, Artificial Sequence
  P1AF8284 and P1AF8285 light chain (D1AF6469)
EIVLTQSPGTLSLSPGERATLSCRASQSVSISGINLMNWYQQKPGQQPKLLIYHASILASGIPDRFSGSGSGTDFTLTISRLEPEDFAVYYCQQTRESPLTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 108 - protein, 447 aa, Artificial Sequence
  P1AF8286 and P1AF8287 HCknob <FAP> 4B9 (D1AF6515)
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 109 - protein, 359 aa, Artificial Sequence; identical to SEQ ID NO: 105
  P1AF8286 HChole Dotam-VL (D1AG3592)

SEQ ID NO: 110 - protein, 369 aa, Artificial Sequence; identical to SEQ ID NO: 106
  P1AF8287 HChole Dotam-VHA (D1AG3591)

SEQ ID NO: 111 - protein, 215 aa, Artificial Sequence
  P1AF8286 and P1AF8287 light chain (D1AB9974)
EIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 112 - protein, 451 aa, Artificial Sequence
  P1AF7782 and P1AF7784 HCknob <CEA> CH1A1A (D1AD3419)
QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 113 - protein, 359 aa, Artificial Sequence
  P1AF7782 HChole Dotam-VL (D1AG2237)
SIQMTQSPSSLSASVGDRVTITCQSSHSVYSDNDLAWYQQKPGKAPKLLIYQASKLASGVPSRFSGSGSGTDFTLTISSLQPEDFATYYCLGGYDDESDTYGFGGGTKVEIKGGGGSGGGGSGGGGSGGSGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 114 - protein, 369 aa, Artificial Sequence
  P1AF7784 HChole Dotam-VH (D1AG2236)
GVTLKESGPVLVKPTETLTLTCTVSGFSLSTYSMSWIRQPPGKALEWLGFIGSRGDTYYASWAKGRLTISKDTSKSQVVLTMTNMDPVDTATYYCARERDPYGGGAYPPHLWGRGTLVTVSSGGGGSGGGGSGGGGSGGSGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 115 - protein, 215 aa, Artificial Sequence; identical to SEQ ID NO: 34
  P1AF7782 and P1AF7784 light chain (D1AD3421)

SEQ ID NO: 116 - protein, 10 aa, Artificial Sequence
  heavy chain CDR1 <Pb-Dotam>  PRIT-0213
GFSLSTYSMS

SEQ ID NO: 117 - protein, 16 aa, Artificial Sequence
  heavy chain CDR2 <Pb-Dotam>  PRIT-0213
FIGSRGDTYYASWAKG

SEQ ID NO: 118 - protein, 14 aa, Artificial Sequence
  heavy chain CDR3 <Pb-Dotam>  PRIT-0213
ERDPYGGGAYPPHL

SEQ ID NO: 119 - protein, 13 aa, Artificial Sequence
  light chain CDR1 <Pb-Dotam>  PRIT-0213
QSSHSVYSDNDLA

SEQ ID NO: 120 - protein, 7 aa, Artificial Sequence
  light chain CDR2 <Pb-Dotam>  PRIT-0213
QASKLAS

SEQ ID NO: 121 - protein, 12 aa, Artificial Sequence
  light chain CDR3 <Pb-Dotam>  PRIT-0213
LGGYDDESDTYG

SEQ ID NO: 122 - protein, 121 aa, Artificial Sequence; identical to SEQ ID NO: 7
  heavy chain variable domain 1  of <Pb-Dotam> PRIT-0213

SEQ ID NO: 123 - protein, 111 aa, Artificial Sequence; identical to SEQ ID NO: 8
  light chain  variable domain <Pb-Dotam> PRIT-0213

SEQ ID NO: 124 - protein, 121 aa, Artificial Sequence; identical to SEQ ID NO: 9
  heavy chain variable domain   <Pb-Dotam> PRIT-0214

SEQ ID NO: 125 - protein, 111 aa, Artificial Sequence; identical to SEQ ID NO: 8
  light chain  variable domain <Pb-Dotam> PRIT-0214

SEQ ID NO: 126 - protein, 591 aa, Artificial Sequence
  heavy chain 1  of bispecific, trivalent <CEA/Pb-Dotam>  PRIT-0214 VH_84.66
QVQLVQSGAEVKKPGSSVKVSCKASGFNIKDTYMHWVRQAPGQGLEWMGRIDPANGNSKYVPKFQGRVTITADTSTSTAYMELSSLRSEDTAVYYCAPFGYYVSDYAMAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSVQLQQWGAGLLKPSETLSLTCAVYGFSLSTYSMSWIRQPPGKGLEWIGFIGSRGDTYYASWAKGRVTISRDTSKNQVSLKLSSVTAADTAVYYCARERDPYGGGAYPPHLWGRGTLVTVSS

SEQ ID NO: 127 - protein, 581 aa, Artificial Sequence; identical to SEQ ID NO: 84
  heavy chain 2  of bispecific, trivalent<CEA/Pb-Dotam>  PRIT-0214 VL_84.66

SEQ ID NO: 128 - protein, 218 aa, Artificial Sequence; identical to SEQ ID NO: 89
  light chain  <CEA>  84.66

SEQ ID NO: 129 - protein, 591 aa, Artificial Sequence
  heavy chain 1  of bispecific, trivalent <CEA/Pb-Dotam>  PRIT-0213 VH_84.66 --> knob
QVQLVQSGAEVKKPGSSVKVSCKASGFNIKDTYMHWVRQAPGQGLEWMGRIDPANGNSKYVPKFQGRVTITADTSTSTAYMELSSLRSEDTAVYYCAPFGYYVSDYAMAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSVTLKESGPVLVKPTETLTLTCTVSGFSLSTYSMSWIRQPPGKALEWLGFIGSRGDTYYASWAKGRLTISKDTSKSQVVLTMTNMDPVDTATYYCARERDPYGGGAYPPHLWGRGTLVTVSS

SEQ ID NO: 130 - protein, 581 aa, Artificial Sequence; identical to SEQ ID NO: 84
  heavy chain 2  of bispecific, trivalent <CEA/Pb-Dotam>  PRIT-0213 VL_84.66 -->hole

SEQ ID NO: 131 - protein, 581 aa, Artificial Sequence
  heavy chain 1  of bispecific, <CEA/Pb-Dotam> Rabbit Dotam _84.66
QVQLVQSGAEVKKPGSSVKVSCKASGFNIKDTYMHWVRQAPGQGLEWMGRIDPANGNSKYVPKFQGRVTITADTSTSTAYMELSSLRSEDTAVYYCAPFGYYVSDYAMAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSGGGGSGGGGSAVLTQTPSPVSPAVGGTVTISCQSSHSVYSDNDLAWYQQKLGQPPKLLIYQASKLASGVSSRFSGSGSGTQFTLTISGVQSDDAATYYCLGGYDDESDTYGFGGGTEVVVK

SEQ ID NO: 132 - protein, 121 aa, Artificial Sequence; identical to SEQ ID NO: 7
  HC5

SEQ ID NO: 133 - protein, 112 aa, Artificial Sequence
  LC1
SIQMTQSPSSLSASVGDRVTITCQSSHSVYSDNDLAWYQQKPGKAPKLLIYQASKLASGVPSRFSGSGSGTDFTLTISSLQPEDFATYYCLGGYDDESDTYGFGGGTKVEIK

SEQ ID NO: 134 - protein, 111 aa, Artificial Sequence
  LC3
AQMTQSPSTLSASVGDRVTITCQSSHSVYSDNDLAWYQQKPGQPPKLLIYQASKLASGVPSRFSGSGSGTEFTLTISSLQPDDFATYYCLGGYDDESDTYGFGGGTKVEIK

SEQ ID NO: 135 - protein, 121 aa, Artificial Sequence
  HC7
VQLVESGGGLVQPGGSLRLSCAASGFSLSTYSMSWVRQAPGKGLEWVGFIGSRGDTYYASWAKGRFTISRDTSKNTAYLQMNSLRAEDTAVYYCARERDPYGGGAYPPHLWGRGTLVTVSS

SEQ ID NO: 136 - protein, 121 aa, Artificial Sequence; identical to SEQ ID NO: 9
  HC10

SEQ ID NO: 137 - protein, 121 aa, Artificial Sequence; identical to SEQ ID NO: 17
  T84.66 VH 1

SEQ ID NO: 138 - protein, 111 aa, Artificial Sequence; identical to SEQ ID NO: 18
  T84.66 VL

SEQ ID NO: 139 - protein, 121 aa, Artificial Sequence; identical to SEQ ID NO: 25
  CH1A1A VH

SEQ ID NO: 140 - protein, 108 aa, Artificial Sequence; identical to SEQ ID NO: 26
  CH1A1A VL

SEQ ID NO: 141 - DNA, 37 bp, Artificial Sequence
  rbHC.up  primer sequence
aagcttgccaccatggagactgggctgcgctggcttc

SEQ ID NO: 142 - DNA, 21 bp, Artificial Sequence
  https//protect-eu.mimecast.com/s/ jhrhC2RNxFYMPVS2Ygk4?domain=rbhcf.do primer sequence
ccattggtgagggtgcccgag

SEQ ID NO: 143 - DNA, 34 bp, Artificial Sequence
  rbLC.up Primer sequence
aagcttgccaccatggacaygagggcccccactc

SEQ ID NO: 144 - DNA, 26 bp, Artificial Sequence
  https//protect-eu.mimecast.com/s/ 2MppC3lXQckn6XCQnhR8?domain=rblc.do Primer sequence
cagagtrctgctgaggttgtaggtac

SEQ ID NO: 145 - protein, 224 aa, Artificial Sequence
  P1AA1227_HC
VTLKESGPVLVKPTETLTLTCTVSGFSLSTYSMSWIRQPPGKALEWLGFIGSRGDTYYASWAKGRLTISKDTSKSQVVLTMTNMDPVDTATYYCARERDPYGGGAYPPHLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 146 - protein, 219 aa, Artificial Sequence
  P1AA1227_LC
SIQMTQSPSSLSASVGDRVTITCQSSHSVYSDNDLAWYQQKPGKAPKLLIYQASKLASGVPSRFSGSGSGTDFTLTISSLQPEDFATYYCLGGYDDESDTYGFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 147 - protein, 594 aa, Artificial Sequence
  D1AE3669 (HCknob <CEA> CH1A1A Dotam-VH-AST)
QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSVTLKESGPVLVKPTETLTLTCTVSGFSLSTYSMSWIRQPPGKALEWLGFIGSRGDTYYASWAKGRLTISKDTSKSQVVLTMTNMDPVDTATYYCARERDPYGGGAYPPHLWGRGTLVTVSSAST

SEQ ID NO: 148 - protein, 10 aa, Artificial Sequence
  CDR H1
GFSLTDYGVH

SEQ ID NO: 149 - protein, 10 aa, Artificial Sequence
  human IgG1 CH1 domain
ASTKGPSVFP

SEQ ID NO: 150 - protein, 122 aa, Artificial Sequence
  heavy chain variable domain with c terminus extension
VTLKESGPVLVKPTETLTLTCTVSGFSLSTYSMSWIRQPPGKALEWLGFIGSRGDTYYASWAKGRLTISKDTSKSQVVLTMTNMDPVDTATYYCARERDPYGGGAYPPHLWGRGTLVTVSSA

SEQ ID NO: 151 - protein, 10 aa, Artificial Sequence
  CDR H1
GFTFTDYYMN

SEQ ID NO: 152 - protein, 20 aa, Artificial Sequence
  flexible linker
GGGGSGGGGSGGGGSGGSGG

SEQ ID NO: 153 - protein, 592 aa, Artificial Sequence
  a variant with c-terminal alanine extension
QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSVTLKESGPVLVKPTETLTLTCTVSGFSLSTYSMSWIRQPPGKALEWLGFIGSRGDTYYASWAKGRLTISKDTSKSQVVLTMTNMDPVDTATYYCARERDPYGGGAYPPHLWGRGTLVTVSSA

SEQ ID NO: 154 - protein, 88 aa, Artificial Sequence
  A5B7 epitope
PKPFITSNNSNPVEDEDAVALTCEPEIQNTTYLWWVNNQSLPVSPRLQLSNDNRTLTLLSVTRNDVGPYECGIQNKLSVDHSDPVILN

SEQ ID NO: 155 - protein, 88 aa, Artificial Sequence
  MFE23 epitope
PKPSISSNNSKPVEDKDAVAFTCEPETQDATYLWWVNNQSLPVSPRLQLSNGNRTLTLFNVTRNDTASYKCETQNPVSARRSDSVILN

SEQ ID NO: 156 - protein, 5 aa, Artificial Sequence
  heavy chain CDR1 <CEA> MFE23
DSYMH

SEQ ID NO: 157 - protein, 17 aa, Artificial Sequence
  heavy chain CDR2 <CEA> MFE23
WIDPENGDTEYAPKFQG

SEQ ID NO: 158 - protein, 17 aa, Artificial Sequence
  heavy chain CDR2 <CEA> MFE23-H26
WIDPENGGTNYAQKFQG

SEQ ID NO: 159 - protein, 11 aa, Artificial Sequence
  heavy chain CDR3 <CEA> MFE23
GTPTGPYYFDY

SEQ ID NO: 160 - protein, 10 aa, Artificial Sequence
  light chain CDR1 <CEA> MFE23
SASSSVSYMH

SEQ ID NO: 161 - protein, 10 aa, Artificial Sequence
  light chain CDR1 <CEA> MFE23-L24, L25
RASSSVSYMH

SEQ ID NO: 162 - protein, 20 aa, Artificial Sequence
  light chain CDR1 <CEA> MFE23-L26
RASQSISSYMRASQSISSYM

SEQ ID NO: 163 - protein, 7 aa, Artificial Sequence
  light chain CDR2 <CEA> MFE23
STSNLAS

SEQ ID NO: 164 - protein, 7 aa, Artificial Sequence
  Light chain CDR2 <CEA> MFE23-L26
YTSNLAS

SEQ ID NO: 165 - protein, 7 aa, Artificial Sequence
  Light chain CDR2 <CEA> MFE23-L29
STSSLQS

SEQ ID NO: 166 - protein, 9 aa, Artificial Sequence
  light chain CDR3 <CEA> MFE23
QQRSSYPLT

SEQ ID NO: 167 - protein, 120 aa, Artificial Sequence
  Heavy chain variable domain <CEA> MFE23
QVKLQQSGAELVRSGTSVKLSCTASGFNIKDSYMHWLRQGPEQGLEWIGWIDPENGDTEYAPKFQGKATFTTDTSSNTAYLQLSSLTSEDTAVYYCNEGTPTGPYYFDYWGQGTTVTVSS

SEQ ID NO: 168 - protein, 106 aa, Artificial Sequence
  Light chain variable domain <CEA> MFE23
ENVLTQSPAIMSASPGEKVTITCSASSSVSYMHWFQQKPGTSPKLWIYSTSNLASGVPARFSGSGSGTSYSLTISRMEAEDAATYYCQQRSSYPLTFGAGTKLELK

SEQ ID NO: 169 - protein, 120 aa, Artificial Sequence
  MFE-H24
QVQLVQSGAEVKKPGASVKVSCKASGFNIKDSYMHWVRQAPGQGLEWMGWIDPENGDTEYAPKFQGRVTMTTDTSISTAYMELSRLRSDDTAVYYCNEGTPTGPYYFDYWGQGTLVTVSS

SEQ ID NO: 170 - protein, 120 aa, Artificial Sequence
  MFE-H25
QVQLVQSGAEVKKPGASVKVSCKASGYTFKDSYMHWVRQAPGQGLEWMGWIDPENGDTEYAPKFQGRVTMTTDTSISTAYMELSRLRSDDTAVYYCNEGTPTGPYYFDYWGQGTLVTVSS

SEQ ID NO: 171 - protein, 120 aa, Artificial Sequence
  MFE-H26
QVQLVQSGAEVKKPGASVKVSCKASGFNIKDSYMHWVRQAPGQGLEWMGWIDPENGGTNYAQKFQGRVTMTTDTSISTAYMELSRLRSDDTAVYYCNEGTPTGPYYFDYWGQGTLVTVSS

SEQ ID NO: 172 - protein, 120 aa, Artificial Sequence
  MFE-H27
QVQLVQSGAEVKKPGASVKVSCKASGFNIKDSYMHWVRQAPGQGLEWMGWIDPENGDTEYAPKFQGRVTMTTDTSISTAYMELSRLRSDDTAVYYCARGTPTGPYYFDYWGQGTLVTVSS

SEQ ID NO: 173 - protein, 120 aa, Artificial Sequence
  MFE-H28
QVQLVQSGAEVKKPGASVKVSCKASGFNIKDSYMHWVRQAPGQGLEWMGWIDPENGDTEYAPKFQGRVTMTRDTSISTAYMELSRLRSDDTAVYYCNEGTPTGPYYFDYWGQGTLVTVSS

SEQ ID NO: 174 - protein, 120 aa, Artificial Sequence
  MFE-H29
QVQLVQSGAEVKKPGSSVKVSCKASGFNIKDSYMHWVRQAPGQGLEWMGWIDPENGDTEYAPKFQGRVTITTDESTSTAYMELSSLRSEDTAVYYCNEGTPTGPYYFDYWGQGTLVTVSS

SEQ ID NO: 175 - protein, 106 aa, Artificial Sequence
  MFE-L24
DIQMTQSPSSLSASVGDRVTITCRASSSVSYMHWYQQKPGKAPKLLIYSTSNLASGVPSRFSGSGSGTDFTLTISSLQPEDFATYYCQQRSSYPLTFGGGTKLEIK

SEQ ID NO: 176 - protein, 106 aa, Artificial Sequence
  MFE-L25
EIQMTQSPSSLSASVGDRVTITCRASSSVSYMHWYQQKPGKAPKLLIYSTSNLASGVPSRFSGSGSGTDFTLTISSLQPEDFATYYCQQRSSYPLTFGGGTKLEIK

SEQ ID NO: 177 - protein, 107 aa, Artificial Sequence
  MFE-L26
EIQMTQSPSSLSASVGDRVTITCRASQSISSYMHWYQQKPGKAPKLLIYSTSNLASGVPSRFSGSGSGTDFTLTISSLQPEDFATYYCQQRSSYPLTFGGGTKLEIK

SEQ ID NO: 178 - protein, 106 aa, Artificial Sequence
  MFE-L27
EIQMTQSPSSLSASVGDRVTITCRASSSVPYMHWYQQKPGKAPKLLIYSTSNLASGVPSRFSGSGSGTDFTLTISSVQPEDFATYYCQQRSSYPLTFGGGTKLEIK

SEQ ID NO: 179 - protein, 106 aa, Artificial Sequence
  MFE-L28
EIQMTQSPSSLSASVGDRVTITCRASSSVPYMHWLQQKPGKAPKLLIYSTSNLASGVPSRFSGSGSGTDFTLTISSVQPEDFATYYCQQRSSYPLTFGGGTKLEIK

SEQ ID NO: 180 - protein, 106 aa, Artificial Sequence
  MFE-L29
EIQMTQSPSSLSASVGDRVTITCRASSSVPYMHWLQQKPGKAPKLLIYSTSSLQSGVPSRFSGSGSGTDFTLTISSVQPEDFATYYCQQRSSYPLTFGGGTKLEIK

(19) United States
(12) Patent Application Publication
(10) Pub. No.: US 2022/0267463 A1
(43) Pub. Date: Aug. 25, 2022

(54) ANTIBODIES WHICH BIND TO CANCER CELLS AND TARGET RADIONUCLIDES TO SAID CELLS

(71) Applicant: Hoffmann-La Roche Inc., Little Falls, NJ (US)

(72) Inventors: Pablo UMANA, Schlieren (CH); Sabine IMHOF-JUNG, Penzberg (DE); Alexander HAAS, Penzberg (DE); Christian KLEIN, Schlieren (CH); Sofia FROST, Schlieren (CH); Felix BORMANN, Penzberg (DE); Guy GEORGES, Penzberg (DE); Sebastian FENN, Penzberg (DE); Florian LIPSMEIER, Penzberg (DE); Daniela MATSCHEKO, Penzberg (DE); Joerg MOELLEKEN, Penzberg (DE); Barbara WEISER, Penzberg (DE)

(73) Assignee: Hoffmann-La Roche Inc., Little Falls, NJ (US)

(21) Appl. No.: 17/625,719

(22) PCT Filed: Jul. 10, 2020
(86) PCT No.: PCT/EP2020/069561

§ 371 Date: Jan. 7, 2022

(30) Foreign Application Priority Data
Jul. 12, 2019 (EP) 19186135.0

(51) Int. Cl. C07K 16/30; C07K 16/44; A61K 51/04; A61P 35/00

(52) U.S. Cl. CPC C07K 16/3007; C07K 16/44; A61K 2039/507; A61K 51/0482; A61P 35/00; A61K 51/0495

(57) ABSTRACT

The present invention relates to antibodies which bind to antigens on target cells and which target radionuclides to said cells, and to methods of using the same.

### FIELD OF THE INVENTION

The present invention relates to antibodies which bind to antigens on target cells and which target radionuclides to said cells, and to methods of using the same.

### BACKGROUND

Monoclonal antibodies have been developed to target drugs to cancer cells. By conjugating a toxic agent to an antibody which binds to a tumour-associated antigen, there is the potential to provide more specific tumour killing with less damage to surrounding tissues.

In pre-targeted radioimmunotherapy (PRIT), use is made of an antibody construct which has affinity for the tumour-associated antigen on the one hand and for a radiolabelled compound on the other. In a first step, the antibody is administered and localises to tumour. Subsequently, the radiolabelled compound is administered. Because the radiolabelled compound is small, it can be delivered quickly to the tumour and is fast-clearing, which reduces radiation exposure outside of the tumour (Goldenberg et al Theranostics 2012, 2(5), 523-540). A similar procedure can also be used for imaging. Pre-targeting can make use of a bispecific antibody or systems using avidin-biotin, although the latter has the disadvantage that avidin/streptavidin is immunogenic.

Methods of pre-targeted radioimmunotherapy or imaging commonly make use of a clearing or blocking agent, which is administered between the step of administering the antibody and the step of administering the radiolabelled compound. The purpose is to clear antibody from the blood and/or to block the binding site of the circulating antibody for the radiolabelled compound (see for instance Karacay et al, Bioconj. Chem., 13(5), 1054-1070 (2002)). The use of a clearing or blocking agent allows for sufficient levels of radioactivity to be administered for an efficient treatment while limiting adverse toxicity, but the timing and dosage must be chosen with care. Thus, the use of a clearing phase is a complicating aspect in pre-targeting methods.

### SUMMARY

The present invention provides sets of antibodies useful in pre-targeting methods, and methods of using the same.

In one aspect, the present invention provides a set of antibodies comprising:
i) a first antibody that binds to an antigen expressed on the surface of a target cell, and which further comprises a V_{H} domain of an antigen binding site for a radiolabelled compound, but which does not comprise a V_{L} domain of an antigen binding site for the radiolabelled compound; and
ii) a second antibody that binds to the antigen expressed on the surface of the target cell, and which further comprises a V_{L} domain of an antigen binding site for the radiolabelled compound, but which does not comprise a V_{H} domain of the antigen binding site for the radiolabelled compound,

wherein said V_{H} domain of the first antibody and said V_{L} domain of the second antibody are together capable of forming a functional antigen binding site for the radiolabelled compound.

Neither the first nor the second antibody comprise, on their own, a functional antigen binding site for a radiolabelled compound. The first antibody has only a V_{H} domain from the functional binding site for the radiolabelled compound, and not the V_{L} domain. The second antibody has only the V_{L} domain, and not the V_{H} domain.

A functional antigen binding site for the radiolabelled compound is formed when the V_{H} and V_{L} domains of the first and second antibodies are associated. This may occur, for example, when the first and second antibodies are bound to the same individual target cell or to adjacent cells.

The first and second antibodies described herein may be referred to herein as “single domain split antibodies”, “split antibodies” or “demibodies”. The V_{H} and V_{L} domain which together form an antigen binding site capable of binding to the radiolabelled compound are split between two antibodies, and not present as part of the same antibody.

The split domain format means that the radiolabelled compound cannot bind to either the first antibody on its own or to the second antibody on its own. In the blood, there is little or no stable association between the first and the second antibody, and so little or no stable binding of the radiolabelled compound.

An antigen expressed on the surface of a target cell may be referred to herein as a “target antigen” or “TA”. According to the present invention, the first and the second antibody described above have a binding site for the same target antigen. (For the avoidance of doubt, where it stated that the antibodies bind the same target antigen, this means that they have a binding site capable of binding to the same target antigen and includes the possibility that the antibodies may bind to two individual antigen molecules that are the same as each other). For example, in one embodiment, both the first and the second antibody bind to CEA.

In some embodiments, the first and second antibody may bind to (have a binding site for) the same epitope of the target antigen. In other embodiments, the first antibody may bind to (have a binding site for) a different epitope of the target antigen from the second antibody.

In some embodiments, the first and second antibody may comprise the same antigen binding site for the target antigen. That is, they may comprise an antigen binding site capable of binding to the target antigen, comprising a V_{L} and V_{H} sequence, where the V_{L} and V_{H} sequences forming this antigen binding site are the same in the first and in the second antibodies.

In some embodiments, each of the first and the second antibodies are bivalent for the target antigen. In some embodiments they are each bivalent and monospecific for an epitope. In other embodiments, each of the first and second antibodies are biparatopic for the target antigen, i.e., the first and the second antibodies each have binding sites for two different epitopes of the target antigen.

In some embodiments, it may be preferred that the first and/or second antibody comprise an Fc region. The presence of an Fc region has benefits in the context of radioimmunotherapy and radioimaging, e.g. prolonging the protein's circulating half-life and/or resulting in higher tumour uptake than may be observed with smaller fragments. The “split domain” format described herein may be particularly advantageous in this context, as it mitigates against the greater possibility of association with radiolabelled compound that would otherwise occur due to the prolonged presence of the circulating antibody.

In some embodiments, the Fc domain is modified to reduce or eliminate effector function.

In another aspect, the present invention provides a pharmaceutical composition comprising the set of antibodies as described herein. In another aspect, the present invention provides a kit comprising two separate pharmaceutical compositions, each comprising one of the antibodies described herein (i.e., the first and second antibody respectively).

In a further aspect, the present invention relates to a polynucleotide or set of polynucleotides encoding any of the antibodies or sets of antibodies described herein. In another aspect, the present invention relates to a vector or set of vectors comprising said polynucleotide or polynucleotides, optionally an expression vector or set of expression vectors. In a further object the present invention relates to a prokaryotic or eukaryotic host cell or a set of host cells comprising a vector or set of vectors of the present invention. In addition there is provided a method of producing an antibody comprising culturing the host cell(s) so that the antibody is produced.

In some embodiments, antibodies as described herein find use in a method of pre-targeted radioimmunotherapy (PRIT) or in a method of pre-targeted radioimaging.

In one aspect, the present invention provides a method of pre-targeted radioimmunotherapy which comprises:
i) administering to a subject a first antibody and a second antibody as described above; and
ii) subsequently administering to said subject a radiolabelled compound.

In another aspect, the present invention provides a first and a second antibody described above for use in a method of treatment comprising administering the first antibody and the second antibody to a subject, and subsequently administering to said subject a radiolabelled compound. In another aspect, the invention provides a first antibody as described above for use in a method of treatment comprising administering the first antibody and the second antibody to a subject, and subsequently administering to said subject a radiolabelled compound. In another aspect the invention provides the second antibody as described above for use in a method of treatment comprising administering the first antibody and the second antibody to a subject, and subsequently administering to said subject a radiolabelled compound.

In another aspect, the present invention provides a method of radioimaging which comprises:
i) administering to a subject a first and a second antibody as described herein, wherein the antibodies bind to the target antigen and localise to the surface of a cell expressing the target antigen;
ii) subsequently administering a radiolabelled compound; and optionally
iii) imaging the tissue or organ where the radionuclide has localised.

In another aspect, the present invention provides a first and a second antibody as described herein for use in a method of diagnosis carried out on the human or animal body, wherein the method comprises
i) administering to subject a first and a second antibody as described herein, wherein the antibodies bind to the target antigen and localise to the surface of a cell expressing the target antigen;
ii) subsequently administering a radiolabelled compound; and optionally
iii) imaging the tissue or organ where the radionuclide has localised.

The imaging step may be followed by a step of forming a diagnosis and optionally a step of delivering that diagnosis to the subject. In some embodiments the method may further comprise determining an appropriate treatment and optionally administering that treatment to the subject.

In each of the above methods/uses, binding of the first and the second antibody to the same or adjacent target cells results in association of the V_{H} and V_{L} domains of an antigen binding site for a radiolabelled compound and the formation of a functional antigen binding site for the radiolabelled compound. Thus, after administration of the radiolabelled compound, the radiolabelled compound binds to the functional antigen binding site formed by association of the V_{H} and V_{L}.

In any of the methods and uses described herein, the first and second antibodies can be administered simultaneously or sequentially, in either order.

Frequently in the art, methods of PRIT or radioimaging involve a clearing step. The clearing step comprises administering an agent between the administration of the antibody and the administration of the radiolabelled compound, wherein the agent increases the rate of removal of the antibody from blood and/or blocks binding of radiolabelled compound to the antibody.

In an embodiment of the methods and uses described herein, the method does not comprise a clearing step. That is, it does not comprise a step of administering a clearing agent or a blocking agent between the administration of the first and second antibodies and the administration of radiolabelled compound (i.e., after the administration of the antibodies but before administration of the radiolabelled compound). In another embodiment, no agent is administered between the administration of the first and second antibodies and the administration of radiolabelled compound, other than optionally a radiosensitizer, immunotherapeutic and/or a chemotherapeutic agent. In another embodiment, no agent is administered between the administration of the first and second antibodies and the administration of radiolabelled compound.

In some embodiments, the antibodies described herein may be administered as part of a combination therapy. For example, they may be administered in combination with one or more radiosensitizers, immunotherapeutics and/or chemotherapeutic agents: the radiosensitizer, immunotherapeutic or chemotherapeutic agent and the antibodies may be administered simultaneously or sequentially, in either order.

The methods of radioimaging and radioimmunotherapy described herein may optionally be combined as discussed further herein.

In a further aspect, the present invention provides a kit comprising:
i) a first and a second antibody as described herein;
ii) a radiolabelled compound which binds to the antigen binding site formed by association of the first and the second antibody.

Optionally the kit may exclude (i.e., does not comprise) a clearing agent or a blocking agent as described herein.

Optionally the kit may further comprise a radiosensitizer, immunotherapeutic or chemotherapeutic agent.

In some embodiments, the first and the second antibody may be present in the same pharmaceutical composition. In other embodiments, the first and second antibody may be present in separate pharmaceutical compositions. In some embodiments, the radiolabelled compound is present in a pharmaceutical composition separate from the antibodies.

### BRIEF DESCRIPTION OF THE FIGURES

FIG. 1 shows the schematic structure of a target antigen (TA)-DOTAM bispecific antibody (TA-DOTAM BsAb) belonging to the comparative examples, and exemplary TA-split-DOTAM-VH/VL antibodies according to the invention.

FIG. 2 is a schematic diagram showing the assembly of the split-VH/VL DOTAM binder on tumour cells. The TA-split-DOTAM-VH/VL antibodies will not significantly bind ²¹²Pb-DOTAM unless bound to tumour antigen (TA) on targeted cells, where the two domains of the DOTAM binder are assembled.

FIG. 3 shows a schematic overview of an example of the Three-Step TA-PRIT concept, involving use of a clearing agent.

FIG. 4 shows a schematic overview of an example of the Two-Step TA-PRIT concept, in which a clearing agent is not used.

FIG. 5 shows binding of split antibodies to MKN45 cells to demonstrate CEA binding competence. Detection of antibodies is done using human IgG specific secondary antibodies

FIG. 6 shows binding of split antibodies to MKN45 cells to demonstrate DOTAM binding competence. Detection of antibodies is done using Pb-DOTAM-FITC.

FIG. 7A shows an exemplary protocol for two-step PRIT with a CEA-split-DOTAM-VH/VL, carried out in in SCID mice carrying SC BxPC3 tumours (h=hours, d=days, w=weeks).

FIG. 7B shows an exemplary protocol for a three-step PRIT control, carried out in SCID mice carrying SC BxPC3 tumours (h=hours, d=days, w=weeks).

FIG. 8 shows the biodistribution of pretargeted ²¹²Pb-DOTAM in SCID mice carrying SC BxPC3 tumors, 6 hours after injection of ²¹²Pb-DOTAM, pretargeted either by CEA-split-DOTAM-VH alone, CEA-split-DOTAM-VL alone, or the two complimentary antibodies combined, or using standard three-step PRIT (% ID/g ±SD, n=4).

FIG. 9 shows CEA-Split-DOTAM-VH/VL phaiivacokinetics after IV injection in SCID mice.

FIG. 10 shows the experimental design of protocol 158, comprising CEA-PRIT in 2 (top) or 3 steps (bottom) in SCID mice carrying SC BxPC3 tumors. *CEA split DOTAM BsAb dose adjusted to compensate for hole/hole impurities in 2/4 constructs.

FIG. 11 shows the biodistribution of pretargeted ²¹²Pb-DOTAM in SCID mice carrying SC BxPC3 tumors (6 h p.i.). The distribution is of ²¹²Pb in tumour-bearing SCID mice, 6 hours after injection of ²¹²Pb-DOTAM, pretargeted by CEA-DOTAM BsAb or bi-paratopic combinations of CEA-split-DOTAM antibodies. The radioactive content in organs and tissues is expressed as average % ID/g ±SD (n=4).

FIG. 12 shows the experimental schedule of protocol 160, comprising one cycle of 3-step CEA-PRIT (top), 2-step CEA-PRIT (middle), or 1-step CEA-RIT in SCID mice carrying SC BxPC3 tumors. Biodistribution (BD) scouts were euthanized 24 hours after the radioactive injection, whereas mice in the efficacy groups were maintained and monitored carefully until the termination criteria were reached.

FIG. 13 shows biodistribution of pretargeted ²¹²Pb-DOTAM and ²¹²Pb-DOTAM-CEA-DOTAM in SCID mice carrying SC BxPC3 tumors (24 h p.i.). The distribution is of ²¹²Pb in tumor-bearing SCID mice 24 hours after injection of CEA-DOTAM-pretargeted ²¹²Pb-DOTAM or pre-incubated ²¹²Pb-DOTAM-CEA-DOTAM. The radioactive content in organs and tissues is expressed as average % ID/g ±SD (n=3).

FIG. 14 shows tumor growth averages with standard error for PRIT-treated groups and control (groups A-E) in the BxPC3 model (n=10). Curves were truncated at n<5. Dotted vertical lines indicate ²¹²Pb-DOTAM administration (20 μCi) for some or all groups, according to the study design.

FIG. 15 shows individual tumor growth curves for PRIT-treated groups and control (groups A-E) in the BxPC3 model (n=10). Dotted vertical lines indicate administration of ²¹²Pb-labeled compounds (20 μCi).

FIG. 16 shows average body weight loss in mice treated with CEA-PRIT and CEA-RIT (groups A-E, n=10) in the BxPC3 model. Curves were truncated at n<5. Dotted vertical lines indicate administration of ²¹²Pb-labeled compounds for some or all groups, according to the study design.

FIG. 17 shows the experimental design of protocol 175, comprising two-step CEA-PRIT in SCID mice carrying SC BxPC3 tumors, with sacrifice and necropsy 24 hours after the ²¹²Pb-DOTAM injection. The CEA-split-DOTAM-VH-AST dose was adjusted to compensate for hole/hole impurities.

FIG. 18 shows distribution of ²¹²Pb in tumor-bearing SCID mice 24 hours after injection of ²¹²Pb-DOTAM, pretargeted by CEA-split-DOTAM-VH/VL antibodies (protocol 175). The radioactive content in organs and tissues is expressed as average % ID/g ±SD (n=4).

FIG. 19 shows the experimental design of protocol 185, comprising two-step CEA-PRIT in SCID mice carrying SC BxPC3 tumors, with sacrifice and necropsy 6 hours after the ²¹²Pb-DOTAM injection. The CEA-split-DOTAM-VH-AST (CH1A1A) dose was adjusted to compensate for hole/hole impurities.

FIG. 20 shows distribution of ²¹²Pb in tumor-bearing SCID mice 6 hours after injection of ²¹²Pb-DOTAM, pretargeted by CEA-split-DOTAM-VH/VL antibodies (protocol 185). The radioactive content in organs and tissues is expressed as average % ID/g ±SD (n=5).

FIG. 21 shows distribution of CEA-split-DOTAM-VH/VL pairs (VH and VL antibodies combined) in two selected SC BxPC3 tumors 7 days after injection. A and B show sections of a tumor from mouse A3, injected with CEA-split-DOTAM-VH/VL targeting T84.66, where A shows the CEA expression, and B shows the corresponding CEA-split-DOTAM-VH/VL distribution. C and D show tumor sections from mouse C5, injected with CEA-split-DOTAM-VH/VL targeting CH1A1A: C showing the CEA expression and D the corresponding CEA-split-DOTAM-VH/VL distribution.

FIG. 22 shows the experimental design of protocol 189, comprising two-step CEA-PRIT in SCID mice carrying SC BxPC3 tumors, with sacrifice and necropsy 6 hours after the ²¹²Pb-DOTAM injection. The CEA-split-DOTAM-VH-AST (CH1A1A) dose was adjusted to compensate for hole/hole impurities.

FIG. 23 shows distribution of ²¹²Pb in tumor-bearing SCID mice 6 hours after injection of ²¹²Pb-DOTAM, pretargeted by bi-paratopic pairs of CEA-split-DOTAM-VH/VL antibodies (T84.66 and CH1A1A), compared with the positive control (CH1A1A only). The radioactive content in organs and tissues is expressed as average % ID/g ±SD.

FIG. 24 shows mean Flurescence Intensity (MFI) as determined by FACS for SPLIT antibodies. Binding of Pb-DOTA-FITC determined by FACS can only be shown for a co-incubation of both SPLIT antibodies with Pb-DOTA-FITC. Single SPLIT antibodies did not give rise to a significant signal.

FIG. 25A-C shows exemplary formats of antibodies as described herein.

FIG. 26 shows results from example 11, experiment 1, assessing binding of individual TA-split-DOTAM-VH and TA-split-DOTAM-VL antibodies to biotinylated DOTAM captured on a chip.

FIG. 27 shows results from example 11, experiment 2, assessing binding of DOTAM to individual TA-split-DOTAM-VH and TA-split-DOTAM-VL antibodies captured on a chip.

FIG. 28 shows results from example 11, experiment 3, assessing binding of DOTAM to TA-split-DOTAM-VH/VL antibodies (antibody pairs), captured on a chip.

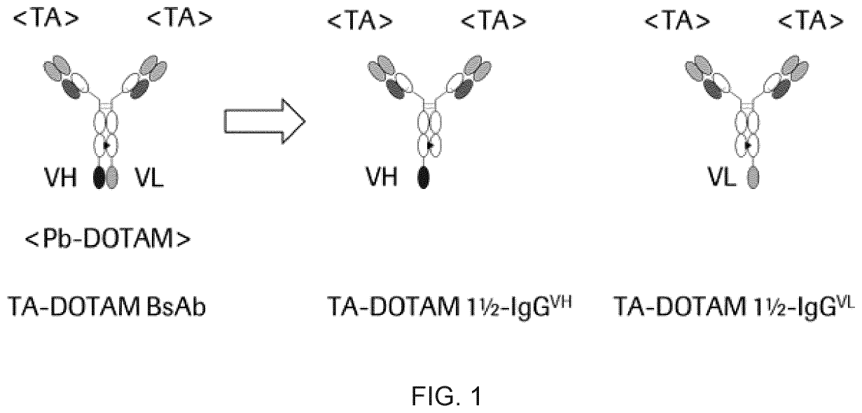

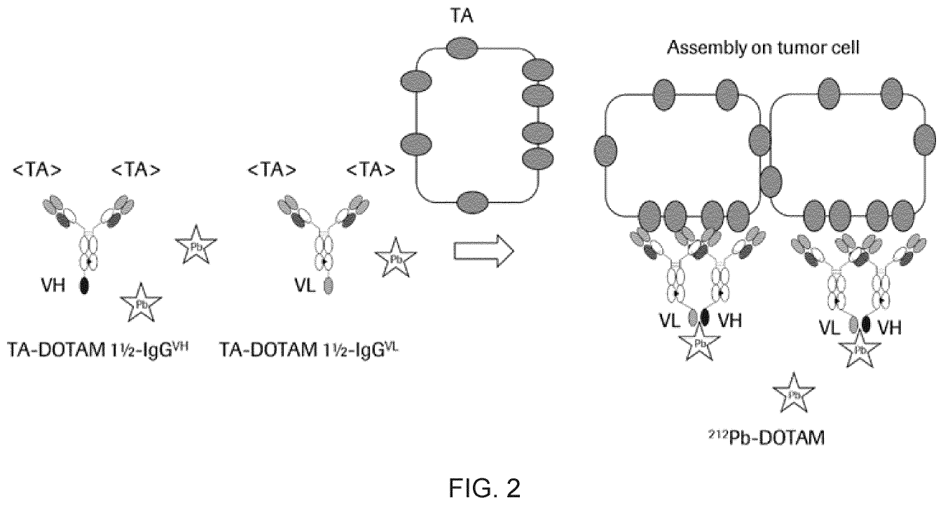

### DETAILED DESCRIPTION OF THE INVENTION

### I. Definitions

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some aspects, the number of amino acid changes are 10 or less, 9 or less, 8 or less, 7 or less, 6 or less, 5 or less, 4 or less, 3 or less, or 2 or less. In some aspects, the VL acceptor human framework is identical in sequence to the VL human immunoglobulin framework sequence or human consensus framework sequence.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured by common methods known in the art, including those described herein. Specific illustrative and exemplary methods for measuring binding affinity are described in the following.

An “affinity matured” antibody refers to an antibody with one or more alterations in one or more complementary determining regions (CDRs), compared to a parent antibody which does not possess such alterations, such alterations resulting in an improvement in the affinity of the antibody for antigen.

The term “an antibody that binds to an antigen expressed on the surface of a target cell” refers to an antibody that is capable of binding said antigen with sufficient affinity such that the antibody is useful as a diagnostic and/or therapeutic agent in targeting said antigen. In one aspect, the extent of binding of the antibody to an unrelated, non antigen protein is less than about 10% of the binding of the antibody to the antigen as measured, e.g., by surface plasmon resonance (SPR). In certain aspects, an antibody that binds to an antigen expressed on the surface of a target cell has a dissociation constant (K_{D}) of ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M). An antibody is said to “specifically bind” to an antigen expressed on the surface of a target cell when the antibody has a K_{D} of 1 μM or less. In certain aspects, the antibody binds to an epitope of said antigen that is conserved among said antigen from different species.

The terms “an antigen binding site for a radiolabelled compound” or “a functional antigen binding site for a radiolabelled compound” refer to an antigen binding site comprising VH and a VL domain, capable of binding to the radiolabelled compound with sufficient affinity such that the antibody is useful as a diagnostic and/or therapeutic agent to associate the radiolabelled compound with the antibody. In one aspect, the extent of binding of the antigen binding site to an unrelated, non antigen-compound is less than about 10% of the binding of the antibody to the radiolabelled compound as measured, e.g., by surface plasmon resonance (SPR). In certain aspects, an antigen binding site that binds to a radiolabelled compound has a dissociation constant (KD) of ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M). It may be preferred that it has a KD of 100 pM, 50 pM, 20 pM, 10 pM, 5 pM, 1 pM or less, e.g, 0.9 pM or less, 0.8 pM or less, 0.7 pM or less, 0.6 pM or less or 0.5 pM or less. For instance, the functional binding site may bind the radiolabelled compound with a Kd of about 1 pM-1 nM, e.g., about 1-10 pM, 1-100 pM, 5-50 pM, 100-500 pM or 500 pM-1 nM. An antigen binding site is said to “specifically bind” to a radiolabelled compound when the antigen binding site has a KD of 1 μM or less.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, cross-Fab, Fab′, Fab′-SH, F(ab′)₂; diabodies; linear antibodies; single-chain antibody molecules (e.g., scFv, and scFab); single domain antibodies (dAbs); and multispecific antibodies formed from antibody fragments. For a review of certain antibody fragments, see Holliger and Hudson, Nature Biotechnology 23:1126-1136 (2005). The term “Fab fragment” thus refers to an antibody fragment comprising a light chain comprising a VL domain and a CL domain, and a heavy chain fragment comprising a VH domain and a CH1 domain. “Fab′ fragments” differ from Fab fragments by the addition of residues at the carboxy terminus of the CH1 domain including one or more cysteines from the antibody hinge region. For discussion of Fab and F(ab′)2 fragments comprising salvage receptor binding epitope residues and having increased in vivo half-life, see U.S. Pat. No. 5,869,046. The term “cross-Fab fragment” or “xFab fragment” or “crossover Fab fragment” refers to a Fab fragment, wherein either the variable regions or the constant regions of the heavy and light chain are exchanged. A cross-Fab fragment comprises a polypeptide chain composed of the light chain variable region (VL) and the heavy chain constant region 1 (CH1), and a polypeptide chain composed of the heavy chain variable region (VH) and the light chain constant region (CL). Asymmetrical Fab arms can also be engineered by introducing charged or non-charged amino acid mutations into domain interfaces to direct correct Fab pairing. See e.g., WO 2016/172485.

A “single-chain variable fragment” or “scFv” is a fusion protein of the variable domains of the heavy (VH) and light chains (VL) of an antibody, connected by a peptide linker. In particular, the linker is a short polypeptide of 10 to 25 amino acids and is usually rich in glycine for flexibility, as well as serine or threonine for solubility, and can either connect the N-terminus of the VH with the C-terminus of the VL, or vice versa. This protein retains the specificity of the original antibody, despite removal of the constant regions and the introduction of the linker. For a review of scFv fragments, see, e.g., Plückthun, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458.

The term “blocking agent” refers to an agent which blocks the binding of an effector molecule, in particular the radiolabelled compound, to a functional binding site for that effector molecule. Generally said blocking agent binds to the functional binding site for the effector molecule, e.g., specifically binds to the said functional binding site.

The term “clearing agent” refers to an agent which increases the rate of clearance of an antibody from the circulation of the subject. Generally the clearing agent binds to the antibody, e.g., specifically binds to the antibody.

The term “clearing step” or “clearing phase” as used herein encompasses the use of either a blocking agent or a clearing agent. Some agents can function as both a clearing and as a blocking agent.

The term “epitope” denotes the site on an antigen, either proteinaceous or non-proteinaceous, to which an antibody binds. Epitopes can be formed both from contiguous amino acid stretches (linear epitope) or comprise non-contiguous amino acids (conformational epitope), e.g., coming in spatial proximity due to the folding of the antigen, i.e. by the tertiary folding of a proteinaceous antigen. Linear epitopes are typically still bound by an antibody after exposure of the proteinaceous antigen to denaturing agents, whereas conformational epitopes are typically destroyed upon treatment with denaturing agents. An epitope comprises at least 3, at least 4, at least 5, at least 6, at least 7, or 8-10 amino acids in a unique spatial conformation.

Screening for antibodies binding to a particular epitope (i.e., those binding to the same epitope) can be done using methods routine in the art such as, e.g., without limitation, alanine scanning, peptide blots (see Meth. Mol. Biol. 248 (2004) 443-463), peptide cleavage analysis, epitope excision, epitope extraction, chemical modification of antigens (see Prot. Sci. 9 (2000) 487-496), and cross-blocking (see “Antibodies”, Harlow and Lane (Cold Spring Harbor Press, Cold Spring Harb., N.Y.).

Antigen Structure-based Antibody Profiling (ASAP), also known as Modification-Assisted Profiling (MAP), allows to bin a multitude of monoclonal antibodies specifically binding to an antigen based on the binding profile of each of the antibodies from the multitude to chemically or enzymatically modified antigen surfaces (see, e.g., US 2004/0101920). The antibodies in each bin bind to the same epitope which may be a unique epitope either distinctly different from or partially overlapping with epitope represented by another bin.

Also competitive binding can be used to easily determine whether an antibody binds to the same epitope as, or competes for binding with, a reference antibody. For example, an “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more. Also for example, to determine if an antibody binds to the same epitope as a reference antibody, the reference antibody is allowed to bind to the antigen under saturating conditions. After removal of the excess of the reference antibody, the ability of an antibody in question to bind to the antigen is assessed. If the antibody in question is able to bind to the antigen after saturation binding of the reference antibody, it can be concluded that the antibody in question binds to a different epitope than the reference antibody. But, if the antibody in question is not able to bind to the antigen after saturation binding of the reference antibody, then the antibody in question may bind to the same epitope as the epitope bound by the reference antibody. To confirm whether the antibody in question binds to the same epitope or is just hampered from binding by steric reasons routine experimentation can be used (e.g., peptide mutation and binding analyses using ELISA, RIA, surface plasmon resonance, flow cytometry or any other quantitative or qualitative antibody-binding assay available in the art). This assay should be carried out in two set-ups, i.e. with both of the antibodies being the saturating antibody. If, in both set-ups, only the first (saturating) antibody is capable of binding to the antigen, then it can be concluded that the antibody in question and the reference antibody compete for binding to the antigen.

In some aspects, two antibodies are deemed to bind to the same or an overlapping epitope if a 1-, 5-, 10-, 20- or 100-fold excess of one antibody inhibits binding of the other by at least 50%, at least 75%, at least 90% or even 99% or more as measured in a competitive binding assay (see, e.g., Junghans et al., Cancer Res. 50 (1990) 1495-1502).

In some aspects, two antibodies are deemed to bind to the same epitope if essentially all amino acid mutations in the antigen that reduce or eliminate binding of one antibody also reduce or eliminate binding of the other. Two antibodies are deemed to have “overlapping epitopes” if only a subset of the amino acid mutations that reduce or eliminate binding of one antibody reduce or eliminate binding of the other.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. In certain aspects, the antibody is of the IgG₁ isotype. In certain aspects, the antibody is of the IgG₁ isotype with the P329G, L234A and L235A mutation to reduce Fc-region effector function. In other aspects, the antibody is of the IgG₂ isotype. In certain aspects, the antibody is of the IgG₄ isotype with the S228P mutation in the hinge region to improve stability of IgG₄ antibody. The heavy chain constant domains that correspond to the different classes of immunoglobulins are called α, δ, ε, γ, and μ, respectively. The light chain of an antibody may be assigned to one of two types, called kappa (κ) and lambda (λ), based on the amino acid sequence of its constant domain.

“Effector functions” refer to those biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g., B cell receptor); and B cell activation.

An “effective amount” of an agent, e.g., a pharmaceutical composition, refers to an amount effective, at dosages and for periods of time necessary, to achieve the desired therapeutic or prophylactic result.

The term “tandem Fab” refers to an antibody comprising two Fab fragments connected via a peptide linker/tether. In some embodiments, a tandem Fab may comprise one Fab fragment and one cross-Fab fragment, connected by a peptide linker/tether.

The term “Fc region” herein is used to define a C-terminal region of an immunoglobulin heavy chain that contains at least a portion of the constant region. The term includes native sequence Fc regions and variant Fc regions. In one aspect, a human IgG heavy chain Fc region extends from Cys226, or from Pro230, to the carboxyl-terminus of the heavy chain. However, antibodies produced by host cells may undergo post-translational cleavage of one or more, particularly one or two, amino acids from the C-terminus of the heavy chain. Therefore an antibody produced by a host cell by expression of a specific nucleic acid molecule encoding a full-length heavy chain may include the full-length heavy chain, or it may include a cleaved variant of the full-length heavy chain. This may be the case where the final two C-terminal amino acids of the heavy chain are glycine (G446) and lysine (K447, numbering according to EU index). Therefore, the C-terminal lysine (Lys447), or the C-terminal glycine (Gly446) and lysine (Lys447), of the Fc region may or may not be present. In one aspect, a heavy chain including an Fc region as specified herein, comprised in an antibody according to the invention, comprises an additional C-terminal glycine-lysine dipeptide (G446 and K447, numbering according to EU index). In one aspect, a heavy chain including an Fc region as specified herein, comprised in an antibody according to the invention, comprises an additional C-terminal glycine residue (G446, numbering according to EU index). Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

“Framework” or “FR” refers to variable domain residues other than complementary determining regions (CDRs). The FR of a variable domain generally consists of four FR domains: FR1, FR2, FR3, and FR4. Accordingly, the CDR and FR sequences generally appear in the following sequence in VH (or VL): FR1-CDR-H1(CDR-L1)-FR2-CDR-H2(CDR-L2)-FR3-CDR-H3(CDR-L3)-FR4.

The terms “full length antibody”, “intact antibody”, and “whole antibody” are used herein interchangeably to refer to an antibody having a structure substantially similar to a native antibody structure or having heavy chains that contain an Fc region as defined herein.

The terms “host cell”, “host cell line”, and “host cell culture” are used interchangeably and refer to cells into which exogenous nucleic acid has been introduced, including the progeny of such cells. Host cells include “transformants” and “transformed cells”, which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda Md. (1991), vols. 1-3. In one aspect, for the VL, the subgroup is subgroup kappa I as in Kabat et al., supra. In one aspect, for the VH, the subgroup is subgroup III as in Kabat et al., supra.

A “humanized” antibody refers to a chimeric antibody comprising amino acid residues from non-human CDRs and amino acid residues from human FRs. In certain aspects, a humanized antibody will comprise substantially all of at least one, and typically two, variable domains, in which all or substantially all of the CDRs correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “hypervariable region” or “HVR” as used herein refers to each of the regions of an antibody variable domain which are hypervariable in sequence and which determine antigen binding specificity, for example “complementarity determining regions” (“CDRs”).

Generally, antibodies comprise six CDRs: three in the VH (CDR-H1, CDR-H2, CDR-H3), and three in the VL (CDR-L1, CDR-L2, CDR-L3). Exemplary CDRs herein include:

(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));

(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, Md. (1991)); and

(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)).

Unless otherwise indicated, the CDRs are determined according to Kabat et al., supra. One of skill in the art will understand that the CDR designations can also be determined according to Chothia, supra, McCallum, supra, or any other scientifically accepted nomenclature system. Instead of the above, the sequence of CDR-H1 as described herein may extend from Kabat26 to Kabat35, e.g., for the Pb-DOTAM binding variable domain.

In one aspect, CDR residues comprise those identified in the sequence tables or elsewhere in the specification.

Unless otherwise indicated, HVR/CDR residues and other residues in the variable domain (e.g., FR residues) are numbered herein according to Kabat et al., supra.

An “immunoconjugate” is an antibody conjugated to one or more heterologous molecule(s), including but not limited to a cytotoxic agent.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain aspects, the individual or subject is a human.

Molecules as described herein may be “isolated”. An “isolated” antibody is one which has been separated from a component of its natural environment. In some aspects, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC) methods. For a review of methods for assessment of antibody purity, see, e.g., Flatman et al., J. Chromatogr. B 848:79-87 (2007).

The term “nucleic acid molecule” or “polynucleotide” includes any compound and/or substance that comprises a polymer of nucleotides. Each nucleotide is composed of a base, specifically a purine- or pyrimidine base (i.e. cytosine (C), guanine (G), adenine (A), thymine (T) or uracil (U)), a sugar (i.e. deoxyribose or ribose), and a phosphate group. Often, the nucleic acid molecule is described by the sequence of bases, whereby said bases represent the primary structure (linear structure) of a nucleic acid molecule. The sequence of bases is typically represented from 5′ to 3′. Herein, the term nucleic acid molecule encompasses deoxyribonucleic acid (DNA) including e.g., complementary DNA (cDNA) and genomic DNA, ribonucleic acid (RNA), in particular messenger RNA (mRNA), synthetic forms of DNA or RNA, and mixed polymers comprising two or more of these molecules. The nucleic acid molecule may be linear or circular. In addition, the term nucleic acid molecule includes both, sense and antisense strands, as well as single stranded and double stranded forms. Moreover, the herein described nucleic acid molecule can contain naturally occurring or non-naturally occurring nucleotides. Examples of non-naturally occurring nucleotides include modified nucleotide bases with derivatized sugars or phosphate backbone linkages or chemically modified residues. Nucleic acid molecules also encompass DNA and RNA molecules which are suitable as a vector for direct expression of an antibody of the invention in vitro and/or in vivo, e.g., in a host or patient. Such DNA (e.g., cDNA) or RNA (e.g., mRNA) vectors, can be unmodified or modified. For example, mRNA can be chemically modified to enhance the stability of the RNA vector and/or expression of the encoded molecule so that mRNA can be injected into a subject to generate the antibody in vivo (see e.g., Stadler et al, Nature Medicine 2017, published online 12 Jun. 2017, doi:10.1038/nm.4356 or EP 2 101 823 B1).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding an antibody” refers to one or more nucleic acid molecules encoding antibody heavy and light chains (or fragments thereof), including such nucleic acid molecule(s) in a single vector or separate vectors, and such nucleic acid molecule(s) present at one or more locations in a host cell.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

A “naked antibody” refers to an antibody that is not conjugated to a heterologous moiety (e.g., a cytotoxic moiety) or radiolabel. The naked antibody may be present in a pharmaceutical composition.

“Native antibodies” refer to naturally occurring immunoglobulin molecules with varying structures. For example, native IgG antibodies are heterotetrameric glycoproteins of about 150,000 daltons, composed of two identical light chains and two identical heavy chains that are disulfide-bonded. From N- to C-terminus, each heavy chain has a variable domain (VH), also called a variable heavy domain or a heavy chain variable region, followed by three constant heavy domains (CH1, CH2, and CH3). Similarly, from N- to C-terminus, each light chain has a variable domain (VL), also called a variable light domain or a light chain variable region, followed by a constant light (CL) domain.

The term “package insert” is used to refer to instructions customarily included in commercial packages of therapeutic products, that contain information about the indications, usage, dosage, administration, combination therapy, contraindications and/or warnings concerning the use of such therapeutic products.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity for the purposes of the alignment. Alignment for purposes of determining percent amino acid sequence identity can be achieved in various ways that are within the skill in the art, for instance, using publicly available computer software such as BLAST, BLAST-2, Clustal W, Megalign (DNASTAR) software or the FASTA program package. Those skilled in the art can determine appropriate parameters for aligning sequences, including any algorithms needed to achieve maximal alignment over the full length of the sequences being compared. Alternatively, the percent identity values can be generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087 and is described in WO 2001/007611.

Unless otherwise indicated, for purposes herein, percent amino acid sequence identity values are generated using the ggsearch program of the FASTA package version 36.3.8c or later with a BLOSUM50 comparison matrix. The FASTA program package was authored by W. R. Pearson and D. J. Lipman (1988), “Improved Tools for Biological Sequence Analysis”, PNAS 85:2444-2448; W. R. Pearson (1996) “Effective protein sequence comparison” Meth. Enzymol. 266:227-258; and Pearson et. al. (1997) Genomics 46:24-36 and is publicly available from www.fasta.bioch.virginia.edu/fasta_www2/fasta_down.shtml or www. ebi.ac.uk/Tools/sss/fasta. Alternatively, a public server accessible at fasta.bioch.virginia.edu/fasta_www2/index.cgi can be used to compare the sequences, using the ggsearch (global protein:protein) program and default options (BLOSUM50; open: −10; ext: −2; Ktup=2) to ensure a global, rather than local, alignment is performed. Percent amino acid identity is given in the output alignment header.

The term “pharmaceutical composition” or “pharmaceutical formulation” refers to a preparation which is in such form as to permit the biological activity of an active ingredient contained therein to be effective, and which contains no additional components which are unacceptably toxic to a subject to which the pharmaceutical composition would be administered.

A “pharmaceutically acceptable carrier” refers to an ingredient in a pharmaceutical composition or formulation, other than an active ingredient, which is nontoxic to a subject. A pharmaceutically acceptable carrier includes, but is not limited to, a buffer, excipient, stabilizer, or preservative.

A reference to a target antigen as used herein, refers to any native target antigen from any vertebrate source, including mammals such as primates (e.g., humans) and rodents (e.g., mice and rats), unless otherwise indicated. The term encompasses “full-length”, unprocessed target antigen as well as any form of target antigen that results from processing in the cell. The term also encompasses naturally occurring variants of the target antigen, e.g., splice variants or allelic variants. For instance, the target antigen CEA may have the amino acid sequence of human CEA, in particular Carcinoembryonic antigen-related cell adhesion molecule 5 (CEACAM5), which is shown in UniProt (www.uniprot.org) accession no. P06731 (version 119), or NCBI (www.ncbi.nlm.nih.gov/) RefSeq NP_004354.2. Another example of a target antigen is Fibroblast Activation Protein (FAP). The amino acid sequence of human FAP is shown in UniProt (www.uniprot.org) accession no. Q12884 (version 149), or NCBI (www.ncbi.nlm.nih.gov/) RefSeq NP_004451.2. Another example of a target antigen is GPRC5D (see UniProt no. Q9NZD1 (version 115); NCBI RefSeq no. NP_061124.1 for the human sequence).

The terms “split antibody”, “split antibodies”, “single domain split antibodies” or “SPLIT PRIT” as referred to herein mean that the VH and VL domain which together form an antigen binding site capable of binding to the radiolabelled compound are split between two antibodies, and not present as part of the same antibody (before assembly in vivo). “CEA-targeted SPLIT PRIT” refers to a split antibody targeting CEA. The term “SPLIT PRIT” may also be used interchangeably with the term “TA-split-DOTAM-VH/VL” (e.g., where “TA” or target antigen is CEA, FAP or GPRC5D). The term “CEA-targeted SPLIT PRIT” may be used interchangeably with the term “CEA-split-DOTAM-VH/VL”.

As used herein, “treatment” (and grammatical variations thereof such as “treat” or “treating”) refers to clinical intervention in an attempt to alter the natural course of a disease in the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some aspects, antibodies of the invention are used to delay development of a disease or to slow the progression of a disease.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three complementary determining regions (CDRs). (See, e.g., Kindt et al. Kuby Immunology, 6^{th} ed., W. H. Freeman and Co., page 91 (2007)) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The term “vector”, as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors”.

The terms “Pb” or “lead” as used herein include ions thereof, e.g., Pb(II). References to other metals also include ions thereof. Thus, the skilled reader understands that, for example, the terms lead, Pb, ²¹²Pb or ²⁰³Pb are intended to encompass ionic forms of the element, in particular, Pb(II).

### II. Compositions and Methods

In one aspect, the invention is based, in part, on a set of antibodies comprising a first and a second antibody, wherein each antibody can bind to an antigen on a target cell, but wherein a functional antigen binding site for an effector agent is formed only when the first and second antibodies are associated with each other. Antibodies of the invention are useful, e.g., for methods of pre-targeted immunotherapy and/or for pre-targeted imaging. In preferred aspects the methods eliminate a step of administering a clearing agent or blocking agent.

### A. Target Antigens

The antigen expressed on the surface of the target cell is also termed herein the “target antigen”.

Insofar as the invention relates to treatment methods and to products for use therein, it is applicable to any condition that is treatable by cytotoxic activity targeted to cells of the patient, e.g., diseased cells. Thus, the target cell is any cell against which it is desired to target cytotoxicity, e.g., any diseased cell. The treatment is preferably of a tumour or cancer. However, the applicability of the invention is not limited to tumours and cancers. For example, the treatment may also be of viral infection (by targeting infected cells) or T-cell driven autoimmune disease (by targeting T cells). Immunotoxins directed against viral antigens expressed on the surface of infected cells have been investigated for a variety of viral infections such as HIV, rabies and EBV. Cai and Berger 2011 Antiviral Research 90(3):143-50 used an immunotoxin containing PE38 for targeted killing of cells infected with Kaposi's sarcoma-associated herpesvirus. In addition, Resimmune® (A-dmDT390-bisFv (UCHT1)) selectively kills human malignant T cells and transiently depletes normal T cell and is considered to have potential for the treatment of T-cell driven autoimmune diseases such as multiple sclerosis and graft-versus-host disease, as well as T cell blood cancers for which it is undergoing clinical trials. Likewise, methods of the invention may be applicable to any cell type for which radioimaging is desirable, including but not limited to cancer or tumour cells.

Thus, suitable target antigens may include cancer cell antigens, viral antigens or microbial antigens.

The antigens are usually normal cell surface antigens which are either over-expressed or expressed at abnormal times. Ideally the target antigen is expressed only on diseased cells (such as tumour cells), however this is rarely observed in practice. As a result, target antigens are usually selected on the basis of differential expression between diseased and healthy tissue.

The cell surface marker or target antigen can be, for example, a tumour-associated antigen.

The term “tumour-associated antigen” or “tumour specific antigen” as used herein refers to any molecule (e.g., protein, peptide, lipid, carbohydrate, etc.) solely or predominantly expressed or over-expressed by tumour cells and/or cancer cells, or by other cells of the stroma of the tumour such as cancer-associated fibroblasts, such that the antigen is associated with the tumour(s) and/or cancer(s). The tumour-associated antigen can additionally be expressed by normal, non-tumour, or non-cancerous cells. However, in such cases, the expression of the tumour-associated antigen by normal, non-tumour, or non-cancerous cells is not as robust as the expression by tumour or cancer cells. In this regard, the tumour or cancer cells can over-express the antigen or express the antigen at a significantly higher level, as compared to the expression of the antigen by normal, non-tumour, or non-cancerous cells. Also, the tumour-associated antigen can additionally be expressed by cells of a different state of development or maturation. For instance, the tumour-associated antigen can be additionally expressed by cells of the embryonic or foetal stage, which cells are not normally found in an adult host. Alternatively, the tumour-associated antigen can be additionally expressed by stem cells or precursor cells, which cells are not normally found in an adult host.

The tumour-associated antigen can be an antigen expressed by any cell of any cancer or tumour, including the cancers and tumours described herein. The tumour-associated antigen may be a tumour-associated antigen of only one type of cancer or tumour, such that the tumour-associated antigen is associated with or characteristic of only one type of cancer or tumour. Alternatively, the tumour-associated antigen may be a tumour-associated antigen (e.g., may be characteristic) of more than one type of cancer or tumour. For example, the tumour-associated antigen may be expressed by both breast and prostate cancer cells and not expressed at all by normal, non-tumour, or non-cancer cells.

Exemplary tumour-associated antigens to which the antibodies of the invention may bind include, but are not limited to, mucin 1 (MUC1; tumour-associated epithelial mucin), preferentially expressed antigen of melanoma (PRAME), carcinoembryonic antigen (CEA), prostate specific membrane antigen (PSMA), PSCA, EpCAM, Trop2 (trophoblast-2, also known as EGP-1), granulocyte-macrophage colony-stimulating factor receptor (GM-CSFR), CD56, human epidermal growth factor receptor 2 (HER2/neu) (also known as erbB-2), CDS, CD7, tyrosinase related protein (TRP) I, and TRP2. In another embodiment, the tumour antigen may be selected from the group consisting of cluster of differentiation (CD) 19, CD20, CD21, CD22, CD25, CD30, CD33 (sialic acid binding Ig-like lectin 3, myeloid cell surface antigen), CD79b, CD123 (interleukin 3 receptor alpha), transferrin receptor, EGF receptor, mesothelin, cadherin, Lewis Y, Glypican-3, FAP (fibroblast activation protein alpha), GPRC5D (G Protein-Coupled Receptor Class C Group 5 Member D), PSMA (prostate specific membrane antigen), CA9=CAIX (carbonic anhydrase IX), L1 CAM (neural cell adhesion molecule L 1), endosialin, HER3 (activated conformation of epidermal growth factor receptor family member 3), Alkl/BMP9 complex (anaplastic lymphoma kinase 1/bone morphogenetic protein 9), TPBG=5T4 (trophoblast glycoprotein), ROR1 (receptor tyrosine kinase-like surface antigen), HER1 (activated conformation of epidermal growth factor receptor), and CLL1 (C-type lectin domain family 12, member A). Mesothelin is expressed in, e.g., ovarian cancer, mesothelioma, non-small cell lung cancer, lung adenocarcinoma, fallopian tube cancer, head and neck cancer, cervical cancer, and pancreatic cancer. CD22 is expressed in, e.g., hairy cell leukaemia, chronic lymphocytic leukaemia (CLL), prolymphocytic leukaemia (PLL), non-Hodgkin's lymphoma, small lymphocytic lymphoma (SLL), and acute lymphatic leukaemia (ALL). CD25 is expressed in, e.g., leukemias and lymphomas, including hairy cell leukaemia and Hodgkin's lymphoma. Lewis Y antigen is expressed in, e.g., bladder cancer, breast cancer, ovarian cancer, colorectal cancer, esophageal cancer, gastric cancer, lung cancer, and pancreatic cancer. CD33 is expressed in, e.g., acute myeloid leukaemia (AML), chronic myelomonocytic leukaemia (CML), and myeloproliferative disorders.

Exemplary antibodies that specifically bind to tumour-associated antigens include, but are not limited to, antibodies against the transferrin receptor (e.g., HB21 and variants thereof), antibodies against CD22 (e.g., RFB4 and variants thereof), antibodies against CD25 (e.g., anti-Tac and variants thereof), antibodies against mesothelin (e.g., SS 1, MORAb-009, SS, HN1, HN2, MN, MB, and variants thereof) and antibodies against Lewis Y antigen (e.g., B3 and variants thereof). In this regard, the targeting moiety (cell-binding agent) may be an antibody selected from the group consisting of B3, RFB4, SS, SS1, MN, MB, HN1, HN2, HB21, and MORAb-009, and antigen binding portions thereof. Further exemplary targeting moieties suitable for use in the inventive chimeric molecules are disclosed e.g., in U.S. Pat. No. 5,242,824 (anti-transferrin receptor); 5,846,535 (anti-CD25); 5,889,157 (anti-Lewis Y); 5,981,726 (anti-Lewis Y); 5,990,296 (anti-Lewis Y); U.S. Pat. No. 7,081,518 (anti-mesothelin); 7,355,012 (anti-CD22 and anti-CD25); U.S. Pat. No. 7,368,110 (anti-mesothelin); 7,470,775 (anti-CD30); 7,521,054 (anti-CD25); and 7,541,034 (anti-CD22); U.S. Patent Application Publication 2007/0189962 (anti-CD22); Frankel et al., Clin. Cancer Res., 6: 326-334 (2000), and Kreitman et al., AAPS Journal, 8(3): E532-E551 (2006), each of which is incorporated herein by reference.

Further antibodies have been raised to target specific tumour related antigens including: Cripto, CD30, CD19, CD33, Glycoprotein NMB, CanAg, Her2 (ErbB2/Neu), CD56 (NCAM), CD22 (Siglec2), CD33 (Siglec3), CD79, CD138, PSCA, PSMA (prostate specific membrane antigen), BCMA, CD20, CD70, E-selectin, EphB2, Melanotransferin, Muc16 and TMEFF2. Any of these, or antigen-binding fragments thereof, may be useful in the present invention, i.e., may be incorporated into the antibodies described herein.

In some embodiments of the present invention, it may be preferred that the tumour-associated antigen is carcinoembryonic antigen (CEA).

CEA is advantageous in the context of the present invention because it is relatively slowly internalized, and thus a high percentage of the antibody will remain available on the surface of the cell after initial treatment, for binding to the radionuclide. Other low internalizing targets/tumour associated antigens may also be preferred. Other examples of tumour-associated antigen include CD20 or HER2. In still further embodiments, the target may be EGP-1 (epithelial glycoprotein-1, also known as trophoblast-2), colon-specific antigen-p (CSAp) or a pancreatic mucin MUC1. See for instance Goldenberg et al 2012 (Theranostics 2(5)), which is incorporated herein by reference. This reference also describes antibodies such as Mu-9 binding to CSAp (see also Sharkey et al Cancer Res. 2003; 63: 354-63), hPAM4 binding to MUC1 (see also Gold et al Cancer Res. 2008: 68: 4819-26), valtuzumab binding to CD20 (see also Sharkey et al Cancer Res. 2008; 68: 5282-90) and hRS7 which binds to EGP-1 (see also Cubas et al Biochim Biophys Acta 2009; 1796: 309-14). Any of these or antigen-binding portions thereof may be useful in the present invention, i.e., may be incorporated into the antibodies described herein. One example of an antibody that has been raised against CEA is T84.66 (as shown in NCBI Acc No: CAA36980 for the heavy chain and CAA36979 for the light chain, or as shown in SEQ ID NO 317 and 318 of WO2016/075278) and humanized and chimeric versions thereof, such as T84.66-LCHA as described in WO2016/075278 A1 and/or WO2017/055389. Another example is CH1A1a, an anti-CEA antibody as described in WO2012/117002 and WO2014/131712, and CEA hMN-14 (see also U.S. Pat. Nos. 6,676,924 and 5,874,540). Another anti-CEA antibody is A5B7 as described in M. J. Banfield et al, Proteins 1997, 29(2), 161-171. Humanized antibodies derived from murine antibody A5B7 have been disclosed in WO 92/01059 and WO 2007/071422. See also co-pending application PCT/EP2020/067582. An example of a humanized version of A5B7 is A5H1EL1 (G54A). A further exemplary antibody against CEA is MFE23 and the humanized versions thereof described in U.S. Pat. No. 7,626,011 and/or co-pending application PCT/EP2020/067582. A still further example of an antibody against CEA is 28A9. Any of these or an antigen binding fragment thereof may be useful to form a CEA-binding moiety in the present invention.

FAP (fibroblast activation protein alpha) or GPRC5D (G Protein-Coupled Receptor Class C Group 5 Member D) may also be preferred in some embodiments. FAP is an established target for imaging and therapy, due to its broad expression in the microenvironment of a number of tumor types, e.g. pancreas, breast, and lung cancer (Lindner, T., Loktev, A., Giesel, F. et al. Targeting of activated fibroblasts for imaging and therapy. EJNMMI radiopharm. chem. 4, 16 (2019)). SPLIT PRIT using FAP-split-DOTAM-VH/VL antibodies would thus be expected to generate specific accumulation of ²¹²Pb-DOTAM on activated cancer-associated fibroblasts. Consequently, the emitted alpha radiation would be expected to negatively affect the immune suppression of FAP-expressing malignant tumors, in addition to a limited direct tumor-killing effect on adjacent tumor cells. G-protein coupled receptor family C group 5 member D (GPRC5D) is overexpressed on multiple myeloma plasma cells (Atamaniuk J, Gleiss A, Porpaczy E, Kainz B, Grunt T W, Raderer M, et al. Overexpression of G protein-coupled receptor 5D in the bone marrow is associated with poor prognosis in patients with multiple myeloma. Eur J Clin Invest. 2012; 42:953-60), with established SC (subcutaneous) in vivo models reflecting the expression found in multiple myeloma patients, e.g. OPM-2 and NCI-H929 (Kodama T, Kochi Y, Nakai W, Mizuno H, Baba T, Habu K, et al. Anti-GPRC5D/CD3 bispecific T-cell-redirecting antibody for the treatment of multiple myeloma. Mol Cancer Ther. (2019) 18:1555-64). We therefore expect SPLIT PRIT using GPRC5D-split-DOTAM-VH/VL antibodies to generate tumor-specific accumulation of 212Pb-DOTAM followed by radiation-induced tumor cell death.

In some embodiments, the antibodies of the invention may bind specifically to the target antigen (e.g., any of the target antigens discussed herein). In some embodiments, they may bind with a dissociation constant (Kd) of ≤1 μM, ≤100 nM, ≤10 nM, ≤1 nM, ≤0.1 nM, ≤0.01 nM, or ≤0.001 nM (e.g. 10⁻⁷M or less, e.g. from 10⁻⁷ to 10⁻¹³, 10⁻⁸ M or less, e.g. from 10⁻⁸ M to 10⁻¹³ M, e.g., from 10⁻⁹ M to 10⁻¹³ M).

The first and second antibody each bind to the same target antigen, which can be termed “antigen A” (i.e., they have binding specificity for the same target antigen). They may each having binding specificity for the same epitope on antigen A. Alternatively, the first antibody may bind to a first epitope on antigen A and the second antibody may bind to a different, second epitope on antigen A. For instance, in one embodiment, one of the antibodies may bind to the T84.66 epitope of CEA and the other may bind to the A5B7 epitope of CEA.

In some embodiments, one or both of the first and/or second antibodies may be biparatopic for antigen A—i.e., each of the individual antibodies may bind to two different epitopes of antigen A. The first antibody may comprise a first and a second binding site, which bind to a first and a second epitope of antigen A respectively, wherein the first and second epitopes are different from each other. Alternatively or additionally, the second antibody may comprise a first and a second binding site, which bind to a first and a second epitope of antigen A, wherein the first and second epitopes are different from each other. In some embodiments, one or both of the epitopes bound by the first antibody may be different from one or both of the epitopes bound by the second antibody. In other embodiments, the two epitopes bound by the first antibody may be the same as the two epitopes bound by the second antibody.

### B. Radiolabelled Compounds

According to the present invention, association of the first and second antibodies forms a functional binding site for an effector molecule. Effector molecules according to the present invention are radiolabelled compounds which comprise a radioisotope, e.g., are a radiolabelled hapten.

In some embodiments, the effector molecule may comprise a chelated radioisotope.

In some embodiments, the functional binding site for the effector molecule may bind to a chelate comprising the chelator and the radioisotope. In other embodiments, the antibody may bind to a moiety which is conjugated to the chelated radioisotope, for instance, histamine-succinyl-glycine (HSG), digoxigenin, biotin or caffeine

The chelator may be, for example, a multidentate molecule such as an aminopolycarboxylic acid or an aminopolythiocarboxylic acid, or a salt or functional variant thereof. The chelator may be, for example, bidentate or tridentate or tetradentate. Examples of suitable metal chelators include molecules comprising EDTA (Ethylenediaminetetraacetic acid, or a salt form such as CaNa₂EDTA), DTPA (Diethylenetriamine Pentaacetic Acid), DOTA (1,4,7,10-tetraazacyclododecane-1,4,7,10-tetraacetic acid), NOTA (2,2′,2″-(1,4,7-Triazanonane-1,4,7-triyl)triacetic acid), IDA (Iminodiacetic acid), MIDA ((Methylimino)diacetic acid), TTHA (3,6,9,12-Tetrakis(carboxymethyl)-3,6,9,12-tetra-azatetradecanedioic acid), TETA (2,2′,2″,2′″-(1,4,8,11-Tetraazacyclotetradecane-1,4,8,11-tetrayl)tetraacetic acid), DOTAM (1,4,7,10-Tetrakis(carbamoylmethyl)-1,4,7,10-tetraazacyclododecane), HEHA (1,4,7,10,13,16-hexaazacyclohexadecane-1,4,7,10,13,16-hexaacetic acid, available from Macrocyclics, Inc., Plano, Tex.), NTA (nitrilotriacetic acid) EDDHA (ethylenediamine-N, N′-bis(2-hydroxyphenylacetic acid), BAL (2,3,-dimercaptopropanol), DMSA (2,3-dimercaptosuccinic acid), DMPS (2,3-dimercapto-1-propanesulfonic acid), D-penicillamine (B-dimethylcysteine), MAG₃ (mercaptoacetyltriglycine), Hynic (6-hydrazinopridine-3-carboxylic acid), p-isothiocyanatobenzyl-desferrioxamine (e.g., labelled with zirkonium for imaging), and salts or functional variants/derivatives thereof capable of chelating the metal. In some embodiments, it may be preferred that the chelator is DOTA or DOTAM or a salt or functional variant/derivative thereof capable of chelating the metal. Thus, the chelator may be or may comprise DOTA or DOTAM with a radioisotope chelated thereto.

The effector molecule may comprise or consist of functional variants or derivatives of the chelators above, together with the radionuclide. Suitable variants/derivatives have a structure that differs to a certain limited extent and retain the ability to function as a chelator (i.e. retains sufficient activity to be used for one or more of the purposes described herein). Functional variants/derivatives may also include a chelator as described above conjugated to one or more additional moieties or substituents, including, a small molecule, a polypeptide or a carbohydrate. This attachment may occur via one of the constituent carbons, for example in a backbone portion of the chelator. A suitable substituent can be, for example, a hydrocarbon group such as alkyl, alkenyl, aryl or alkynyl; a hydroxy group; an alcohol group; a halogen atom; a nitro group; a cyano group; a sulfonyl group; a thiol group; an amine group; an oxo group; a carboxy group; a thiocarboxy group; a carbonyl group; an amide group; an ester group; or a heterocycle including heteroaryl groups. The substituent may be, for example, one of those defined for group “R¹” below. A small molecule can be, for example, a dye (such as Alexa 647 or Alexa 488), biotin or a biotin moiety, or a phenyl or benzyl moiety. A polypeptide may be, for example, an oligo peptide, e.g., an oligopeptide of two or three amino acids. Exemplary carbohydrates include dextran, linear or branched polymers or co-polymers (e.g. polyalkylene, poly(ethylene-lysine), polymethacrylate, polyamino acids, poly- or oligosaccharides, dendrimers). Derivatives may also include multimers of the chelator compounds in which compounds as set out above are linked through a linker moiety. Derivatives may also include functional fragments of the above compounds, which retain the ability to chelate the metal ion.

Particular examples of derivatives include benzyl-EDTA and hydroxyethyl-thiourido-benzyl EDTA, DOTA-benzene (e.g., (S-2-(4-aminobenzyl)-1,4,7,10-tetraazacyclododecane tetraacetic acid), DOTA-biotin, and DOTA-TyrLys-DOTA.

In some embodiments of the present invention, the functional binding site formed by association of the first and second antibody binds to a metal chelate comprising DOTAM and a metal, e.g., lead (Pb). As mentioned above, “DOTAM” has the chemical name:

1,4,7,10-Tetrakis(carbamoylmethyl)-1,4,7,10-tetraazacyclododecane,

which is a compound of the following formula:

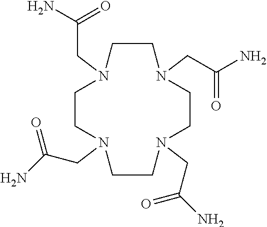 [SMILES: NC(=O)CN1CCN(CC(N)=O)CCN(CC(N)=O)CCN(CC(N)=O)CC1]

The present invention may in certain aspects and embodiments also make use of functional variants or derivatives of DOTAM incorporating a metal ion. Suitable variants/derivatives of DOTAM have a structure that differs to a certain limited extent from the structure of DOTAM and retain the ability to function (i.e. retains sufficient activity to be used for one or more of the purposes described herein). In such aspects and embodiments, the DOTAM or functional variant/derivative of DOTAM may be one of the active variants disclosed in WO 2010/099536. Suitable functional variants/derivatives may be a compound of the following formula:

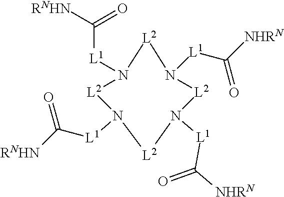

or a pharmaceutically acceptable salt thereof; wherein

R^{N} is H, C₁₋₆ alkyl, C₁₋₆ haloalkyl, C₂₋₆ alkenyl, C₂₋₆ alkynyl, C₃₋₇ cycloalkyl, C₃₋₇ cycloalkyl-C₁₋₄ alkyl, C₂₋₇ heterocycloalkyl, C₂₋₇ heterocycloalkyl-C₁₋₄ alkyl, phenyl, phenyl-C₁₋₄-alkyl, C₁₋₇ heteroaryl, and C₁₋₇heteroaryl-C₁₋₄-alkyl; wherein C₁₋₆ alkyl, C₁₋₆ haloalkyl, C₂₋₆ alkenyl, and C₂₋₆ alkynyl are each optionally substituted by 1, 2, 3, or 4 independently selected R^{w} groups; and wherein said C₃₋₇ cycloalkyl, C₃₋₇ cycloalkyl-C₁₋₄alkyl, C₂₋₇ heterocycloalkyl, C₂₋₇ heterocycloalkyl-C₁₋₄ alkyl, phenyl, phenyl-C₁₋₄-alkyl, C₁₋₇ heteroaryl, and C₁₋₇ heteroaryl-C₁₋₄-alkyl are each optionally substituted by 1, 2, 3, or 4 independently selected R^{x} groups;

L¹ is independently C₁₋₆ alkylene, C₁₋₆ alkenylene, or C₁₋₆ alkynylene, each of which is optionally substituted by 1, 2, or 3 groups independently selected R¹ groups;

L² is C₂₋₄ straight chain alkylene, which is optionally substituted by an independently selected R¹ group; and which is optionally substituted by 1, 2, 3, or 4 groups independently selected from C₁₋₄ alkyl and or C₁₋₄ haloalkyl;

R¹ is independently selected from D¹-D²-D³, halogen, cyano, nitro, hydroxyl, C₁₋₆ alkoxy, C₁₋₆ haloalkoxy, C₁₋₆ alkylthio, C₁-6 alkylsulfinyl, C₁₋₆ alkylsulfonyl, amino, C₁₋₆ alkylamino, di-C₁₋₆ alkylamino, C₁₋₄ alkylcarbonyl, carboxy, C₁₋₆ alkoxycarbonyl, C₁₋₆ alkylcarbonylamino, di-C₁₋₆ alkylcarbonylamino, C₁₋₆ alkoxycarbonylamino, C₁₋₆ alkoxycarbonyl-(C₁₋₆ alkyl)amino, carbamyl, C₁₋₆ alkylcarbamyl, and di-C₁₋₆ alkylcarbamyl;

each D¹ is independently selected from C₆₋₁₀ aryl-C₁₋₄ alkyl, C₁₋₉ heteroaryl-C₁₋₄ alkyl, C₃₋₁₀ cycloalkyl-C₁₋₄ alkyl, C₂₋₉ heterocycloalkyl-C₁₋₄ alkyl, C₁₋₈ alkylene, C₁₋₈ alkenylene, and C₁₋₈ alkynylene; wherein said C₁₋₈ alkylene, C₁₋₈ alkenylene, and C₁₋₈ alkynylene are optionally substituted by 1, 2, 3, or 4 independently selected R⁴ groups; and wherein said C₆₋₁₀ aryl-C₁₋₄ alkyl, C₁₋₉ heteroaryl-C₁₋₄ alkyl, C₃₋₁₀ cycloalkyl-C₁₋₄ alkyl, C₂₋₉ heterocycloalkyl-C₁₋₄ alkyl are each optionally substituted by 1, 2, 3, or 4 independently selected R⁵ groups;

each D² is independently absent or C₁₋₂₀ straight chain alkylene, wherein from 1 to 6 non-adjacent methylene groups of said C₁₋₂₀ straight chain alkylene are each optionally replaced by an independently selected —D⁴— moiety, provided that at least one methylene unit in said C₁₋₂₀ straight chain alkylene is not optionally replaced by a —D⁴— moiety; wherein said C₁₋₂₀ straight chain alkylene is optionally substituted by one or more groups independently selected from halogen, cyano, nitro, hydroxyl, C₁₋₄ alkyl, C₁₋₄ haloalkyl, C₁₋₄ alkoxy, C₁₋₄ haloalkoxy, amino, C₁₋₄ alkylamino, di-C₁₋₄ alkylamino, C₁₋₄ alkylcarbonyl, carboxy, C₁₋₄ alkoxycarbonyl, C₁₋₄ alkylcarbonylamino, di-C₁₋₄ alkylcarbonylamino, C₁₋₄ alkoxycarbonylamino, C₁₋₄ alkoxycarbonyl-(C₁₋₄ alkyl)amino, carbamyl, C₁₋₄ alkylcarbamyl, and di-C₁₋₄ alkylcarbamyl;

each D³ is independently selected from H, halogen, cyano, nitro, hydroxyl, C₁₋₆ alkyl, C₁₋₆ haloalkyl, C₂₋₆ alkenyl, C₂₋₆ alkynyl, C₃₋₁₄ cycloalkyl, C₃₋₁₄ cycloalkyl-C₁₋₄ alkyl, C₂₋₁₄ heterocycloalkyl, C₂₋₁₄ heterocycloalkyl-C₁₋₄ alkyl, C₆₋₁₄ aryl, C₆₋₁₄ aryl-C₁₋₄ alkyl, C₁₋₁₃ heteroaryl, C₁₋₁₃ heteroaryl-C₁₋₄ alkyl; wherein said C₁₋₆ alkyl, C₁₋₆ haloalkyl, C₂₋₆ alkenyl, C₂₋₆ alkynyl are each optionally substituted by 1, 2, 3, or 4 independently selected R⁶ groups; and wherein said C₃₋₁₄ cycloalkyl, C₃₋₁₄ cycloalkyl-C₁₋₄ alkyl, C₂₋₁₄ heterocycloalkyl, C₂₋₁₄ heterocycloalkyl-C₁₋₄ alkyl, C₆₋₁₄ aryl, C₆₋₁₄ aryl-C₁₋₄ alkyl, C₁₋₁₃ heteroaryl, C₁₋₁₃ heteroaryl-C₁₋₄ alkyl are each optionally substituted by 1, 2, 3 or 4 independently selected R⁷ groups;

each D⁴ is independently selected from —O—, —S—, —NR^{a}C(═O)—, —NR^{a}C(═S)—, —NR^{b}C(═O)NR^{c}—, —NR^{b}C(═S)NR^{c}—, —S(═O)—, —S(═O)₂—, —S(═O)NR^{a}—, —C(═O)—, —C(═S)—, —C(═O)O—, —OC(═O)NR^{a}—, —OC(═S)NR^{a}—, —NR^{a}—, —NR^{b}S(═O)NR^{c}—, and NR^{b}S(═O)₂NR^{O}—;

each R⁴ and R⁶ is independently selected from halogen, cyano, nitro, hydroxyl, C₁₋₄ alkoxy, C₁₋₄ haloalkoxy, C₁₋₄ alkylthio, C₁₋₄ alkylsulfinyl, C₁₋₄ alkylsulfonyl, amino, C₁₋₄ alkylamino, di-C₁₋₄ alkylamino, C₁₋₄ alkylcarbonyl, carboxy, C₁₋₄ alkoxycarbonyl, C₁₋₄ alkylcarbonylamino, di-C₁₋₄ alkylcarbonylamino, C₁₋₄ alkoxycarbonylamino, C₁₋₄ alkoxycarbonyl-(C₁₋₄ alkyl)amino, carbamyl, C₁₋₄ alkylcarbamyl, and di-C₁₋₄ alkylcarbamyl;

each R⁵ is independently selected from halogen, cyano, cyanate, isothiocyanate, nitro, hydroxyl, C₁₋₄ alkyl, C₂₋₄ alkenyl, C₂₋₄ alkynyl, C₁₋₄ alkoxy, C₁₋₄ haloalkoxy, C₁₋₄ alkylthio, C₁₋₄ alkylsulfinyl, C₁₋₄ alkylsulfonyl, amino, C₁₋₄ alkylamino, di-C₁₋₄ alkylamino, C₁₋₄ alkylcarbonyl, carboxy, C₁₋₄ alkoxycarbonyl, C₁₋₄ alkylcarbonylamino, di-C₁₋₄ alkylcarbonylamino, C₁₋₄ alkoxycarbonylamino, C₁₋₄ alkoxycarbonyl-(C₁₋₄ alkyl)amino, carbamyl, C₁₋₄ alkylcarbamyl, and di-C₁₋₄ alkylcarbamyl;

each R⁷ is independently selected from halogen, cyano, nitro, hydroxyl, C₁₋₆ alkyl, C₂₋₆ alkenyl, C₂₋₆ alkynyl, C₃₋₇ cycloalkyl, C₃₋₇ cycloalkyl-C₁₋₄ alkyl, C₂₋₇ heterocycloalkyl, C₂₋₇ heterocycloalkyl-C₁₋₄ alkyl, phenyl, phenyl-C₁₋₄ alkyl, C₁₋₇ heteroaryl, C₁₋₇ heteroaryl-C₁₋₄ alkyl, —OR^{O}, —SR^{O}, —S(═O)R^{P}, —S(═O)₂R^{P}, —S(═O)NR^{s}R^{t}, —C(═O)R^{P}, —C(═O)OR^{P}, —C(═O)NR^{s}R^{t}, —OC(═O)R^{P}, —OC(═O)NR^{s}R^{t}, —NR^{s}R^{t}, —NR^{q}C(═O)R^{r}, —NR^{q}C(═O)OR^{r}, —NR⁴C(═O)NR^{r}, —NR⁹S(═O)₂W, and —NR^{P}S(═O)₂NR^{s}R^{t}; wherein said C₁₋₆ alkyl, C₂₋₆ alkenyl, C₂₋₆ alkynyl are each optionally substituted by 1, 2, 3, or 4 independently selected R′ groups; and wherein said C₃₋₇ cycloalkyl, C₃₋₇ cycloalkyl-C₁₋₄ alkyl, C₂₋₇ heterocycloalkyl, C₂₋₇ heterocycloalkyl-C₁₋₄ alkyl, phenyl, phenyl-C₁₋₄ alkyl, C₁₋₇ heteroaryl, C₁₋₇ heteroaryl-C₁₋₄ alkyl are each optionally substituted by 1, 2, 3, or 4 independently selected R″ groups;

each R^{a}, R^{b}, and R^{c} is independently selected from H, C₁₋₆ alkyl, C₁₋₆ haloalkyl, C₂₋₆ alkenyl, C₂₋₆ alkynyl, C₃₋₇ cycloalkyl, C₃₋₇ cycloalkyl-C₁₋₄ alkyl, C₂₋₇ heterocycloalkyl, C₂₋₇ heterocycloalkyl-C₁₋₄ alkyl, phenyl, phenyl-C₁₋₄ alkyl, C₁₋₇ heteroaryl, C₁₋₇ heteroaryl-C₁₋₄ alkyl; wherein said C₁₋₆ alkyl, C₁₋₆ haloalkyl, C₂₋₆ alkenyl, C₂₋₆ alkynyl are each optionally substituted by 1, 2, 3, or 4 independently selected R^{w} groups; and wherein said C₃₋₇ cycloalkyl, C₃₋₇ cycloalkyl-C₁₋₄ alkyl, C₂₋₇ heterocycloalkyl, C₂₋₇ heterocycloalkyl-C₁₋₄ alkyl, phenyl, phenyl-C₁₋₄ alkyl, C₁₋₇ heteroaryl, C₁₋₇ heteroaryl-C₁₋₄ alkyl are each optionally substituted by 1, 2, 3, or 4 independently selected R^{x} groups;

each R^{o}, R^{p}, R^{q}, R^{r}, R^{s} and R^{t} is independently selected from H, C₁₋₆ alkyl, C₁₋₆ haloalkyl, C₂₋₆ alkenyl, C₂₋₆ alkynyl, C₃₋₇ cycloalkyl, C₃₋₇ cycloalkyl-C₁₋₄ alkyl, C₂₋₇ heterocycloalkyl, C₂₋₇ heterocycloalkyl-C₁₋₄ alkyl, phenyl, phenyl-C₁₋₄ alkyl, C₁₋₇ heteroaryl, C₁₋₇ heteroaryl-C₁₋₄ alkyl; wherein said C₁₋₆ alkyl, C₁₋₆ haloalkyl, C₂₋₆ alkenyl, C₂₋₆ alkynyl are each optionally substituted by 1, 2, 3, or 4 independently selected RY groups; and wherein said C₃₋₇ cycloalkyl, C₃₋₇ cycloalkyl-C₁₋₄ alkyl, C₂₋₇ heterocycloalkyl, C₂₋₇ heterocycloalkyl-C₁₋₄ alkyl, phenyl, phenyl-C₁₋₄ alkyl, C₁₋₇ heteroaryl, C₁₋₇ heteroaryl-C₁₋₄ alkyl are each optionally substituted by 1, 2, 3, or 4 independently selected R^{z} groups;

each R′, R^{w} and R^{y} is independently selected from hydroxyl, cyano, nitro, C₁₋₄ alkoxy, C₁₋₄ haloalkoxy, amino, C₁₋₄ alkylamino, and di-C₁₋₄ alkylamino; and

each R″, R^{x}, and R^{z} is independently selected from hydroxyl, halogen, cyano, nitro, C₁₋₄ alkyl, C₁₋₄ haloalkyl, C₁₋₄ alkoxy, C₁₋₄ haloalkoxy, amino, C₁₋₄ alkylamino, and di-C₁₋₄ alkylamino;

provided that the valency of each atom in the optionally substituted moieties is not exceeded.

Suitably, the functional variants/derivatives of the above formula have an affinity for an antibody of the present invention which is comparable to or greater than that of DOTAM, and have a binding strength for Pb which is comparable to or greater than that of DOTAM (“affinity” being as measured by the dissociation constant, as described above). For example, the dissociation constant of the functional/variant derivative with the antibody of the present invention or/Pb may be 1.1 times or less, 1.2 times or less, 1.3 times or less, 1.4 times or less, 1.5 times or less, or 2 times or less than the dissociation constant of DOTAM with the same antibody/Pb.

Each R^{N} may be H, C₁₋₆ alkyl, or C₁₋₆ haloalkyl; preferably H, C₁₋₄ alkyl, or C₁₋₄ haloalkyl. Most preferably, each R^{N} is H.

For DOTAM variants, it is preferred that 1, 2, 3 or most preferably each L² is C₂ alkylene. Advantageously, the C₂ alkylene variants of DOTAM can have particularly high affinity for Pb. The optional substituents for L² may be R¹, C₁₋₄ alkyl, or C₁₋₄ haloalkyl. Suitably, the optional substituents for L² may be C₁₋₄ alkyl or C₁₋₄ haloalkyl.

Optionally, each L² may be unsubstituted C₂ alkylene —CH2CH2-.

Each L¹ is preferably C₁₋₄ alkylene, more preferably C₁ alkylene such as —CH2-.

The functional variant/derivative of DOTAM may be a compound of the following formula:

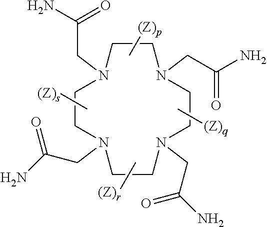

wherein each Z is independently R¹ as defined above; p, q, r, and s are 0, 1 or 2; and p+q+r+s is 1 or greater. Preferably, p, q, r, and s are 0 or 1 and/or p+q+r+s is 1. For example, the compound may have p+q+r+s=1, where Z is p-SCN-benzyl moiety—such a compound is commercially available from Macrocyclics, Inc. (Plano, Tex.).

Radionuclides useful in the invention may include radioisotopes of metals, such as of lead (Pb), lutetium (Lu), or yttrium (Y).

Radionuclides particularly useful in imaging applications may be radionuclides that are gamma emitters. For instance, they may be selected from ²⁰³Pb or ²⁰⁵Bi.

Radionuclides particularly useful in therapeutic applications be radionuclides that are alpha or beta emitters. For instance, they may be selected from ²¹²Pb, ²¹²Bi, ²¹³Bi, ⁹⁰Y, ¹⁷⁷Lu, ²²⁵Ac, ²¹¹At, ²²⁷Th, ²²³Ra.

In some embodiments, it may be preferred that DOTAM (or salts or functional variants thereof) is chelated with Pb or Bi such as one of the Pb or Bi radioisotopes listed above. It other embodiments, it may be preferred that DOTA (or salts or functional variants thereof) is chelated with Lu or Y such as one of the Lu or Y radioisotopes listed above.

In some embodiments, methods and uses may comprise combined methods of therapy and imaging, which make use of a mixture of radioisotopes, e.g., a radioisotope suitable for therapy and a radioisotope suitable for imaging. For instance, these may be different radioisotopes of the same metal, chelated by the same chelator. In one embodiment, the method may comprise administering ²⁰³Pb-DOTAM and ²¹²Pb-DOTAM as a mixture. In another embodiment, the method may comprise a first cycle of dosimetry using a gamma emitter such as ²⁰³Pb or ²⁰⁵Bi followed by one or more rounds of treatment using an alpha or beta emitter such as ²¹²Pb, ²¹²Bi, ²¹³Bi, ⁹⁰Y, ¹⁷⁷Lu, ²²⁵Ac, ²¹¹At, ²²⁷Th, or ²²³Ra. Such methods are described further below.

In some embodiments, the functional binding site formed by association of the first and the second antibody may bind to a Pb-DOTAM chelate.

In some embodiments, the functional binding site formed by association of the first and the second antibody may specifically bind to the radiolabelled compound. In some embodiments, it may bind to the radiolabelled compound, such as the Pb-DOTAM chelate, with a dissociation constant (Kd) to Pb-DOTAM and/or the target of ≤1 μM, ≤100 nM, ≤10 nM, ≤1 nM, ≤0.1 nM, ≤0.01 nM, or ≤0.001 nM (e.g. 10⁻⁷M or less, e.g. from 10⁻⁷ to 10⁻¹³, 10⁻⁸ M or less, e.g. from 10⁻⁸ M to 10-13 M, e.g., from 10⁻⁹ M to 10⁻¹³ M). It some embodiments it may be preferred that it binds with a Kd value of the binding affinity of 100 pM, 50 pM, 20 pM, 10 pM, 5 pM, 1 pM or less, e.g., 0.9 pM or less, 0.8 pM or less, 0.7 pM or less, 0.6 pM or less or 0.5 pM or less. For instance, the functional binding site may bind the metal chelate with a Kd of about 1 pM-1 nM, e.g., about 1-10 pM, 1-100 pM, 5-50 pM, 100-500 pM or 500 pM-1 nM.

### C. Exemplary Antigen Binding Sites for DOTA

In one particular embodiment of the invention, the first and second antibody associate to form a functional binding site for DOTA (or a functional derivative or variant thereof), e.g., DOTA chelated with Lu or Y (e.g., ¹⁷⁷Lu or ⁹⁰Y). For instance, the functional binding site may bind the radiolabelled compound with a Kd of about 1 pM-1 nM, e.g., about 1-10 pM, 1-100 pM, 5-50 pM, 100-500 pM or 500 pM-1 nM.

C825 is a known scFv with high affinity for DOTA-Bn (S-2-(4-aminobenzyl)-1,4,7,10-tetraazacyclododecane tetraacetic acid) complexed with radiometals such as ¹⁷⁷Lu and ⁹⁰Y (see for instance Cheal et al 2018, Theranostics 2018, and WO2010099536, incorporated herein by reference). The CDR sequences and the VL and VH sequences of C825 are provided herein. In one embodiment, the heavy chain variable region forming part of the antigen binding site for the radiolabelled compound may comprise at least one, two or all three CDRs selected from (a) CDR-H1 comprising the amino acid sequence of 35; (b) CDR-H2 comprising the amino acid sequence of 36; (c) CDR-H3 comprising the amino acid sequence of 37. In an alternative embodiment, CDR-H1 may have the sequence GFSLTDYGVH (SEQ ID NO.: 148). The light chain variable region forming part of the binding site for the radiolabelled compound may comprise at least one, two or all three CDRs selected from (d) CDR-L1 comprising the amino acid sequence of 38; (e) CDR-L2 comprising the amino acid sequence of 39; and (0 CDR-L3 comprising the amino acid sequence of 40.

In another embodiment, the heavy chain variable domain forming part of the functional antigen binding site for the radiolabelled compound (on the first antibody) comprises the amino acid sequence of SEQ ID NO: 41, or a variant thereof comprising an amino acid sequence having at least 90, 91, 92, 93, 94, 95, 96, 97, 98, or 99% identity to SEQ ID NO: 41. In certain embodiments, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but a binding site comprising that sequence retains the ability to bind to DOTA complexed with Lu or Y, preferably with an affinity as described herein. In certain embodiments, a total of 1 to 10 amino acids have been substituted, inserted and/or deleted in SEQ ID NO:41. In certain embodiments, substitutions, insertions, or deletions occur in regions outside the CDRs (i.e., in the FRs). Optionally, the antibody comprises the VH sequence in SEQ ID NO:41, including post-translational modifications of that sequence. In a particular embodiment, the VH comprises one, two or three CDRs selected from: (a) CDR-H1 comprising the amino acid sequence of SEQ ID NO:35 or the sequence GFSLTDYGVH (SEQ ID NO.: 148), (b) CDR-H2 comprising the amino acid sequence of SEQ ID NO:36, and (c) CDR-H3 comprising the amino acid sequence of SEQ ID NO:37.

Optionally, the light chain variable domain forming part of the functional antigen binding site for the radiolabelled compound (on the second antibody) comprises an amino acid sequence of SEQ ID NO: 42 or a variant thereof comprising an amino acid sequence having at least 90, 91, 92, 93, 94, 95, 96, 97, 98, or 99% identity to SEQ ID NO: 42. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but a binding site comprising that sequence retains the ability to bind to DOTA complexed with Lu or Y, preferably with an affinity as described herein. In certain embodiments, a total of 1 to 10 amino acids have been substituted, inserted and/or deleted in SEQ ID NO: 42. In certain embodiments, the substitutions, insertions, or deletions occur in regions outside the CDRs (i.e., in the FRs). Optionally, the antibody comprises the VL sequence in SEQ ID NO:42, including post-translational modifications of that sequence. In a particular embodiment, the VL comprises one, two or three CDRs selected from (a) CDR-L1 comprising the amino acid sequence of SEQ ID NO:38; (b) CDR-L2 comprising the amino acid sequence of SEQ ID NO:39; and (c) CDR-L3 comprising the amino acid sequence of SEQ ID NO:40.

Embodiments concerned with the heavy chain variable region and the light chain variable region are explicitly contemplated in combination. Thus, the functional antigen binding site may be formed from a heavy chain variable region as defined above and a light chain variable region as defined above, on the first and second antibody respectively.

In any of the above embodiments, the light and heavy chain variable regions forming the binding site for the DOTA complex may be humanized. In one embodiment, the light and heavy chain variable region comprise CDRs as in any of the above embodiments, and further comprise an acceptor human framework, e.g. a human immunoglobulin framework or a human consensus framework.

In some embodiments, the heavy chain variable domain may be extended by one or more C-terminal residues such as one or more C-terminal alanine residues, or one or more residues from the N-terminus of the CH1 domain, as discussed further below.

### D. Exemplary Antigen Binding Sites for DOTAM

In another particular embodiment of the invention, the first and second antibody associate to form a functional antigen binding site for a Pb-DOTAM chelate (Pb-DOTAM).

In certain embodiments, the functional antigen-binding site that binds to Pb-DOTAM may have one or more of the following properties:
Binds specifically to Pb-DOTAM and to Bi-DOTAM;
Is selective for Pb-DOTAM (and optionally Bi-DOTAM) as compared to other chelated metals, such as Cu-DOTAM;
Binds to Pb-DOTAM with a very high affinity;
Binds to the same epitope on Pb-DOTAM as antibodies described herein, e.g., PRIT-0213 or PRIT-0214 and/or has the same contact residues as said antibodies.

Radioisotopes of Pb are useful in methods of diagnosis and therapy. Particular radioisotopes of lead which may be of use in the present invention include ²¹²Pb and ²⁰³Pb.

Radionuclides which are α-particle emitters have the potential for more specific tumour cell killing with less damage to the surrounding tissue than β-emitters because of the combination of short path length and high linear energy transfer. ²¹²Bi is an α-particle emitter but its short half-life hampers its direct use. ²¹²Pb is the parental radionuclide of ²¹²Bi and can serve as an in vivo generator of ²¹²Bi, thereby effectively overcoming the short half-life of ²¹²Bi (Yong and Brechbiel, Dalton Trans. 2001 Jun. 21; 40 (23) 6068-6076).

²⁰³Pb is useful as an imaging isotope. Thus, an antibody bound to ²⁰³Pb-DOTAM may have utility in radioimmunoimaging (RIO.

Generally, radiometals are used in chelated form. In certain aspects of the present invention, DOTAM is used as the chelating agent. DOTAM is a stable chelator of Pb(II) (Yong and Brechbiel, Dalton Trans. 2001 Jun. 21; 40 (23) 6068-6076; Chappell et al Nuclear Medicine and Biology, Vol. 27, pp. 93-100, 2000). Thus, DOTAM is particularly useful in conjunction with isotopes of lead as discussed above, such as ²¹²Pb and ²⁰³Pb.

In some embodiments, it may be preferred that the antibodies bind Pb-DOTAM with a Kd value of the binding affinity of 100 pM, 50 pM, 20 pM, 10 pM, 5 pM, 1 pM or less, e.g, 0.9 pM or less, 0.8 pM or less, 0.7 pM or less, 0.6 pM or less or 0.5 pM or less. For instance, the functional binding site may bind the radiolabelled compound with a Kd of about 1 pM-1 nM, e.g., about 1-10 pM, 1-100 pM, 5-50 pM, 100-500 pM or 500 pM-1 nM.

In certain embodiment, the antibodies additionally bind to Bi chelated by DOTAM. In some embodiments, it may be preferred that the antibodies bind Bi-DOTAM (i.e., a chelate comprising DOTAM complexed with bismuth, also termed herein a “Bi-DOTAM chelate”) with a Kd value of the binding affinity of 1 nM, 500 pM, 200 pM, 100 pM, 50 pM, 10 pM or less, e.g., 9 pM, 8 pM, 7 pM, 6 pM, 5 pM or less. For instance, the functional binding site may bind a metal chelate with a Kd of about 1 pM-1 nM, e.g., about 1-10 pM, 1-100 pM, 5-50 pM, 100-500 pM or 500 pM-1 nM.

In some embodiments, the antibodies may bind to Bi-DOTAM and to Pb-DOTAM with a similar affinity. For instance, it may be preferred that the ratio of affinity, e.g., the ratio of Kd values, for Bi-DOTAM/Pb-DOTAM is in the range of 0.1-10, for example 1-10.

In one embodiment, the heavy chain variable region forming part of the antigen binding site for Pb-DOTAM may comprise at least one, two or all three CDRs selected from (a) CDR-H1 comprising the amino acid sequence of GFSLSTYSMS (SEQ ID NO:1); (b) CDR-H2 comprising the amino acid sequence of FIGSRGDTYYASWAKG (SEQ ID NO:2); (c) CDR-H3 comprising the amino acid sequence of ERDPYGGGAYPPHL (SEQ ID NO:3). The light chain variable region forming part of the binding site for Pb-DOTAM may comprise at least one, two or all three CDRs selected from (d) CDR-L1 comprising the amino acid sequence of QSSHSVYSDNDLA (SEQ ID NO:4); (e) CDR-L2 comprising the amino acid sequence of QASKLAS (SEQ ID NO:5); and (f) CDR-L3 comprising the amino acid sequence of LGGYDDESDTYG (SEQ ID NO:6).

In some embodiments, the antibodies may comprise one or more of CDR-H1, CDR-H2 and/or CDR-H3, or one or more of CDR-L1, CDR-L2 and/or CDR-L3, having substitutions as compared to the amino acid sequences of SEQ ID NOs: 1-6, respectively, e.g., 1, 2 or 3 substitutions.

In some embodiments, antibodies may share the same contact residues as the described herein: e.g., these residues may be invariant. These residues may include the following:
a) in heavy chain CDR2: Phe50, Asp56 and/or Tyr58, and optionally also Gly52 and/or Arg 54;
b) in heavy chain CDR3: Glu95, Arg96, Asp97, Pro98, Tyr99, Ala100C and/or Tyr100D and optionally also Pro100E;
c) in light chain CDR1: Tyr28 and/or Asp32;
d) in light chain CDR3: Gly91, Tyr92, Asp93, Thr95c and/or Tyr96;
e) in light chain CDR2: optionally G1n50;
all numbered according to Kabat.

For example, in some embodiments, CDR-H2 may comprise the amino acid sequence FIGSRGDTYYASWAKG (SEQ ID NO:2), or a variant thereof having up to 1, 2, or 3 substitutions in SEQ ID NO: 2, wherein these substitutions do not include Phe50, Asp56 and/or Tyr58, and optionally also do not include Gly52 and/or Arg 54, all numbered according to Kabat.

In some embodiments, CDR-H2 may be substituted at one or more positions as shown below. Here and in the substitution tables that follow, substitutions are based on the germline residues (underlined) or by amino acids which theoretically sterically fit and also occur in the crystallized repertoire at the site. In some embodiments, the residues as mentioned above may be fixed and other residues may be substituted according to the table below: in other embodiments, substitutions of any residue may be made according to the table below.

| WolfGuy | Kabat | AA | Substitution |
|---|---|---|---|
| 251 | 50 | F | Y, H |
| 252 | 51 | I | |
| 253 | 52 | G | |
| 254 | 53 | S | A, G, T, I, N |
| 288 | 54 | R | A, D, G, N, S, T, F, Y |
| 289 | 55 | G | D, S, Y, T, A, N, R, V |
| 290 | 56 | D | |
| 291 | 57 | T | K, I, A, P, S |
| 292 | 58 | Y | F, W, H |
| 293 | 59 | Y | N, F, H, L, S |
| 294 | 60 | A | G, N, S, T |
| 295 | 61 | S | A, G, N, Q, T |
| 296 | 62 | W | K, P, S, A, T, D, N, R, Q |
| 297 | 63 | A | F, L, V, M, I |
| 298 | 64 | K | N, Q, R, E |
| 299 | 65 | G | S, T, D, N, A |

Optionally, CDR-H3 may comprise the amino acid sequence ERDPYGGGAYPPHL (SEQ ID NO:3), or a variant thereof having up to 1, 2, or 3 substitutions in SEQ ID NO: 3, wherein these substitutions do not include Glu95, Arg96, Asp97, Pro98, and optionally also do not include Ala100C, Tyr100D, and/or Pro100E and/or optionally also do not include Tyr99. For instance, in some embodiments the substitutions do not include Glu95, Arg96, Asp97, Pro98, Tyr99 Ala100C and Tyr100D.

In certain embodiments, CDR-H3 may be substituted at one or more positions as shown below. In some embodiments, the residues as mentioned above may be fixed and other residues may be substituted according to the table below: in other embodiments, substitutions of any residue may be made according to the table below.

| WolfGuy | Kabat | AA | Substitution |
|---|---|---|---|
| 351 | 95 | E | |
| 352 | 96 | R | K, E |
| 353 | 97 | D | |
| 354 | 98 | P | |
| 355 | 99 | Y | F, G, S, T, D |
| 356 | 100 | G | |
| 392 | 100A | G | |
| 393 | 100B | G | |
| 394 | 100C | A | S, T |
| 395 | 100D | Y | F |
| 396 | 100E | P | |
| 397 | 100F | P | |
| 398 | 101 | H | A, T, V, D |
| 399 | 102 | L | Y, V, I, H, F |

Optionally, CDR-L1 may comprise the amino acid sequence QSSHSVYSDNDLA (SEQ ID NO:4) or a variant thereof having up to 1, 2, or 3 substitutions in SEQ ID NO: 4, wherein these substitutions do not include Tyr28 and/or Asp32 (Kabat numbering).

In certain embodiments, CDR-L1 may be substituted at one or more positions as shown below. Again, in some embodiments, the residues as mentioned above may be fixed and other residues may be substituted according to the table below: in other embodiments, substitutions of any residue may be made according to the table below.

| WolfGuy | Kabat | AA | Substitution |
|---|---|---|---|
| 551 | 24 | Q | R, K |
| 552 | 25 | S | A, G |
| 554 | 26 | S | T |
| 555 | 27 | H | Q, S, R, K |
| 556 | 27A | S | Q |
| 557 | 27B | V | I, D, N |
| 561 | 28 | Y | F |
| 562 | 29 | S | T, V |
| 571 | 30 | D | R, S, N, G |
| 572 | 31 | N | K |
| 597 | 32 | D | |
| 598 | 33 | L | I, V, M |
| 599 | 34 | A | S |

Optionally, CDR-L3 may comprise the amino acid sequence LGGYDDESDTYG (SEQ ID NO:6) or a variant thereof having up to 1, 2, or 3 substitutions in SEQ ID NO: 6, wherein these substitutions do not include Gly91, Tyr92, Asp93, Thr95c and/or Tyr96 (Kabat).

In certain embodiments, CDR-L3 may be substituted at the following positions as shown below. (Since most residues are solvent exposed and without antigen contacts, many substitutions are conceivable). Again, in some embodiments, the residues as mentioned above may be fixed and other residues may be substituted according to the table below: in other embodiments, substitutions of any residue may be made according to the table below.

| WolfGuy | Kabat | AA | Substitution |
|---|---|---|---|
| 751 | 89 | L | A, V, Q |
| 752 | 90 | G | A |
| 753 | 91 | G | |
| 754 | 92 | Y | A, D, E, F, G, H, I, |
| | | | K, L, N, Q, R, S, T, |
| | | | V |
| 755 | 93 | D | A, E, F, G, H, I, K, |
| | | | L, M, N, Q, R, S, T, |
| | | | V, W, Y |
| 756 | 94 | D | A, E, F, G, H, I, K, |
| | | | L, M, N, Q, R, S, T, |
| | | | V, W, Y |
| 794 | 95 | E | A, D, F, G, H, I, K, |
| | | | L, M, N, Q, R, S, T, |
| | | | V, W, Y |
| 795 | 95A | S | A, F, G, H, I, K, L, |
| | | | M, N, Q, R, T, V, W, |
| | | | Y |
| 796 | 95B | D | A, E, F, G, H, I, L, |
| | | | M, N, Q, S, T, V, W, |
| | | | Y |
| 797 | 95C | T | S |
| 798 | 96 | Y | F, H, R |
| 799 | 97 | G | A, E, I, K, L, M, N, |
| | | | Q, S, T, V |

The antibody may further comprise CDR-H1 or CDR-L2, optionally having the sequence of SEQ ID NO: 1 or SEQ ID NO: 5 respectively, or a variant thereof having at least 1, 2 or 3 substitutions relative thereto, optionally conservative substitutions.

Thus, the heavy chain variable domain forming part of the antigen binding site for Pb-DOTAM may comprise at least:

a) heavy chain CDR2 comprising the amino acid sequence FIGSRGDTYYASWAKG (SEQ ID NO:2), or a variant thereof having up to 1, 2, or 3 substitutions in SEQ ID NO: 2, wherein these substitutions do not include Phe50, Asp56 and/or Tyr58, and optionally also do not include Gly52 and/or Arg54;
b) heavy chain CDR3 comprising the amino acid sequence ERDPYGGGAYPPHL (SEQ ID NO:3), or a variant thereof having up to 1, 2, or 3 substitutions in SEQ ID NO: 3, wherein these substitutions do not include Glu95, Arg96, Asp97, Pro98, and optionally also do not include Ala100C, Tyr100D, and/or Pro100E and/or optionally also do not include Tyr99.

In some embodiments, the heavy chain variable domain additionally includes a heavy chain CDR1 which is optionally:

c) a heavy chain CDR1 comprising the amino acid sequence GFSLSTYSMS (SEQ ID NO:1) or a variant thereof having up to 1, 2, or 3 substitutions in SEQ ID NO: 1.

In some embodiments, the heavy chain variable domain additionally includes a C-terminal alanine (e.g. Ala114 according Kabat numbering system) to avoid the binding of pre-existing antibodies recognizing the free VH region. As reported in Holland M C et al J. Clin Immunol (2013), a free C-terminus appears to be important for binding of HAVH (human anti-VH domain) autoantibodies to VH domain antibodies, since HAVH autoantibodies do not bind to intact IgG or IgG fragments (fAb or modified VH molecules) containing the same VH framework sequences, or to VK domain antibodies. Cordy J C et al Clinical and Experimental Immunology (2015) notes the existence of a cryptic epitope at the C-terminal epitope of VH dAbs, which is not naturally accessible to HAVH antibodies in full IgG molecules.

Thus, where the antibody comprises a free VH region (not fused to any other domain at its C-terminus), the sequence may be extended by one or more C-terminal residue. The extension may prevent the binding of antibodies recognizing the free VH region. For instance, the extension may be by 1-10 residues, e.g., 1, 2, 3, 4, 5, 6, 7, 8, 9 or 10 residues. In one embodiment, the VH sequence may be extended by one or more C-terminal alanine residues. The VH sequence may also be extended by an N-terminal portion of the CH1 domain, e.g., by 1-10 residues from the N-terminus of the CH1 domain, e.g., from the human IgG1 CH1 domain. (The first ten residues of the human IgG1 CH1 domain are ASTKGPSVFP (SEQ ID NO.: 149), and so in one embodiment, from 1-10 residues may be taken from the N-terminus of this sequence). For instance, in one embodiment, the peptide sequence AST (corresponding to the first 3 residues of the IgG1 CH1 domain) is added to the C-terminus of the VH region.

In another embodiment, the light chain variable domain forming part of the antigen binding site for Pb-DOTAM comprises at least:
d) light chain CDR1 comprising the amino acid sequence QSSHSVYSDNDLA (SEQ ID NO:4) or a variant thereof having up to 1, 2, or 3 substitutions in SEQ ID NO: 4, wherein these substitutions do not include Tyr28 and Asp32;
e) light chain CDR3 comprising the amino acid sequence LGGYDDESDTYG (SEQ ID NO:6) or a variant thereof having up to 1, 2, or 3 substitutions in SEQ ID NO: 6, wherein these substitutions do not include Gly91, Tyr92, Asp93, Thr95c and Tyr96.

In some embodiments, the light chain variable domain additionally includes a light chain CDR2 which is optionally:
f) a light chain CDR2 comprising the amino acid sequence QASKLAS (SEQ ID NO: 5) or a variant thereof having at least 1, 2 or 3 substitutions in SEQ ID NO: 5, optionally not including Gln50.

In any embodiments of the present invention which include variants of a sequence comprising the CDRs as set out above (e.g., of a variable domain), the protein may be invariant in one or more of the CDR residues as set out above.

Optionally, the heavy chain variable domain forming part of the functional antigen binding site for Pb-DOTAM (on the first antibody) comprises an amino acid sequence selected from the group consisting of SEQ ID NO: 7 and SEQ ID NO 9, or a variant thereof comprising an amino acid sequence having at least 90, 91, 92, 93, 94, 95, 96, 97, 98, or 99% identity to SEQ ID NO: 7 or SEQ ID NO: 9. In certain embodiments, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but a binding site comprising that sequence retains the ability to bind to Pb-DOTAM, preferably with an affinity as described herein. The VH sequence may retain the invariant residues as set out above. In certain embodiments, a total of 1 to 10 amino acids have been substituted, inserted and/or deleted in SEQ ID NO: 7 or SEQ ID NO 9. In certain embodiments, substitutions, insertions, or deletions occur in regions outside the CDRs (i.e., in the FRs). Optionally, the antibody comprises the VH sequence in SEQ ID NO:7 or SEQ ID NO: 9, including post-translational modifications of that sequence, optionally with a C-terminal Ala. In a particular embodiment, the VH comprises one, two or three CDRs selected from: (a) CDR-H1 comprising the amino acid sequence of SEQ ID NO:1, (b) CDR-H2 comprising the amino acid sequence of SEQ ID NO:2, and (c) CDR-H3 comprising the amino acid sequence of SEQ ID NO:3.

In some embodiments, as mentioned above, in some variants SEQ ID NO: 7 or 9 may be extended by one or more additional C-terminal residues, e.g., by one or more alanine residues, optionally a single alanine residue. Thus, for instance, in one specific variant, the sequence of SEQ ID NO: 7 may be extended to be:

(SEQ ID NO.: 150)
VTLKESGPVLVKPTETLTLTCTVSGFSLSTYSMSWIRQPPGKA
LEWLGFIGSRGDTYYASWAKGRLTISKDTSKSQVVLTMTNMDP
VDTATYYCARERDPYGGGAYPPHLWGRGTLVTVSSA

In other embodiments, the extension may be by an N-terminal portion of the CH1 domain as described above, e.g., by 1-10 residues from the N-terminus of the CH1 domain, e.g., from the human IgG1 CH1 domain. For instance, the extension may be by the peptide sequence AST.

Optionally, the light chain variable domain forming part of the functional antigen binding site for Pb-DOTAM (on the second antibody) comprises an amino acid sequence of SEQ ID NO: 8, or a variant thereof comprising an amino acid sequence having at least 90, 91, 92, 93, 94, 95, 96, 97, 98, or 99% identity to SEQ ID NO: 8. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-Pb-DOTAM binding site comprising that sequence retains the ability to bind to Pb-DOTAM, preferably with an affinity as described herein. The VL sequence may retain the invariant residues as set out above. In certain embodiments, a total of 1 to 10 amino acids have been substituted, inserted and/or deleted in SEQ ID NO:8. In certain embodiments, the substitutions, insertions, or deletions occur in regions outside the CDRs (i.e., in the FRs). Optionally, the anti-Pb-DOTAM antibody comprises the VL sequence in SEQ ID NO:8, including post-translational modifications of that sequence. In a particular embodiment, the VL comprises one, two or three CDRs selected from (a) CDR-L1 comprising the amino acid sequence of SEQ ID NO:4; (b) CDR-L2 comprising the amino acid sequence of SEQ ID NO:5; and (c) CDR-L3 comprising the amino acid sequence of SEQ ID NO:6.

Embodiments concerned with the heavy chain variable region and the light chain variable region are explicitly contemplated in combination. Thus, the functional antigen binding site for Pb-DOTAM may be formed from a heavy chain variable region as defined above and a light chain variable region as defined above, on the first and second antibody respectively.

Optionally, the antigen binding site specific for the Pb-DOTAM chelate may be formed from a heavy chain variable domain comprising an amino acid sequence selected from the group consisting of SEQ ID NO: 7 or SEQ ID NO: 9, or a variant thereof as defined above (including a variant with a C-terminal extension as discussed above), and a light chain variable domain comprising an amino acid sequence of SEQ ID NO: 8, or a variant thereof as defined above. For example, the antigen binding site specific for the Pb-DOTAM chelate may comprise a heavy chain variable domain comprising the amino acid sequence of SEQ ID NO: 7 or a variant thereof, and a light chain variable domain comprising the amino acid sequence of SEQ ID NO: 8 or a variant thereof, including post-translational modifications of those sequences. In another embodiment, it may comprise a heavy chain variable domain comprising the amino acid sequence of SEQ ID NO: 9 or a variant thereof (including a variant with a C-terminal extension as discussed above) and a light chain variable domain comprising the amino acid sequence of SEQ ID NO: 8 or a variant thereof, including post-translational modifications of those sequences.

In any of the above embodiments, the light and heavy chain variable regions forming the anti-Pb-DOTAM binding site may be humanized. In one embodiment, the light and heavy chain variable region comprise CDRs as in any of the above embodiments, and further comprise an acceptor human framework, e.g. a human immunoglobulin framework or a human consensus framework. In another embodiment, the light and/or heavy chain variable regions comprise CDRs as in any of the above embodiments, and further comprises framework regions derived from vk 1 39 and/or vh 2 26. For vk 1 39, in some embodiments there may be no back mutations. For vh 2 26, the germline Ala49 residue may be backmutated to Gly49.

### E. Exemplary Antigen Binding Sites for CEA

In another particular embodiment of the present invention, which may be combined with the embodiments discussed above, the target antigen bound by the first and second antibody may be CEA (carcinoembryonic antigen). Antibodies that have been raised against CEA include T84.66 and humanized and chimeric versions thereof, such as T84.66-LCHA as described in WO2016/075278 A1 and/or WO2017/055389, CH1Ala, an anti-CEA antibody as described in WO2012/117002 and WO2014/131712, and CEA hMN-14 or labetuzimab (e.g., as described in U.S. Pat. Nos. 6,676,924 and 5,874,540). Another exemplary antibody against CEA is A5B7 (e.g., as described in M. J. Banfield et al, Proteins 1997, 29(2), 161-171), or a humanized antibody derived from murine A5B7 as described in WO 92/01059 and WO 2007/071422. See also co-pending application PCT/EP2020/067582. An example of a humanized version of A5B7 is A5H1EL1 (G54A). A further exemplary antibody against CEA is MFE23 and the humanized versions thereof described in U.S. Pat. No. 7,626,011 and/or co-pending application PCT/EP2020/067582. A still further example of an anti-CEA antibody is 28A9. Any of these or antigen binding fragments thereof may be used to form a CEA-binding moiety in the present invention.

Optionally, the antigen-binding site which binds to CEA may bind with a Kd value of 1 nM or less, 500 pM or less, 200 pM or less, or 100 pM or less for monovalent binding.

In some embodiments, the first and/or second antibody may bind to the CH1A1a epitope, the A5B7 epitope, the MFE23 epitope, the T84.66 epitope or the 28A9 epitope of CEA.

In some embodiments, at least one of the first and second antibodies binds to a CEA epitope which is not present on soluble CEA (sCEA). Soluble CEA is a part of the CEA molecule which is cleaved by GPI phospholipase and released into the blood. An example of an epitope not found on soluble CEA is the CH1A1A epitope. Optionally, one of the first and/or second antibody binds to an epitope which is not present on soluble CEA, and the other binds to an epitope which is present on soluble CEA.

The epitope for CH1A1a and its parent murine antibody PR1A3 is described in WO2012/117002A1 and Durbin H. et al., Proc. Natl. Scad. Sci. USA, 91:4313-4317, 1994. An antibody which binds to the CH1A1a epitope binds to a conformational epitope within the B3 domain and the GPI anchor of the CEA molecule. In one aspect, the antibody binds to the same epitope as the CH1A1a antibody having the VH of SEQ ID NO: 25 and VL of SEQ ID NO 26 herein. The A5B7 epitope is described in co-pending application PCT/EP2020/067582. An antibody which binds to the A5B7 epitope binds to the A2 domain of CEA, i.e., to the domain comprising the amino acids of SEQ ID NO: 154:

(SEQ ID NO: 154)
PKPFITSNNSNPVEDEDAVALTCEPEIQNTTYLWWVNNQSLPV
SPRLQLSNDNRTLTLLSVTRNDVGPYECGIQNKLSVDHSDPVI
LN.

In one aspect, the antibody binds to the same epitope as the A5B7 antibody having the VH of SEQ ID NO: 49 and VL of SEQ ID NO: 50 herein.

In one aspect, the antibody binds to the same epitope as the T84.66 described in WO2016/075278. The antibody may bind to the same epitope as the antibody having the VH of SEQ ID NO: 17 and VL of SEQ ID NO:18 herein.

The MFE23 epitope is described in co-pending application PCT/EP2020/067582. An antibody which binds to the MFE23 epitope binds to the A1 domain of CEA, i.e., to the domain comprising the amino acids of SEQ ID NO: 155:

(SEQ ID NO: 155)
PKPSISSNNSKPVEDKDAVAFTCEPETQDATYLWWVNNQSLPV
SPRLQLSNGNRTLTLFNVTRNDTASYKCETQNPVSARRSDSVI
LN.

In one aspect, the antibody may bind to the same epitope as an antibody having the VH domain of SEQ ID NO: 167 and the VL domain of SEQ ID NO: 168 herein.

In some embodiments, the first and/or second antibody may bind to the same CEA-epitope as an antibody provided herein, e.g., P1AD8749, P1AD8592, P1AE4956, P1AE4957, P1AF0709, P1AF0298, P1AF0710 or P1AF0711.

In some embodiments, the first and the second antibody bind the same epitope of CEA as each other. Thus, for example, the first and the second antibody may both bind to the CH1A1a epitope, the A5B7 epitope, the MFE23 epitope, the T84.66 epitope or the 28A9 epitope.

In some embodiments, both the first and second antibody may have CEA binding sequences (i.e., CDRs and/or VH/VL domains) from CH1A1A; or, the first and the second antibody may both have CEA binding sequences from A5B7 or a humanized version thereof; or, the first and the second antibody may both have CEA binding sequences from T84.66 or a humanized version thereof; or the first and the second antibody may both have CEA binding sequences from MFE23 or a humanized version thereof; or the first and second antibody may both have CEA binding sequences from 28A9 or a humanized version thereof. Exemplary sequences are disclosed herein.

In other embodiments, the first and the second antibodies bind to different epitopes of CEA. Thus, for example, i) one antibody may bind the CH1A1A epitope and the other may bind the A5B7 epitope, the T84.66 epitope, the MFE23 epitope or the 28A9 epitope; ii) one antibody may bind the A5B7 epitope and the other may bind the CH1A1A epitope, T84.66 epitope, MFE23 epitope or 28A9 epitope; iii) one antibody may bind the MFE23 epitope and the other may bind the CH epitope, A5B7 epitope, T84.66 epitope or 28A9 epitope; iv) one antibody may bind the T84.66 epitope and the other may bind the CH1A1A epitope, A5B7 epitope, MFE23 epitope or 28A9 epitope; or v) one antibody may bind the 28A9 epitope and the other may bind the CH1A1a epitope, the A5B7 epitope, the MFE23 epitope, or the T84.66 epitope.

In some embodiments, i) one antibody may have CEA binding sequences (i.e., CDRs or VH/VL domains) from CH1A1A and the other may have CEA binding sequences from A5B7 or a humanized version thereof, from T84.66 or a humanized version thereof, from MFE23 or a humanized version thereof, or from 28A9 or a humanized version thereof; ii) one antibody may have CEA binding sequences from A5B7 or a humanized version thereof and the other may have CEA binding sequences from CH1A1A, from T84.66 or a humanized version thereof, from MFE23 or a humanized version thereof, or from 28A9 or a humanized version thereof; iii) one antibody may have CEA binding sequences from MFE23 or a humanized version thereof and the other may have CEA binding sequences from CH1A1A, from A5B7 or a humanized version thereof, from T84.66 or a humanized version thereof, or from 28A9 or a humanized version thereof; iv) one antibody may have CEA binding sequences from T84.66 or a humanized version thereof and the other may have CEA binding sequences from CH1A1A, from A5B7 or a humanized version thereof, from MFE23 or a humanized version thereof, or from 28A9 or a humanized version; v) one antibody may have CEA-binding sequences from 28A9 or a humanized version thereof and the other may have CEA binding sequences from CH1A1A, from A5B7 or a humanized version thereof, from T84.66 or a humanized version thereof, or from MFE23 or a humanized version thereof.

In one particular embodiment, one antibody may bind the CH1A1A epitope and the other may bind the A5B7 epitope. The first antibody may have CEA binding sequences from the antibody CH and the second antibody may have CEA binding sequences from A5B7 (including a humanized version thereof); or, the first antibody may have CEA binding sequences from the antibody A5B7 (including a humanized version thereof) and the second antibody may have CEA binding sequences from CH1A1A.

In another particular embodiment, one antibody may bind the CH epitope and the other may bind the T84.66 epitope. The first antibody may have CEA binding sequences from the antibody CH and the second antibody may have CEA binding sequences from T84.66 (including a humanized version thereof); or, the first antibody may have CEA binding sequences from the antibody T84.66 (including a humanized version thereof) and the second antibody may have CEA binding sequences from CH1A1A. In some embodiments, a first antibody may bind the T84.66 epitope and/or have an antigen binding site as described in (i) below, and the second antibody may bind the CH1A1A epitope and/or have an antigen binding site as described in (ii) below.

Exemplary CEA-binding sequences i)-v) are disclosed below. These provide examples of CEA-binding sequences from i) T84.66, ii) CH1A1A, iii) A5B7, iv) 28A9 and v) MFE23 (or from humanized versions thereof).

i). In one embodiment, the antigen-binding site which binds to CEA may comprise at least one, two, three, four, five, or six CDRs selected from (a) CDR-H1 comprising the amino acid sequence of SEQ ID NO:11; (b) CDR-H2 comprising the amino acid sequence of SEQ ID NO:12; (c) CDR-H3 comprising the amino acid sequence of SEQ ID NO:13; (d) CDR-L1 comprising the amino acid sequence of SEQ ID NO:14; (e) CDR-L2 comprising the amino acid sequence of SEQ ID NO:15; and (f) CDR-L3 comprising the amino acid sequence of SEQ ID NO:16.

Optionally, the antigen-binding site which binds to CEA may comprise at least one, at least two, or all three VH CDR sequences selected from (a) CDR-H1 comprising the amino acid sequence of SEQ ID NO:11; (b) CDR-H2 comprising the amino acid sequence of SEQ ID NO:12; and (c) CDR-H3 comprising the amino acid sequence of SEQ ID NO:13.

Optionally, the antigen-binding site which binds to CEA comprises at least one, at least two, or all three VL CDRs sequences selected from (a) CDR-L1 comprising the amino acid sequence of SEQ ID NO:14; (b) CDR-L2 comprising the amino acid sequence of SEQ ID NO:15; and (c) CDR-L3 comprising the amino acid sequence of SEQ ID NO:16.

Optionally, the antigen-binding site which binds to CEA comprises (a) a VH domain comprising at least one, at least two, or all three VH CDR sequences selected from (i) CDR-H1 comprising the amino acid sequence of SEQ ID NO:11, (ii) CDR-H2 comprising the amino acid sequence of SEQ ID NO:12, and (iii) CDR-H3 comprising an amino acid sequence selected from SEQ ID NO:13; and (b) a VL domain comprising at least one, at least two, or all three VL CDR sequences selected from (i) CDR-L1 comprising the amino acid sequence of SEQ ID NO:14, (ii) CDR-L2 comprising the amino acid sequence of SEQ ID NO:15, and (c) CDR-L3 comprising the amino acid sequence of SEQ ID NO:16.

In another aspect, the antigen-binding site which binds to CEA comprises (a) CDR-H1 comprising the amino acid sequence of SEQ ID NO:11; (b) CDR-H2 comprising the amino acid sequence of SEQ ID NO:12; (c) CDR-H3 comprising the amino acid sequence of SEQ ID NO:13; (d) CDR-L1 comprising the amino acid sequence of SEQ ID NO:14; (e) CDR-L2 comprising the amino acid sequence of SEQ ID NO:15; and (f) CDR-L3 comprising the amino acid sequence of SEQ ID NO:16.

In any of the above embodiments, the multispecific antibody may be humanized. In one embodiment, the anti-CEA antigen binding site comprises CDRs as in any of the above embodiments, and further comprises an acceptor human framework, e.g. a human immunoglobulin framework or a human consensus framework.

In another embodiment, the antigen-binding site which binds to CEA comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO:17. In certain embodiments, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but the antigen binding site comprising that sequence retains the ability to bind to CEA, preferably with the affinity as set out above. In certain embodiments, a total of 1 to 10 amino acids have been substituted, inserted and/or deleted in SEQ ID NO:17. In certain embodiments, substitutions, insertions, or deletions occur in regions outside the HVRs (i.e., in the FRs). Optionally, the antigen-binding site which binds to CEA comprises the VH sequence in SEQ ID NO:17, including post-translational modifications of that sequence. In a particular embodiment, the VH comprises one, two or three CDRs selected from: (a) CDR-H1 comprising the amino acid sequence of SEQ ID NO:11, (b) CDR-H2 comprising the amino acid sequence of SEQ ID NO:12, and (c) CDR-H3 comprising the amino acid sequence of SEQ ID NO:13.

In another embodiment, the antigen-binding site which binds to CEA comprises a light chain variable domain (VL) having at least 90%, 91%, 92%, 93%, 94%, 95%, 96%, 97%, 98%, 99%, or 100% sequence identity to the amino acid sequence of SEQ ID NO:18. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but the antigen-binding site comprising that sequence retains the ability to bind to CEA, preferably with the affinity set out above. In certain embodiments, a total of 1 to 10 amino acids have been substituted, inserted and/or deleted in SEQ ID NO:18. In certain embodiments, the substitutions, insertions, or deletions occur in regions outside the HVRs (i.e., in the FRs). Optionally, the antigen-binding site for CEA comprises the VL sequence in SEQ ID NO:18, including post-translational modifications of that sequence. In a particular embodiment, the VL comprises one, two or three CDRs selected from (a) CDR-L1 comprising the amino acid sequence of SEQ ID NO:14; (b) CDR-L2 comprising the amino acid sequence of SEQ ID NO:15; and (c) CDR-L3 comprising the amino acid sequence of SEQ ID NO:16.

In another embodiment, the antigen-binding site which binds to CEA comprises a VH as in any of the embodiments provided above, and a VL as in any of the embodiments provided above. In one embodiment, the antibody comprises the VH and VL sequences in SEQ ID NO:17 and SEQ ID NO:18, respectively, including post-translational modifications of those sequences.

ii). In further particular embodiment, the antigen-binding site which binds to CEA may comprise at least one, two, three, four, five, or six CDRs selected from (a) CDR-H1 comprising the amino acid sequence of SEQ ID NO:19; (b) CDR-H2 comprising the amino acid sequence of SEQ ID NO:20; (c) CDR-H3 comprising the amino acid sequence of SEQ ID NO:21; (d) CDR-L1 comprising the amino acid sequence of SEQ ID NO:22; (e) CDR-L2 comprising the amino acid sequence of SEQ ID NO:23; and (f) CDR-L3 comprising the amino acid sequence of SEQ ID NO:24.

Optionally, the antigen-binding site which binds to CEA may comprise at least one, at least two, or all three VH CDR sequences selected from (a) CDR-H1 comprising the amino acid sequence of SEQ ID NO:19; (b) CDR-H2 comprising the amino acid sequence of SEQ ID NO:20; and (c) CDR-H3 comprising the amino acid sequence of SEQ ID NO:21.

Optionally, the antigen-binding site which binds to CEA comprises at least one, at least two, or all three VL CDRs sequences selected from (a) CDR-L1 comprising the amino acid sequence of SEQ ID NO:22; (b) CDR-L2 comprising the amino acid sequence of SEQ ID NO:23; and (c) CDR-L3 comprising the amino acid sequence of SEQ ID NO:24.

Optionally, the antigen-binding site which binds to CEA comprises (a) a VH domain comprising at least one, at least two, or all three VH CDR sequences selected from (i) CDR-H1 comprising the amino acid sequence of SEQ ID NO:19, (ii) CDR-H2 comprising the amino acid sequence of SEQ ID NO:20, and (iii) CDR-H3 comprising an amino acid sequence selected from SEQ ID NO:21; and (b) a VL domain comprising at least one, at least two, or all three VL CDR sequences selected from (i) CDR-L1 comprising the amino acid sequence of SEQ ID NO:22, (ii) CDR-L2 comprising the amino acid sequence of SEQ ID NO:23, and (c) CDR-L3 comprising the amino acid sequence of SEQ ID NO:24.

In another aspect, the antigen-binding site which binds to CEA comprises (a) CDR-H1 comprising the amino acid sequence of SEQ ID NO:19; (b) CDR-H2 comprising the amino acid sequence of SEQ ID NO:20; (c) CDR-H3 comprising the amino acid sequence of SEQ ID NO:21; (d) CDR-L1 comprising the amino acid sequence of SEQ ID NO:22; (e) CDR-L2 comprising the amino acid sequence of SEQ ID NO:23; and (f) CDR-L3 comprising the amino acid sequence of SEQ ID NO:24.

In any of the above embodiments, the multispecific antibody may be humanized. In one embodiment, the anti-CEA antigen binding site comprises CDRs as in any of the above embodiments, and further comprises an acceptor human framework, e.g. a human immunoglobulin framework or a human consensus framework.

In another embodiment, the antigen-binding site which binds to CEA comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO:25. In certain embodiments, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but the antigen binding site comprising that sequence retains the ability to bind to CEA, preferably with the affinity as set out above. In certain embodiments, a total of 1 to 10 amino acids have been substituted, inserted and/or deleted in SEQ ID NO:25. In certain embodiments, substitutions, insertions, or deletions occur in regions outside the HVRs (i.e., in the FRs). Optionally, the antigen-binding site which binds to CEA comprises the VH sequence in SEQ ID NO:25, including post-translational modifications of that sequence. In a particular embodiment, the VH comprises one, two or three CDRs selected from: (a) CDR-H1 comprising the amino acid sequence of SEQ ID NO:19, (b) CDR-H2 comprising the amino acid sequence of SEQ ID NO:20, and (c) CDR-H3 comprising the amino acid sequence of SEQ ID NO:21.

In another embodiment, the antigen-binding site which binds to CEA comprises a light chain variable domain (VL) having at least 90%, 91%, 92%, 93%, 94%, 95%, 96%, 97%, 98%, 99%, or 100% sequence identity to the amino acid sequence of SEQ ID NO:26. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but the antigen-binding site comprising that sequence retains the ability to bind to CEA, preferably with the affinity set out above. In certain embodiments, a total of 1 to 10 amino acids have been substituted, inserted and/or deleted in SEQ ID NO:26. In certain embodiments, the substitutions, insertions, or deletions occur in regions outside the HVRs (i.e., in the FRs). Optionally, the antigen-binding site for CEA comprises the VL sequence in SEQ ID NO:26, including post-translational modifications of that sequence. In a particular embodiment, the VL comprises one, two or three CDRs selected from (a) CDR-L1 comprising the amino acid sequence of SEQ ID NO:22; (b) CDR-L2 comprising the amino acid sequence of SEQ ID NO:23; and (c) CDR-L3 comprising the amino acid sequence of SEQ ID NO:24.

In another embodiment, the antigen-binding site which binds to CEA comprises a VH as in any of the embodiments provided above, and a VL as in any of the embodiments provided above. In one embodiment, the antibody comprises the VH and VL sequences in SEQ ID NO:25 and SEQ ID NO:26, respectively, including post-translational modifications of those sequences.

iii) In further particular embodiment, the antigen-binding site which binds to CEA may comprise at least one, two, three, four, five, or six CDRs selected from (a) CDR-H1 comprising the amino acid sequence of SEQ ID NO:43; (b) CDR-H2 comprising the amino acid sequence of SEQ ID NO:44; (c) CDR-H3 comprising the amino acid sequence of SEQ ID NO:45; (d) CDR-L1 comprising the amino acid sequence of SEQ ID NO:46; (e) CDR-L2 comprising the amino acid sequence of SEQ ID NO:47; and (f) CDR-L3 comprising the amino acid sequence of SEQ ID NO:48. In some embodiments, CDR-H1 may have the sequence GFTFTDYYMN (SEQ ID NO.: 151).

Optionally, the antigen-binding site which binds to CEA may comprise at least one, at least two, or all three VH CDR sequences selected from (a) CDR-H1 comprising the amino acid sequence of SEQ ID NO:43; (b) CDR-H2 comprising the amino acid sequence of SEQ ID NO:44; and (c) CDR-H3 comprising the amino acid sequence of SEQ ID NO:45. In some embodiments, CDR-H1 may have the sequence GFTFTDYYMN (SEQ ID NO.: 151).

Optionally, the antigen-binding site which binds to CEA comprises at least one, at least two, or all three VL CDRs sequences selected from (a) CDR-L1 comprising the amino acid sequence of SEQ ID NO:46; (b) CDR-L2 comprising the amino acid sequence of SEQ ID NO:47; and (c) CDR-L3 comprising the amino acid sequence of SEQ ID NO:48.

Optionally, the antigen-binding site which binds to CEA comprises (a) a VH domain comprising at least one, at least two, or all three VH CDR sequences selected from (i) CDR-H1 comprising the amino acid sequence of SEQ ID NO:43, (ii) CDR-H2 comprising the amino acid sequence of SEQ ID NO:44, and (iii) CDR-H3 comprising an amino acid sequence selected from SEQ ID NO:45; and (b) a VL domain comprising at least one, at least two, or all three VL CDR sequences selected from (i) CDR-L1 comprising the amino acid sequence of SEQ ID NO:46, (ii) CDR-L2 comprising the amino acid sequence of SEQ ID NO:47, and (c) CDR-L3 comprising the amino acid sequence of SEQ ID NO:48. In some embodiments, CDR-H1 may have the sequence GFTFTDYYMN (SEQ ID NO.: 151).

In another aspect, the antigen-binding site which binds to CEA comprises (a) CDR-H1 comprising the amino acid sequence of SEQ ID NO:43; (b) CDR-H2 comprising the amino acid sequence of SEQ ID NO:44; (c) CDR-H3 comprising the amino acid sequence of SEQ ID NO:45; (d) CDR-L1 comprising the amino acid sequence of SEQ ID NO:46; (e) CDR-L2 comprising the amino acid sequence of SEQ ID NO:47; and (f) CDR-L3 comprising the amino acid sequence of SEQ ID NO:48. In some embodiments, CDR-H1 may have the sequence GFTFTDYYMN (SEQ ID NO.: 151).

In any of the above embodiments, the multispecific antibody may be humanized. In one embodiment, the anti-CEA antigen binding site comprises CDRs as in any of the above embodiments, and further comprises an acceptor human framework, e.g. a human immunoglobulin framework or a human consensus framework.

In another embodiment, the antigen-binding site which binds to CEA comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO:49. In certain embodiments, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but the antigen binding site comprising that sequence retains the ability to bind to CEA, preferably with the affinity as set out above. In certain embodiments, a total of 1 to 10 amino acids have been substituted, inserted and/or deleted in SEQ ID NO:49. In certain embodiments, substitutions, insertions, or deletions occur in regions outside the HVRs (i.e., in the FRs). Optionally, the antigen-binding site which binds to CEA comprises the VH sequence in SEQ ID NO:49, including post-translational modifications of that sequence. In a particular embodiment, the VH comprises one, two or three CDRs selected from: (a) CDR-H1 comprising the amino acid sequence of SEQ ID NO:43 or the sequence GFTFTDYYMN (SEQ ID NO.: 151), (b) CDR-H2 comprising the amino acid sequence of SEQ ID NO:44, and (c) CDR-H3 comprising the amino acid sequence of SEQ ID NO:45.

In another embodiment, the antigen-binding site which binds to CEA comprises a light chain variable domain (VL) having at least 90%, 91%, 92%, 93%, 94%, 95%, 96%, 97%, 98%, 99%, or 100% sequence identity to the amino acid sequence of SEQ ID NO:50. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but the antigen-binding site comprising that sequence retains the ability to bind to CEA, preferably with the affinity set out above. In certain embodiments, a total of 1 to 10 amino acids have been substituted, inserted and/or deleted in SEQ ID NO:50. In certain embodiments, the substitutions, insertions, or deletions occur in regions outside the HVRs (i.e., in the FRs). Optionally, the antigen-binding site for CEA comprises the VL sequence in SEQ ID NO:50, including post-translational modifications of that sequence. In a particular embodiment, the VL comprises one, two or three CDRs selected from (a) CDR-L1 comprising the amino acid sequence of SEQ ID NO:46; (b) CDR-L2 comprising the amino acid sequence of SEQ ID NO:47; and (c) CDR-L3 comprising the amino acid sequence of SEQ ID NO:48.

In another embodiment, the antigen-binding site which binds to CEA comprises a VH as in any of the embodiments provided above, and a VL as in any of the embodiments provided above. In one embodiment, the antibody comprises the VH and VL sequences in SEQ ID NO:49 and SEQ ID NO:50, respectively, including post-translational modifications of those sequences.

iv) In a still further particular embodiment, the antigen-binding site which binds to CEA may comprise at least one, two, three, four, five, or six CDRs selected from (a) CDR-H1 comprising the amino acid sequence of SEQ ID NO:59; (b) CDR-H2 comprising the amino acid sequence of SEQ ID NO:60; (c) CDR-H3 comprising the amino acid sequence of SEQ ID NO:61; (d) CDR-L1 comprising the amino acid sequence of SEQ ID NO:62; (e) CDR-L2 comprising the amino acid sequence of SEQ ID NO:63; and (f) CDR-L3 comprising the amino acid sequence of SEQ ID NO:64.

Optionally, the antigen-binding site which binds to CEA may comprise at least one, at least two, or all three VH CDR sequences selected from (a) CDR-H1 comprising the amino acid sequence of SEQ ID NO:59; (b) CDR-H2 comprising the amino acid sequence of SEQ ID NO:60; and (c) CDR-H3 comprising the amino acid sequence of SEQ ID NO:61.

Optionally, the antigen-binding site which binds to CEA comprises at least one, at least two, or all three VL CDRs sequences selected from (a) CDR-L1 comprising the amino acid sequence of SEQ ID NO:62; (b) CDR-L2 comprising the amino acid sequence of SEQ ID NO:63; and (c) CDR-L3 comprising the amino acid sequence of SEQ ID NO:64.

Optionally, the antigen-binding site which binds to CEA comprises (a) a VH domain comprising at least one, at least two, or all three VH CDR sequences selected from (i) CDR-H1 comprising the amino acid sequence of SEQ ID NO:59, (ii) CDR-H2 comprising the amino acid sequence of SEQ ID NO:60, and (iii) CDR-H3 comprising an amino acid sequence selected from SEQ ID NO:61; and (b) a VL domain comprising at least one, at least two, or all three VL CDR sequences selected from (i) CDR-L1 comprising the amino acid sequence of SEQ ID NO:62, (ii) CDR-L2 comprising the amino acid sequence of SEQ ID NO:63, and (c) CDR-L3 comprising the amino acid sequence of SEQ ID NO:64.

In another aspect, the antigen-binding site which binds to CEA comprises (a) CDR-H1 comprising the amino acid sequence of SEQ ID NO:59; (b) CDR-H2 comprising the amino acid sequence of SEQ ID NO:60; (c) CDR-H3 comprising the amino acid sequence of SEQ ID NO:61; (d) CDR-L1 comprising the amino acid sequence of SEQ ID NO:62; (e) CDR-L2 comprising the amino acid sequence of SEQ ID NO:63; and (f) CDR-L3 comprising the amino acid sequence of SEQ ID NO:64.

In any of the above embodiments, the multispecific antibody may be humanized. In one embodiment, the anti-CEA antigen binding site comprises CDRs as in any of the above embodiments, and further comprises an acceptor human framework, e.g. a human immunoglobulin framework or a human consensus framework.

In another embodiment, the antigen-binding site which binds to CEA comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO:65. In certain embodiments, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but the antigen binding site comprising that sequence retains the ability to bind to CEA, preferably with the affinity as set out above. In certain embodiments, a total of 1 to 10 amino acids have been substituted, inserted and/or deleted in SEQ ID NO:65. In certain embodiments, substitutions, insertions, or deletions occur in regions outside the HVRs (i.e., in the FRs). Optionally, the antigen-binding site which binds to CEA comprises the VH sequence in SEQ ID NO:65, including post-translational modifications of that sequence. In a particular embodiment, the VH comprises one, two or three CDRs selected from: (a) CDR-H1 comprising the amino acid sequence of SEQ ID NO:59, (b) CDR-H2 comprising the amino acid sequence of SEQ ID NO:60, and (c) CDR-H3 comprising the amino acid sequence of SEQ ID NO:61.

In another embodiment, the antigen-binding site which binds to CEA comprises a light chain variable domain (VL) having at least 90%, 91%, 92%, 93%, 94%, 95%, 96%, 97%, 98%, 99%, or 100% sequence identity to the amino acid sequence of SEQ ID NO:66. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but the antigen-binding site comprising that sequence retains the ability to bind to CEA, preferably with the affinity set out above. In certain embodiments, a total of 1 to 10 amino acids have been substituted, inserted and/or deleted in SEQ ID NO:66. In certain embodiments, the substitutions, insertions, or deletions occur in regions outside the HVRs (i.e., in the FRs). Optionally, the antigen-binding site for CEA comprises the VL sequence in SEQ ID NO:66, including post-translational modifications of that sequence. In a particular embodiment, the VL comprises one, two or three CDRs selected from (a) CDR-L1 comprising the amino acid sequence of SEQ ID NO:62; (b) CDR-L2 comprising the amino acid sequence of SEQ ID NO:63; and (c) CDR-L3 comprising the amino acid sequence of SEQ ID NO:64.

In another embodiment, the antigen-binding site which binds to CEA comprises a VH as in any of the embodiments provided above, and a VL as in any of the embodiments provided above. In one embodiment, the antibody comprises the VH and VL sequences in SEQ ID NO:65 and SEQ ID NO:66, respectively, including post-translational modifications of those sequences.

v). In a still further particular embodiment, the antigen-binding site which binds to CEA may comprise at least one, two, three, four, five, or six CDRs selected from (a) CDR-H1 comprising the amino acid sequence of SEQ ID NO:156; (b) CDR-H2 comprising the amino acid sequence of SEQ ID NO:157 or 158; (c) CDR-H3 comprising the amino acid sequence of SEQ ID NO:159; (d) CDR-L1 comprising the amino acid sequence of SEQ ID NO:160, 161 or 162; (e) CDR-L2 comprising the amino acid sequence of SEQ ID NO:163, 164 or 165; and (0 CDR-L3 comprising the amino acid sequence of SEQ ID NO:166.

Optionally, the antigen-binding site which binds to CEA may comprise:

VH CDR sequences (a) CDR-H1 comprising the amino acid sequence of SEQ ID NO:156; (b) CDR-H2 comprising the amino acid sequence of SEQ ID NO:157 or 158; and (c) CDR-H3 comprising the amino acid sequence of SEQ ID NO:159; and/or

VL CDRs sequences (a) CDR-L1 comprising the amino acid sequence of SEQ ID NO:160, 161 or 162; (b) CDR-L2 comprising the amino acid sequence of SEQ ID NO:163, 164 or 165; and (c) CDR-L3 comprising the amino acid sequence of SEQ ID NO:166.

In one embodiment, the antigen binding site for CEA comprises a heavy chain variable region (VH) comprise the amino acid sequence of SEQ ID NO: 167, or (more preferably) selected from SEQ ID NO: 169, 170, 171, 172, 173 or 174, and a light chain variable region (VL) comprising the amino acid sequence of SEQ ID NO: 168 or (more preferably) selected from SEQ ID NO: 175, 176, 177, 178, 179 or 180.

In any of the above embodiments, the multispecific antibody may be humanized. In one embodiment, the anti-CEA antigen binding site comprises CDRs as in any of the above embodiments, and further comprises an acceptor human framework, e.g. a human immunoglobulin framework or a human consensus framework.

In a particular aspect, the antigen binding domain capable of binding to CEA comprises:

(a) a VH domain comprising an amino acid sequence of SEQ ID NO:169 and a VL domain comprising an amino acid sequence of SEQ ID NO:179, or

(b) a VH domain comprising an amino acid sequence of SEQ ID NO:173 and a VL domain comprising an amino acid sequence of SEQ ID NO:179, or

(c) a VH domain comprising an amino acid sequence of SEQ ID NO:170 and a VL domain comprising an amino acid sequence of SEQ ID NO:179, or

(d) a VH domain comprising an amino acid sequence of SEQ ID NO:174 and a VL domain comprising an amino acid sequence of SEQ ID NO:178, or

(e) a VH domain comprising an amino acid sequence of SEQ ID NO:173 and a VL domain comprising an amino acid sequence of SEQ ID NO:178, or

(f) a VH domain comprising an amino acid sequence of SEQ ID NO:171 and a VL domain comprising an amino acid sequence of SEQ ID NO:178, or

(g) a VH domain comprising an amino acid sequence of SEQ ID NO:169 and a VL domain comprising an amino acid sequence of SEQ ID NO:178.

In another embodiment, the antigen-binding site which binds to CEA comprises a heavy chain variable domain (VH) sequence having at least 90%, 91%, 92%, 93%, 94%, 95%, 96%, 97%, 98%, 99%, or 100% sequence identity to the amino acid sequence as mentioned in a) to g) above. In certain embodiments, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but the antigen binding site comprising that sequence retains the ability to bind to CEA, preferably with the affinity as set out above. In certain embodiments, a total of 1 to 10 amino acids have been substituted, inserted and/or deleted. In certain embodiments, substitutions, insertions, or deletions occur in regions outside the HVRs (i.e., in the FRs).

In another embodiment, the antigen-binding site which binds to CEA comprises a light chain variable domain (VL) having at least 90%, 91%, 92%, 93%, 94%, 95%, 96%, 97%, 98%, 99%, or 100% sequence identity to the amino acid sequence as mentioned in a) to g) above. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but the antigen-binding site comprising that sequence retains the ability to bind to CEA, preferably with the affinity set out above. In certain embodiments, a total of 1 to 10 amino acids have been substituted, inserted and/or deleted. In certain embodiments, the substitutions, insertions, or deletions occur in regions outside the HVRs (i.e., in the FRs).

In another embodiment, the antigen-binding site which binds to CEA comprises a VH as in any of the embodiments provided above, and a VL as in any of the embodiments provided above.

### F. Exemplary Antigen Binding Sites for Other Targets

In another particular embodiment of the present invention, which may be combined with the embodiments discussed above (e.g., the binding sites for DOTA or DOTAM), the target antigen bound by the first and second antibody may be GPRC5D or FAP.

Optionally, the antigen-binding site which binds to GPRC5D or FAP may bind with a Kd value of 1 nM or less, 500 pM or less, 200 pM or less, or 100 pM or less for monovalent binding.

Exemplary GPRC5D-binding sequences are described below.

In one embodiment, the antigen-binding site which binds to GPRC5D may comprise at least one, two, three, four, five, or six CDRs selected from (a) CDR-H1 comprising the amino acid sequence of SEQ ID NO:67; (b) CDR-H2 comprising the amino acid sequence of SEQ ID NO:68; (c) CDR-H3 comprising the amino acid sequence of SEQ ID NO:69; (d) CDR-L1 comprising the amino acid sequence of SEQ ID NO:70; (e) CDR-L2 comprising the amino acid sequence of SEQ ID NO:71; and (f) CDR-L3 comprising the amino acid sequence of SEQ ID NO:72.

Optionally, the antigen-binding site which binds to GPRC5D may comprise at least one, at least two, or all three VH CDR sequences selected from (a) CDR-H1 comprising the amino acid sequence of SEQ ID NO:67; (b) CDR-H2 comprising the amino acid sequence of SEQ ID NO:68; and (c) CDR-H3 comprising the amino acid sequence of SEQ ID NO:69. Optionally, the antigen-binding site which binds to GPRC5D comprises at least one, at least two, or all three VL CDRs sequences selected from (a) CDR-L1 comprising the amino acid sequence of SEQ ID NO:70; (b) CDR-L2 comprising the amino acid sequence of SEQ ID NO:71; and (c) CDR-L3 comprising the amino acid sequence of SEQ ID NO:72.

Optionally, the antigen-binding site which binds to GPRC5D comprises (a) a VH domain comprising at least one, at least two, or all three VH CDR sequences selected from (i) CDR-H1 comprising the amino acid sequence of SEQ ID NO:67, (ii) CDR-H2 comprising the amino acid sequence of SEQ ID NO:68, and (iii) CDR-H3 comprising an amino acid sequence selected from SEQ ID NO:69; and (b) a VL domain comprising at least one, at least two, or all three VL CDR sequences selected from (i) CDR-L1 comprising the amino acid sequence of SEQ ID NO:70, (ii) CDR-L2 comprising the amino acid sequence of SEQ ID NO:71, and (c) CDR-L3 comprising the amino acid sequence of SEQ ID NO:72.

In another aspect, the antigen-binding site which binds to GPRC5D comprises (a) CDR-H1 comprising the amino acid sequence of SEQ ID NO:67; (b) CDR-H2 comprising the amino acid sequence of SEQ ID NO:68; (c) CDR-H3 comprising the amino acid sequence of SEQ ID NO:69; (d) CDR-L1 comprising the amino acid sequence of SEQ ID NO:70; (e) CDR-L2 comprising the amino acid sequence of SEQ ID NO:71; and (f) CDR-L3 comprising the amino acid sequence of SEQ ID NO:72.

In any of the above embodiments, the multispecific antibody may be humanized. In one embodiment, the anti-GPRC5D antigen binding site comprises CDRs as in any of the above embodiments, and further comprises an acceptor human framework, e.g. a human immunoglobulin framework or a human consensus framework.

In another embodiment, the antigen-binding site which binds to GPRC5D comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO:73. In certain embodiments, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but the antigen binding site comprising that sequence retains the ability to bind to GPRC5D, preferably with the affinity as set out above. In certain embodiments, a total of 1 to 10 amino acids have been substituted, inserted and/or deleted in SEQ ID NO:73. In certain embodiments, substitutions, insertions, or deletions occur in regions outside the HVRs (i.e., in the FRs). Optionally, the antigen-binding site which binds to GPRC5D comprises the VH sequence in SEQ ID NO:73, including post-translational modifications of that sequence. In a particular embodiment, the VH comprises one, two or three CDRs selected from: (a) CDR-H1 comprising the amino acid sequence of SEQ ID NO:67, (b) CDR-H2 comprising the amino acid sequence of SEQ ID NO:68, and (c) CDR-H3 comprising the amino acid sequence of SEQ ID NO:69.

In another embodiment, the antigen-binding site which binds to GPRC5D comprises a light chain variable domain (VL) having at least 90%, 91%, 92%, 93%, 94%, 95%, 96%, 97%, 98%, 99%, or 100% sequence identity to the amino acid sequence of SEQ ID NO:74. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but the antigen-binding site comprising that sequence retains the ability to bind to GPRC5D, preferably with the affinity set out above. In certain embodiments, a total of 1 to 10 amino acids have been substituted, inserted and/or deleted in SEQ ID NO:74. In certain embodiments, the substitutions, insertions, or deletions occur in regions outside the HVRs (i.e., in the FRs). Optionally, the antigen-binding site for GPRC5D comprises the VL sequence in SEQ ID NO:74, including post-translational modifications of that sequence. In a particular embodiment, the VL comprises one, two or three CDRs selected from (a) CDR-L1 comprising the amino acid sequence of SEQ ID NO:70; (b) CDR-L2 comprising the amino acid sequence of SEQ ID NO:71; and (c) CDR-L3 comprising the amino acid sequence of SEQ ID NO:72.

In another embodiment, the antigen-binding site which binds to GPRC5D comprises a VH as in any of the embodiments provided above, and a VL as in any of the embodiments provided above. In one embodiment, the antibody comprises the VH and VL sequences in SEQ ID NO:73 and SEQ ID NO:74, respectively, including post-translational modifications of those sequences.

Exemplary FAP-binding sequences are described below.

In one embodiment, the antigen-binding site which binds to FAP may comprise at least one, two, three, four, five, or six CDRs selected from (a) CDR-H1 comprising the amino acid sequence of SEQ ID NO:75; (b) CDR-H2 comprising the amino acid sequence of SEQ ID NO:76; (c) CDR-H3 comprising the amino acid sequence of SEQ ID NO:77; (d) CDR-L1 comprising the amino acid sequence of SEQ ID NO:78; (e) CDR-L2 comprising the amino acid sequence of SEQ ID NO:79; and (f) CDR-L3 comprising the amino acid sequence of SEQ ID NO:80.

Optionally, the antigen-binding site which binds to FAP may comprise at least one, at least two, or all three VH CDR sequences selected from (a) CDR-H1 comprising the amino acid sequence of SEQ ID NO:75; (b) CDR-H2 comprising the amino acid sequence of SEQ ID NO:76; and (c) CDR-H3 comprising the amino acid sequence of SEQ ID NO:77.

Optionally, the antigen-binding site which binds to FAP comprises at least one, at least two, or all three VL CDRs sequences selected from (a) CDR-L1 comprising the amino acid sequence of SEQ ID NO:78; (b) CDR-L2 comprising the amino acid sequence of SEQ ID NO:79; and (c) CDR-L3 comprising the amino acid sequence of SEQ ID NO:80.

Optionally, the antigen-binding site which binds to FAP comprises (a) a VH domain comprising at least one, at least two, or all three VH CDR sequences selected from (i) CDR-H1 comprising the amino acid sequence of SEQ ID NO:75, (ii) CDR-H2 comprising the amino acid sequence of SEQ ID NO:76, and (iii) CDR-H3 comprising an amino acid sequence selected from SEQ ID NO:77; and (b) a VL domain comprising at least one, at least two, or all three VL CDR sequences selected from (i) CDR-L1 comprising the amino acid sequence of SEQ ID NO:78, (ii) CDR-L2 comprising the amino acid sequence of SEQ ID NO:79, and (c) CDR-L3 comprising the amino acid sequence of SEQ ID NO:80.

In another aspect, the antigen-binding site which binds to FAP comprises (a) CDR-H1 comprising the amino acid sequence of SEQ ID NO:75; (b) CDR-H2 comprising the amino acid sequence of SEQ ID NO:76; (c) CDR-H3 comprising the amino acid sequence of SEQ ID NO:77; (d) CDR-L1 comprising the amino acid sequence of SEQ ID NO:78; (e) CDR-L2 comprising the amino acid sequence of SEQ ID NO:79; and (f) CDR-L3 comprising an amino acid sequence SEQ ID NO:80.

In any of the above embodiments, the multispecific antibody may be humanized. In one embodiment, the anti-FAP antigen binding site comprises CDRs as in any of the above embodiments, and further comprises an acceptor human framework, e.g. a human immunoglobulin framework or a human consensus framework.

In another embodiment, the antigen-binding site which binds to FAP comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO:81. In certain embodiments, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but the antigen binding site comprising that sequence retains the ability to bind to FAP, preferably with the affinity as set out above. In certain embodiments, a total of 1 to 10 amino acids have been substituted, inserted and/or deleted in SEQ ID NO:81. In certain embodiments, substitutions, insertions, or deletions occur in regions outside the HVRs (i.e., in the FRs). Optionally, the antigen-binding site which binds to FAP comprises the VH sequence in SEQ ID NO:81, including post-translational modifications of that sequence. In a particular embodiment, the VH comprises one, two or three CDRs selected from: (a) CDR-H1 comprising the amino acid sequence of SEQ ID NO:75, (b) CDR-H2 comprising the amino acid sequence of SEQ ID NO:76, and (c) CDR-H3 comprising the amino acid sequence of SEQ ID NO:77.

In another embodiment, the antigen-binding site which binds to FAP comprises a light chain variable domain (VL) having at least 90%, 91%, 92%, 93%, 94%, 95%, 96%, 97%, 98%, 99%, or 100% sequence identity to the amino acid sequence of SEQ ID NO:82. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but the antigen-binding site comprising that sequence retains the ability to bind to FAP, preferably with the affinity set out above. In certain embodiments, a total of 1 to 10 amino acids have been substituted, inserted and/or deleted in SEQ ID NO:82. In certain embodiments, the substitutions, insertions, or deletions occur in regions outside the HVRs (i.e., in the FRs). Optionally, the antigen-binding site for FAP comprises the VL sequence in SEQ ID NO:82, including post-translational modifications of that sequence. In a particular embodiment, the VL comprises one, two or three CDRs selected from (a) CDR-L1 comprising the amino acid sequence of SEQ ID NO:78; (b) CDR-L2 comprising the amino acid sequence of SEQ ID NO:79; and (c) CDR-L3 comprising the amino acid sequence of SEQ ID NO:80.

In another embodiment, the antigen-binding site which binds to FAP comprises a VH as in any of the embodiments provided above, and a VL as in any of the embodiments provided above. In one embodiment, the antibody comprises the VH and VL sequences in SEQ ID NO:81 and SEQ ID NO:82, respectively, including post-translational modifications of those sequences.

### G. Antibody Formats

As described above, the present invention relates to a set of antibodies comprising:

i) a first antibody that binds to an antigen expressed on the surface of a target cell, and which further comprises a VH domain of an antigen binding site for a radiolabelled compound, but which does not comprise a VL domain of an antigen binding site for the radiolabelled compound; and

ii) a second antibody that binds to the antigen expressed on the surface of the target cell, and which further comprises a VL domain of an antigen binding site for the radiolabelled compound, but which does not comprise a VH domain of the antigen binding site for the radiolabelled compound,

wherein said VH domain of the first antibody and said VL domain of the second antibody are together capable of forming a functional antigen binding site for the radiolabelled compound.

In some embodiments, the first and second antibody may each comprise an Fc domain. The presence of an Fc region has benefits in the context of radioimmunotherapy and radioimaging, e.g. prolonging the protein's circulating half-life and/or resulting in higher tumour uptake than may be observed with smaller fragments.

In some embodiments, where the Fc region is present, it may be preferred that the Fc region is engineered to reduce or eliminate effector function. This may include substitution of one or more of Fc region residues 234, 235, 238, 265, 269, 270, 297, 327 and/or 329, e.g., one or more of 234, 235 and/or 329. In some embodiments, the Fc region may be engineered to include the substitution of Pro 329 to Gly, Leu 234 to Ala and/or Leu 235 to Ala (numbering according to EU index).

In some embodiments, as discussed above, where the VH domain of an antigen binding site for a radiolabelled compound is free at its C-terminus (e.g., is not fused to another domain via its C-terminus), then it may be extended by one or more residues to avoid binding of HAVH autoantibodies. For instance, the extension may be by 1-10 residues, e.g., 1, 2, 3, 4, 5, 6, 7, 8, 9 or 10 residues. In one embodiment, it may be extended by one or more alanine residues, optionally by one alanine residue. The VH sequence may also be extended by an N-terminal portion of the CH1 domain, e.g., by 1-10 residues from the N-terminus of the CH1 domain, e.g., from the human IgG1 CH1 domain. (The first ten residues of the human IgG1 CH1 domain are ASTKGPSVFP (SEQ ID NO.: 149), and so in one embodiment, from 1-10 residues may be taken from the N-terminus of this sequence). For instance, in one embodiment, the peptide sequence AST (corresponding to the first 3 residues of the IgG1 CH1 domain) is added to the C-terminus of the VH region. In some embodiments, the first and/or the second antibody may each be multivalent, e.g., bivalent for the target antigen (e.g., the tumour associated antigen). This has the advantage of increasing avidity.

In some embodiments, it may be preferred that when the first and second antibody are associated, they form an antibody complex which is monovalent for the radiolabelled compound. Thus, the first antibody may comprise only one VH domain of an antigen binding site for the radiolabelled compound, and the second antibody may comprise only one VL domain of an antigen binding site for a radiolabelled compound, so that together they form only one complete functional binding site for the radiolabelled compound.

The antibodies may each comprise i) at least one antibody fragment comprising an antigen binding site specific for the target antigen, ii) either a VL domain or a VH domain of the antigen binding site for the radiolabelled compound, and iii) optionally a Fc region. The antibody fragment may be for example at least one Fv, scFv, Fab or cross-Fab fragment, comprising an antigen binding site specific for the target antigen. The antibody fragment may be fused to a) either a VL domain or a VH domain of the antigen binding site for the radiolabelled compound or b) if the antibodies comprise a Fc region, to a Fc region which is fused to either a VL domain or a VH domain of the antigen binding site for the radiolabelled compound. In some embodiments, the C-terminus of the Fc region is fused to the N-terminus of the VL domain or VH domain.

The fusion may be direct or indirect. In some embodiments, the fusion may be via a linker. For instance, the Fc region may be fused to the antibody fragment via the hinge region or another suitable linker. Similarly, the connection of the VL or VH domain of the antigen binding site for the radiolabelled compound to the rest of the antibody structure may be made via a linker. The linker may be a peptide of at least 5 amino acids, preferably 5 to 100, more preferably 10 to 50 or 25 to 50 amino acids. The linker may be a rigid linker or a flexible linker. In some embodiments, it is a flexible linker comprising or consisting of Thr, Ser, Gly and/or Ala residues. For example, it may comprise or consist of Gly and Ser residues. In some embodiments it may have a repeating motif such as (Gly-Gly-Gly-Gly-Ser)n, where n is for instance 1, 2, 3, 4, 5, 6, 7, 8, 9 or 10. In another embodiment said peptide linker is (GxS)n or (GxS)nGm with G=glycine, S=serine, and (x=3, n=3, 4, 5 or 6, and m=0, 1, 2 or 3) or (x=4, n=2, 3, 4 or 5 and m=0, 1, 2 or 3), e.g., x=4 and n=2 or 3, e.g., with x=4, n=2. In some embodiments, the linker may be or may comprise the sequence GGGGSGGGGSGGGGSGGGGS (SEQ ID NO.: 31). Other linkers may be used and could be identified by the skilled person.

In one particular embodiment, the first antibody may comprise or consist of:

a) an scFv fragment, wherein the scFv fragment binds the target antigen; and

b) a polypeptide comprising or consisting of
i) an antibody heavy chain variable domain (VH); or
ii) an antibody heavy chain variable domain (VH) and an antibody heavy chain constant domain, wherein the C-terminus of the VH domain is fused to the N terminus of the constant domain;

wherein said polypeptide is fused by the N-terminus of the VH domain, preferably via a peptide linker, to the C-terminus of scFv fragment.

The second antibody may comprise or consist of:

c) a second scFv binding the target antigen; and

d) a polypeptide comprising or consisting of
i) an antibody light chain variable domain (VL); or
ii) an antibody light chain variable domain (VL) and an antibody light chain constant domain, wherein the C-terminus of the VL domain is fused to the N-terminus of the constant domain;
   wherein said polypeptide is fused by the N-terminus of the VL domain, preferably via a peptide linker, to the C-terminus of scFv fragment.

The antibody heavy chain variable domain (VH) of the first antibody and the antibody light chain variable domain (VL) of the second antibody together form a functional antigen-binding site for the radiolabelled compound, upon association of the two antibodies.

Optionally, the polypeptide of part b (i) may additionally comprise one or more residues at the C-terminus of the VH domain, optionally, one or more alanine residues, optionally a single alanine residue. Optionally, the additional residues may be an N-terminal portion of the CH1 domain as described above, e.g., 1-10 residues from the N-terminus of the CH1 domain, e.g., from the human IgG1 CH1 domain. For instance, the additional residues may be AST.

The target antigen-recognizing variable domains of the heavy and light chain of an scFv can be connected by a peptide tether. Such a peptide tether may comprise 1 to 25 amino acids, preferably 12 to 20 amino acids, preferably 12 to 16 or 15 to 20 amino acids. The above described tether may comprise one or more (G3S) and/or (G4S) motifs, in particular 1, 2, 3, 4, 5 or 6 (G3S) and/or (G4S) motives, preferably 3 or 4 (G3S) and/or (G4S) motives, more preferably 3 or 4 (G4S) motives.

Optionally, the first antibody may consist essentially of or consist of the components (a) and (b) listed above and the second antibody may consist or consist essentially of the components (c) and (d) listed above. In any event, the first antibody does not comprise an antibody light chain variable domain (VL) capable of forming a functional antigen-binding site for the radiolabelled compound in association with component (b) of the first antibody; and the second antibody does not comprise an antibody heavy chain variable (VH) domain capable of forming a functional antigen-binding site for the radiolabelled compound in association with component (d) of the second antibody.

In another particular embodiment, the first antibody may comprise or consist of:

a) a Fab fragment binding the target antigen, and

b) a polypeptide comprising or consisting of
i) an antibody heavy chain variable domain (VH) of an antigen binding site for a radiolabelled compound, or
ii) an antibody heavy chain variable domain (VH) of an antigen binding site for a radiolabelled compound and an antibody heavy chain constant domain, wherein the C-terminus of VH domain is fused to the N-terminus of the constant domain;
wherein the polypeptide is fused by the N-terminus of the VH domain, preferably via a peptide linker, to the C terminus of the CL or CH1 domain of the Fab fragment.
   The second antibody may comprise or consist of:

c) a Fab fragment binding the target antigen, and

d) a polypeptide comprising or consisting of
iii) an antibody light chain variable domain (VL) of an antigen binding site for a radiolabelled compound, or
iv) an antibody light chain variable domain (VL) of an antigen binding site for a radiolabelled compound and an antibody light chain constant domain, wherein the C-terminus of the VL domain is fused to the N-terminus of the constant domain;

wherein the polypeptide is fused by the N-terminus of the VL domain, preferably via a peptide linker, to the C-terminus of the CL or CH1 domain of the Fab fragment.

The antibody heavy chain variable domain (VH) of the polypeptide of (b) and antibody light chain variable domain (VL) of polypeptide of (d) together form a functional antigen-binding site for the radiolabelled compound (i.e., upon association of the two antibodies).

Optionally, the polypeptide of part b (i) may additionally comprise one or more residues at the C-terminus of the VH domain as described above, optionally, one or more alanine residues, optionally a single alanine residue. Optionally, the additional residues may be an N-terminal portion of the CH1 domain as described above, e.g., 1-10 residues from the N-terminus of the CH1 domain, e.g., from the human IgG1 CH1 domain. For instance, the additional residues may be AST.

Optionally, the first antibody may consist essentially of or consist of the components (a) and (b) listed above and the second antibody may consist or consist essentially of the components (c) and (d) listed above. In any event, the first antibody does not comprise an antibody light chain variable domain (VL) capable of forming a functional antigen-binding site for the radiolabelled compound in association with component (b) of the first antibody; and the second antibody does not comprise an antibody heavy chain variable (VH) domain capable of forming a functional antigen-binding site for the radiolabelled compound in association with component (d) of the second antibody.

The chain of the Fab fragment which is fused to the polypeptide can be independently selected for the first and for the second anybody. Thus, in one embodiment, the polypeptide of (b) is fused to the C-terminus of the CH1 domain of the Fab fragment of the first antibody, and the polypeptide of (d) is fused to the C-terminus of the CH1 domain of the Fab fragment of the second antibody. In another embodiment, the polypeptide of (b) is fused to the C-terminus of the CL domain of the Fab fragment of the first antibody, and the polypeptide of (d) is fused to the C-terminus of the CL domain of the Fab fragment of the second antibody. In another embodiment, the polypeptide of (b) is fused to the C-terminus of the CH1 domain of the Fab fragment of the first antibody, and polypeptide of (d) is fused to the C-terminus of the CL domain of the Fab fragment of the second antibody. In a further embodiment, polypeptide of (b) is fused to the C-terminus of the CL domain of the Fab fragment of the first antibody, and the polypeptide of (d) is fused to the C-terminus of the CH1 domain of the Fab fragment of the second antibody.

As noted above, in some embodiments, the first and/or the second antibody may each be multivalent, e.g., bivalent for the target antigen (e.g., the tumour associated antigen). This has the advantage of increasing avidity. The antibodies may be multivalent, e.g., bivalent, and may each be monospecific for a particular epitope (which may be the same epitope for the first and second antibody, or may be different for the first and second antibody). Thus, in some embodiments, the first antibody may comprise i) two or more antibody fragments comprising an antigen binding site specific for the same epitope of the target antigen, ii) either a VL domain or a VH domain of the antigen binding site for the radiolabelled compound (but not both), and iii) optionally a Fc region. The second antibody may comprise i) two or more antibody fragments comprising an antigen binding site specific for the same epitope of the target antigen, ii) either a VL domain or a VH domain of the antigen binding site for the radiolabelled compound (but not both), and iii) optionally a Fc region. As stated above, the epitope may be the same for the first and second antibody, or may be different for the first and second antibody

For example, each of the first and the second antibody may comprise a tandem Fab, i.e., two Fab fragments, which are connected via a peptide tether (Fab-tether-Fab), wherein the first Fab is connected via its C-terminus to the N-terminus of the second Fab.

In one embodiment, the first antibody comprises

a) a tandem Fab comprising two Fab fragments, wherein the first and the second Fab fragment bind the same target antigen (“target antigen A”) and the epitope bound by the first Fab fragment is the same as the epitope bound by the second Fab fragment, and wherein the first and the second Fab fragment are connected via a peptide tether, wherein the first Fab is connected via its C-terminus to the N-terminus of the second Fab; and

b) a polypeptide comprising or consisting of
i) an antibody heavy chain variable domain (VH); or
ii) an antibody heavy chain variable domain (VH) and an antibody constant domain (CH1), wherein the C-terminus of VH domain is fused to the N-terminus of the CH1 domain;

wherein said polypeptide is fused by the N-terminus of the VH domain, preferably via a peptide linker, to the C-terminus of the CL or CH1 domain of the second Fab fragment;

and the second antibody comprises

c) a tandem Fab comprising two Fab fragments, wherein the first and the second Fab fragment bind target antigen A and the epitope bound by the first Fab fragment is the same as the epitope bound by the second Fab fragment, and wherein the first and the second Fab fragment are connected via a peptide tether, wherein the first Fab is connected via its C-terminus to the N-terminus of the second Fab; and

d) a polypeptide comprising or consisting of

i) an antibody light chain variable domain (VL); or

ii) an antibody light chain variable domain (VL) and an antibody light chain constant domain (CL), wherein the C-terminus of VH domain is fused to the N-terminus of the constant domain;

wherein said polypeptide is fused by the N-terminus of the VL domain, preferably via a peptide linker, to the C-terminus of the CL or CH1 domain of the second Fab fragment.

The antibody heavy chain variable domain (VH) of part b (in the first antibody) and the antibody light chain variable domain (VL) of part (d) (in the second antibody) together form a functional antigen-binding site for the radiolabelled compound, i.e., upon association of the two antibodies.

Optionally, the polypeptide of part b (i) may additionally comprise one or more residues at the C-terminus of the VH domain, optionally, one or more alanine residues, optionally a single alanine residue. Optionally, the additional residues may be an N-terminal portion of the CH1 domain as described above, e.g., 1-10 residues from the N-terminus of the CH1 domain, e.g., from the human IgG1 CH1 domain. For instance, the additional residues may be AST.

The chain of the Fab tandem which is fused to the polypeptide (i.e., whether the polypeptide is fused to the CL or the CH1 domain of the second Fab fragment) can be independently selected for the first and for the second anybody.

As described above, the first Fab fragment of the Fab tandem is connected to the N-terminus of the second Fab fragment. In one embodiment, the C-terminus of the heavy chain fragment of the first Fab fragment is connected to the N-terminus of the heavy-chain fragment or light chain fragment of the second Fab fragment. In another embodiment, the C-terminus light chain fragment of the first Fab fragment is connected to the N-terminus of the heavy-chain fragment or light chain fragment of the second Fab fragment. Thus, in some embodiments the Fab tandem of the first and/or second antibody may comprise three chains as follows:

1) the light chain fragment ((VLCL)1) of the first Fab fragment, the heavy chain fragment of the first Fab fragment connected to the heavy chain fragment of the second Fab fragment via a peptide tether ((VHCH1)1-tether-(VHCH1)2) and the light chain fragment of the second Fab fragment ((VLCL)2); or

2) the light chain fragment of the first Fab fragment ((VLCL)1), the heavy chain fragment of the first Fab fragment connected to the light chain fragment of the second Fab fragment via a peptide tether ((VHCH1)1-tether-(VLCL)2) and the heavy chain fragment of the second Fab fragment ((VH-CH1)2); or

3) the heavy chain fragment (VHCH1) of the first Fab fragment, the light chain fragment of the first Fab fragment connected to the light chain fragment of the second Fab fragment via a peptide tether ((VLCL)1-tether-(VLCL)2) and the heavy chain fragment of the second Fab fragment; or

4) the heavy chain fragment (VHCH1) of the first Fab fragment, the light chain fragment of the first Fab fragment connected to the heavy chain fragment of the second Fab fragment via a peptide tether ((VLCL)1-tether-(VHCH1)2) and the light chain fragment of the second Fab fragment ((VLCL)2).

In another embodiment, the first and/or second antibody may each bind more than one, optionally two, different epitopes of the target antigen. Thus, one or both of the antibodies may be biparatopic for the target antigen. In some embodiments, the first and second antibody may each comprise i) an antibody fragment comprising an antigen binding site specific for a first epitope of target antigen A; ii) an antibody fragment comprising an antigen binding site for a second epitope of target antigen A, iii) either a VL domain or a VH domain of the antigen binding site for the radiolabelled compound (but not both), and iv) optionally a Fc region.

In such embodiments, correct assembly of the light chains with their respective heavy chain can be assisted by using cross-mab technology. For instance, in one embodiment, each antibody may comprise a tandem Fab comprising one Fab and one cross-Fab, in which one fragment selected from the Fab and the cross-Fab is specific for a first epitope, and the other is specific for a second epitope.

In one particular example, the first antibody may comprise:

a) a tandem Fab comprising a first fragment and a second fragment, wherein the first fragment is connected by its C-terminus via a peptide tether to the N-terminus of the second fragment, wherein the first fragment binds a first epitope of target antigen A and the second fragment binds a second epitope of target antigen A, and wherein one of the fragments selected from the first and second fragments is a Fab and the other is a cross-Fab,

b) a polypeptide comprising or consisting of
i) an antibody heavy chain variable domain (VH); or
ii) an antibody heavy chain variable domain (VH) and an antibody heavy chain constant domain (CH1), wherein the C-terminus of VH domain is fused to the N-terminus of the CH1 domain;

wherein said polypeptide is fused by the N-terminus of the VH domain, preferably via a peptide linker, to the C-terminus of one of the chains of the second fragment.

The second antibody may comprise

c) a tandem Fab comprising a first fragment and a second fragment, wherein the first fragment is connected by its C-terminus to the N-terminus of the second fragment, wherein the first fragment binds a first epitope of target antigen A and the second fragment binds a second epitope of target antigen A, and wherein one of the fragments selected from the first and second fragments is a Fab and the other is a cross-Fab; and

d) a polypeptide comprising or consisting of

i) an antibody light chain variable domain (VL); or

ii) an antibody light chain variable domain (VL) and an antibody light chain constant domain (CL), wherein the C-terminus of VL domain is fused to the N-terminus of the light chain constant domain

wherein said polypeptide is fused by the N-terminus of the VL domain, preferably via a peptide linker, to the C-terminus of one of the chains of the second fragment.

The antibody heavy chain variable domain (VH) of the first antibody and the antibody light chain variable domain (VL) of the second antibody together form a functional antigen-binding site for the radiolabelled compound.

Optionally, the polypeptide of part b (i) may additionally comprise one or more residues at the C-terminus of the VH domain, optionally, one or more alanine residues, optionally a single alanine residue. Optionally, the additional residues may be an N-terminal portion of the CH1 domain as described above, e.g., 1-10 residues from the N-terminus of the CH1 domain, e.g., from the human IgG1 CH1 domain. For instance, the additional residues may be AST.

Either the first or second fragment can be the cross-Fab, as long as the tandem Fab comprises one conventional Fab and one cross Fab.

In any of the tandem Fab embodiments described above (including those involving cross-Fabs), optionally, the first antibody may consist essentially of or consist of the components (a) and (b) and the second antibody may consist or consist essentially of the components (c) and (d). In any event, the first antibody does not comprise an antibody light chain variable domain (VL) capable of forming a functional antigen-binding site for the radiolabelled compound in association with component (b) of the first antibody; and the second antibody does not comprise an antibody heavy chain variable (VH) domain capable of forming a functional antigen-binding site for the radiolabelled compound in association with component (d) of the second antibody.

In any of the tandem Fab embodiments (including those involving cross-Fabs), the peptide tether connecting the Fab fragments in the first and second antibody may be a peptide with an amino acid sequence with a length of at least 5 amino acids, preferably with a length of 5 to 100, more preferably of 10 to 50 amino acids. In one embodiment said peptide linker is (GxS)n or (GxS)nGm with G=glycine, S=serine, and (x=3, n=3, 4, 5 or 6, and m=0, 1, 2 or 3) or (x=4, n=2, 3, 4 or 5 and m=0, 1, 2 or 3), preferably x=4 and n=2 or 3, more preferably with x=4, n=2. In one embodiment said peptide tether is (G4S)₂.

As noted above, in some embodiments, the first and second antibody may each comprise an Fc domain, optionally engineered to reduce or eliminate effector function.

In one embodiment, each of the first and second antibody may comprise i) an Fc domain, ii) at least one antibody fragment, such as an scFv, Fv, Fab or cross-Fab fragment, comprising an antigen binding site specific for the target antigen and iii) either a VL domain or a VH domain of the antigen binding site for the radiolabelled compound (but not both).

Optionally, the antibodies comprising the Fc domain may be monovalent in respect of binding to the target antigen. In other embodiments, they may be multivalent, e.g., bivalent. The first and second antibodies may each be multivalent and monospecific for the same epitope of the target antigen. In still other embodiments, the first and second antibodies may each have binding sites for different epitopes of the target antigen—e.g., they may be biparatopic.

The antibody fragment may be an scFv. Thus, in one embodiment, the first antibody may comprise or consist of:

a) an scFv fragment, wherein the scFv fragment binds the target antigen;

b) an Fc domain; and

c) a polypeptide comprising or consisting of

i) an antibody heavy chain variable domain (VH); or

ii) an antibody heavy chain variable domain (VH) and an antibody heavy chain constant domain (CH1), wherein the C-terminus of the VH domain is fused to the N-terminus of the constant domain;

wherein the scFv of (a) is fused to the N-terminus of the Fc domain, and wherein the polypeptide of c) is fused by the N-terminus of the VH domain to the C-terminus of the Fc domain, preferably via a peptide linker.

Optionally, the polypeptide of part c (i) may additionally comprise one or more residues at the C-terminus of the VH domain, optionally, one or more alanine residues, optionally a single alanine residue. Optionally, the additional residues may be an N-terminal portion of the CH1 domain as described above, e.g., 1-10 residues from the N-terminus of the CH1 domain, e.g., from the human IgG₁ CH1 domain. For instance, the additional residues may be AST.

The second antibody may comprise or consist of:

d) a second scFv binding the target antigen;

e) an Fc domain; and

f) a polypeptide comprising or consisting of

i) an antibody light chain variable domain (VL); or

ii) an antibody light chain variable domain (VL) and an antibody light chain constant domain (CL), wherein the C-terminus of the VL domain is fused to the N-terminus of the constant domain;

wherein the scFv of (d) is fused to the N-terminus of the Fc domain, and wherein the polypeptide of (0 is fused by the N-terminus of the VH domain to the C-terminus of the Fc domain, preferably via a peptide linker.

In another embodiment, the first and second antibody may each be a one-armed IgG comprising a Fab for the target antigen (e.g., a single Fab for the target antigen) and an Fc domain. Thus, the first antibody may comprise or consist of:

i) a complete light chain fragment;

ii) a complete heavy chain;

iii) an additional Fc chain lacking Fd; and

iv) a polypeptide comprising or consisting of the VH domain of the antigen binding site for the radiolabeled compound;

wherein the light chain of (i) and the heavy chain of (ii) together provide an antigen binding site for the target antigen; and wherein the polypeptide comprising or consisting of the VH domain of the antigen binding site for the radiolabeled compound is fused by its N-terminus, preferably via a linker, to the C-terminus of either (ii) or (iii).

The second antibody may comprise or consist of

v) a complete light chain fragment;

vi) a complete heavy chain;

vii) an additional Fc chain lacking Fd; and

viii) a polypeptide comprising or consisting of the VL domain of the antigen binding site for the radiolabeled compound;

wherein the light chain of (v) and the heavy chain of (vi) together provide an antigen binding site for the target antigen; and wherein the polypeptide comprising or consisting of the VL domain of the antigen binding site for the radiolabeled compound is fused by its N-terminus, preferably via a linker, to the C-terminus of either (vi) or (vii).

The polypeptide comprising or consisting of the VH domain of the antigen binding site for the radiolabeled compound may be a polypeptide comprising or consisting of

i) an antibody heavy chain variable domain (VH), in which case the polypeptide may additionally comprise one or more residues at the C-terminus of the VH domain, optionally, one or more alanine residues, optionally a single alanine residue, or optionally an N-terminal portion of the CH1 domain as described above; or

ii) an antibody heavy chain variable domain (VH) and an antibody heavy chain constant domain (CH1), wherein the C-terminus of VH domain is fused to the N-terminus of the CH1 domain.

The polypeptide comprising or consisting of the VL domain of the antigen binding site for the radiolabeled compound may be a polypeptide comprising or consisting of
i) an antibody heavy chain variable domain (VL); or
ii) an antibody heavy chain variable domain (VL) and an antibody light chain constant domain, wherein the C-terminus of VL domain is fused to the N-terminus of the constant domain.

When the first and second antibodies are heterodimers, e.g., as for one-armed IgGs, their assembly may be assisted by the use of knob-into-hole technology, as described further below.

In another embodiment, the antibodies may each comprise a tandem Fab as described above (e.g., comprising two Fab fragments, wherein the first and the second Fab fragment both bind the same epitope of target antigen A; or comprising a Fab and a cross Fab wherein one of them binds a first epitope of target antigen A and the other binds a second epitope of target antigen A), wherein the Fab tandem is fused (e.g., via its C-terminus) to the N-terminus of an Fc domain, and wherein peptide comprising or consisting of the VH or VL domain of the antigen binding site for the radiolabelled compound is fused (e.g., via its N-terminus) to the C-terminus of the Fc domain.

Thus, the first antibody may comprise or consist of:

a) a tandem Fab selected from
i) a tandem Fab comprising two Fab fragments, wherein the first and the second Fab fragment bind target antigen A and the epitope bound by the first Fab fragment is the same as the epitope bound by the second Fab fragment, and wherein the first and the second Fab fragment are connected via a peptide tether, wherein the first Fab is connected via its C-terminus to the N-terminus of the second Fab; and
ii) a tandem Fab comprising a first fragment and a second fragment, wherein the first fragment is connected by its C-terminus via a peptide tether to the N-terminus of the second fragment, wherein the first fragment binds a first epitope of target antigen A and the second fragment binds a second epitope of target antigen A, and wherein one of the fragments selected from the first and second fragments is a Fab and the other is a cross-Fab;

b) an Fc domain; and

c) a polypeptide comprising or consisting of:
i) an antibody heavy chain variable domain (VH); or
ii) an antibody heavy chain variable domain (VH) and an antibody heavy chain constant domain (CH1), wherein the C-terminus of VH domain is fused to the N-terminus of the CH1 domain,
   wherein the tandem Fab is fused to the N-terminus of one of the chains of the Fc domain, and the polypeptide of c) is fused by the N-terminus of the VH domain to the C-terminus of one of the chains of the Fc domain, preferably via a peptide linker.

Optionally, the polypeptide of part c (i) may additionally comprise one or more residues at the C-terminus of the VH domain, optionally, one or more alanine residues, optionally a single alanine residue. Optionally, the additional residues may be an N-terminal portion of the CH1 domain as described above, e.g., 1-10 residues from the N-terminus of the CH1 domain, e.g., from the human IgG1 CH1 domain. For instance, the additional residues may be AST.

The second antibody may comprise or consist of:

d) a tandem Fab selected from:
i) a tandem Fab comprising two Fab fragments, wherein the first and the second Fab fragment bind target antigen A and the epitope bound by the first Fab fragment is the same as the epitope bound by the second Fab fragment, and wherein the first and the second Fab fragment are connected via a peptide tether, wherein the first Fab is connected via its C-terminus to the N-terminus of the second Fab; and
ii) a tandem Fab comprising a first fragment and a second fragment, wherein the first fragment is connected by its C-terminus via a peptide tether to the N-terminus of the second fragment, wherein the first fragment binds a first epitope of target antigen A and the second fragment binds a second epitope of target antigen A, and wherein one of the fragments selected from the first and second fragments is a Fab and the other is a cross-Fab;

e) an Fc domain; and

f) a polypeptide comprising or consisting of:
i) an antibody heavy chain variable domain (VL); or
ii) an antibody heavy chain variable domain (VL) and an antibody light chain constant domain, wherein the C-terminus of VL domain is fused to the N-terminus of the light chain constant domain,

wherein the tandem Fab of (d) is fused to the N-terminus one of the chains of the Fc domain, and the polypeptide of (f) is fused by the N-terminus of the VL domain to the C-terminus of one of the chains of the Fc domain, preferably via a peptide linker.

The VH domain of the first antibody and the VL domain of the second antibody together form an antigen binding site for the radiolabelled compound, i.e., upon association of the two antibodies.

If the first antibody comprises a tandem Fab according to (a)(i), then it will generally be the case that the second antibody will comprise a tandem Fab according to d (i); if the first antibody comprises a tandem Fab according to (a)(ii), then it will generally be the case that the second antibody will comprise a tandem Fab according to d (ii).

The tandem Fab may be generally as described above. For instance, the tether linking the two fragments of the tandem Fab may be as described above. The tandem Fab may be composed of any of the sets of chains set out above. Generally, the heavy chain fragment of the second Fab (which may be a cross-Fab) can be linked to the Fc domain.

In a further embodiment, each of the first and second antibody may comprise a) an Fc domain b) at least one antibody fragment, such as an scFv, Fv, Fab or cross-Fab fragment, comprising an antigen binding site for the target antigen and c) a polypeptide comprising either a VL domain or a VH domain of the antigen binding site for the radiolabelled compound (but not both), wherein the C-terminus of the antibody fragment of (b) is fused to the N-terminus of one chain of the Fc domain, and the C-terminus of the polypeptide of (c) is fused to the N-terminus of the other chain of the Fc domain. The fusion of the antibody fragment of (b) is preferably via the hinge region. The fusion of the polypeptide of (c) may be via a linker positioned between the C-terminus of polypeptide and the N-terminus of the Fc region and/or via some or all of the upper hinge region (e.g., the Asp221 and residues C-terminal thereto according to the EU numbering index). In one embodiment, the antibody fragment of (b) may be a Fab fragment. In one embodiment, in the first antibody, the polypeptide of (c) consists of the VH domain of the antigen binding site for the radiolabelled compound; and in the second antibody the polypeptide of (c) consists of the VL domain of the antigen binding site for the radiolabelled compound.

Thus, in one embodiment, the first antibody may comprise or consist of:

i) a complete light chain;

ii) a complete heavy chain;

iii) an additional Fc chain; and

iv) a polypeptide comprising or consisting of the VH domain of the antigen binding site for the radiolabeled compound;

wherein the light chain of (i) and the heavy chain of (ii) together provide an antigen binding site for the target antigen; and wherein the polypeptide comprising or consisting of the VH domain of the antigen binding site for the radiolabeled compound is fused by its C-terminus, preferably via a linker, to the N-terminus of (iii).

The second antibody may comprise or consist of

v) a complete light chain;

vi) a complete heavy chain;

vii) an additional Fc chain; and

viii) a polypeptide comprising or consisting of the VL domain of the antigen binding site for the radiolabeled compound;

wherein the light chain of (v) and the heavy chain of (vi) together provide an antigen binding site for the target antigen; and wherein the polypeptide comprising or consisting of the VL domain of the antigen binding site for the radiolabeled compound is fused by its c-terminus, preferably via a linker, to the N-terminus of (vii).

The linker may comprise any flexible linker as known to the person skilled in the art, e.g., the linker GGGGSGGGGSGGGGSGGSGG (SEQ ID NO.: 152). The linker may further include part of all of the upper hinge region, e.g., may extend from Asp221 to the start of the Fc chain (e.g., at Cys226).

In a still further embodiment, the first and/or second antibody each comprise a full length antibody having an antigen binding site for the target antigen, and further comprise either a VL domain or a VH domain of the antigen binding site for the radiolabelled compound.

In one particular embodiment, the first antibody may comprise:

a) a first full length antibody consisting of two antibody heavy chains and two antibody light chains, wherein at least one arm of the full length antibody binds to target antigen A; and

b) a polypeptide comprising or consisting of
i) an further antibody heavy chain variable domain (VH); or
ii) a further antibody heavy chain variable domain (VH) and an further antibody constant domain (CH1), wherein the C-terminus of VH domain is fused to the N-terminus of the CH1 domain,
wherein said polypeptide is fused by the N-terminus of the VH domain, preferably via a peptide linker, to the C-terminus of one of the two heavy chains of said first full-length antibody.

The second antibody may comprise

c) a second full length antibody consisting of two antibody heavy chains and two antibody light chains, wherein at least one arm of the full length antibody binds to target antigen A; and

d) a polypeptide comprising or consisting of
i) a further antibody light chain variable domain (VL); or
ii) a further antibody light chain variable domain (VL) and a further antibody light chain constant domain (CL), wherein the C-terminus of VL domain is fused to the N-terminus of the CL domain,
wherein said polypeptide is fused by the N-terminus of the VL domain, preferably via a peptide linker, to the C-terminus of one of the two heavy chains of said second full-length antibody.

The antibody heavy chain variable domain (VH) of the first antibody and the antibody light chain variable domain (VL) of the second antibody together form a functional antigen-binding site for the radiolabelled compound, i.e., upon association of the two antibodies.

Optionally, the polypeptide of part b (i) may additionally comprise one or more residues at the C-terminus of the VH domain, optionally, one or more alanine residues, optionally a single alanine residue. Optionally, the additional residues may be an N-terminal portion of the CH1 domain as described above, e.g., 1-10 residues from the N-terminus of the CH1 domain, e.g., from the human IgG1 CH1 domain. For instance, the additional residues may be AST.

Optionally, the first antibody may consist essentially of or consist of the components (a) and (b) listed above, and the second antibody may consist essentially of or consist of the components (c) and (d) listed above. In any event, the first antibody does not comprise an antibody light chain variable domain (VL) capable of forming a functional antigen-binding site for the radiolabelled compound in association with component (b) of the first antibody; and the second antibody does not comprise an antibody heavy chain variable (VH) domain capable of forming a functional antigen-binding site for the radiolabelled compound in association with component (b) of the second antibody.

It may be preferred that both arms of the full length antibody have binding specificity for target antigen A. Where the antibody is bivalent for the target antigen, both arms of the full length antibody may bind to the same epitope of target antigen A.

In another embodiment, the antibody may be biparatopic for the target antigen; e.g., one arm of the full length antibody may bind to a first epitope of target antigen A and one arm may bind to a second epitope of target antigen A. In such embodiments, one arm of the antibody may comprise a Fab and one arm may comprise a cross-Fab, to assist in correct assembly of the light chains with their respective heavy chain. Thus, in one embodiment, the first heavy chain of the full length antibody may comprise a VL domain in place of the VH domain (e.g., VL-CH1-hinge-CH2-CH3) and the first light chain may comprise a VH domain exchanged for the VL domain (e.g., VH-CL), or the first heavy chain may comprise a CL domain in place of the HC1 domain (e.g., VH-CL-hinge-CH2-CH3) and the first light chain may comprise a CH1 domain in place of the CL domain (e.g., VL-CH1). In this embodiment, the second heavy chain and the second light chain have the conventional domain structure (e.g., VH-CH1-hinge-CH2-CH3 and VL-CL, respectively). In an alternative embodiment, the second heavy chain of the full length antibody may comprise a VL domain in place of the VH domain (e.g., VL-CH1-hinge-CH2-CH3) and the second light chain may comprise a VH domain exchanged for the VL domain (e.g., VH-CL), or the second heavy chain may comprise a CL domain in place of the HC1 domain (e.g., VH-CL-hinge-CH2-CH3) and the second light chain may comprise a CH1 domain in place of the CL domain (e.g., VL-CH1). In this embodiment, the first heavy chain and the first light chain have the conventional domain structure.

In some embodiments, correct assembly of the light chains with their respective heavy chain can additionally or alternatively be assisted by using charge modification, as discussed further below.

Correct assembly of heterodimeric heavy chains can be assisted by knob-into-hole technology.

As used herein, the term “full length antibody” denotes an antibody consisting of two “full length antibody heavy chains” and two “full length antibody light chains”. A “full length antibody heavy chain” may be a polypeptide consisting in N-terminal to C-terminal direction of an antibody heavy chain variable domain (VH), an antibody constant heavy chain domain 1 (CH1), an antibody hinge region (HR), an antibody heavy chain constant domain 2 (CH2), and an antibody heavy chain constant domain 3 (CH3), abbreviated as VH-CH1-HR-CH2-CH3; and optionally an antibody heavy chain constant domain 4 (CH4) in case of an antibody of the subclass IgE. Preferably the “full length antibody heavy chain” is a polypeptide consisting in N-terminal to C-terminal direction of VH, CH1, HR, CH2 and CH3. The possibility of cross-Mab formation is not intended to be excluded by the reference to “full length”—thus, the heavy chain may have the VH domain swapped for a VL domain, or the CH1 domain swapped for a CL domain. A “full length antibody light chain” may be a polypeptide consisting in N-terminal to C-terminal direction of an antibody light chain variable domain (VL), and an antibody light chain constant domain (CL), abbreviated as VL-CL. Alternatively, in the case of a cross-Mab, the VL domain may be swapped for a VH domain or the CL domain may be swapped for a CH1 domain. The antibody light chain constant domain (CL) can be κ (kappa) or γ (lambda). The two full length antibody chains are linked together via inter-polypeptide disulfide bonds between the CL domain and the CH1 domain and between the hinge regions of the full length antibody heavy chains. Examples of typical full length antibodies are natural antibodies like IgG (e.g. IgG1 and IgG2), IgM, IgA, IgD, and IgE.) The full length antibodies according to the invention can be from a single species e.g. human, or they can be chimerized or humanized antibodies. The full length antibodies described herein comprise two antigen binding sites each formed by a pair of VH and VL, which may in some embodiments both specifically bind to the same antigen, or may bind to different antigens. The C-terminus of the heavy or light chain of said full length antibody denotes the last amino acid at the C-terminus of said heavy or light chain.

The N-terminus of the antibody heavy chain variable domain (VH) of the polypeptide under b) and the antibody light chain variable domain (VL) of the polypeptide under d) denotes the last amino acid at the N-terminus of VH or VL domain.

Techniques which are known for making multispecific antibodies can also be used to make any of the heterodimers described herein. These include, but are not limited to, recombinant co-expression of two immunoglobulin heavy chain-light chain pairs having different specificities (see Milstein and Cuello, Nature 305: 537 (1983)) and “knob-in-hole” engineering (see, e.g., U.S. Pat. No. 5,731,168, and Atwell et al., J. Mol. Biol. 270:26 (1997)). Other methods include engineering electrostatic steering effects for making antibody Fc-heterodimeric molecule (see, e.g., WO 2009/089004); cross-linking two or more antibodies or fragments (see, e.g., U.S. Pat. No. 4,676,980, and Brennan et al., Science, 229: 81 (1985)); using leucine zippers (see, e.g., Kostelny et al., J. Immunol., 148(5):1547-1553 (1992) and WO 2011/034605); and using the common light chain technology for circumventing the light chain mis-pairing problem (see, e.g., WO 98/50431).

The CH3 domains of the full length antibody as described above can be altered by the “knob-into-holes” technology which is described in detail with several examples in e.g. WO 96/027011, Ridgway, J. B., et al., Protein Eng 9 (1996) 617-621; and Merchant, A. M., et al., Nat Biotechnol 16 (1998) 677-681. In this method the interaction surfaces of the two CH3 domains are altered to increase the heterodimerisation of both heavy chains containing these two CH3 domains. Each of the two CH3 domains (of the two heavy chains) can be the “knob”, while the other is the “hole”. For instance one comprises called “knob mutations” (T366W and optionally one of S354C or Y349C) and the other comprises the so-called “hole mutations” (T366S, L368A and Y407V and optionally Y349C or 5354C) (see, e.g., Carter, P. et al., Immunotechnol. 2 (1996) 73) according to EU index numbering.

The introduction of a disulfide bridge may additionally or alternatively be used to stabilize the heterodimers (Merchant, A. M., et al., Nature Biotech 16 (1998) 677-681; Atwell, S., et al., J. Mol. Biol. 270 (1997) 26-35) and increase the yield.

Thus in some embodiments the first and/or second antibody is further characterized in that: the CH3 domain of one heavy chain of the full length antibody and the CH3 domain of the other heavy chain of the full length antibody each meet at an interface which comprises an original interface between the antibody CH3 domains; wherein said interface is altered to promote the formation of the antibody, wherein the alteration is characterized in that:

a) the CH3 domain of one heavy chain is altered, so that within the original interface the CH3 domain of one heavy chain that meets the original interface of the CH3 domain of the other heavy chain within the antibody, an amino acid residue is replaced with an amino acid residue having a larger side chain volume, thereby generating a protuberance within the interface of the CH3 domain of one heavy chain which is positionable in a cavity within the interface of the CH3 domain of the other heavy chain and

b) the CH3 domain of the other heavy chain is altered, so that within the original interface of the second CH3 domain that meets the original interface of the first CH3 domain within the antibody an amino acid residue is replaced with an amino acid residue having a smaller side chain volume, thereby generating a cavity within the interface of the second CH3 domain within which a protuberance within the interface of the first CH3 domain is positionable.

Said amino acid residue having a larger side chain volume may optionally be selected from the group consisting of arginine (R), phenylalanine (F), tyrosine (Y), tryptophan (W). Said amino acid residue having a smaller side chain volume may optionally be selected from the group consisting of alanine (A), serine (S), threonine (T), valine (V).

Optionally, in some embodiments, both CH3 domains are further altered by the introduction of cysteine (C) as amino acid in the corresponding positions of each CH3 domain such that a disulfide bridge between both CH3 domains can be formed.

The multispecific (e.g., biparatopic) antibodies of the invention may comprise amino acid substitutions in Fab molecules (including cross-Fab molecules) comprised therein which are particularly efficient in reducing mispairing of light chains with non-matching heavy chains (Bence-Jones-type side products), which can occur in the production of Fab-based bi-/multispecific antigen binding molecules with a VH/VL exchange in one (or more, in case of molecules comprising more than two antigen-binding Fab molecules) of their binding arms (see also PCT publication no. WO 2015/150447, particularly the examples therein, incorporated herein by reference in its entirety). The ratio of a desired multispecific antibodies compared to undesired side products, in particular Bence Jones-type side products occurring in one of their binding arms, can be improved by the introduction of charged amino acids with opposite charges at specific amino acid positions in the CH1 and CL domains of a Fab molecule (sometimes referred to herein as “charge modifications”).

Therefore, in some embodiments, the antibodies of the present invention comprising Fab molecules, comprises at least one Fab with a heavy chain constant domain CH1 domain comprising charge modifications as described herein, and a light chain constant CL domain comprising charge modifications as described herein.

Charge modifications can be made either in the conventional Fab molecule(s) comprised in the antibodies of the present invention, or in the crossover Fab molecule(s) comprised in the antibodies of the present invention (but not in both). In particular embodiments, the charge modifications are made in the conventional Fab molecule(s) comprised in the antibodies of the present invention.

In some embodiments, in a Fab or cross-Fab comprising a light chain constant domain CL comprising charge modifications and a heavy chain constant domain CH1 comprising charge modifications, charge modifications in the light chain constant domain CL are at position 124 and optionally at position 123 (numbering according to Kabat), and charge modifications in the heavy chain constant domain CH1 are at position 147 and/or 213 (numbering according to Kabat EU Index). In some embodiments, in the light chain constant domain CL the amino acid at position 124 is substituted independently by lysine (K), arginine (R) or histidine (H) (numbering according to Kabat) (in one preferred embodiment independently by lysine (K)), and in the heavy chain constant domain CH1 the amino acid at position 147 and/or the amino acid at position 213 is substituted independently by glutamic acid (E) or aspartic acid (D) (numbering according to Kabat EU index.

### H. Exemplary Antibodies

Aspects and embodiments concerning target binding (e.g., CEA-binding, FAP-binding or GPRC5D-binding) and aspects and embodiments concerning DOTA binding can in some embodiments be combined. That is, it may be preferred that the first and second antibody each comprise a binding site for CEA, FAP or GPRC5D, e.g., comprising any of the sequences as described above, and that the first and second antibodies associate to form a binding site for a DOTA chelate having any of the sequences as described above. It is also expressly contemplated that aspects and embodiments concerning CEA binding, FAP or GPRC5D and/or DOTA binding can be combined with preferred formats for the antibody as described above—i.e., in any of the preferred formats, the part that binds the target antigen may comprise CDRs or variable regions sequences as described above, and/or the part that binds the radionuclide-labelled compound may be a DOTA binder having CDRs and/or variable region sequences as described above.

In one particular embodiment, the first antibody may comprise:

a) a first full length antibody specifically binding to CEA and consisting of two antibody heavy chains and two antibody light chains; and

b) a polypeptide comprising or consisting of an antibody heavy chain variable domain (VH) wherein the heavy chain variable domain comprises heavy chain CDRs of SEQ ID NOs 35-37 (or wherein CDR-H1 has the sequence GFSLTDYGVH (SEQ ID NO.: 148)), and/or wherein the heavy chain variable domain has at least 90, 91, 92, 93, 94, 95, 96, 97, 98, 99 or 100% identity to SEQ ID NO 41;

wherein said polypeptide is fused with the N-terminus of the VH domain, preferably via a peptide linker, to the C-terminus of one of the two heavy chains of said first full-length antibody.

The first antibody does not comprise a light chain domain which associates with the polypeptide of (b) to form a functional binding domain for a radiolabelled compound.

It may be preferred that the polypeptide of (b) further comprises one or more residues at the C-terminus of the VH domain, e.g., 1-10 residues. Optionally, these may be one or more alanine residues, optionally a single alanine residue. In another embodiment, the additional residues may be an N-terminal portion of the CH1 domain as described above, e.g., 1-10 residues from the N-terminus of the CH1 domain, e.g., from the human IgG₁ CH1 domain. For instance, the additional residues may be AST.

In some embodiments, the two antibody heavy chains in part (a) have identical variable domains, optionally identical variable, CH1 and/or CH2 domains. They may optionally differ only in their CH3 domains, e.g., by the creation of knob into hole mutations and other mutations intended to promote the correct association of heterodimers.

The second antibody may comprise:

c) a second full length antibody specifically binding CEA and consisting of two antibody heavy chains and two antibody light chains; and

d) a polypeptide comprising or consisting of an antibody light chain variable domain (VL) wherein the light chain variable domain comprises CDRs of SEQ ID NO: 38-40 and/or wherein the light chain variable domain has at least 90, 91, 92, 93, 94, 95, 96, 97, 98, 99 or 100% identity to SEQ ID NO 42;

wherein said polypeptide is fused with the N-terminus of the VL domain, preferably via a peptide linker, to the C-terminus of one of the two heavy chains of said second full-length antibody and wherein the second antibody does not comprise a heavy chain domain which associates with the polypeptide of (d) to form a functional binding domain for a radiolabelled compound.

In some embodiments, the two antibody heavy chains in part (c) have identical variable domains to each other, optionally identical variable, CH1 and/or CH2 domains. They may optionally differ only in their CH3 domains, e.g., by the creation of knob into hole mutations and other mutations intended to promote the correct association of heterodimers.

The CEA-binding sites/sequences may be any of the CEA-binding sites/sequences described above.

In one particular embodiment, the first antibody may have CEA binding sequences (i.e., CDRs or VH/VL domains) from the antibody CH1A1A.

For example, the two light chains in (a) may comprise the CDRs of SEQ ID Nos 22-24 and/or may comprise light chains variable domains having at least 90, 91, 92, 93, 94, 95, 96, 97, 98, 99 or 100% identity to SEQ ID NO 26. In some embodiments they may have at least 90, 91, 92, 93, 94, 95, 96, 97, 98, 99 or 100% identity to SEQ ID NO 103. In some embodiments, it may be preferred that the two light chains in (a) are identical to each other.

The two antibody heavy chains in part (a) may comprise the CDRs of SEQ ID NOs: 19-21 and/or the two antibody heavy chains in part (a) comprise a variable domain having at least 90, 91, 92, 93, 94, 95, 96, 97, 98, 99 or 100% identity to SEQ ID NO 25. In one embodiment, one heavy chain in part (a) has the sequence of SEQ ID NO: 100 and the other has the sequence of SEQ ID NO: 102.

In one specific embodiment, the first antibody may comprise a first heavy chain of SEQ ID NO: 100, and second heavy chain of SEQ ID NO: 101 (wherein the C-terminal AST is optional and may be absent or substituted with anther C-terminal extension as described herein) and a light chain of SEQ ID NO: 103.

The second antibody may also have CEA binding sequences (i.e., CDRs or VH/VL domains) from the antibody CH1A1A.

For example, the two light chain in (c) may comprise the CDRs of SEQ ID Nos 22-24 and/or may comprise light chains variable domains having at least 90, 91, 92, 93, 94, 95, 96, 97, 98, 99 or 100% identity to SEQ ID NO 26. In some embodiments they may have at least 90, 91, 92, 93, 94, 95, 96, 97, 98, 99 or 100% identity to SEQ ID NO: 103. In some embodiments, it may be preferred that the two light chains in (c) are identical to each other. In some embodiments, it may be preferred that the two light chains in (c) have the same sequence as the light chains in (a) of the first antibody, e.g., that all said light chains in parts (a) and (c) have the same sequence.

In some embodiments, the two antibody heavy chains in part (c) comprise the CDRs of SEQ ID NOs: 19-21 and/or the two antibody heavy chains in part (c) comprise a variable domain having at least 90, 91, 92, 93, 94, 95, 96, 97, 98, 99 or 100% identity to SEQ ID NO 25. In one embodiment, one heavy chain of part (c) has the sequence of SEQ ID NO: 97 and the other has the sequence of SEQ ID NO: 99.

In one specific embodiment, the second antibody may comprise a first heavy chain of SEQ ID NO: 97, and second heavy chain of SEQ ID NO: 98 and a light chain of SEQ ID NO: 103.

Similarly, aspects and embodiments concerning target binding (e.g., CEA-binding, FAP-binding or GPRC5D-binding) and aspects and embodiments concerning Pb-DOTAM binding can in some embodiments be combined. That is, it may be preferred that the first and second antibody each comprise a binding site for CEA, FAP or GPRC5D, e.g., comprising any of the sequences as described above, and that the first and second antibodies associate to form a binding site for a Pb-DOTAM chelate having any of the sequences as described above. It is also expressly contemplated that aspects and embodiments concerning CEA binding, FAP or GPRC5D and/or Pb-DOTAM binding can be combined with preferred formats for the antibody as described above—i.e., in any of the preferred formats, the part that binds the target antigen may comprise CDRs or variable regions sequences as described above, and/or the part that binds the radionuclide-labelled compound may be a Pb-DOTAM binder having CDRs and/or variable region sequences as described above.

In one particular embodiment, the first antibody may comprise:

a) a first full length antibody specifically binding to CEA and consisting of two antibody heavy chains and two antibody light chains; and

b) a polypeptide comprising or consisting of an antibody heavy chain variable domain (VH) wherein the heavy chain variable domain comprises heavy chain CDRs of SEQ ID NOs 1-3, and/or wherein the heavy chain variable domain has at least 90, 91, 92, 93, 94, 95, 96, 97, 98, 99 or 100% identity to SEQ ID NO 7;

wherein said polypeptide is fused with the N-terminus of the VH domain, preferably via a peptide linker, to the C-terminus of one of the two heavy chains of said first full-length antibody.

The first antibody does not comprise a light chain domain which associates with the polypeptide of (b) to form a functional binding domain for a radiolabelled compound.

It may be preferred that the polypeptide of (b) further comprises one or more residues at the C-terminus of the VH domain, optionally, one or more alanine residues, optionally a single alanine residue. For example, the polypeptide of (b) may comprise or consists of SEQ ID NO: 7 with a C-terminal alanine extension, i.e., the sequence

(SEQ ID NO.: 150)
VTLKESGPVLVKPTETLTLTCTVSGFSLSTYSMSWIRQPPGKA
LEWLGFIGSRGDTYYASWAKGRLTISKDTSKSQVVLTMTNMDP
VDTATYYCARERDPYGGGAYPPHLWGRGTLVTVSSA.

In another embodiment, the additional residues may be an N-terminal portion of the CH1 domain as described above, e.g., 1-10 residues from the N-terminus of the CH1 domain, e.g., from the human IgG1 CH1 domain. For instance, the additional residues may be AST.

In some embodiments, the two antibody heavy chains in part (a) have identical variable domains, optionally identical variable, CH1 and/or CH2 domains. They may optionally differ only in their CH3 domains, e.g., by the creation of knob into hole mutations and other mutations intended to promote the correct association of heterodimers.

The second antibody may comprise:

c) a second full length antibody specifically binding CEA and consisting of two antibody heavy chains and two antibody light chains; and

d) a polypeptide comprising or consisting of an antibody light chain variable domain (VL) wherein the light chain variable domain comprises CDRs of SEQ ID NO: 4-6 and/or wherein the light chain variable domain has at least 90, 91, 92, 93, 94, 95, 96, 97, 98, 99 or 100% identity to SEQ ID NO 8;

wherein said polypeptide is fused with the N-terminus of the VL domain, preferably via a peptide linker, to the C-terminus of one of the two heavy chains of said second full-length antibody and wherein the second antibody does not comprise a heavy chain domain which associates with the polypeptide of (d) to form a functional binding domain for a radiolabelled compound.

In some embodiments, the two antibody heavy chains in part (c) have identical variable domains to each other, optionally identical variable, CH1 and/or CH2 domains. They may optionally differ only in their CH3 domains, e.g., by the creation of knob into hole mutations and other mutations intended to promote the correct association of heterodimers.

In a particular embodiment, the first antibody may have CEA binding sequences (i.e., CDRs or VH/VL domains) from the antibody CH1A1A.

For example, the two light chains in (a) may comprise the CDRs of SEQ ID Nos 22-24 and/or may comprise light chains variable domains having at least 90, 91, 92, 93, 94, 95, 96, 97, 98, 99 or 100% identity to SEQ ID NO 26. In some embodiments they may have at least 90, 91, 92, 93, 94, 95, 96, 97, 98, 99 or 100% identity to SEQ ID NO 34. In some embodiments, it may be preferred that the two light chains in (a) are identical to each other.

The two antibody heavy chains in part (a) may comprise the CDRs of SEQ ID NOs: 19-21 and/or the two antibody heavy chains in part (a) comprise a variable domain having at least 90, 91, 92, 93, 94, 95, 96, 97, 98, 99 or 100% identity to SEQ ID NO 25. In one embodiment, one heavy chain in part (a) has the sequence of SEQ ID NO: 27 and the other has the sequence of SEQ ID NO: 28.

In one specific embodiment, the first antibody may comprise a first heavy chain of SEQ ID NO: 28, and second heavy chain of SEQ ID NO: 32 (or a variant thereof comprising an additional C-terminal alanine or other C-terminal extension as described herein, such as an extension with AST) and a light chain of SEQ ID NO: 34. A variant of SEQ ID NO: 32 with a C-terminal alanine extension is shown below:

(SEQ ID NO.: 153)
QVQLVQSGAEVKKPGASVKVSCKASGYTFTEFGMNWVRQAPGQGL
EWMGWINTKTGEATYVEEFKGRVTFTTDTSTSTAYMELRSLRSDD
TAVYYCARWDFAYYVEAMDYWGQGTTVTVSSASTKGPSVFPLAPS
SKSTSGGTAALGCLVKDYFPEPVTVSWNSGALTSGVHTFPAVLQS
SGLYSLSSVVTVPSSSLGTQTYICNVNHKPSNTKVDKKVEPKSCD
KTHTCPPCPAPEAAGGPSVFLFPPKPKDTLMISRTPEVTCVVVDV
SHEDPEVICFNWYVDGVEVHNAKTKPREEQYNSTYRVVSVLTVLH
QDWLNGKEYKCKVSNKALGAPIEKTISKAKGQPREPQVYTLPPCR
DELTKNQVSLWCLVKGFYPSDIAVEWESNGQPENNYKTTPPVLDS
DGSFFLYSKLTVDKSRWQQGNVFSCSVMHEALHNHYTQKSLSLSP
GGGGGSGGGGSGGGGSGGGGSVTLKESGPVLVKPTETLTLTCTVS
GFSLSTYSMSWIRQPPGKALEWLGFIGSRGDTYYASWAKGRLTIS
KDTSKSQVVLTMTNMDPVDTATYYCARERDPYGGGAYPPHLWGRG
TLVTVSSA

In another particular embodiment, the first antibody may have CEA binding sequences (i.e., CDRs or VH/VL domains) from the antibody A5B7 (including a humanized version thereof).

For example, the two light chains in (a) may comprise the CDRs of SEQ ID Nos 46-48 and/or may comprise light chains variable domains having at least 90, 91, 92, 93, 94, 95, 96, 97, 98, 99 or 100% identity to SEQ ID NO 50. In some embodiments they may have at least 90, 91, 92, 93, 94, 95, 96, 97, 98, 99 or 100% identity to SEQ ID NO: 54. In some embodiments, it may be preferred that the two light chains in (a) are identical to each other.

In some embodiments, the two antibody heavy chains in part (a) may comprise the CDRs of SEQ ID NOs: 43-45 and/or the two antibody heavy chains in part (a) comprise a variable domain having at least 90, 91, 92, 93, 94, 95, 96, 97, 98, 99 or 100% identity to SEQ ID NO 49. In one embodiment, one heavy chain in part (a) has the sequence of SEQ ID NO: 51 and the other has the sequence of SEQ ID NO: 53.

In one specific embodiment, the first antibody may comprise a first heavy chain of SEQ ID NO: 51, and second heavy chain of SEQ ID NO: 52 (or a variant thereof with a C-terminal alanine extension or other C-terminal extension as described herein, such as an extension with AST) and a light chain of SEQ ID NO: 54.

In another particular embodiment, the first antibody may have CEA binding sequences (i.e., CDRs or VH/VL domains) from the antibody T84.66 (including a humanized version thereof).

For example, the two light chains in (a) may comprise the CDRs of SEQ ID Nos 14-16 and/or may comprise light chains variable domains having at least 90, 91, 92, 93, 94, 95, 96, 97, 98, 99 or 100% identity to SEQ ID NO 18. In some embodiments they may have at least 90, 91, 92, 93, 94, 95, 96, 97, 98, 99 or 100% identity to SEQ ID NO: 89. In some embodiments, it may be preferred that the two light chains in (a) are identical to each other.

In some embodiments, the two antibody heavy chains in part (a) may comprise the CDRs of SEQ ID NOs: 11-13 and/or the two antibody heavy chains in part (a) comprise a variable domain having at least 90, 91, 92, 93, 94, 95, 96, 97, 98, 99 or 100% identity to SEQ ID NO 17. In one embodiment, one heavy chain in part (a) has the sequence of SEQ ID NO: 86 and the other has the sequence of SEQ ID NO: 88.

In one specific embodiment, the first antibody may comprise a first heavy chain of SEQ ID NO: 86, and second heavy chain of SEQ ID NO: 87 (or a variant thereof in which the C-terminal “AST” is absent or substituted by a different C-terminal extension as disclosed herein) and a light chain of SEQ ID NO: 89.

In another particular embodiment, the first antibody may have CEA binding sequences (i.e., CDRs or VH/VL domains) from the antibody 28A9 (including a humanized version thereof).

For example, the two light chains in (a) may comprise the CDRs of SEQ ID Nos 62-64 and/or may comprise light chains variable domains having at least 90, 91, 92, 93, 94, 95, 96, 97, 98, 99 or 100% identity to SEQ ID NO: 66. In some embodiments they may have at least 90, 91, 92, 93, 94, 95, 96, 97, 98, 99 or 100% identity to SEQ ID NO: 96. In some embodiments, it may be preferred that the two light chains in (a) are identical to each other.

In some embodiments, the two antibody heavy chains in part (a) may comprise the CDRs of SEQ ID NOs: 59-61 and/or the two antibody heavy chains in part (a) comprise a variable domain having at least 90, 91, 92, 93, 94, 95, 96, 97, 98, 99 or 100% identity to SEQ ID NO 65. In one embodiment, one heavy chain in part (a) has the sequence of SEQ ID NO: 93 and the other has the sequence of SEQ ID NO: 95.

In one specific embodiment, the first antibody may comprise a first heavy chain of SEQ ID NO: 93, and second heavy chain of SEQ ID NO: 94 (or a variant thereof without the C-terminal “AST” or with a different C-terminal extension as described herein) and a light chain of SEQ ID NO: 96.

In some embodiments, the second antibody may have CEA binding sequences (i.e., CDRs or VH/VL domains) from the antibody CH1A1A.

For example, the two light chain in (c) may comprise the CDRs of SEQ ID Nos 22-24 and/or may comprise light chains variable domains having at least 90, 91, 92, 93, 94, 95, 96, 97, 98, 99 or 100% identity to SEQ ID NO 26. In some embodiments they may have at least 90, 91, 92, 93, 94, 95, 96, 97, 98, 99 or 100% identity to SEQ ID NO 34. In some embodiments, it may be preferred that the two light chains in (c) are identical to each other. In some embodiments, it may be preferred that the two light chains in (c) have the same sequence as the light chains in (a) of the first antibody, e.g., that all said light chains in parts (a) and (c) have the same sequence.

In some embodiments, the two antibody heavy chains in part (c) comprise the CDRs of SEQ ID NOs: 19-21 and/or the two antibody heavy chains in part (c) comprise a variable domain having at least 90, 91, 92, 93, 94, 95, 96, 97, 98, 99 or 100% identity to SEQ ID NO 25. In one embodiment, one heavy chain of part (c) has the sequence of SEQ ID NO: 29 and the other has the sequence of SEQ ID NO: 30.

In one specific embodiment, the second antibody may comprise a first heavy chain of SEQ ID NO: 30, and second heavy chain of SEQ ID NO: 33 and a light chain of SEQ ID NO: 34.

In another particular embodiment, the second antibody may have CEA binding sequences (i.e., CDRs or VH/VL domains) from A5B7 (including a humanized version thereof).

For example, the two light chain in (c) may comprise the CDRs of SEQ ID Nos 46-48 and/or may comprise light chains variable domains having at least 90, 91, 92, 93, 94, 95, 96, 97, 98, 99 or 100% identity to SEQ ID NO 50. In some embodiments they may have at least 90, 91, 92, 93, 94, 95, 96, 97, 98, 99 or 100% identity to SEQ ID NO 58. In some embodiments, it may be preferred that the two light chains in (c) are identical to each other. In some embodiments, it may be preferred that the two light chains in (c) have the same sequence as the light chains in (a) of the first antibody, e.g., that all said light chains in parts (a) and (c) have the same sequence.

In some embodiments, the two antibody heavy chains in part (c) comprise the CDRs of SEQ ID NOs: 43-45 and/or the two antibody heavy chains in part (c) comprise a variable domain having at least 90, 91, 92, 93, 94, 95, 96, 97, 98, 99 or 100% identity to SEQ ID NO 49. In one embodiment, one heavy chain of part (c) has the sequence of SEQ ID NO: 55 and the other has the sequence of SEQ ID NO: 57.

In one specific embodiment, the second antibody may comprise a first heavy chain of SEQ ID NO: 55, and second heavy chain of SEQ ID NO: 56 and a light chain of SEQ ID NO: 58.

In another particular embodiment, the second antibody may have CEA binding sequences (i.e., CDRs or VH/VL domains) from the antibody T84.66 (including a humanized version thereof).

For example, the two light chains in (c) may comprise the CDRs of SEQ ID Nos 14-16 and/or may comprise light chains variable domains having at least 90, 91, 92, 93, 94, 95, 96, 97, 98, 99 or 100% identity to SEQ ID NO 18. In some embodiments they may have at least 90, 91, 92, 93, 94, 95, 96, 97, 98, 99 or 100% identity to SEQ ID NO: 89. In some embodiments, it may be preferred that the two light chains in (c) are identical to each other.

In some embodiments, the two antibody heavy chains in part (c) may comprise the CDRs of SEQ ID NOs: 11-13 and/or the two antibody heavy chains in part (c) comprise a variable domain having at least 90, 91, 92, 93, 94, 95, 96, 97, 98, 99 or 100% identity to SEQ ID NO 17. In one embodiment, one heavy chain in part (c) has the sequence of SEQ ID NO: 83 and the other has the sequence of SEQ ID NO: 85.

In one specific embodiment, the second antibody may comprise a first heavy chain of SEQ ID NO: 83, and second heavy chain of SEQ ID NO: 84 and a light chain of SEQ ID NO: 89.

In another particular embodiment, the second antibody may have CEA binding sequences (i.e., CDRs or VH/VL domains) from the antibody 28A9 (including a humanized version thereof).

For example, the two light chains in (c) may comprise the CDRs of SEQ ID Nos 62-64 and/or may comprise light chains variable domains having at least 90, 91, 92, 93, 94, 95, 96, 97, 98, 99 or 100% identity to SEQ ID NO 66. In some embodiments they may have at least 90, 91, 92, 93, 94, 95, 96, 97, 98, 99 or 100% identity to SEQ ID NO: 96. In some embodiments, it may be preferred that the two light chains in (c) are identical to each other.

In some embodiments, the two antibody heavy chains in part (c) may comprise the CDRs of SEQ ID NOs: 59-61 and/or the two antibody heavy chains in part (a) comprise a variable domain having at least 90, 91, 92, 93, 94, 95, 96, 97, 98, 99 or 100% identity to SEQ ID NO 65. In one embodiment, one heavy chain in part (c) has the sequence of SEQ ID NO: 90 and the other has the sequence of SEQ ID NO: 92.

In one specific embodiment, the second antibody may comprise a first heavy chain of SEQ ID NO: 90, and second heavy chain of SEQ ID NO: 91 and a light chain of SEQ ID NO: 96.

In some embodiments, the first and the second antibody bind the same epitope of CEA. Thus, for example, the first and the second antibody may both have CEA binding sequences from the antibody CH1A1A; or, the first and the second antibody may both have CEA binding sequences from A5B7 (including a humanized version thereof); or, the first and the second antibody may both have CEA binding sequences from T84.66 (including a humanized version thereof); or, the first and the second antibody may both have CEA binding sequences from 28A9 (including a humanized version thereof); or, the first and the second antibody may both have CEA binding sequences from MFE23 (including a humanized version thereof).

Thus, for example:

i) the first antibody may comprise a first heavy chain of SEQ ID NO: 28, a second heavy chain of SEQ ID NO: 32 (optionally with a C-terminal extension as described herein, e.g., AST) and a light chain of SEQ ID NO: 34; and the second antibody may comprise a first heavy chain of SEQ ID NO: 30, a second heavy chain of SEQ ID NO: 33 and a light chain of SEQ ID NO: 34;

ii) the first antibody may comprise a first heavy chain of SEQ ID NO: 51, a second heavy chain of SEQ ID NO: 52 (optionally with a C-terminal extension as described herein, e.g., AST) and a light chain of SEQ ID NO: 54; and the second antibody may comprise a first heavy chain of SEQ ID NO: 55, a second heavy chain of SEQ ID NO: 56 and a light chain of SEQ ID NO: 58;

iii) the first antibody may comprise a first heavy chain of SEQ ID NO: 86, a second heavy chain of SEQ ID NO: 87 (wherein the C-terminal AST residues are optional and may be absent or substituted by an alternative C-terminal extension) and a light chain of SEQ ID NO: 89; and the second antibody may comprise a first heavy chain of SEQ ID NO: 83, a second heavy chain of SEQ ID NO: 84 and a light chain of SEQ ID NO: 89; or

iv) the first antibody may comprise a first heavy chain of SEQ ID NO: 93, a second heavy chain of SEQ ID NO: 94 (wherein the C-terminal AST residues are optional and may be absent or substituted by an alternative C-terminal extension) and a light chain of SEQ ID NO: 96; and the second antibody may comprise a first heavy chain of SEQ ID NO: 90, a second heavy chain of SEQ ID NO: 91 and a light chain of SEQ ID NO: 96.

In other embodiments, the first and the second antibodies bind to different epitopes of CEA, as discussed above. Thus, for instance, the first antibody may have CEA binding sequences from the antibody CH1A1A and the second antibody may have CEA binding sequences from A5B7; or, the first antibody may have CEA binding sequences from the antibody A5B7 and the second antibody may have CEA binding sequences from CH1A1A. An example of the use of bi-paratopic (CH1A1A and A5B7) pairs is described in Example 6c.

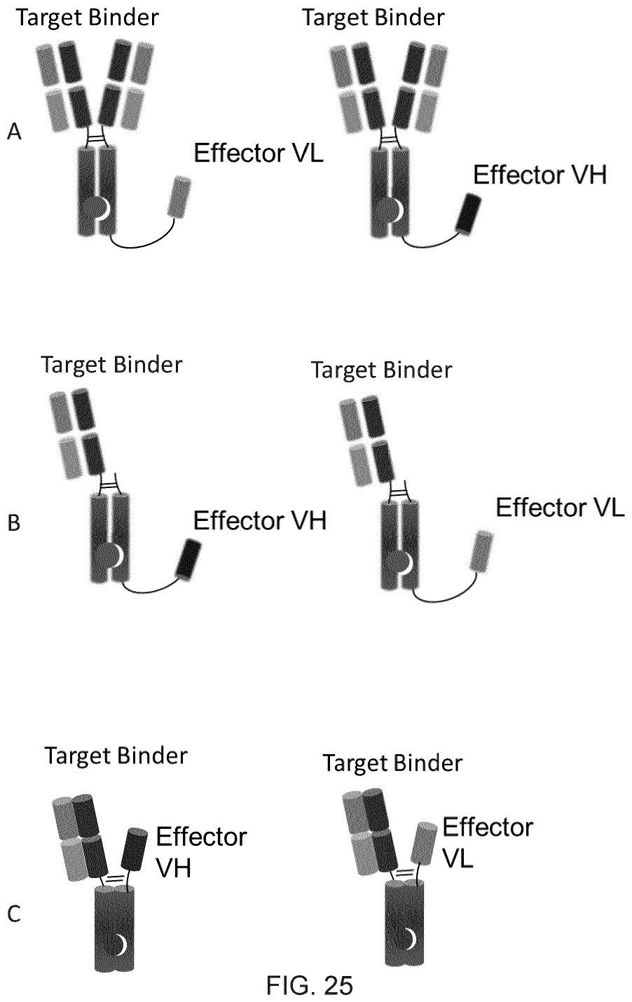

In still further specific embodiments, the target antigen may be GPRC5D or FAP and the format may be as shown in FIG. 25B. Optionally, the first and second antibody associate to form a functional antigen binding site for a Pb-DOTAM chelate (Pb-DOTAM).

Thus, in one embodiment (where the target antigen is GPRC5D):

i) the first antibody comprises a first heavy chain of SEQ ID NO: 104, a second heavy chain of SEQ ID NO:106 (wherein the C-terminal alanine is optional and may be absent or replaced by an alternative C-terminal extension as described herein) and a light chain of SEQ ID NO: 107; and

ii) the second antibody comprises a first heavy chain of SEQ ID NO: 104, a second heavy chain of SEQ ID NO: 105 and a light chain of SEQ ID NO: 107.

In another embodiment (where the target antigen is FAP):

i) the first antibody comprises a first heavy chain of SEQ ID NO: 108, a second heavy chain of SEQ ID NO:110 (wherein the C-terminal alanine is optional and may be absent or replaced by an alternative C-terminal extension as described herein) and a light chain of SEQ ID NO: 111; and

ii) the second antibody comprises a first heavy chain of SEQ ID NO: 108, a second heavy chain of SEQ ID NO:109 and a light chain of SEQ ID NO: 111.

In still further specific embodiments, the target may be CEA, e.g., having CEA binding sequences from the antibody CH1A1A, and the format may be as shown in FIG. 25C. Optionally, the first and second antibody associate to form a functional antigen binding site for a Pb-DOTAM chelate (Pb-DOTAM). Thus, in one specific embodiment:

i) the first antibody comprises a first heavy chain of SEQ ID NO:112, a second heavy chain of SEQ ID NO: 114 and a light chain of SEQ ID NO: 115; and

ii) the second antibody comprises a first heavy chain of SEQ ID NO:112, a second heavy chain of SEQ ID NO: 113 and a light chain of SEQ ID NO: 115.

### I. Antibody Variants

In certain embodiments, amino acid sequence variants of the antibodies provided herein are contemplated. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

### Substitution, Insertion, and Deletion Variants

In certain embodiments, antibody variants having one or more amino acid substitutions are provided. Sites of interest for substitutional mutagenesis include the HVRs (CDRs) and FRs. Conservative substitutions are shown in Table 1 under the heading of “preferred substitutions.” More substantial changes are provided in Table 1 under the heading of “exemplary substitutions,” and as further described below in reference to amino acid side chain classes. Amino acid substitutions may be introduced into an antibody of interest and the products screened for a desired activity, e.g., retained/improved antigen binding, decreased immunogenicity, or reduced or eliminated ADCC or CDC.

TABLE 1

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile, Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:

(1) hydrophobic: Norleucine, Met, Ala, Val, Leu, Ile;

(2) neutral hydrophilic: Cys, Ser, Thr, Asn, Gln;

(3) acidic: Asp, Glu;

(4) basic: His, Lys, Arg;

(5) residues that influence chain orientation: Gly, Pro;

(6) aromatic: Trp, Tyr, Phe.

Non-conservative substitutions will entail exchanging a member of one of these classes for another class.

One type of substitutional variant involves substituting one or more hypervariable region residues of a parent antibody (e.g., a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more. CDR residues are mutated and the variant antibodies displayed on phage and screened for a particular biological activity (e.g., binding affinity).

Alterations (e.g., substitutions) may be made in CDRs, e.g., to improve antibody affinity. Such alterations may be made in CDR “hotspots”, i.e., residues encoded by codons that undergo mutation at high frequency during the somatic maturation process (see, e.g., Chowdhury, Methods Mal. Biol. 207:179-196 (2008)), and/or residues that contact antigen, with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, N.J., (2001).) In some aspects of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves CDR-directed approaches, in which several CDR residues (e.g., 4-6 residues at a time) are randomized. CDR residues involved in antigen binding may be specifically identified, e.g., using alanine scanning mutagenesis or modelling. CDR-H3 and CDR-L3 in particular are often targeted.

In certain aspects, substitutions, insertions, or deletions may occur within one or more CDRs so long as such alterations do not substantially reduce the ability of the antibody to bind antigen. For example, conservative alterations (e.g., conservative substitutions as provided herein) that do not substantially reduce binding affinity may be made in the CDRs. Such alterations may, for example, be outside of antigen contacting residues in the CDRs. In certain variant VH and VL sequences provided above, each CDR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex may be used to identify contact points between the antibody and antigen. Such contact residues and neighbouring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT (antibody directed enzyme prodrug therapy)) or a polypeptide which increases the serum half-life of the antibody.

### Glycosylation Variants

In certain aspects, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the oligosaccharide attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some aspects, modifications of the oligosaccharide in an antibody of the invention may be made in order to create antibody variants with certain improved properties.

In one aspect, antibody variants are provided having a non-fucosylated oligosaccharide, i.e. an oligosaccharide structure that lacks fucose attached (directly or indirectly) to an Fc region. Such non-fucosylated oligosaccharide (also referred to as “afucosylated” oligosaccharide) particularly is an N-linked oligosaccharide which lacks a fucose residue attached to the first GlcNAc in the stem of the biantennary oligosaccharide structure. In one aspect, antibody variants are provided having an increased proportion of non-fucosylated oligosaccharides in the Fc region as compared to a native or parent antibody. For example, the proportion of non-fucosylated oligosaccharides may be at least about 20%, at least about 40%, at least about 60%, at least about 80%, or even about 100% (i.e. no fucosylated oligosaccharides are present). The percentage of non-fucosylated oligosaccharides is the (average) amount of oligosaccharides lacking fucose residues, relative to the sum of all oligosaccharides attached to Asn 297 (e. g. complex, hybrid and high mannose structures) as measured by MALDI-TOF mass spectrometry, as described in WO 2006/082515, for example. Asn297 refers to the asparagine residue located at about position 297 in the Fc region (EU numbering of Fc region residues); however, Asn297 may also be located about ±3 amino acids upstream or downstream of position 297, i.e., between positions 294 and 300, due to minor sequence variations in antibodies. Such antibodies having an increased proportion of non-fucosylated oligosaccharides in the Fc region may have improved FcγRIIIa receptor binding and/or improved effector function, in particular improved ADCC function. See, e.g., US 2003/0157108; US 2004/0093621.

Examples of cell lines capable of producing antibodies with reduced fucosylation include Lec13 CHO cells deficient in protein fucosylation (Ripka et al. Arch. Biochem. Biophys. 249:533-545 (1986); US 2003/0157108; and WO 2004/056312, especially at Example 11), and knockout cell lines, such as alpha-1,6-fucosyltransferase gene, FUT8, knockout CHO cells (see, e.g., Yamane-Ohnuki et al. Biotech. Bioeng. 87:614-622 (2004); Kanda, Y. et al., Biotechnol. Bioeng., 94(4):680-688 (2006); and WO 2003/085107), or cells with reduced or abolished activity of a GDP-fucose synthesis or transporter protein (see, e.g., US2004259150, US2005031613, US2004132140, US2004110282).

In a further aspect, antibody variants are provided with bisected oligosaccharides, e.g., in which a biantennary oligosaccharide attached to the Fc region of the antibody is bisected by GlcNAc. Such antibody variants may have reduced fucosylation and/or improved ADCC function as described above. Examples of such antibody variants are described, e.g., in Umana et al., Nat Biotechnol 17, 176-180 (1999); Ferrara et al., Biotechn Bioeng 93, 851-861 (2006); WO 99/54342; WO 2004/065540, WO 2003/011878.

Antibody variants with at least one galactose residue in the oligosaccharide attached to the Fc region are also provided. Such antibody variants may have improved CDC function. Such antibody variants are described, e.g., in WO 1997/30087; WO 1998/58964; and WO 1999/22764.

It may be preferred that the antibody is modified to reduce the extent of glycosylation. In some embodiments the antibody may be aglycosylated or deglycosylated. The antibody may include a substitution at N297, e.g., N297D/A.

### Fc Region Variants

In certain embodiments, one or more amino acid modifications may be introduced into the Fc region of an antibody provided herein, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

In certain embodiments, the invention contemplates an antibody variant with reduced effector function, e.g., reduced or eliminated CDC, ADCC and/or FcγR binding. In certain aspects, the invention contemplates an antibody variant that possesses some but not all effector functions, which make it a desirable candidate for applications in which the half life of the antibody in vivo is important yet certain effector functions (such as complement-dependent cytotoxicity (CDC) and antibody-dependent cell-mediated cytotoxicity (ADCC)) are unnecessary or deleterious.

In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g., Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, Calif.; and CytoTox 96® non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half life determinations can also be performed using methods known in the art (see, e.g., Petkova, S. B. et al., Int'l. Immunol. 18(12):1759-1769 (2006); WO 2013/120929 A1).

Antibodies with reduced effector function include those with substitution of one or more of Fc region residues 238, 265, 269, 270, 297, 327 and 329 (U.S. Pat. No. 6,737,056), e.g., P329G. Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

In certain aspects, an antibody variant comprises an Fc region with one or more amino acid substitutions which diminish FcγR binding, e.g., substitutions at positions 234 and 235 of the Fc region (EU numbering of residues). In one aspect, the substitutions are L234A and L235A (LALA). In certain aspects, the antibody variant further comprises D265A and/or P329G in an Fc region derived from a human IgG1 Fc region. In one aspect, the substitutions are L234A, L235A and P329G (LALA-PG) in an Fc region derived from a human IgG1 Fc region. (See, e.g., WO 2012/130831). In another aspect, the substitutions are L234A, L235A and D265A (LALA-DA) in an Fc region derived from a human IgG1 Fc region.

In other embodiments, it may be possible to use a IgG subtype with reduced effector function such as IgG4 or IgG2.

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).)

In some embodiments, alterations are made in the Fc region that result in altered (i.e., either improved or diminished, preferably diminished) C₁q binding and/or Complement Dependent Cytotoxicity (CDC), e.g., as described in U.S. Pat. No. 6,194,551, WO 99/51642, and Idusogie et al. J. Immunol. 164: 4178-4184 (2000).

In certain aspects, an antibody variant comprises an Fc region with one or more amino acid substitutions, which reduce FcRn binding, e.g., substitutions at positions 253, and/or 310, and/or 435 of the Fe-region (EU numbering of residues). In certain aspects, the antibody variant comprises an Fc region with the amino acid substitutions at positions 253, 310 and 435. In one aspect, the substitutions are I253A, H310A and H435A in an Fe region derived from a human IgG1 Fe-region. See, e.g., Grevys, A., et al., J. Immunol. 194 (2015) 5497-5508.

In certain aspects, an antibody variant comprises an Fc region with one or more amino acid substitutions, which reduce FcRn binding, e.g., substitutions at positions 310, and/or 433, and/or 436 of the Fc region (EU numbering of residues). In certain aspects, the antibody variant comprises an Fc region with the amino acid substitutions at positions 310, 433 and 436. In one aspect, the substitutions are H310A, H433A and Y436A in an Fc region derived from a human IgG1 Fc-region. (See, e.g., WO 2014/177460 A1). For instance, in some embodiments, normal FcRn binding may be used.

See also Duncan & Winter, Nature 322:738-40 (1988); U.S. Pat. Nos. 5,648,260; 5,624,821; and WO 94/29351 concerning other examples of Fc region variants.

The C-terminus of a heavy chain of the full-length antibody as reported herein can be a complete C-terminus ending with the amino acid residues PGK. The C-terminus of the heavy chain can be a shortened C-terminus in which one or two of the C terminal amino acid residues have been removed. The C-terminus of the heavy chain may be a shortened C-terminus ending PG. In one aspect of all aspects as reported herein, an antibody comprising a heavy chain including a C-terminal CH3 domain, as specified herein, comprises a C-terminal glycine residue (G446, EU index numbering of amino acid positions). This is still explicitly encompassed with the term “full length antibody” or “full length heavy chain” as used herein.

### Antibody Derivatives

In certain aspects, an antibody provided herein may be further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

### J. Recombinant Methods and Compositions

Antibodies may be produced using recombinant methods and compositions, e.g., as described in U.S. Pat. No. 4,816,567. In one embodiment, an isolated nucleic acid or a set of isolated nucleic acids encoding a set of antibodies described herein is provided.

For instance, a set of nucleic acids may comprise the following nucleic acids encoding the first antibody:
i) a nucleic acid encoding a first heavy chain of the first antibody, wherein said first heavy chain comprises a heavy chain of a full length antibody specifically binding the target antigen, fused via its C-terminus to a polypeptide comprising a VH domain of an antigen binding site for the radiolabelled compound;
ii) a nucleic acid encoding a second heavy chain of the first antibody, wherein said second heavy chain comprises a heavy chain of a full length antibody specifically binding the target antigen and does not comprise a VL domain of an antigen binding site for the radiolabelled compound (optionally, the second heavy chain consists of the heavy chain of a full length antibody specifically binding the target antigen);
iii) a nucleic acid encoding the light chain of the first antibody.
   A set of nucleic acids according to the invention may additionally or alternatively comprise the following nucleic acids encoding the second antibody:
iv) a nucleic acid encoding a first heavy chain of the second antibody, wherein said first heavy chain comprises a heavy chain of a full length antibody specifically binding the target antigen, fused via its C-terminus to a polypeptide comprising a VL domain of an antigen binding site for the radiolabelled compound;
v) a nucleic acid encoding a second heavy chain of the second antibody, wherein said second heavy chain comprises a heavy chain of a full length antibody specifically binding the target antigen and does not comprise a VH domain of an antigen binding site for the radiolabelled compound (optionally, the second heavy chain consists of the heavy chain of a full length antibody specifically binding the target antigen);
vi) a nucleic acid encoding the light chain of the second antibody.

In some embodiments, certain of these nucleic acids may be the same as each other. For instance, the nucleic acid in (iii) may the same as in (vi), such that the overall set comprises only 5 distinct nucleic acid sequences.

The nucleic acids can be comprised in one or more nucleic acid molecules or expression vectors.

Thus, in a further embodiment, one or more vectors (e.g., expression vectors) comprising such nucleic acid(s) are provided. In one embodiment, each respective heavy and light chain is expressed from an individual plasmid.

In a further embodiment, a host cell or a set of host cells comprising such nucleic acid(s) or vector(s) is provided. In one embodiment, a first host cell is provided expressing the first antibody, and a second host cell is provided expressing the second antibody.

In one such embodiment, a first host cell comprises (e.g., has been transformed with): (1) a vector comprising nucleic acids (i)-(iii) above, or (2) a first vector comprising nucleic acid (i), a second vector comprising nucleic acid (ii) and a third vector comprising nucleic acid (iii); or (3) two vectors which collectively comprise nucleic acids (i)-(iii) above. A second host cell comprises (e.g., has been transformed with): (1) a vector comprising nucleic acids (iv)-(vi) above, or (2) a first vector comprising nucleic acid (iv), a second vector comprising nucleic acid (v) and a third vector comprising nucleic acid (vi); or (3) two vectors which collective comprise nucleic acids (iv)-(vi) above.

In one embodiment, the host cell is eukaryotic, e.g. a Chinese Hamster Ovary (CHO) cell or lymphoid cell (e.g., Y0, NS0, Sp20 cell). In one embodiment, a method of making an antibody according to the invention is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an antibody, nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, K. A., In: Methods in Molecular Biology, Vol. 248, Lo, B. K. C. (ed.), Humana Press, Totowa, N.J. (2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized”, resulting in the production of an antibody with a partially or fully human glycosylation pattern. See Gerngross, T. U., Nat. Biotech. 22 (2004) 1409-1414; and Li, H. et al., Nat. Biotech. 24 (2006) 210-215.

Suitable host cells for the expression of (glycosylated) antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293T cells as described, e.g., in Graham, F. L. et al., J. Gen Virol. 36 (1977) 59-74); baby hamster kidney cells (BHK); mouse sertoli cells (TM4 cells as described, e.g., in Mather, J. P., Biol. Reprod. 23 (1980) 243-252); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells (as described, e.g., in Mather, J. P. et al., Annals N.Y. Acad. Sci. 383 (1982) 44-68); MRC 5 cells; and FS4 cells. Other useful mammalian host cell lines include Chinese hamster ovary (CHO) cells, including DHFR-CHO cells (Urlaub, G. et al., Proc. Natl. Acad. Sci. USA 77 (1980) 4216-4220); and myeloma cell lines such as Y0, NS0 and Sp2/0. For a review of certain mammalian host cell lines suitable for antibody production, see, e.g., Yazaki, P. and Wu, A. M., Methods in Molecular Biology, Vol. 248, Lo, B. K. C. (ed.), Humana Press, Totowa, N.J. (2004), pp. 255-268.

In one aspect, the host cell is eukaryotic, e.g., a Chinese Hamster Ovary (CHO) cell or lymphoid cell (e.g., Y0, NS0, Sp20 cell).

### K. Assays

Antibodies provided herein may be identified, screened for, or characterized for their physical/chemical properties and/or biological activities by various assays known in the art.

In one aspect, an antibody of the invention is tested for its antigen binding activity, e.g., by known methods such as ELISA, Western blot, etc.

### Antibody Affinity

In certain embodiments, an antibody provided herein has a dissociation constant (Kd) for the target antigen of ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M), or as otherwise stated herein.

In certain embodiments, an antigen binding site for the radiolabelled compound has a dissociation constant (Kd) for the radiolabelled compound of ≤1 μM, ≤100 nM, ≤10 nM, ≤1 nM, ≤0.1 nM, ≤0.01 nM, or ≤0.001 nM (e.g., 10-8 M or less, e.g., from 10-8 M to 10-13 M, e.g., from 10-9 M to 10-13 M). In some embodiments, the Kd is 1 nM or less, 500 pM or less, 200 pM or less, 100 pM or less, 50 pM or less, 20 pM or less, 10 pM or less, 5 pM or less or 1 pM or less, or as otherwise stated herein. For instance, the functional binding site may bind the radiolabelled compound/metal chelate with a Kd of about 1 pM-1 nM, e.g., about 1-10 pM, 1-100 pM, 5-50 pM, 100-500 pM or 500 pM-1 nM.

In one embodiment, Kd is measured by a radiolabelled antigen binding assay (RIA). In one embodiment, an RIA is performed with the Fab version of an antibody of interest and its antigen. For example, solution binding affinity of Fabs for antigen is measured by equilibrating Fab with a minimal concentration of (¹²⁵I)-labelled antigen in the presence of a titration series of unlabelled antigen, then capturing bound antigen with an anti-Fab antibody-coated plate (see, e.g., Chen et al., J. Mol. Biol. 293:865-881(1999)). To establish conditions for the assay, MICROTITER® multi-well plates (Thermo Scientific) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 pM or 26 pM [¹²⁵I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20) in PBS. When the plates have dried, 150 μl/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another embodiment, Kd is measured using a BIACORE® surface plasmon resonance assay. For example, an assay using a BIACORE®-2000 or a BIACORE®-3000 (BIAcore, Inc., Piscataway, N.J.) is performed at 25° C. with immobilized antigen CM5 chips at ˜10 response units (RU). In one embodiment, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/m11 (˜0.2 μM) before injection at a flow rate of 5 μl/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of approximately 25 μl/min. Association rates (kₒₙ) and dissociation rates (k_{off}) are calculated using a simple one-to-one Langmuir binding model (BIACORE® Evaluation Software version 3.2) by simultaneously fitting the association and dissociation sensorgrams. The equilibrium dissociation constant (Kd) is calculated as the ratio k_{off}lₒₙ. See, e.g., Chen et al., J. Mol. Biol. 293:865-881 (1999). If the on-rate exceeds 10⁶ M⁻¹ s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

In another embodiment, Kd is measured using a SET (solution equilibration titration) assay. According to this assay, test antibodies are typically applied in a constant concentration and mixed with serial dilutions of the test antigen. After incubation to establish an equilibrium, the portion of free antibodies is captured on an antigen coated surface and detected with labelled/tagged anti-species antibody, generally using electochemiluminescence (e.g., as described in Haenel et al Analytical Biochemistry 339 (2005) 182-184).

For example, in one embodiment 384-well streptavidin plates (Nunc, Microcoat #11974998001) are incubated overnight at 4° C. with 25 μl/well of an antigen-Biotin-Isomer Mix in PBS-buffer at a concentration of 20 ng/ml. For equilibration of antibody samples with free antigen: 0.01 nM-1 nM of antibody is titrated with the relevant antigen in 1:3, 1:2 or 1:1.7 dilution steps starting at a concentration of 2500 nM, 500 nM or 100 nM of antigen. The samples are incubated at 4° C. overnight in sealed REMP Storage polypropylene microplates (Brooks). After overnight incubation, streptavidin plates are washed 3× with 90 μl PBST per well. 15 μl of each sample from the equilibration plate is transferred to the assay plate and incubated for 15 min at RT, followed by 3×90 μl washing steps with PBST buffer. Detection is carried out by adding 25 μl of a goat anti-human IgG antibody-POD conjugate (Jackson, 109-036-088, 1:4000 in OSEP), followed by 6×90 μl washing steps with PBST buffer. 25 μl of TMB substrate (Roche Diagnostics GmbH, Cat. No.: 11835033001) are added to each well. Measurement takes place at 370/492 nm on a Safire2 reader (Tecan).

In another embodiment, Kd is measured using a KinExA (kinetic exclusion) assay. According to this assay, the antigen is typically titrated into a constant concentration of antibody binding sites, the samples are allowed to equilibrate, and then drawn quickly through a flow cell where free antibody binding sites are captured on antigen-coated beads, while the antigen-saturated antibody complex is washed away. The bead-captured antibody is then detected with a labelled anti-species antibody, e.g., fluorescently labelled (Bee et al PloS One, 2012; 7 (4): e36261). For example, in one embodiment, KinExA experiments are performed at room temperature (RT) using PBS pH 7.4 as running buffer. Samples are prepared in running buffer supplemented with 1 mg/ml BSA (“sample buffer”). A flow rate of 0.25 ml/min is used. A constant amount of antibody with 5 pM binding site concentration is titrated with antigen by twofold serial dilution starting at 100 pM (concentration range 0.049 pM-100 pM). One sample of antibody without antigen serves as 100% signal (i.e. without inhibition). Antigen-antibody complexes are incubated at RT for at least 24 h to allow equilibrium to be reached. Equilibrated mixtures are then drawn through a column of antigen-coupled beads in the KinExA system at a volume of 5 ml permitting unbound antibody to be captured by the beads without perturbing the equilibrium state of the solution. Captured antibody is detected using 250 ng/ml Dylight 650®-conjugated anti-human Fc-fragment specific secondary antibody in sample buffer. Each sample is measured in duplicates for all equilibrium experiments. The KD is obtained from non-linear regression analysis of the data using a one-site homogeneous binding model contained within the KinExA software (Version 4.0.11) using the “standard analysis” method.

### L. Therapeutic Methods and Compositions

The set of antibodies as described herein may be used in therapeutic methods. In one aspect a set of antibodies as described herein is provided for use as a medicament. In certain aspects, a set of antibodies for use in a method of treatment is provided.

As discussed above, in some aspects, sets of antibodies according to the present invention are suitable for any treatment in which it is desired to deliver a radionuclide to a target cell in a subject. For example, there is provided a set of antibodies as described herein for use in a method of pre-targeted radioimmunotherapy, e.g., for cancer treatment.

In certain aspects, the invention provides the set of antibodies for use in a method of pre-targeted radioimmunotherapy in an individual comprising administering to the individual an effective amount of the set of antibodies. An “individual” according to any of the above aspects is preferably a human.

As noted above, the treatment may be of any condition that is treatable by cytotoxic activity targeted to diseased cells of the patient. The treatment is preferably of a tumour or cancer. However, the applicability of the invention is not limited to tumours and cancers. For example, the treatment may also be of viral infection, or infection by another pathogenic organism, e.g., a prokaryote. Optionally, targeting may also be to T-cells for treatment of T-cell driven autoimmune disease or T-cell blood cancers. Thus, conditions to be treated may include viral infections such as HIV, rabies, EBV and Kaposi's sarcoma-associated herpesvirus, and autoimmune diseases such as multiple sclerosis and graft-versus-host disease drugs.

The term “cancer” as used herein include both solid and hematologic cancers, such as lymphomas, lymphocytic leukemias, lung cancer, non small cell lung (NSCL) cancer, bronchioloalviolar cell lung cancer, bone cancer, pancreatic cancer including pancreatic ductal adenocarcinoma (PDAC), skin cancer, cancer of the head or neck, cutaneous or intraocular melanoma, uterine cancer, ovarian cancer, cancer of the anal region, stomach cancer, gastric cancer, colorectal cancer, which may be colon cancer and/or rectal cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, hepatocellular cancer, biliary cancer, neoplasms of the central nervous system (CNS), spinal axis tumours, brain stem glioma, glioblastoma multiforme, astrocytomas, schwanomas, ependymomas, medulloblastomas, meningiomas, squamous cell carcinomas, pituitary adenoma and Ewings sarcoma, including refractory versions of any of the above cancers, checkpoint-inhibitor experienced versions of any of the above cancers, or a combination of one or more of the above cancers.

A method of targeting a radioisotope to a cell, tissue or organ for therapy may comprise:
i) administering to the subject a first and a second antibody as described herein (simultaneously or sequentially, in either order), wherein the antibodies bind to the target antigen and localise to the surface of a cell expressing the target antigen; and wherein association of the first and second antibody forms a functional binding site for the radiolabelled compound;
and
ii) subsequently administering a radiolabelled compound, wherein the radiolabelled compound binds to functional binding site for the radiolabelled compound.

The radiolabelled compound is labelled with a radioisotope which is cytotoxic to cells. Suitable radioisotopes include alpha and beta emitters as discussed above.

In methods of pre-targeted radioimmunotherapy which make use of a bispecific antibody (i.e., not a “split” antibody according to the present invention) it is common practice to administer a clearing agent or a blocking agent, between administration of the antibody and administration of the radiolabelled compound. Clearing agents bind to the antibodies and enhance their rate of clearance from the body. They include anti-idiotype antibodies. Blocking agents are typically agents which bind to the antigen binding site for the radiolabelled compound, but which are not themselves radiolabelled. For example, where the radiolabelled compound comprises a chelator loaded with a radioisotope of a certain chemical element (e.g., a metal), the blocking agent may comprise the same chelator loaded with a non-radioactive isotope of the same element (e.g., metal), or may comprise a non-loaded chelator or a chelator loaded with a different non-radioactive moiety (e.g., a non-radioactive isotope of a different element), provided that it can still be bound by the antigen-binding site. It some cases, the blocking agent may additionally comprise a moiety which increases the size and/or hydrodynamic radius of the molecule. These hinder the ability of the molecule to access the tumour, without interfering with the ability of the molecule to bind to the antibody in the circulation. Exemplary moieties include hydrophilic polymers. The moiety may be a polymer or co-polymer e.g., of dextran, dextrin, PEG, polysialic acids (PSAs), hyaluronic acid, hydroxyethyl-starch (HES) or poly(2-ethyl 2-oxazoline) (PEOZ). In other embodiments the moiety may be a non-structured peptide or protein such as XTEN polypeptides (unstructured hydrophilic protein polymers), homo-amino acid polymer (HAP), proline-alanine-serine polymer (PAS), elastin-like peptide (ELP), or gelatin-like protein (GLK). Further exemplary moieties include proteins such as albumin e.g., bovine serum albumin, or IgG. Suitable molecular weights for the moieties/polymers may be in the range e.g., of at least 50 kDa, for example between 50 kDa to 2000 kDa. For example, the molecular weight may be 200-800 kDa, optionally greater than 300, 350, 400 or 450 kDa, and optionally less than 700, 650, 600 or 550 kDa, optionally about 500 kDa.

According to certain aspects of the present invention, there is no step of administering a clearing agent or a blocking agent to the subject. In certain aspects, there is no step of administering any agent which binds to the first or the second antibody, between the administration of the antibodies and the administration of the radiolabelled compound. In certain aspects, there is no step of administering any agent between the administration of the antibodies and the radiolabelled compound, except optionally a compound selected from a chemotherapeutic agent, immunotherapeutic and a radiosensitizer. In some embodiments, no agent is administered between the administration of the antibodies and the administration of the radiolabelled compound. In some embodiments there may be no injection or infusion of any other agent to the subject, between the administration of the antibody and the administration of the radiolabelled compound.

In some embodiments, the method may be a two-step method of pre-targeted radioimmunotherapy consisting or consisting essentially of the steps of i) administering the set of antibodies (wherein the first and second antibody may be administered simultaneously or sequentially in either order) and ii) subsequently administering the radiolabelled compound. The treatment may involve multiple cycles of such therapy, i.e., multiple cycles of these two steps. An exemplary treatment cycle duration is 28 days, in which the set of antibodies is administered on day 1 of the cycle, and the radiolabelled compound is optionally administered on day 1, 2, 3, 4, 5, 6, 7, or 8 of the cycle, e.g., on day 7. The number of therapeutic cycles may vary. In one embodiment, there may be 4, 5, or 6 treatment cycles.

The present inventors have surprisingly determined that using antibodies according to the invention, it is possible to obtain therapeutically effective uptake of the radiolabelled compound into the tumour, while avoiding excessive accumulation of radioactivity in normal tissues. Indeed, in the examples the level of accumulation of radioactivity in non-target tissues was found to be lower than in a three-step PRIT method, using bispecific antibodies and a clearing step, while also making use of a simpler procedure.

In some embodiments, the radiolabelled compound may be administered to the subject once the first and second antibody have been given a suitable period of time to localise to the target cells. For instance, in some embodiments, the radiolabelled compound may be administered to the subject immediately after the first and second antibodies or at least 4 hours, 8 hours, 1 day, or 2 days, after the first and second antibodies. Optionally, it may be administered no more than 3 days, 5 days, or 7 days after the first and second antibodies. In one particular embodiment, the radiolabelled compound may be administered to the subject 2 to 7 days after the first and second antibodies.

In some embodiments, the antibodies described herein may be administered as part of a combination therapy. For example, they may be administered in combination with one or more chemotherapeutic agents: the chemotherapeutic agent and the antibody may be administered simultaneously or sequentially, in either order. Additionally or alternatively, they may be administered in combination with one or more immunotherapeutic: the immunotherapeutic agent and the antibody may be administered simultaneously or sequentially, in either order.

In some embodiments, the antibodies described herein may additionally or alternatively be administered in combination with radiosensitizers. The radiosensitizer and the antibody may be administered simultaneously or sequentially, in either order.

Antibodies of the invention (and any additional therapeutic agent, e.g., the radiolabelled compound) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g., by injections, such as intravenous or subcutaneous injections.

In some embodiments, one or more dosimetry cycles may be used prior to one or more treatment cycles as described above. A dosimetry cycle may comprise the steps of i) administering the set of antibodies (wherein the first and second antibody may be administered simultaneously or sequentially in either order) and ii) subsequently administering a compound suitable for imaging radiolabelled with a gamma-emitter (wherein said radiolabelled compound binds to functional binding site for the radiolabelled compound). The compound may be the same as the compound used in the subsequent treatment cycles, except that it is labelled with a gamma emitter rather than an alpha or beta emitter. For example, in one embodiment, the radiolabelled compound used in the dosimetry cycle may be ²⁰³Pb-DOTAM and the radiolabelled compound used in the treatment cycle may be ²¹²Pb-DOTAM. The patient may be subject to imaging to determine the uptake of the compound into the tumour and/or to estimate the absorbed dose of the compound. This information may be used to estimate the expected radiation exposure in subsequent treatment steps and to adjust the dose of the radiolabelled compound used in the treatment steps to a safe level.

### M. Pharmaceutical Formulations

The first and second antibody described herein may be formulated in a single pharmaceutical composition or in separate pharmaceutical compositions. Thus, in a further aspect, the present invention provides a pharmaceutical composition comprising the first and second antibodies of the invention, or a first pharmaceutical formulation comprising the first antibody of the invention and a second pharmaceutical composition comprising the second antibody of the invention, e.g., for use in any of the therapeutic or diagnostic methods described herein. In one embodiment, the pharmaceutical composition further comprises a pharmaceutically acceptable carrier. In another embodiment, the pharmaceutical composition further comprises at least one additional therapeutic agent, e.g., as described below.

Pharmaceutical formulations of antibodies as described herein may be prepared by mixing such antibody having the desired degree of purity with one or more optional pharmaceutically acceptable carriers (Remington's Pharmaceutical Sciences 16th edition, Osol, A. Ed. (1980)), in the form of lyophilized formulations or aqueous solutions.

Pharmaceutically acceptable carriers are generally nontoxic to recipients at the dosages and concentrations employed, and include, but are not limited to: buffers such as histidine, phosphate, citrate, acetate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Halozyme, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized antibody compositions are described in U.S. Pat. No. 6,267,958. Aqueous antibody compositions include those described in U.S. Pat. No. 6,171,586 and WO 2006/044908, the latter compositions including a histidine-acetate buffer.

The formulation herein may also contain more than one active ingredients as necessary for the particular indication being treated, preferably those with complementary activities that do not adversely affect each other. For example, it may be desirable to further provide chemotherapeutic agents, immunotherapeutic agents and/or radiosensitizers as discussed above. Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody, which matrices are in the form of shaped articles, e.g. films, or microcapsules.

The formulations to be used for in vivo administration are generally sterile. Sterility may be readily accomplished, e.g., by filtration through sterile filtration membranes.

### N. Methods and Compositions for Diagnosis and Detection

The set of antibodies as described herein may also be used in methods of diagnosis or imaging, preferably methods of or comprising pre-targeted radioimmunoimaging. Accordingly, the present invention provides methods of diagnosis and imaging. It further provides use of the set of antibodies in a method of imaging as described herein, and a set of antibodies as described herein (i.e., the first and second antibody as described herein) for use in a method of diagnosis carried out on a subject, e.g., on the human or animal body.

The imaging methods are suitable for imaging the presence and/or distribution of the target antigen in the body. For example, the method may be a method of imaging cells expressing an antigen associated with a disease, such as any of the disease conditions discussed above. Optionally the method is for imaging tumours or cancer. The method may be for the purpose of diagnosing a subject suspected of having a proliferative disorder such as cancer, or an infectious disease.

In some embodiments it may be preferred that the subject is a human.

A method of targeting a radioisotope to a tissue or organ for imaging or diagnosis may comprise:
i) administering to the subject a first and a second antibody as described herein (simultaneously or sequentially, in either order), wherein the antibodies bind to a target antigen and localise to the surface of a cell expressing the target antigen, wherein association of the first and second antibody forms a functional binding site for the radiolabelled compound;
and
ii) subsequently administering a radiolabelled compound, wherein the radiolabelled compound binds to the functional binding site for the radiolabelled compound.

Optionally, the method may further comprise:
iii) imaging the tissue or organ where the radiolabelled compound has localised, or is expected to be localised.

Optionally, the method may further comprise one or more steps of forming a diagnosis, delivering a diagnosis to the subject, and/or determining and/or administering a suitable treatment on the basis of the diagnosis.

In another embodiment, a method of the invention may comprise imaging a tissue or organ of a subject, wherein the subject has been previously administered with:
i) a first and a second antibody as described herein (simultaneously or sequentially, in either order), wherein the antibodies bind to a target antigen and localise to the surface of a cell expressing the target antigen, and wherein association of the first and second antibody forms a functional binding site for the radiolabelled compound; and
ii) a radiolabelled compound, wherein the radiolabelled compound binds to the antigen binding site for said radiolabelled compound formed by association of the first and second antibody.

In imaging and/or diagnostic methods as described herein, the radiolabelled compound is labelled with a radioisotope which is suitable for imaging. Suitable radioisotopes include gamma emitters as discussed above.

In conventional methods of pre-targeted radioimaging, it is common practice to administer a clearing or blocking agent between the administration of the antibody and the administration of the radiolabelled compound, e.g., a clearing or blocking agent as described above.

In certain embodiments of the present invention, there is no step of administering a clearing agent or a blocking agent. In certain aspects, there is no step of administering any agent which binds to the first or the second antibody, between the administration of the antibodies and the administration of the radiolabelled compound. In certain aspects, there is no step of administering any agent between the administration of the antibodies and the radiolabelled compound, except optionally a compound selected from a chemotherapeutic agent, immunotherapeutic agent and a radiosensitizer. In some embodiments, no agent is administered between the administration of the antibodies and the administration of the radiolabelled compound. In some embodiments there may be no injection or infusion of any other agent to the subject, between the administration of the antibody and the administration of the radiolabelled compound.

In some embodiments, the radiolabelled compound may be administered to the subject once the first and second antibody have been given a suitable period of time to localise to the target cells. For instance, in some embodiments, the radiolabelled compound may be administered to the subject immediately after the first and second antibodies or at least 4 hours, 8 hours, 1 day, or 2 days after the first and second antibodies. Optionally, it may be administered no more than 3 days, 5 days, or 7 days after the first and second antibodies. In one particular embodiment, the radiolabelled compound may be administered to the subject 2 to 7 days after the first and second antibodies.

In some embodiments, the imaging method may be a method of pre-targeted radioimaging consisting or consisting essentially of the steps of i) administering the set of antibodies (wherein the first and second antibody may be administered simultaneously or sequentially in either order) ii) subsequently administering the radiolabelled compound and iii) imaging the tissue or organ of interest. A diagnostic method may consist or consist essentially of said steps followed by steps of forming a diagnosis, which may then be delivered to the patient and may be used as the basis for selected and/or administering a treatment regimen.

The target antigen may be any target antigen as discussed herein. In some embodiments, the target antigen may be a tumour-specific antigen as discussed above, and the imaging may be a method of imaging a tumour or tumours. The individual may be known to or suspected of having a tumour.

For example, the method may be a method of imaging tumours in an individual having or suspected of having lung cancer, non small cell lung (NSCL) cancer, bronchioloalviolar cell lung cancer, bone cancer, pancreatic cancer including PDAC, skin cancer, cancer of the head or neck, cutaneous or intraocular melanoma, uterine cancer, ovarian cancer, colorectal cancer which may be rectal cancer and/or colon cancer, cancer of the anal region, stomach cancer, gastric cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, hepatocellular cancer, biliary cancer, neoplasms of the central nervous system (CNS), spinal axis tumours, brain stem glioma, glioblastoma multiforme, astrocytomas, schwanomas, ependymomas, medulloblastomas, meningiomas, squamous cell carcinomas, pituitary adenoma and Ewings sarcoma, including refractory versions of any of the above cancers, or check-point inhibitor experienced versions of any of these cancers, or a combination of one or more of the above cancers.

### III. Sequences

| SEQ | | |
|---|---|---|
| ID | | |
| NO | Description | Sequence |
| 1 | heavy chain | gfslstysms |
| | CDR1, <Pb- | |
| | Dotam> | |
| 2 | heavy chain | figsrgdtyyaswakg |
| | CDR2 <Pb- | |
| | Dotam> | |
| 3 | heavy chain | erdpygggaypphl |
| | CDR3 <Pb- | |
| | Dotam> | |
| 4 | light chain | qsshsvysdndla |
| | CDR1, | |
| | <Pb-Dotam> | |
| 5 | light chain | qasklas |
| | CDR2 | |
| | <Pb-Dotam> | |
| 6 | light chain | lggyddesdtyg |
| | CDR3 | |
| | <Pb-Dotam> | |
| 7 | heavy chain | vtlkesgpvl vkptetltlt |
| | variable domain | ctvsgfslst ysmswirqpp |
| | | gkalewlgfi gsrgdtyyas |
| | | wakgrltisk dtsksqwlt |
| | <Pb-Dotam> | mtnmdpvdta tyycarerdp |
| | PRIT-0213 | ygggaypphl wgrgtlvtvs |
| | | s |
| 8 | light chain | iqmtqspssl sasvgdrvti |
| | variable domain | tcqsshsvys dndlawyqqk |
| | <Pb-Dotam> | pgkapklliy qasklasgvp |
| | PRIT-0213 | srfsgsgsgt dftltisslq |
| | | pedfatyycl ggyddesdty |
| | | gfgggtkvei k |
| 9 | heavy chain | vqlqqwgagl Ikpsetlslt |
| | variable domain | cavygfslst ysmswirqpp |
| | <Pb-Dotam> | gkglewigfi gsrgdtyyas |
| | PRIT-0214 | wakgrvtisr dtsknqvslk |
| | | lssvtaadta vyycarerdp |
| | | ygggaypphl wgrgtlvtvs |
| | | s |
| 10 | light chain | iqmtqspssl sasvgdrvti |
| | variable domain | tcqsshsvys dndlawyqqk |
| | <Pb-Dotam> | pgkapklliy qasklasgvp |
| | PRIT-0214 | srfsgsgsgt dftltisslq |
| | | pedfatyycl ggyddesdty |
| | | gfgggtkvei k |
| 11 | heavy chain | GFNIKDTYMH |
| | CDR1 <CEA> | |
| | T84.66 | |
| 12 | heavy chain | RIDPANGNSKYVPKFQG |
| | CDR2 <CEA> | |
| | T84.66 | |
| 13 | heavy chain | FGYYVSDYAMAY |
| | CDR3 <CEA> | |
| | T84.66 | |
| 14 | light chain CDR1 | RAGESVDIFGVGFLH |
| | <CEA> T84.66 | |
| 15 | light chain CDR2 | RASNRAT |
| | <CEA> T84.66 | |
| 16 | light chain CDR3 | QQTNEDPYT |
| | <CEA> T84.66 | |
| 17 | heavy chain | qvqlvqsgaevkkpgssvkvsc |
| | variable domain | kasgfnikdtymhwvrqapgqg |
| | <CEA> T84.66 | lewmgridpangnskyvpkfqg |
| | | rvtitadtststaymelsslrs |
| | | edtavyycapfgyyvsdyamay |
| | | wgqgtlvtvss |
| 18 | light chain | eivltqspatlslspgeratls |
| | variable domain | cragesvdifgvgflhwyqqkp |
| | <CEA> T84.66 | gqaprlliyrasnratgiparf |
| | | sgsgsgtdftltisslcpcdfa |
| | | vyycqqtncdpytfgqgtklei |
| | | k |
| 19 | heavy chain | GYTFTEFGMN |
| | CDR1 <CEA> | |
| | CH1A1A | |
| 20 | heavy chain | WINTKTGEATYVEEFKG |
| | CDR2 <CEA> | |
| | CH1A1A | |
| 21 | heavy chain | WDFAYYVEAMDY |
| | CDR3 <CEA> | |
| | CH1A1A | |
| 22 | light chain | KASAAVGTYVA |
| | CDR1 | |
| | <CEA> CH1A1A | |
| 23 | light chain | SASYRKR |
| | CDR2 | |
| | <CEA> CH1A1A | |
| 24 | light chain | HQYYTYPLFT |
| | CDR3 | |
| | <CEA> CH1A1A | |
| 25 | heavy chain | QVQLVQSGAEVKKPGASVKVSCKAS |
| | variable | GYTFTEFGMNWVRQAPGQGLEWMGW |
| | domain | INTKTGEATYVEEFKGRVTFTTDTS |
| | <CEA> | TSTAYMELRSLRSDDTAVYYCARWD |
| | CH1A1A | FAYYVEAMDYWGQGTTVTVSS |
| 26 | light chain | DIQMTQSPSSLSASVGDRVTITCKA |
| | variable | SAAVGTYVAWYQQKPGKAPKLLIYS |
| | domain | ASYRKRGVPSRFSGSGSGTDFTLTI |
| | <CEA> | SSLQPEDFATYYCHQYYTYPLFTFG |
| | CH1A1A | QGTKLEIK |
| 27 | Heavy chain | QVQUVQSGAEVKKPGASVKVSCKAS |
| | <CEA> of | GYTFTEFGMNWVRQAPGQGLEWMGW |
| | P1AD8749 | RNTKTGEATYVEEFKGRVTFTTDTS |
| | without | TSTAYMELRSLRSDDTAVYYCARWD |
| | linker and | FAYYVEAMDYWGQGTTVTVSSASTK |
| | <DOTAM- | GPSVFPLAPSSKSTSGGTAALGCLV |
| | VH>→ Same | KDYFPEPVTVSWNSGALTSGVHTFP |
| | Plasmid as | AVLQSSGLYSLSSVVTVPSSSLGTQ |
| | SeqID32, | TYICNVNHKPSNTKVDKKVEPKSCD |
| | lacking | KTHTCPPCPAPEAAGGPSVFLFPPK |
| | linker and | PKDTLMISRTPEVTCVVVDVSHEDP |
| | <DOTAM> | EVKFNWYVDGVEVHNAKTKPREEQY |
| | | NSTYRVVSVLTVLHQDWLNGKEYKC |
| | | KVSNKALGAPIEKTISKAKGQPREP |
| | | QVYTLPPCRDELTKNQVSLWCLVKG |
| | | FYPSDIAVEWESNGQPENNYKTTPP |
| | | VLDSDGSFFLYSKLTVDKSRWQQGN |
| | | VFSCSVMHEALHNHYTQKSLSLSPG |
| 28 | P1AD8749 | QVQLVQSGAEVKKPGASVKVSCKAS |
| | Heavy | GYTFTEFGMNWVRQAPGQGLEWMGW |
| | chain hole | INTKTGEATYVEEFKGRVTFTTDTS |
| | <CEA> | TSTAYMELRSLRSDDTAVYYCARWD |
| | CH1A1A | FAYYVEAMDYWGQGTTVTVSSASTK |
| | | GPSVFPLAPSSKSTSGGTAALGCLV |
| | | KDYFPEPVTVSWNSGALTSGVHTFP |
| | | AVLQSSGLYSLSSVVTVPSSSLGTQ |
| | | TYICNVNHKPSNTKVDKKVEPKSCD |
| | | KTHTCPPCPAPEAAGGPSVFLFPPK |
| | | PKDTLMISRTPEVTCVVVDVSHEDP |
| | | EVKFNWYVDGVEVHNAKTKPREEQY |
| | | NSTYRVVSVLTVLHQDWLNGKEYKC |
| | | KVSNKALGAPIEKTISKAKGQPREP |
| | | QVCTLPPSRDELTKNQVSLSCAVKG |
| | | FYPSDIAVEWESNGQPENNYKTTPP |
| | | VLDSDSFFLVSKLTVDKSRWQQGNV |
| | | FSCSVMHEALHNHYTQKSLSLSPG |
| 29 | Heavy chain | QVQLVQSGAEVKKPGASVKVSCKAS |
| | <CEA> of | GYTFTEFGMNWVRQAPGQGLEWMGW |
| | P1AD8592 | INTKTGEATYVEEFKGRVTFTTDTS |
| | without | TSTAYMELRSLRSDDTAVYYCARWD |
| | linker and | FAYYVEAMDYWGQGTTVTVSSASTK |
| | <DOTAM- | GPSVFPLAPSSKSTSGGTAALGCLV |
| | VL>→ Same | KDYFPEPVTVSWNSGALTSGVHTFP |
| | Plasmid as | AVLQSSGLYSLSSVVTVPSSSLGTQ |
| | SeqID33, | TYICNVNHKPSNTKVDKKVEPKSCD |
| | lacking | KTHTCPPCPAPEAAGGPSVFLFPPK |
| | linker and | PKDTLMISRTPEVTCVVVDVSHEDP |
| | <DOTAM> | EVKFNWYVDGVEVHNAKTKPREEQY |
| | | NSTYRVVSVLTVLHQDWLNGKEYKC |
| | | KVSNKALGAPIEKTISKAKGQPREP |
| | | QVCTLPPSRDELTKNQVSLSCAVKG |
| | | FYPSDIAVEWESNGQPENNYKTTPP |
| | | VLDSDGSFFLVSKLTVDKSRWQQGN |
| | | VFSCSVMHEALHNHYTQKSLSLSPG |
| 30 | P1AD8592 | QVQLVQSGAEVKKPGASVKVSCKAS |
| | Heavy | GYTFTEFGMNWVRQAPGQGLEWMGW |
| | chain Knob | IMTKTGEATYVEEFKGRVTFTTDTS |
| | <CEA>CH1A1A | TSTAYMELRSLRSDDTAVYYCARWD |
| | | FAYYVEAMDYWGQGTTVTVSSASTK |
| | | GPSVFPLAPSSKSTSGGTAALGCLV |
| | | KDYFPEPVTVSWNSGALTSGVHTFP |
| | | AVLQSSGLYSLSSVVTVPSSSLGTQ |
| | | TYICNVNHKPSNTKVDKKVEPKSCD |
| | | KTHTCPPCPAPEAAGGPSVFLFPPK |
| | | PKDTLMISRTPEVTCVVVDVSHEDP |
| | | EVKFNWYVDGVEVFINAKTKPREEO |
| | | YNSTYRVVSVLTVLHQDWLNGKEYK |
| | | CKVSNKALGAPIEKTISKAKGQPRE |
| | | PQVYTLPPCRDELTKNQVSLWCLVK |
| | | GFYPSDIAVWESNGQPENNYKTTPP |
| | | VLDSDGSFFLYSKLTVDKSRWQQGN |
| | | VFSCSVMHEALHNHYTQKSLSLSPG |
| 31 | Linker | GGGGSGGGGSGGGGSGGGGS |
| 32 | P1AD8749 | QVQLVQSGAEVKKPGASVKVSCKAS |
| | heavy | GYTFTEFGMNWVRQAPGQGLEWMGW |
| | chain knob | INTKTGEATYVEEFKGRVTFTTDTS |
| | <CEA>CH1A1A | TSTAYMELRSLRSDDTAVYYCARWD |
| | <Dotam-VH> | FAYYVEAMDYWGQGTTVTVSSASTK |
| | | GPSVFPLAPSSKSTSGGTAALGCLV |
| | | KDYFPEPVTVSWNSGALTSGVHTFP |
| | | AVLQSSGLYSLSSWTVPSSSLGTQT |
| | | YICNVNHKPSNTKVDKKVEPKSCDK |
| | | THTCPPCPAPEAAGGPSVFLFPPKP |
| | | KDTLMISRTPEVTCVVVDVSHEDPE |
| | | VKFNWYVDGVEVHNAKTKPREEQYN |
| | | STYRVVSVLTVLHQDWLNGKEYKCK |
| | | VSNKALGAPIEKTISKAKGQPREPQ |
| | | VYTLPPCRDELTKNQVSLWCLVKGF |
| | | YPSDIAVEWESNGQPENNYKTTPPV |
| | | LDSDGSFFLYSKLTVDKSRWQQGNV |
| | | FSCSVMHEALHNHYTQKSLSLSP |
| | | [symbol] |
| | | VTLKES |
| | | GPVLVKPTETLTLTCTVSGFSLSTY |
| | | SMSWIRQPPGKALEWLGFIGSRGDT |
| | | YYASWAKGRLTISKDTSKSQVVLTM |
| | | TNMDPVDTATYYCARERDPYGGGAY |
| | | PPHLWGRGTLVTVSS |
| 33 | P1AD8592 heavy | QVQLVQSGAEVKKPGASVKVSCKAS |
| | chain hole | GYTFTEFGMNWVRQAPGQGLEWMGW |
| | <CEA>CH1A1A | INTKTGEATYVEEFKGRVTFTTDTS |
| | <Dotam-VL> | TSTAYMELRSLRSDDTAVYYCARWD |
| | | FAYYVEAMDYWGQGTTVTVSSASTK |
| | | GPSVFPLAPSSKSTSGGTAALGCLV |
| | | KDYFPEPVTVSWNSGALTSGVHTFP |
| | | AVLQSSGLYSLSSVVTVPSSSLGTQ |
| | | TYICNVNHKPSNTKVDKKVEPKSCD |
| | | KTHTCPPCPAPEAAGGPSVFLFPPK |
| | | PKDTLMISRTPEVTCVVVDVSHEDP |
| | | EVKFNWYVDGVEVHNAKTKPREEQY |
| | | NSTYRVVSVLTVLHQDWLNGKEYKC |
| | | KVSNKALGAPIEKTISKAKGQPREP |
| | | QVCTLPPSRDELTKNQVSLSCAVKG |
| | | FYPSDIAVEWESNGQPENNYKTTPP |
| | | VLDSDGSFFLVSKLTVDKSRWQQGN |
| | | VFSCSVMHEALHNHYTQKSLSLSPG |
| | | [symbol] IQMTQ |
| | | SPSSLSASVGDRVTITCQSSHSVYS |
| | | DNDLAWYQQKPGKAPKLLIYQASKL |
| | | ASGVPSRFSGSGSGTDFTLTISSLQ |
| | | PEDFATYYCLGGYDDESDTYGFGGG |
| | | TKVEIK |
| 34 | P1AD8749 and | DIQMTQSPSSLSASVGDRVTITCKA |
| | P1AD8592 | SAAVGTYVAWYQQKPGKAPKLLIYS |
| | light chain | ASYRKRGVPSRFSGSGSGTDFTLTI |
| | <CEA> CH1A1A | SSLQPEDFATYYCHQYYTYPLFTFG |
| | | QGTKLEIKRTVAAPSVFIFPPSDEQ |
| | | LKSGTASVVCLLNNFYPREAKVQWK |
| | | VDNALQSGNSQESVTEQDSKDSTYS |
| | | LSSTLTLSKADYEKHKVYACEVTHQ |
| 35 | Heavy chain | DYGVH |
| | CDR 1, | |
| | <C825> | |
| 36 | Heavy chain | VIWSGGGTAYNTALIS |
| | CDR 2, | |
| | <C825> | |
| 37 | Heavy chain | RGSYPYNYFDA |
| | CDR 3, | |
| | <C825> | |
| 38 | Light chain | GSSTGAVTASNYAN |
| | CDR 1, | |
| | <C825> | |
| 39 | Light chain | GHNNRPP |
| | CDR 2, | |
| | <C825> | |
| 40 | Light chain | ALWYSDHWV |
| | CDR 3, | |
| | <C825> | |
| 41 | Heavy chain | HVKLQESGPGLVQPSQSLSLTCTVS |
| | variable | GFSLTDYGVHWVRQSPGKGLEWLGV |
| | domain | IWSGGGTAYNTALISRLNIYRDNSK |
| | <C825> | NQVFLEMNSLQAEDTAMYYCARRGS |
| | | YPYNYFDAWGQGTTVTVSS |
| 42 | Light chain | QAVVIQESALTTPPGETVTLTCGSS |
| | variable | TGAVTASNYANWVQEKPDHLFTGLI |
| | domain, | GGHNNRPPGVPARFSGSLIGDKAAL |
| | <C825> | TIAGTQTEDEAIYFCALWYSDHWVI |
| | | GGGTKLTVL |
| 43 | heavy chain | DYYMN |
| | CDR1 <CEA> | |
| | A5B7 | |
| 44 | heavy chain | FIGNKANAYTTEYSASVKG |
| | CDR2 <CEA> | |
| | A5B7 | |
| 45 | heavy chain | DRGLRFYFDY |
| | CDR3 <CEA> | |
| | A5B7 | |
| 46 | light chain | RASSSVTYIH |
| | CDR1 <CEA> | |
| | A5B7 | |
| 47 | light chain | ATSNLAS |
| | CDR2 <CEA> | |
| | A5B7 | |
| 48 | light chain | QHWSSKPPT |
| | CDR3 <CEA> | |
| | A5B7 | |
| 49 | heavy chain | EVQLLESGGGLVQPGGSLRLSCAAS |
| | variable domain | GFTFTDYYMNWVRQAPGKGLEWLGF |
| | <CEA> A5B7 | IGNKANAYTTEYSASVKGRFTISRD |
| | | KSKNTLYLQMNSLRAEDTATYYCTR |
| | | DRGLRFYFDYWGQGTTVTVSS |
| 50 | light chain | EIVLTQSPATLSLSPGERATLSCRA |
| | variable domain | SSSVTYIHWYQQKPGQAPRSWIYAT |
| | <CEA> A5B7 | SNLASGIPARFSGSGSGTDFTLTIS |
| | | SLEPEDFAVYYCQHWSSKPPTFGQG |
| | | TKLEIK |
| 51 | P1AE4956 | EVQLLESGGGLVQPGGSLRLSCAAS |
| | heavy | GFTFTDYYMNWVRQAPGKGLEWLGF |
| | chain hole | IGNKANAYTTEYSASVKGRFTISRD |
| | <CEA> A5B7 | KSKNTLYLQMNSLRAEDTATYYCTR |
| | | DRGLRFYFDYWGQGTTVTVSSASTK |
| | | GPSVFPLAPSSKSTSGGTAALGCLV |
| | | KDYFPEPVTVSWNSGALTSGVHTFP |
| | | AVLQSSGLYSLSSVVTVPSSSLGTQ |
| | | TYICNVNHKPSNTKVDKKVEPKSCD |
| | | KTHTCPPCPAPEAAGGPSVFLFPPK |
| | | PKDTLMISRTPEVTCVVVDVSHEDP |
| | | EVKFNWYVDGVEVHNAKTKPREEQY |
| | | NSTYRVVSVLTVLHQDWLNGKEYKC |
| | | KVSNKALGAPIEKTISKAKGQPREP |
| | | QVCTLPPSRDELTKNQVSLSCAVKG |
| | | FYPSDIAVEWESNGQPENNYKTTPP |
| | | VLDSDGSFFLVSKLTVDKSRWQQGN |
| | | VFSCSVMHEALHNHYTQKSLSLSPG |
| 52 | P1AE4956 | EVQLLESGGGLVQPGGSLRLSCAAS |
| | heavy | GFTFTDYYMNWVRQAPGKGLEWLGF |
| | chain knob | IGNKANAYTTEYSASVKGRFTISRD |
| | <CEA> A5B7 | KSKNTLYLQMNSLRAEDTATYYCTR |
| | <Dolam-VH> | DRGLRFYFDYWGQGTTVTVSSASTK |
| | | GPSVFPLAPSSKSTSGGTAALGCLV |
| | | KDYFPEPVTVSWNSGALTSGVHTFP |
| | | AVLQSSGLYSLSSVVTVPSSSLGTQ |
| | | TYICNVNHKPSNTKVDKKVEPKSCD |
| | | KTHTCPPCPAPEAAGGPSVFLFPPK |
| | | PKDTLMISRTPEVTCVVVDVSHEDP |
| | | EVKFNWYVDGVEVHNAKTKPREEQY |
| | | NSTYRVVSVLTVLHQDWLNGKEYKC |
| | | KVSNKALGAPIEKTISKAKGQPREP |
| | | QVYTLPPCRDELTKNQVSLWCLVKG |
| | | FYPSDIAVEWESNGQPENNYKTTPP |
| | | VLDSDGSFFLYSKLTVDKSRWQQGN |
| | | VFSCSVMHEALHKHYTQKSLSLSPG |
| | | [symbol] |
| | | VTLKE |
| | | SGPVLVKPTETLTLTCTVSGFSLST |
| | | YSMSWIRQPPGKALEWLGFIGSRGD |
| | | TYYASWAKGRLTISKDTSKSQVVLT |
| | | MTNMDPVDTATYYCARERDPYGGGA |
| | | YPPHLWGRGTLVTVSS |
| 53 | Heavy chain | EVQLLESGGGLVQPGGSLRLSCAAS |
| | <CEA> of | GFTFTDYYMNWVRQAPGKGLEWLGF |
| | P1AE4956 | IGNKANAYTTEYSASVKGRFTISRD |
| | without | KSKNTLYLQMNSLRAEDTATYYCTR |
| | linker and | DRGLRFYFDYWGQGTTVTVSSASTK |
| | DOTAM- | GPSVFPLAPSSKSTSGGTAALGCLV |
| | VH>→ Same | KDYFPEPVTVSWNSGALTSGVHTFP |
| | Plasmid | AVLQSSGLYSLSSVVTVPSSSLGTQ |
| | as SeqID 52, | TYICNVNHKPSNTKVDKKVEPKSCD |
| | lacking | KTHTCPPCPAPEAAGGPSVFLFPPK |
| | linker and | PKDTLMISRTPEVTCVVVDVSHEDP |
| | <DOTAM> | EVKFNWYVDGVEVHNAKTKPREEQY |
| | | NSTYRVVSVLTVLHQDWLNGKEYKC |
| | | KVSNKALGAPIEKTISKAKGQPREP |
| | | QVYTLPPCRDELTKNQVSLWCLVKG |
| | | FYPSDIAVEWESNGQPENNYKTTPP |
| | | VLDSDGSFFLYSKLTVDKSRWQQGN |
| | | VFSCSVMHEALHNHYTQKSLSLSP |
| 54 | P1AE4956 | EIVLTQSPATLSLSPGERATLSCRA |
| | light | SSSVTYIHWYQQICPGQAPRSWIYA |
| | chain <CEA> | TSNLASGIPARFSGSGSGTDFTLTI |
| | A5B7 | SSLEPEDFAVYYCQHWSSKPPTFGQ |
| | | GTKLEIKRTVAAPSVFIFPPSDEQL |
| | | KSGTASVVCLLNNFYPREAKVQWKV |
| | | DNALQSGNSQESVTEQDSKDSTYSL |
| | | SSTLTLSKADYEKHKVYACEVTHQG |
| | | LSSPVTKSFNRGEC |
| 55 | P1AE4957 | EVQLLESGGGLVQPGGSLRLSCAAS |
| | heavy chain | GFTFTDYYMNWVRQAPGKGLEWLGF |
| | knob <CEA> | IGNKANAYTTEYSASVKGRFTISRD |
| | A5B7 | KSKNTLYLQMNSLRAEDTATYYCTR |
| | | DRGLRFYFDYWGQGTTVTVSSASTK |
| | | GPSVFPLAPSSKSTSGGTAALGCLV |
| | | KDYFPEPVTVSWNSGALTSGVHTFP |
| | | AVLQSSGLYSLSSVVTVPSSSLGTQ |
| | | TYICNVNHKPSNTKVDKKVEPKSCD |
| | | KTHTCPPCPAPEAAGGPSVFLFPPK |
| | | PKDTLMISRTPEVTCVVVDVSHEDP |
| | | EVKFNWYVDGVEVHNAKTKPREEQY |
| | | NSTYRVVSVLTVLHQDWLNGKEYKC |
| | | KVSNKALGAPIEKTISKAKGQPREP |
| | | QVYTLPPCRDELTKNQVSLWCLVKG |
| | | FYPSDIAVEWESNGQPENNYKTTPP |
| | | VLDSDGSFFLYSKLTVDKSRWQQGN |
| | | VFSCSVMHEALHNHYTQKSLSLSPG |
| 56 | P1AE4957 | EVQLLESGGGLVQPGGSLRLSCAAS |
| | heavy chain | GFTFTDYYMNWVRQAPGKGLEWLGF |
| | hole | IGNKANAYTTEYSASVKGRFTISRD |
| | <CEA> A5B7 | KSKNTLYLQMNSLRAEDTATYYCTR |
| | <Dotam-VL> | DRGLRFYFDYWGQGTTVTVSSASTK |
| | | GPSVFPLAPSSKSTSGGTAALGCLV |
| | | KDYFPEPVTVSWNSGALTSGVHTFP |
| | | AVLQSSGLYSLSSWTVPSSSLGTQT |
| | | YICNVNHKPSNTKVDKKVEPKSCDK |
| | | THTCPPCPAPEAAGGPSVFLFPPKP |
| | | KDTLMISRTPEVTCVVVDVSHEDPE |
| | | VKFNWYVDGVEVHNAKTKPREEQYN |
| | | STYRVVSVLTVLHQDWLNGKEYKCK |
| | | VSNKALGAPIEKTISKAKGQPREPQ |
| | | VCTLPPSRDELTKNQVSLSCAVKGF |
| | | YPSDIAVEWESNGQPENNYKTTPPV |
| | | LDSDGSFFLVSKLTVDKSRWOOGNV |
| | | FSCSVMHEALHNHYTOKSLSLSPGG |
| | | [symbol] |
| | | IQMTQS |
| | | PSSLSASVGDRVTITCQSSHSVYSD |
| | | NDLAWYQQKPGKAPKLLIYQASKLA |
| | | SGVPSRFSGSGSGTDFTLTISSLQP |
| | | EDFATYYCLGGYDDESDTYGFGGGT |
| | | KVEIK |
| 57 | Heavy chain | EVQLLESGGGLVQPGGSLRLSCAAS |
| | <CEA> of | GFTFTDYYMNWVRQAPGKGLEWLGF |
| | P1AE4957 | IGNKANAYTTEYSASVKGRFTISRD |
| | without | KSKNTLYLQMNSLRAEDTATYYCTR |
| | linker | DRGLRFYFDYWGQGTTVTVSSASTK |
| | and DOTAM- | GPSVFPLAPSSKSTSGGTAALGCLV |
| | VL>→ Same | KDYFPEPVTVSWNSGALTSGVHTFP |
| | Plasmid as | AVLQSSGLYSLSSVVTVPSSSLGTQ |
| | SeqID 56, | TYICNVNHKPSNTKVDKKVEPKSCD |
| | lacking | KTHTCPPCPAPEAAGGPSVFLFPPK |
| | linker | PKDTLMISRTPEVTCVVVDVSHEDP |
| | and <DOTAM> | EVKFNWYVDGVEVHNAKTKPREEQY |
| | | NSTYRVVSVLTVLHQDWLNGKEYKC |
| | | KVSNKALGAPIEKTISKAKGQPREP |
| | | QVCTLPPSRDELTKNQVSLSCAVKG |
| | | FYPSDIAVEWESNGQPENNYKTTPP |
| | | VLDSDGSFFLVSKLTVDKSRWQQGN |
| | | VFSCSVMHEALHNHYTQKSLSLSP |
| 58 | P1AE4957 | EIVLTQSPATLSLSPGERATLSCRA |
| | light | SSSVTYIHWYQQKPGQAPRSWIYAT |
| | chain <CEA> | SNLASGIPARFSGSGSGTDFTLTIS |
| | A5B7 | SLEPEDFAVYYCQHWSSKPPTFGQ |
| | | GTKLEIKRTVAAPSVFIFPPSDEQL |
| | | KSGTASVVCLLNNFYPREAKVQWKV |
| | | DNALQSGNSQESVTEQDSKDSTYS |
| | | LSSTLTLSKADYEKHKVYACEVTHQ |
| | | GLSSPVTKSFNRGEC |
| 59 | heavy chain | GGTFSYYAIS |
| | CDR1 <CEA> | |
| | 28A9 | |
| 60 | heavy chain | GILPAFGAANYAQKFQG |
| | CDR2 <CEA> | |
| | 28A9 | |
| 61 | heavy chain | LPPLPGAGLDY |
| | CDR3 <CEA> | |
| | 28A9 | |
| 62 | light chain | RASQSISSWLA |
| | CDR1 <CEA> | |
| | 28A9 | |
| 63 | light chain | DASSLES |
| | CDR2 <CEA> | |
| | 28A9 | |
| 64 | light chain | QQNTQYPMT |
| | CDR3 <CEA> | |
| | 28A9 | |
| 65 | heavy chain | QVQLVQSGAEVKKPGSSVKVSCKAS |
| | variable | GGTFSYYAISWVRQAPGQGLEWMGG |
| | domain | ILPAFGAANYAQKFQGRVTITADK |
| | <CEA> 28A9 | STSTAYMELSSLRSEDTAVYYCARL |
| | | PPLPGAGLDYWGQGTTVTVSS |
| 66 | light chain | DIQMTQSPSTLSASVGDRVTITCRA |
| | variable | SQSISSWLAWYQQKPGKAPKLLIYD |
| | domain | ASSLESGVPSRFSGSGSGTEFTLTI |
| | <CEA> 28A9 | SSLQPDDFATYYCQQNTQYPMTFGQ |
| | | GTKVEIK |
| 67 | heavy chain | GFTFSKYAMA |
| | CDR1 | |
| | <GPRC5D> | |
| 68 | heavy chain | SISTGGVNTYYADSVKG |
| | CDR2 | |
| | <GPRC5D> | |
| 69 | heavy chain | HTGDYFDY |
| | CDR3 | |
| | <GPRC5D> | |
| 70 | light chain | RASQSVSISGINLMN |
| | CDR1 | |
| | <GPRC5D> | |
| 71 | light chain | HASILAS |
| | CDR2 | |
| | <GPRC5D> | |
| 72 | light chain | QQTRESPLT |
| | CDR3 | |
| | <GPRC5D> | |
| 73 | Heavy chain | EVQLLESGGGLVQPGGSLRLSCAAS |
| | variable | GFTFSKYAMAWVRQAPGKGLEWVAS |
| | domain | ISTGGVNTYYADSVKGRFTISRDNS |
| | <GPRC5D> | KNTLYLQMNSLRAEDTAVYYCATHT |
| | | GDYFDYWGQGTMVTVSS |
| 74 | Light chain | EIVLTQSPGTLSLSPGERATLSCRA |
| | variable | SQSVSISGINLMNWYQQKPGQQPKL |
| | domain | LIYHASILASGIPDRFSGSGSGTDF |
| | <GPRC5D> | TLTISRLEPEDFAVYYCQQTRESPL |
| | | TFGQGTRLEIK |
| 75 | heavy chain | GFTFSSYAMS |
| | CDR1 <FAP> | |
| | 4B9 | |
| 76 | heavy chain | AIIGSGASTYYADSVKG |
| | CDR2 <FAP> | |
| | 4B9 | |
| 77 | heavy chain | GWFGGFNY |
| | CDR3 <FAP> | |
| | 4B9 | |
| 78 | light chain | RASQSVTSSYLA |
| | CDR1 <FAP> | |
| | 4B9 | |
| 79 | light chain | VGSRRAT |
| | CDR2 <FAP> | |
| | 4B9 | |
| 80 | light chain | QQGIMLPPT |
| | CDR3 <FAP> | |
| | 4B9 | |
| 81 | Heavy chain | EVQLLESGGGLVQPGGSLRLSCAAS |
| | variable | GFTFSSYAMSWVRQAPGKGLEWVSA |
| | domain | IIGSGASTYYADSVKGRFTISRDNS |
| | <FAP> 4B9 | KNTLYLQMNSLRAEDTAVYYCAKGW |
| | | FGGFNYWGQGTLVTVSS |
| 82 | Light chain | EIVLTQSPGTLSLSPGERATLSCRA |
| | variable | SQSVTSSYLAWYQQKPGQAPRLLIN |
| | domain | VGSRRATGIPDRFSGSGSGTDFTLT |
| | <FAP> 4B9 | ISRLEPEDFAVYYCQQGIMLPPTFG |
| | | QGTKVEIK |
| 83 | P1AF0709 | QVQLVQSGAEVKKPGSSVKVSCKAS |
| | HCknob <CEA> | GFNIKDTYMHWVRQAPGQGLEWMGR |
| | T84.66 | IDPANGNSKYVPKFQGRVTITADTS |
| | (D1AE4688) | TSTAYMELSSLRSEDTAVYYCAPFG |
| | | YYVSDYAMAYWGQGTLVTVSSASTK |
| | | GPSVFPLAPSSKSTSGGTAALGCLV |
| | | KDYFPEPVTVSWNSGALTSGVHTFP |
| | | AVLQSSGLYSLSSVVTVPSSSLGTQ |
| | | TYICNVNHKPSNTKVDKKVEP |
| | | KSCDKTHTCPPCPAPEAAGGPSVFL |
| | | FPPKPKDTLMISRTPEVTCVVVDVS |
| | | HEDPEVKFNWYVDGVEVHNAKTKPR |
| | | EEQYNSTYRVVSVLTVLHQDWLNGK |
| | | EYKCKVSNKALGAPIEKTISKAKGQ |
| | | PREPQVYTLPPCRDELTKNQVSLWC |
| | | LVKGFYPSDIAVEWESNGQPENNYK |
| | | TTPPVLDSDGSFFLYSKLTVDKSRW |
| | | QQGNVFSCSVMHEALHNHYTQKSLS |
| | | LSPG |
| 84 | P1AF0709 | QVQLVQSGAEVKKPGSSVKVSCKAS |
| | HChole <CEA> | GFNIKDTYMHWVRQAPGQGLEWMGR |
| | T84.66 Dotam- | IDPANGNSKYVPKFQGRVTITADTS |
| | VL | TSTAYMELSSLRSEDTAVYYCAPFG |
| | (D1AA4920) | YYVSDYAMAYWGQGTLVTVSSASTK |
| | | GPSVFPLAPSSKSTSGGTAALGCLV |
| | | KDYFPEPVTVSWNSGALTSGVHTFP |
| | | AVLQSSGLYSLSSVVTVPSSSLGTQ |
| | | TYICNVNHKPSNTKVDKKVEPKSCD |
| | | KTHTCPPCPAPEAAGGPSVFLFPPK |
| | | PKDTLMISRTPEVTCVVVDVSHEDP |
| | | EVKFNWYVDGVEVHNAKTKPREEQY |
| | | NSTYRVVSVLTVLHQDWLNGKEYKC |
| | | KVSNKALGAPIEKTISKAKGQPREP |
| | | QVCTLPPSRDELTKNQVSLSCAVKG |
| | | FYPSDIAVEWESNGQPENNYKTTPP |
| | | VLDSDGSFFLVSKLTVDKSRWQQGN |
| | | VFSCSVMHEALHNHYTQKSLSLSPG |
| | | GGGGSGGGGSGGGGSGGGGSIQMTQ |
| | | SPSSLSASVGDRVTITCQSSHSVYS |
| | | DNDLAWYQQKPGKAPKLLIYQASKL |
| | | ASGVPSRFSGSGSGTDFTLTISSLQ |
| | | PEDFATYYCLGGYDDESDTYGFGGG |
| | | TKVEflC |
| 85 | P1AF0709 | QVQLVQSGAEVKKPGSSVKVSCKAS |
| | HChole <CEA> | GFNIKDTYMHWVRQAPGQGLEWMGR |
| | T84.66 | IDPANGNSKYVPKFQGRVTITADTS |
| | without | TSTAYMELSSLRSEDTAVYYCAPFG |
| | linker and | YYVSDYAMAYWGQGTLVTVSSASTK |
| | DOTAM | GPSVFPLAPSSKSTSGGTAALGCLV |
| | | KDYFPEPVTVSWNSGALTSGVHTFP |
| | | AVLQSSGLYSLSSVVTVPSSSLGTQ |
| | | TYICNVNHKPSNTKVDKXVEPKSCD |
| | | KTHTCPPCPAPEAAGGPSVFLFPPK |
| | | PKDTLMISRTPEVTCVVVDVSHEDP |
| | | EVKFNWYVDGVEVHNAKTKPREEQY |
| | | NSTYRVVSVLTVLHQDWLNGKEYKC |
| | | KVSNKALGAPIEKTISKAKGQPREP |
| | | QVCTLPPSRDELTKNQVSLSCAVKG |
| | | FYPSDIAVEWESNGQPENNYKTTPP |
| | | VLDSDGSFFLVSKLTVDKSRWQQGN |
| | | VFSCSVMHEALHNHYTQKSLSLSPG |
| 86 | P1AF0298 | QVQLVQSGAEVKKPGSSVKVSCKAS |
| | HCHole <CEA> | GFNTKDTYMHWVRQAPGQGLEWMGR |
| | T84.66 | IDPANGNSKYVPKFQGRVTITADTS |
| | (D1AE4687) | TSTAYMELSSLRSEDTAVYYCAPFG |
| | | YYVSDYAMAYWGQGTLVTVSSASTK |
| | | GPSVFPLAPSSKSTSGGTAALGCLV |
| | | KDYFPEPVTVSWNSGALTSGVHTFP |
| | | AVLQSSGLYSLSSVVTVPSSSLGTQ |
| | | TYICNVNHKPSNTKVDKKVEPKSCD |
| | | KTHTCPPCPAPEAAGGPSVFLFPPK |
| | | PKDTLMISRTPEVTCVVVDVSHEDP |
| | | EVKFNWYVDGVEVHNAKTKPREEQY |
| | | NSTYRVVSVLTVLHQDWLNGKEYKC |
| | | KVSNKALGAPIEKTISKAKGQPREP |
| | | QVCTLPPSRDELTKNQVSLSCAVKG |
| | | FYPSDIAVEWESNGQPENNYKTTPP |
| | | VLDSDGSFFLVSKLTVDKSRWQQGN |
| | | VFSCSVMHEALHNHYTQKSLSLSPG |
| 87 | P1AF0298 | QVQLVQSGAEVKKPGSSVKVSCKAS |
| | HCknob <CEA> | GFNIKDTYMHWVRQAPGQGLEWMGR |
| | T84.66 | IDPANGNSKYVPKFQGRVTITADTS |
| | Dotam- | TSTAYMELSSLRSEDTAVYYCAPFG |
| | VH-AST | YYVSDYAMAYWGQGTLVTVSSASTK |
| | (D1AE3668) | GPSVFPLAPSSKSTSGGTAALGCLV |
| | | KDYFPEPVTVSWNSGALTSGVHTFP |
| | | AVLQSSGLYSLSSVVTVPSSSLGTQ |
| | | TYICNVNHKPSNTKVDKKVEPKSCD |
| | | KTHTCPPCPAPEAAGGPSVFLFPPK |
| | | PKDTLMISRTPEVTCVVVDVSHEDP |
| | | EVKFNWYVDGVEVHNAKTKPREEQY |
| | | NSTYRVVSVLTVLHQDWLNGKEYKC |
| | | KVSNKALGAPIEKTISKAKGQPREP |
| | | QVYTLPPCRDELTKNQVSLWCLVKG |
| | | FYPSDIAVEWESNGQPENNYKTTPP |
| | | VLDSDGSFFLYSKLTVDKSRWQQGN |
| | | VFSCSVMHEALHNHYTQKSLSLSPG |
| | | GGGGSGGGGSGGGGSGGGGSVTLKE |
| | | SGPVLVKPTETLTLTCTVSGFSLST |
| | | YSMSWIRQPPGKALEWLGFIGSRGD |
| | | TYYASWAKGRLTISKDTSKSQVVLT |
| | | MTNMDPVDTATYYCARERDPYGGGA |
| | | YPPHLWGRGTLVTVSSAST |
| 88 | P1AF0298 | QVQLVQSGAEVKKPGSSVKVSCKAS |
| | HCknob <CEA> | GFNIKDTYMHWVRQAPGQGLEWMGR |
| | T84.66 | IDPANGNSKYVPKFQGRVTITADTS |
| | without | TSTAYMELSSLRSEDTAVYYCAPFG |
| | linker and | YYVSDYAMAYWGQGTLVTVSSASTK |
| | DOTAM | GPSVFPLAPSSKSTSGGTAALGCLV |
| | | KDYFPEPVTVSWNSGALTSGVHTFP |
| | | AVLQSSGLYSLSSVVTVPSSSLGTQ |
| | | TYICNVNHKPSNTKVDKKVEPKSCD |
| | | KTHTCPPCPAPEAAGGPSVFLFPPK |
| | | PKDTLMISRTPEVTCVVVDVSHEDP |
| | | EVKFNWYVDGVEVHNAKTKPREEQY |
| | | NSTYRVVSVLTVLHQDWLNGKEYKC |
| | | KVSNKALGAPIEKTISKAKGQPREP |
| | | QVYTLPPCRDELTKNQVSLWCLVKG |
| | | FYPSDIAVEWESNGQPENNYKTTPP |
| | | VLDSDGSFFLYSKLTVDKSRWQQGN |
| | | VFSCSVMHEALHNHYTQKSLSLSPG |
| 89 | P1AF0709 and | EIVLTQSPATLSLSPGERATLSCRA |
| | P1AF0298 | GESVDIFGVGFLHWYQQKPGQAPRL |
| | light | LIYRASNRATGIPARFSGSGSGTDF |
| | chain | TLTISSLEPEDFAVYYCQQTNEDPY |
| | (D1AA4120) | TFGQGTKLEIKRTVAAPSVFIFPPS |
| | | DEQLKSGTASVVCLLNNFYPREAKV |
| | | QWKVDNALQSGNSQESVTEQDSKDS |
| | | TYSLSSTLTLSKADYEKHKVYACEV |
| | | THQGLSSPVTKSFNRGEC |
| 90 | P1AF0710 | QVQLVQSGAEVKKPGSSVKVSCKAS |
| | HCknob <CEA> | GGTFSYYAISWVRQAPGQGLEWMGG |
| | 28A9 | ILPAFGAANYAQKFQGRVTITADKS |
| | (D1AE4690) | TSTAYMELSSLRSEDTAVYYCARLP |
| | | PLPGAGLDYWGQGTTVTVSSASTKG |
| | | PSVFPLAPSSKSTSGGTAALGCLVK |
| | | DYPPEPVTVSWNSGALTSGVHTFPA |
| | | VLQSSGLYSLSSWTVPSSSLGTQTY |
| | | ICNVNHKPSNTKVDKKVEPKSCDKT |
| | | HTCPPCPAPEAAGGPSVFLFPPKPK |
| | | DTLMISRTPEVTCVVVDVSHEDPEV |
| | | KFNWYVDGVEVHNAKTKPREEQYNS |
| | | TYRVVSVLTVLHQDWLNGKEYKCKV |
| | | SNKALGAPIEKTISKAKGQPREPQV |
| | | YTLPPCRDELTKNQVSLWCLVKGFY |
| | | PSDIAVEWESNGQPENNYKTTPPVL |
| | | DSDGSFFLYSKLTVDKSRWQQGNVF |
| | | SCSVMHEALHNHYTQKSLSLSPG |
| 91 | P1AF0710 | QVQLVQSGAEVKKPGSSVKVSCKAS |
| | HChole <CEA> | GGTFSYYAISWVRQAPGQGLEWMGG |
| | 28A9 Dotam- | ILPAFGAANYAQKFQGRVTITADKS |
| | VL | TSTAYMELSSLRSEDTAVYYCARLP |
| | (D1AC3172) | PLPGAGLDYWGQGTTVTVSSASTKG |
| | | PSVFPLAPSSKSTSGGTAALGCLVK |
| | | DYFPEPVTVSWNSGALTSGVHTFPA |
| | | VLQSSGLYSLSSVVTVPSSSLGTQT |
| | | YICNVNHKPSNTKVDKKVEPKSCDK |
| | | THTCPPCPAPEAAGGPSVFLFPPKP |
| | | KDTLMISRTPEVTCVVVDVSHEDPE |
| | | VKFNWYVDGVEVHNAKTKPREEQYN |
| | | STYRVVSVLTVLHQDWLNGKEYKCK |
| | | VSNKALGAPIEKTISKAKGQPREPQ |
| | | VCTLPPSRDELTKNQVSLSCAVKGF |
| | | YPSDIAVEWESNGQPENNYKTTPPV |
| | | LDSDGSFFLVSKLTVDKSRWQQGNV |
| | | FSCSVMHEALHNHYTQKSLSLSPGG |
| | | GGGSGGGGSGGGGSGGGGSIQMTQS |
| | | PSSLSASVGDRVTITCQSSHSVYSD |
| | | NDLAWYQQKPGKAPKLLIYQASKLA |
| | | SGVPSRFSGSGSGTDFTLTISSLQP |
| | | EDFATYYCLGGYDDESDTYGFGGGT |
| | | KVEEK |
| 92 | P1AF0710 | QVQLVQSGAEVKKPGSSVKVSCKAS |
| | HChole <CEA> | GGTFSYYAISWVRQAPGQGLEWMGG |
| | 28A9 without | ILPAFGAANYAQKFQGRVTLTADKS |
| | linker or | TSTAYMELSSLRSEDTAVYYCARLP |
| | DOTAM | PLPGAGLDYWGQGTTVTVSSASTKG |
| | | PSVFPLAPSSKSTSGGTAALGCLVK |
| | | DYFPEPVTVSWNSGALTSGVHTFPA |
| | | VLQSSGLYSLSSVVTVPSSSLGTQT |
| | | YICNVNHKPSNTKVDKKVEPKSCDK |
| | | THTCPPCPAPEAAGGPSVFLFPPKP |
| | | KDTLMISRTPEVTCVVVDVSHEDPE |
| | | VKFNWYVDGVEVHNAKTKPREEQYN |
| | | STYRVVSVLTVLHQDWLNGKEYKCK |
| | | VSNKALGAPIEKTISKAKGQPREPQ |
| | | VCTLPPSRDELTKNQVSLSCAVKGF |
| | | YPSDIAVEWESNGQPENNYKTTPPV |
| | | LDSDGSFFLVSKLTVDKSRWQQGNV |
| | | FSCSVMHEALHNHYTQKSLSLSPG |
| 93 | P1AF0711 | QVQLVQSGAEVKKPGSSVKVSCKAS |
| | HChole <CEA> | GGTFSYYAISWVRQAPGQGLEWMGG |
| | 28A9 | ILPAFGAANYAQKFQGRVTITADKS |
| | (D1AE4689) | TSTAYMELSSLRSEDTAVYYCARLP |
| | | PLPGAGLDYWGQGTTVTVSSASTKG |
| | | PSVFPLAPSSKSTSGGTAALGCLVK |
| | | DYFPEPVTVSWNSGALTSGVHTFPA |
| | | VLQSSGLYSLSSVVTVPSSSLGTQT |
| | | YICNVNHKPSNTKVDKKVEPKSCDK |
| | | THTCPPCPAPEAAGGPSVFLFPPKP |
| | | KDTLMISRTPEVTCVVVDVSHEDPE |
| | | VKFNWYVDGVEVHNAKTKPREEQYN |
| | | STYRVVSVLTVLHQDWLNGKEYKCK |
| | | VSNKALGAPIEKTISKAKGQPREPQ |
| | | VCTLPPSRDELTKNQVSLSCAVKGF |
| | | YPSDIAVEWESNGQPENNYKTTPPV |
| | | LDSDGSFFLVSKLTVDKSRWQQGNV |
| | | FSCSVMHEALHNHYTQKSLSLSPG |
| 94 | P1AF0711 | QVQLVQSGAEVKKPGSSVKVSCKAS |
| | HCknob <CEA> | GGTFSYYAISWVRQAPGQGLEWMGG |
| | 28A9 Dotam- | ILPAFGAANYAQKFQGRVTITADKS |
| | VH-AST | TSTAYMELSSLRSEDTAVYYCARLP |
| | (D1AE3671) | PLPGAGLDYWGQGTTVTVSSASTKG |
| | | PSVFPLAPSSKSTSGGTAALGCLVK |
| | | DYFPEPVTVSWNSGALTSGVHTFPA |
| | | VLQSSGLYSLSSVVTVPSSSLGTQT |
| | | YICNVNHKPSNTKVDKKVEPKSCDK |
| | | THTCPPCPAPEAAGGPSVFLFPPKP |
| | | KDTLMISRTPEVTCVVVDVSHEDPE |
| | | VKFNWYVDGVEVHNAKTKPREEQYN |
| | | STYRVVSVLTVLHQDWLNGKEYKCK |
| | | VSNKALGAPIEKTISKAKGQPREPQ |
| | | VYTLPPCRDELTKNQVSLWCLVKGF |
| | | YPSDIAVEWESNGQPENNYKTTPPV |
| | | LDSDGSFFLYSKLTVDKSRWQQGNV |
| | | FSCSVMHEALHNHYTQKSLSLSPGG |
| | | GGGSGGGGSGGGGSGGGGSVTLKES |
| | | GPVLVKPTETLTLTCTVSGFSLSTY |
| | | SMSWIRQPPGKALEWLGFIGSRGDT |
| | | YYASWAKGRLTISKDTSKSQVVLTM |
| | | TNMDPVDTATYYCARERDPYGGGAY |
| | | PPHLWGRGTLVTVSSAST |
| 95 | P1AF0711 | QVQLVQSGAEVKKPGSSVKVSCKAS |
| | HCknob <CEA> | GGTFSYYAISWVRQAPGQGLEWMGG |
| | 28A9 without | ILPAFGAANYAQKFQGRVTITADKS |
| | linker and | TSTAYMELSSLRSEDTAVYYCARLP |
| | DOTAM | PLPGAGLDYWGQGTTVTVSSASTKG |
| | | PSVFPLAPSSKSTSGGTAALGCLVK |
| | | DYFPEPVTVSWNSGALTSGVHTFPA |
| | | VLQSSGLYSLSSVVTVPSSSLGTQT |
| | | YICNVNHKPSNTKVDKKVEPKSCDK |
| | | THTCPPCPAPEAAGGPSVFLFPPKP |
| | | KDTLMISRTPEVTCVVVDVSHEDPE |
| | | VKFNWYVDGVEVHNAKTKPREEQYN |
| | | STYRVVSVLTVLHQDWLNGKEYKCK |
| | | VSNKALGAPIEKTISKAKGQPREPQ |
| | | VYTLPPCRDELTKNQVSLWCLVKGF |
| | | YPSDIAVEWESNGQPENNYKTTPPV |
| | | LDSDGSFFLYSKLTVDKSRWQQGNV |
| | | FSCSVMHEALHNHYTQKSLSLSPG |
| 96 | P1AF0710 and | DIQMTQSPSTLSASVGDRVTITCRA |
| | P1AF0711 | SQSISSWLAWYQQKPGKAPKLLIYD |
| | light | ASSLESGVPSRFSGSGSGTEFTLTI |
| | chain | SSLQPDDFATYYCQQNTQYPMTFGQ |
| | (D1AA2299) | GTKVEIKRTVAAPSVFIFPPSDEQL |
| | | KSGTASVVCLLNNFYPREAKVQWKV |
| | | DNALQSGNSQESVTEQDSKDSTYSL |
| | | SSTLTLSKADYEKHKVYACEVTHQG |
| | | LSSPVTKSFNRGEC |
| 97 | P1AF0712 | QVQLVQSGAEVKKPGASVKVSCKAS |
| | HCknob <CEA> | GYTFTEFGMNWVRQAPGQGLEWMGW |
| | CH1Al A | INTKTGEATYVEEFKGRVTFTT |
| | (D1AC4023) | DTSTSTAYMELRSLRSDDTAVYYCA |
| | | RWDFAYYVEAMDYWGQGTTVTVSSA |
| | | STKGPSVFPLAPSSKSTSGGTAA |
| | | LGCLVKDYFPEPVTVSWNSGALTSG |
| | | VHTFPAVLQSSGLYSLSSVVTVPSS |
| | | SLGTQTYICNVNHKPSNTKVDKKVE |
| | | PKSCDKTHTCPPCPAPEAAGGPSVF |
| | | LFPPKPKDTLMISRTPEVTCVVVDV |
| | | SHEDPEVKFNWYVDGVEVHNAKTK |
| | | PREEQYNSTYRVVSVLTVLHQDWLN |
| | | GKEYKCKVSNKALGAPIEKTISKAK |
| | | GQPREPQVYTLPPCRDELTKNQVS |
| | | LWCLVKGFYPSDIAVEWESNGQPEN |
| | | NYKTTPPVLDSDGSFFLYSKLTVDK |
| | | SRWQQGNVFSCSVMHEALHNHYT |
| | | QKSLSLSPG |
| 98 | P1AF0712 | QVQLVQSGAEVKKPGASVKVSCKAS |
| | HChole <CEA> | GYTFTEFGMNWVRQAPGQGLEWMGW |
| | CH1A1A | INTKTGEATYVEEFKGRVTFTTDTS |
| | DOTA-VL | TSTAYMELRSLRSDDTAVYYCARWD |
| | (D1AE4684) | FAYYVEAMDYWGQGTTVTVSSASTK |
| | | GPSVFPLAPSSKSTSGGTAALGCLV |
| | | KDYFPEPVTVSWNSGALTSGVHTFP |
| | | AVLQSSGLYSLSSVVTVPSSSLGTQ |
| | | TYICNVNHKPSNTKVDKKVEPKSCD |
| | | KTHTCPPCPAPEAAGGPSVFLFPPK |
| | | PKDTLMISRTPEVTCVVVDVSHEDP |
| | | EVKFNWYVDGVEVHNAKTKPREEQY |
| | | NSTYRVVSVLTVLHQDWLNGKEYKC |
| | | KVSNKALGAPIEKTISKAKGQPREP |
| | | QVCTLPPSRDELTKNQVSLSCAVKG |
| | | FYPSDIAVEWESNGQPENNYKTTPP |
| | | VLDSDGSFFLVSKLTVDKSRWQQGN |
| | | VFSCSVMHEALHNHYTQKSLSLSPG |
| | | GGGGSGGGGSGGGGSGGGGSQAVVI |
| | | QESALTTPPGETVTLTCGSSTGAVT |
| | | ASNYANWVQEKPDHLFTGLIGGHNN |
| | | RPPGVPARFSGSLIGDKAALTIAGT |
| | | QTEDEAIYFCALWYSDHWVIGGGTK |
| | | LTVL |
| 99 | P1AF0712 | QVQLVQSGAEVKKPGASVKVSCKAS |
| | HChole <CEA> | GYTFTEFGMNWVRQAPGQGLEWMGW |
| | without | INTKTGEATYVEEFKGRVTFTT |
| | linker or | DTSTSTAYMELRSLRSDDTAVYYCA |
| | DOTA | RWDFAYYVEAMDYWGQGTTVTVSSA |
| | | STKGPSVFPLAPSSKSTSGGTAA |
| | | LGCLVKDYFPEPVTVSWNSGALTSG |
| | | VHTFPAVLQSSGLYSLSSVVTVPSS |
| | | SLGTQTYICNVNHKPSNTKVDKKVE |
| | | PKSCDKTHTCPPCPAPEAAGGPSVF |
| | | LFPPKPKDTLMISRTPEVTCVVVDV |
| | | SHEDPEVKFNWYVDGVEVHNAKTK |
| | | PREEQYNSTYRVVSVLTVLHQDWLN |
| | | GKEYKCKVSNKALGAPIEKTISKAK |
| | | GQPREPQVCTLPPSRDELTKNQVS |
| | | LSCAVKGFYPSDIAVEWESNGQPEN |
| | | NYKTTPPVLDSDGSFFLVSKLTVDK |
| | | SRWQQGNVFSCSVMHEALHNHYTQK |
| | | SLSLSPG |
| 100 | P1AF0713 | QVQLVQSGAEVKKPGASVKVSCKAS |
| | HCHole <CEA> | GYTFTEFGMNWVRQAPGQGLEWMGW |
| | CH1A1A | INTKTGEATYVEEFKGRVTFTTDTS |
| | (D1AC4022) | TSTAYMELRSLRSDDTAVYYCARWD |
| | | FAYYVEAMDYWGQGTTVTVSSASTK |
| | | GPSVFPLAPSSKSTSGGTAALGCLV |
| | | KDYFPEPVTVSWNSGALTSGVHTFP |
| | | AVLQSSGLYSLSSVVTVPSSSLGTQ |
| | | TYICNVNHKPSNTKVDKKVEPKSCD |
| | | KTHTCPPCPAPEAAGGPSVFLFPPK |
| | | PKDTLMISRTPEVTCVVVDVSHEDP |
| | | EVKFNWYVDGVEVHNAKTKPREEQY |
| | | NSTYRVVSVLTVLHQDWLNGKEYKC |
| | | KVSNKALGAPIEKTISKAKGQPREP |
| | | QVCTLPPSRDELTKNQVSLSCAVKG |
| | | FYPSDIAVEWESNGQPENNYKTTPP |
| | | VLDSDGSFFLVSKLTVDKSRWQQGN |
| | | VFSCSVMHEALHNHYTQKSLSLSPG |
| 101 | P1AF0713 | QVQLVQSGAEVKKPGASVKVSCKAS |
| | HCknob <CEA> | GYTFTEFGMNWVRQAPGQGLEWMGW |
| | CH1A1A | INTKTGEATYVEEFKGRVTFTTDTS |
| | DOTA-VH-AST | TSTAYMELRSLRSDDTAVYYCARWD |
| | (D1AE3670) | FAYYVEAMDYWGQGTTVTVSSASTK |
| | | GPSVFPLAPSSKSTSGGTAALGCLV |
| | | KDYFPEPVTVSWNSGALTSGVHTFP |
| | | AVLQSSGLYSLSSVVTVPSSSLGTQ |
| | | TYICNVNHKPSNTKVDKKVEPKSCD |
| | | KTHTCPPCPAPEAAGGPSVFLFPPK |
| | | PKDTLMISRTPEVTCVVVDVSHEDP |
| | | EVKFNWYVDGVEVHNAKTKPREEQY |
| | | NSTYRVVSVLTVLHQDWLNGKEYKC |
| | | KVSNKALGAPIEKTISKAKGQPREP |
| | | QVYTLPPCRDELTKNQVSLWCLVKG |
| | | FYPSDIAVEWESNGQPENNYKTTPP |
| | | VLDSDGSFFLYSKLTVDKSRWQQGN |
| | | VFSCSVMHEALHNHYTQKSLSLSPG |
| | | GGGGSGGGGSGGGGSGGGGSHVKLQ |
| | | ESGPGLVQPSQSLSLTCTVSGFSLT |
| | | DYGVHWVRQSPGKGLEWLGVIWSGG |
| | | GTAYNTALISRLNIYRDNSKNQVFL |
| | | EMNSLQAEDTAMYYCARRGSYPYNY |
| | | FDAWGQGTTVTVSSAST |
| 102 | P1AF0713 | QVQLVQSGAEVKKPGASVKVSCKAS |
| | HCknob <CEA> | GYTFTEFGMNWVRQAPGQGLEWMGW |
| | CH1A1A | INTKTGEATYVEEFKGRVTFTTDTS |
| | without | TSTAYMELRSLRSDDTAVYYCARWD |
| | linker | FAYYVEAMDYWGQGTTVTVSSASTK |
| | and DOTA | GPSVFPLAPSSKSTSGGTAALGCLV |
| | | KDYFPEPVTVSWNSGALTSGVHTFP |
| | | AVLQSSGLYSLSSVVTVPSSSLGTQ |
| | | TYICNVNHKPSNTKVDKKVEPKSCD |
| | | KTHTCPPCPAPEAAGGPSVFLFPPK |
| | | PKDTLMISRTPEVTCVVVDVSHEDP |
| | | EVKFNWYVDGVEVHNAKTKPREEQY |
| | | NSTYRVVSVLTVLHQDWLNGKEYKC |
| | | KVSNKALGAPIEKTISKAKGQPREP |
| | | QVYTLPPCRDELTKNQVSLWCLVKG |
| | | FYPSDIAVEWESNGQPENNYKTTPP |
| | | VLDSDGSFFLYSKLTVDKSRWQQGN |
| | | VFSCSVMHEALHNHYTQKSLSLSPG |
| 103 | P1AF0712 | DIQMTQSPSSLSASVGDRVTITCKA |
| | and | SAAVGTYVAWYQQKPGKAPKLLIYS |
| | P1AF0713 | ASYRKRGVPSRFSGSGSGTDFTLTI |
| | light | SSLQPEDFATYYCHQYYTYPLFTFG |
| | chain | QGTKLEIKRTVAAPSVFIFPPSDEQ |
| | (D1AA3384) | LKSGTASWCLLNNFYPREAKVQWKV |
| | | DNALQSGNSQESVTEQDSKDSTYSL |
| | | SSTLTLSKADYEKHKVYACEVTHQG |
| | | LSSPVTKSFNRGEC |
| 104 | P1AF8284 and | EVQLLESGGGLVQPGGSLRLSCAAS |
| | P1AF8285 | GFTFSKYAMAWVRQAPGKGLEWVAS |
| | HCknob | ISTGGVNTYYADSVKGRFTISRDNS |
| | <GPRC5D> | KNTLYLQMNSLRAEDTAVYYCATHT |
| | (D1AF6517) | GDYFDYWGQGTMVTVSSASTKGPSV |
| | | FPLAPSSKSTSGGTAALGCLVKDYF |
| | | PEPVTVSWNSGALTSGVHTFPAVLQ |
| | | SSGLYSLSSVVTVPSSSLGTQTYIC |
| | | NVNHKPSNTKVDKKVEPKSCDKTHT |
| | | CPPCPAPEAAGGPSVFLFPPKPKDT |
| | | LMISRTPEVTCVVVDVSHEDPEVKF |
| | | NWYVDGVEVHNAKTKPREEQYNSTY |
| | | RVVSVLTVLHQDWLNGKEYKCKVSN |
| | | KALGAPIEKTISKAKGQPREPQVYT |
| | | LPPCRDELTKNQVSLWCLVKGFYPS |
| | | DIAVEWESNGQPENNYKTTPPVLDS |
| | | DGSFFLYSKLTVDKSRWQQGNVFSC |
| | | SVMHEALIINIIYTQKSLSLSPGK |
| 105 | P1AF8284 | DKTHTCPPCPAPEAAGGPSVFLFPP |
| | HChole Dotam- | KPKDTLMISRTPEVTCVVVDVSHED |
| | VL | PEVKFNWYVDGVEVHNAKTKPREEQ |
| | (D1AG3592) | YNSTYRWSVLTVLHQDWLNGKEYKC |
| | | KVSNKALGAPIEKTISKAKGQPREP |
| | | QVCTLPPSRDELTKNQVSLSCAVKG |
| | | FYPSDIAVEWESNGQPENNYKTTPP |
| | | VLDSDGSFFLVSKLTVDKSRWQQGN |
| | | VFSCSVMHEALHNHYTQKSLSLSPG |
| | | KGGGGSGGGGSGGGGSGGGGSSIQM |
| | | TQSPSSLSASVGDRVTITCQSSHSV |
| | | YSDNDLAWYQQKPGKAPKLLIYQAS |
| | | KLASGVPSRFSGSGSGTDFTLTISS |
| | | LQPEDFATYYCLGGYDDESDTYGFG |
| | | GGTKVEEK |
| 106 | P1AF8285 | DKTHTCPPCPAPEAAGGPSVFLFPP |
| | Hhole Dotam- | KPKDTLMISRTPEVTCVVVDVSHED |
| | VHA | PEVKFNWYVDGVEVHNAKTKPREEQ |
| | (D1AG3591) | YNSTYRVVSVLTVLHQDWLNGKEYK |
| | | CKVSNKALGAPIEKTISKAKGQPRE |
| | | PQVCTLPPSRDELTKNQVSLSCAVK |
| | | GFYPSDLAVEWESNGQPENNYKTTP |
| | | PVLDSDGSFFLVSKLTVDKSRWQQG |
| | | NVFSCSVMHEALHNHYTQKSLSLSP |
| | | GKGGGGSGGGGSGGGGSGGGGSVTL |
| | | KESGPVLVKPTETLTLTCTVSGFSL |
| | | STYSMSWIRQPPGKALEWLGFIGSR |
| | | GDTYYASWAKGRLTISKDTSKSQVV |
| | | LTMTNMDPVDTATYYCARERDPYGG |
| | | GAYPPHLWGRGTLVTVSSA |
| 107 | P1AF8284 | EIVLTQSPGTLSLSPGERATLSCRA |
| | and | SQSVSISGINLMNWYQQKPGQQPKL |
| | P1AF8285 | LIYHASILASGIPDRFSGSGSGTDF |
| | light | TLTISRLEPEDFAVYYCQQTRESPL |
| | chain | TFGQGTRLEIKRTVAAPSVFIFPPS |
| | (D1AF6469) | DEQLKSGTASVVCLLNNFYPREAIC |
| | | VQWKVDNALQSGNSQESVTEQDSKD |
| | | STYSLSSTLTLSKADYEKHKVYACE |
| | | VTHQGLSSPVTKSFNRGEC |
| 108 | P1AF8286 | EVQLLESGGGLVQPGGSLRLSCAAS |
| | and | GFTFSSYAMSWVRQAPGKGLEWVSA |
| | P1AF8287 | IIGSGASTYYADSVKGRFTISRDNS |
| | Hcknob | KNTLYLQMNSLRAEDTAVYYCAKGW |
| | <FAP> | FGGFNYWGQGTLVTVSSASTKGPSV |
| | 4B9 | FPLAPSSKSTSGGTAALGCLVKDYF |
| | (D1AF6515) | PEPVTVSWNSGALTSGVHTFPAVLQ |
| | | SSGLYSLSSWTVPSSSLGTQTYICN |
| | | VNHKPSNTKVDKKVEPKSCDKTHTC |
| | | PPCPAPEAAGGPSVFLFPPKPKDTL |
| | | MISRTPEVTCVVVDVSHEDPEVKFN |
| | | WYVDGVEVHNAKTKPREEQYNSTYR |
| | | VVSVLTVLHQDWLNGKEYKCKVSNK |
| | | ALGAPIEKTISKAKGQPREPQVYTL |
| | | PPCRDELTKNQVSLWCLVKGFYPSD |
| | | IAVEWESNGQPENNYKTTPPVLDSD |
| | | GSFFLYSKLTVDKSRWQQGNVFSCS |
| | | VMHEALHNHYTQKSLSLSPGK |
| 109 | P1AF8286 | DKTIITCPPCPAREAAGGPSVFLFP |
| | HChole Dotam- | PKPKDTLMISRTPEVTCVVVDVSII |
| | VL | NDPEVKPNWYVDGVEVHNAXTKPRE |
| | (D1AG3592) | EQYNSTYRVVSVLTVLHQDWLNGKE |
| | | YKCKVSNKALGAPIEKTISKAKGQP |
| | | REPQVCTLPPSRDELTKNQVSLSCA |
| | | VKGFYPSDIAVEWESNGQPENNYKT |
| | | TPPVLDSDGSFFLVSKLTVDKSRWQ |
| | | QGNVFSCSVMHEALHNHYTQKSLSL |
| | | SPGKGGGGSGGGGSGGGGSGGGGSS |
| | | IQMTQSPSSLSASVGDRVTITCQSS |
| | | HSVYSDNDLAWYQQKPGKAPKLLIY |
| | | QASKLASGVPSRFSGSGSGTDFTLT |
| | | ISSLQPEDFATYYCLGGYDDESDTY |
| | | GFGGGTKVEIK |
| 110 | P1AF8287 | DKTHTCPPCPAPEAAGGPSVFLFPP |
| | Hchole | KPKDTLMISRTPEVTCVVVDVSHED |
| | Dotam- | PEVKFNWTVDGVEVHNAKTKPREEQ |
| | VHA | YNSTYRVVSVLTVLHQDWLNGKEYK |
| | (D1AG3591) | CKVSNKALGAPIEKTISKAKGQPRE |
| | | PQVCTLPPSRDE |
| | | LTKNQVSLSCAVKGFYPSDIAVEWE |
| | | SNGQPENNYKTTPPVLDSDGSFFLV |
| | | SKLTVDKSRWQQGNVFSCSVMHEAL |
| | | HNHYTQKSLSLSPGKGGGGSGGGGS |
| | | GGGGSGGGGSVTLKESGPVLVKPTE |
| | | TLTLTCTVSGFSLSTYSMSWIRQPP |
| | | GKALEWLGFIGSRGDTYYASWAKGR |
| | | LTISKDTSKSQVVLTMTNMDPVDTA |
| | | TYYCARERDPYGGGAYPPHLWGRGT |
| | | LVTVSSA |
| 111 | P1AF8286 | EIVLTQSPGTLSLSPGERATLSCRA |
| | and | SQSVTSSYLAWYQQKPGQAPRLLIN |
| | P1AF8287 | VGSRRATGIPDRFSGSGSGTDFTLT |
| | light | ISRLEPEDFAVYYCQQGIMLPPTFG |
| | chain | QGTKVEIKRTVAAPSVFIFPPSDEQ |
| | (D1AB9974) | LKSGTASVVCLLNNFYPREAKVQWT |
| | | CVDN |
| | | ALQSGNSQESVTEODSKDSTYSLSS |
| | | TLTLSKADYEKHKVYACEVTHQGLS |
| | | SPVTKSFNRGEC |
| 112 | P1AF7782 and | QVQLVQSGAEVKKPGASVKVSCKAS |
| | P1AF7784 | GYTFTEFGMNWVRQAPGQGLEWMGW |
| | HCknob <CEA> | INTKTGEATYVEEFKGRVTFTTDTS |
| | CH1A1A | TSTAYMELRSLRSDDTAVYYCARWD |
| | (D1AD3419) | FAYYVEAMDYWGQGTTVTVSSASTK |
| | | GPSVFPLAPSSKSTSGGTAALGCLV |
| | | KDYFPEPVTVSWNSGALTSGVHTFP |
| | | AVLQSSGLYSLSSVVTVPSSSLGTQ |
| | | TYICNVNHKPSNTKVDKKVEPKSCD |
| | | KTHTCPPCPAPEAAGGPSVFLFPPK |
| | | PKDTLMISRTPEVTCVVVDVSHEDP |
| | | EVKFNWYVDGVEVHNAKTKPREEQY |
| | | NSTYRVVSVLTVLHQDWLNGKEYKC |
| | | KVSNKALGAPIEKTISKAKGQPREP |
| | | QVYTLPPCRDELTKNQVSLWCLVKG |
| | | FYPSDIAVEWESNGQPENNYKTTPP |
| | | VLDSDGSFFLYSKLTVDKSRWQQGN |
| | | VFSCSVMHEALHNHYTQKSLSLSPG |
| | | K |
| 113 | P1AF7782 | SIQMTQSPSSLSASVGDRVTITCQS |
| | Hchole | SHSVYSDNDLAWTQQKPGKAPKLLI |
| | Dotam- | YQASKLASGVPSRFSGSGSGTDFTL |
| | VL | TISSLQPEDFATYYCLGGYDDESDT |
| | (D1AG2237) | YGFGGGTKVEIKGGGGSGGGGSGGG |
| | | GSGGSGGDKTHTCPPCPAPEAAGGP |
| | | SVFLFPPKPKDTLMISRTPEVTCVV |
| | | VDVSHEDPEVKFNWYVDGVEVHNAK |
| | | TKPREEQYNSTYRVVSVLTVLHQDW |
| | | LNGKEYKCKVSNKALGAPIEKTISK |
| | | AKGQPREPQVCTLPPSRDELTKNQV |
| | | SLSCAVKGFYPSDLAVEWESNGQPE |
| | | NNYKTTPPVLDSDGSFFLVSKLTVD |
| | | KSRWQQGNVFSCSVMHEALHNHYTQ |
| | | KSLSLSPGK |
| 114 | P1AF7784 | GVTLKESGPVLVKPTETLTLTCTVS |
| | Hchole | GFSLSTYSMSWIRQPPGKALEWLGF |
| | Dotam- | IGSRGDTYYASWAKGRLTISKDTSK |
| | VH | S |
| | (D1AG2236) | QVVLTMTNMDPVDTATYYCARERDP |
| | | YGGGAYPPHLWGRGTLVTVSSGGGG |
| | | SGGGGSGGGGSGGSGGDKTHT |
| | | CPPCPAPEAAGGPSVFLFPPKPKDT |
| | | LMISRTPEVTCVVVDVSHEDPEVKF |
| | | NWYVDGVEVHNAKTKPREEQYNST |
| | | YRVVSVLTVLHQDWLNGKEYKCKVS |
| | | NKALGAPIEKTISKAKGQPREPQVC |
| | | TLPPSRDELTKNQVSLSCAVKGFYP |
| | | SDIAVEWESNGQPENNYKTTPPVLD |
| | | SDGSFFLVSKLTVDKSRWQQGNVFS |
| | | CSVMHEALITNITYTQKSLSLSPGK |
| 115 | P1AF7782 | DIQMTQSPSSLSASVGDRVTITCKA |
| | and | SAAVGTYVAWYQQKPGKAPKLLIYS |
| | P1AF7784 | ASYRKRGVPSRFSGSGSGTDFTLTI |
| | light | SSLQPEDFATYYCHQYYTYPLFTFG |
| | chain | QGTKLEIKRTVAAPSVFIFPPSDEQ |
| | (D1AD3421) | LKSGTASVVCLLNNFYPREAKVQWK |
| | | VDNALQSGNSQESVTEQDSKDSTYS |
| | | LSSTLTLSKADYEKHKVYACEVTHQ |
| | | GLSSPVTKSFNRGEC |
| 156 | heavy chain | DSYMH |
| | CDR1 <CEA> | |
| | MFE23 | |
| 157 | heavy chain | WrDPENGDTEYAPKFQG |
| | CDR2 <CEA> | |
| | MFE23 | |
| 158 | heavy chain | WIDPENGGTNYAQKFQG |
| | CDR2 <CEA> | |
| | MFE23-H26 | |
| 159 | heavy chain | GTPTGPYYFDY |
| | CDR3 <CEA> | |
| | MFE23 | |
| 160 | light chain | SASSSVSYMH |
| | CDR1 <CEA> | |
| | MFE23 | |
| 161 | light chain | RASSSVSYMH |
| | CDR1 <CEA> | |
| | MFE23-L24, | |
| | L25 | |
| 162 | light chain | RASQSISSYM |
| | CDR1<CEA> | |
| | MFE23-L26 | |
| 163 | light chain | STSNLAS |
| | CDR2<CEA> | |
| | MFE23 | |
| 164 | Light chain | YTSNLAS |
| | CDR2<CEA> | |
| | MFE23-L26 | |
| 165 | Light chain | STSSLQS |
| | CDR2<CEA> | |
| | MFE23-L29 | |
| 166 | light chain | QQRSSYPLT |
| | CDR3<CEA> | |
| | MFE23 | |
| 167 | Heavy chain | QVKLQQSGAELVRSGTSVKLSCTAS |
| | variable | GFNTKDSYMHWLRQGPEQGLEWIGW |
| | domain | IDPENGDTEYAPKFQGKATFTTDTS |
| | <CEA> MFE23 | SNTAYLQLSSLTSEDTAVYYCNEGT |
| | | PTGPYYFDYWGQGTTVTVSS |
| 168 | Light chain | ENVLTQSPAIMSASPGEKVTITCSA |
| | variable | SSSVSYMHWFQQKPGTSPKLWTYST |
| | domain | SNLASGVPARFSGSGSGTSYSLTIS |
| | <CEA> MFE23 | RMEAEDAATYYCQQRSSYPLTFGAG |
| | | TKLELK |
| 169 | MFE-H24 | QVQLVQSGAEVKKPGASVKVSCKAS |
| | | GFNIKDSYMHWVRQAPGQGLEWMGW |
| | | IDPENGDTEYAPKFQGRVTMTTDTS |
| | | ISTAYMELSRLRSDDTAVYYCNEGT |
| | | PTGPYYFDYWGQGTLVTVSS |
| 170 | MFE-H25 | QVQLVQSGAEVKKPGASVKVSCKAS |
| | | GYTFKDSYMHWVRQAPGQGLEWMGW |
| | | IDPENGDTEYAPKFQGRVTMTTDTS |
| | | ISTAYMELSRLRSDDTAVYYCNEGT |
| | | PTGPYYFDYWGQGTLVTVSS |
| 171 | MFE-H26 | QVQLVQSGAEVKKPGASVKVSCKAS |
| | | GFNIKDSYMHWVRQAPGQGLEWMGW |
| | | IDPENGGTNYAQKFQGRVTMTTDTS |
| | | ISTAYMELSRLRSDDTAVYYCNEGT |
| | | PTGPYYFDYWGQGTLVTVSS |
| 172 | MFE-H27 | QVQLVQSGAEVKKPGASVKVSCKAS |
| | | GFNIKDSYMHWVRQAPGQGLEWMGW |
| | | IDPENGDTEYAPKFQGRVTMTTDTS |
| | | ISTAYMELSRLRSDDTAVYYCARGT |
| | | PTGPYYFDYWGQGTLVTVSS |
| 173 | MFE-H28 | QVQLVQSGAEVKKPGASVKVSCKAS |
| | | GFNIKDSYMHWVRQAPGQGLEWMGW |
| | | IDPENGDTEYAPKFQGRVTMTRDTS |
| | | ISTAYMELSRLRSDDTAVYYCNEGT |
| | | PTGPYYFDYWGQGTLVTVSS |
| 174 | MFE-H29 | QVQLVQSGAEVKKPGSSVKVSCKAS |
| | | GFNIKDSYMHWVRQAPGQGLEWMGW |
| | | IDPENGDTEYAPKFQGRVTNTDEST |
| | | STAYMELSSLRSEDTAVYYCNEGTP |
| | | TGPYYFDYWGQGTLVTVSS |
| 175 | MFE-L24 | DIQMTQSPSSLSASVGDRVTITCRA |
| | | SSSVSYMHWYQQKPGKAPKLLIYST |
| | | SNLASGVPSRFSGSGSGTDFTLTIS |
| | | SLQPEDFATYYCQQRSSYPLTFGGG |
| | | TKLEIK |
| 176 | MFE-L25 | EIQMTQSPSSLSASVGDRVTITCRA |
| | | SSSVSYMHWYQQKPGKAPKLLIYST |
| | | SNLASGVPSRFSGSGSGTDFTLTIS |
| | | SLQPEDFATYYCQQRSSYPLTFGGG |
| | | TKLEIK |
| 177 | MFE-L26 | EIQMTQSPSSLSASVGDRVTITCRA |
| | | SQSISSYMHWYQQKPGKAPKLLIYS |
| | | TSNLASGVPSRFSGSGSGTDFTLTI |
| | | SSLQPEDFATYYCQQRSSYPLTFGG |
| | | GTKLEIK |
| 178 | MFE-L27 | EIQMTQSPSSLSASVGDRVTITCRA |
| | | SSSVPYMHWYQQKPGKAPKLLIYST |
| | | SNLASGVPSRFSGSGSGTDFTLTIS |
| | | SVQPEDFATYYCQQRSSYPLTFGGG |
| | | TKLEIK |
| 179 | MFE-L28 | EIQMTQSPSSLSASVGDRVTITCRA |
| | | SSSVPYMHWLQQKPGKAPKLLIYST |
| | | SNLASGVPSRFSGSGSGTDFTLTIS |
| | | SVQPEDFATYYCQQRSSYPLTFGGG |
| | | TKLEIK |
| 180 | MFE-L29 | EIQMTQSPSSLSASVGDRVTITCRA |
| | | SSSVPYMHWLQQKPGKAPKLLIYST |
| | | SSLQSGVPSRFSGSGSGTDFTLTIS |
| | | SVQPEDFATYYCQQRSSYPLTFGGG |
| | | TKLEIK |

### IV. Examples

The following are examples of methods and compositions of the invention. It is understood that various other embodiments may be practiced, given the general description provided above.

### Glossary of Abbreviations

ADA Anti-drug antibody
AST Alanine, serine, threonine
BsAb Bispecific antibody
CA Clearing agent
CEA Carcinoembryonic antigen
DOTAM 1,4,7,10-tetrakis(carbamoylmethyl)-1,4,7,10-tetraazacyclododecane
ID Injected dose
ELISA Enzyme-linked immunosorbent assay
FAP Fibroblast activation protein
GPRC5D G-protein coupled receptor family C group 5 member D

### IV Intravenous

MW Molecular weight
PBS Phosphate-buffered saline
p.i. Post injection

### PK Pharmacokinetic

PRIT Pretargeted radioimmunotherapy

### RIT Radioimmunotherapy

RT Room temperature

### SC Subcutaneous

SCID Severe combined immunodeficiency
SD Standard deviation
SOPF Specific and opportunistic pathogen-free
TA Target antigen
TGI Tumor growth inhibition
TR Tumor regression

### Example 1: Generation of Rabbit DOTAM Binding Antibodies

### Example 1a: Immunization of Rabbits

A 1:1 mix of the 2 enantiomeric Pb-DOTAM-alkyl-PEG₄-KLH fractions (MS2-DOTAM KLH Fraction 1 and MS2-DOTAM KLH Fraction 2) was used for the immunization of New Zealand White rabbits or transgenic rabbits comprising a human immunoglobulin locus as reported in WO 2000/46251, WO 2002/12437, WO 2005/007696, WO 2006/047367, US 2007/0033661, and WO 2008/027986. Each rabbit was immunized with 500 ug of the immunogen mix, emulsified with complete Freund's adjuvant, at day 0 by intradermal application and 500 ug each at days 7, 14, 28, 56 by alternating intramuscular and subcutaneous applications. Thereafter, rabbits received monthly subcutaneous immunizations of 500 ug, and small samples of blood were taken 7 days after immunization for the determination of serum titers. A larger blood sample (10% of estimated total blood volume) was taken during the third and during the ninth month of immunization (at 5-7 days after immunization), and peripheral mononuclear cells were isolated, which were used as a source of antigen-specific B cells in the B cell cloning process.

### Determination of Serum Titers (ELISA)

Each of the 2 enantiomeric Pb-DOTAM fractions (PJRD05.133F1 or PJRD05.133F2) was immobilized on a 96-well NUNC Maxisorp plate at 1 ug/ml, 100 ul/well, in PBS, followed by: blocking of the plate with 2% Crotein C in PBS, 200 ul/well; application of serial dilutions of antisera, in duplicates, in 0.5% Crotein C in PBS, 100 ul/well; detection with HRP-conjugated donkey anti-rabbit IgG antibody (Jackson Immunoresearch/Dianova 711-036-152; 1/16 000) and streptavidin-HRP; each diluted in 0.5% Crotein C in PBS, 100 ul/well. For all steps, plates were incubated for 1 h at 37° C. Between all steps, plates were washed 3 times with 0.05% Tween 20 in PBS. Signal was developed by addition of BM Blue POD Substrate soluble (Roche), 100 ul/well; and stopped by addition of 1 M HC1, 100 ul/well. Absorbance was read out at 450 nm, against 690 nm as reference. Titer was defined as dilution of antisera resulting in half-maximal signal.

### Example 1B: B-Cell Cloning from Rabbits

### Isolation of Rabbit Peripheral Blood Mononuclear Cells (PBMCs)

Blood samples were taken of immunized rabbits. EDTA containing whole blood was diluted twofold with 1×PBS (PAA, Pasching, Austria) before density centrifugation using lympholyte mammal (Cedarlane Laboratories, Burlington, Ontario, Canada) according to the specifications of the manufacturer. The PBMCs were washed twice with 1×PBS.

### EL-4 B5 Medium

RPMI 1640 (Pan Biotech, Aidenbach, Germany) supplemented with 10% FCS (Hyclone, Logan, Utah, USA), 2 mM Glutamin, 1% penicillin/streptomycin solution (PAA, Pasching, Austria), 2 mM sodium pyruvate, 10 mM HEPES (PAN Biotech, Aidenbach, Germany) and 0.05 mM b-mercaptoethanole (Gibco, Paisley, Scotland) was used.

### Coating of Plates

Sterile cell culture 6-well plates were coated with 2 μg/ml KLH in carbonate buffer (0,1 M sodium bicarbonate, 34 mM Disodiumhydrogencarbonate, pH 9,55) overnight at 4° C. Plates were washed in sterile PBS three times before use. Sterile streptavidin coated 6-well plates (Microcoat, Bernried, Germany) were coated with a 1+1 enantiomer mixture of biotinylated TCMC-Pb-dPEC3-Biotin Isomer A (1 μg/ml) and B (1 μg/ml) in PBS for 3 h at room temperature. Prior to the panning step these 6-well plates were washed three times with sterile PBS.

### Depletion of Macrophages/Monocytes

The PBMCs were seeded on sterile KLH-coated 6-well-plates to deplete macrophages and monocytes through unspecific adhesion and to remove cells binding to KLH. Each well was filled at maximum with 4 ml medium and up to 6×10e6 PBMCs from the immunized rabbit and were allowed to bind for 1 h at 37° C. and 5% CO2. The cells in the supernatant (peripheral blood lymphocytes (PBLs)) were used for the antigen panning step.

### Enrichment of B Cells on the Pb-Containing TCMC Enantiomer

6-well plates coated with the enantiomer mixture of TCMC-Pb-dPEC3-Biotin Isomer A and B were seeded with up to 6×10e6 PBLs per 4 ml medium and allowed to bind for 1 h at 37° C. and 5% CO2. Non-adherent cells were removed by carefully washing the wells 1-3 times with 1×PBS. The remaining sticky cells were detached by trypsin for 10 min at 37° C. and 5% CO2. Trypsination was stopped with EL-4 B5 medium. The cells were kept on ice until the immune fluorescence staining.

### Immune Fluorescence Staining and Flow Cytometry

The anti-IgG FITC (AbD Serotec, Düsseldorf, Germany) was used for single cell sorting. For surface staining, cells from the depletion and enrichment step were incubated with the anti-IgG FITC antibody in PBS and incubated for 45 min in the dark at 4° C. After staining the PBMCs were washed two times with ice cold PBS. Finally the PBMCs were resuspended in ice cold PBS and immediately subjected to the FACS analyses. Propidium iodide in a concentration of 5 μg/ml (BD Pharmingen, San Diego, Calif., USA) was added prior to the FACS analyses to discriminate between dead and live cells.

A Becton Dickinson FACSAria equipped with a computer and the FACSDiva software (BD Biosciences, USA) were used for single cell sort.

### B-Cell Cultivation

The cultivation of the rabbit B cells was prepared by a method described by Lightwood et al (J Immunol Methods, 2006, 316: 133-143). Briefly, single sorted rabbit B cells were incubated in 96-well plates with 200 μl/well EL-4 B5 medium containing Pansorbin Cells (1:100000) (Calbiochem (Merck), Darmstadt, Deutschland), 5% rabbit thymocyte supernatant (MicroCoat, Bernried, Germany) and gamma-irradiated murine EL-4 B5 thymoma cells (5×10e5 cells/well) for 7 days at 37° C. in the incubator. The supernatants of the B-cell cultivation were removed for screening and the remaining cells were harvested immediately and were frozen at −80° C. in 100 μl RLT buffer (Qiagen, Hilden, Germany).

### Example 1C: Expression of Rabbit Antibody

### PCR Amplification of V-Domains

Total RNA was prepared from B cells lysate (resuspended in RLT buffer—Qiagen—Cat. N^{o} 79216) using the NucleoSpin 8/96 RNA kit (Macherey&Nagel; 740709.4, 740698) according to manufacturer's protocol. RNA was eluted with 60 μl RNase free water. 6 μl of RNA was used to generate cDNA by reverse transcriptase reaction using the Superscript III First-Strand Synthesis SuperMix (Invitrogen 18080-400) and an oligo dT-primer according to the manufacturers' instructions. All steps were performed on a Hamilton ML Star System. 4 μl of cDNA were used to amplify the immunoglobulin heavy and light chain variable regions (VH and VL) with the AccuPrime Supermix (Invitrogen 12344-040) in a final volume of 50 μl using the primers rbHC.up and rbHC.do for the heavy chain and rbLC.up and rbLC.do for the light chain (Table below). All forward primers were specific for the signal peptide (of respectively VH and VL) whereas the reverse primers were specific for the constant regions (of respectively VH and VL). The PCR conditions for the RbVH+RbVL were as follows: Hot start at 94° C. for 5 min; 35 cycles of 20 s at 94° C., 20 s at 70° C., 45 s at 68° C., and a final extension at 68° C. for 7 min.

### Primer Sequences

| rbHC.up | AAGCTTGCCACCATGGAGACTGGGCTGCGCTGGCTTC |
|---|---|
| | (SEQ ID NO.: 141) |
| rbHCf.do | CCATTGGTGAGGGTGCCCGAG (SEQ ID NO.: 142) |
| rbLC.up | AAGCTTGCCACCATGGACAYGAGGGCCCCCACTC |
| | (SEQ ID NO.: 143) |
| rbLC.do | CAGAGTRCTGCTGAGGTTGTAGGTAC (SEQ ID NO.: |
| | 144) |

8 μl of 50 μl PCR solution were loaded on a 48 E-Gel 2% (Invitrogen G8008-02). Positive PCR reactions were cleaned using the NucleoSpin Extract II kit (Macherey&Nagel; 740609250) according to manufacturer's protocol and eluted in 50 μl elution buffer. All cleaning steps were performed on a Hamilton ML Starlet System.

### Recombinant Expression of Rabbit Monoclonal Bivalent Antibodies

For recombinant expression of rabbit monoclonal bivalent antibodies, PCR-products coding for VH or VL were cloned as cDNA into expression vectors by the overhang cloning method (R S Haun et al., Biotechniques (1992) 13, 515-518; MZ Li et al., Nature Methods (2007) 4, 251-256). The expression vectors contained an expression cassette consisting of a 5′ CMV promoter including intron A, and a 3′ BGH poly adenylation sequence. In addition to the expression cassette, the plasmids contained a pUC18-derived origin of replication and a beta-lactamase gene conferring ampicillin resistance for plasmid amplification in E. coli. Three variants of the basic plasmid were used: one plasmid containing the rabbit IgG constant region designed to accept the VH regions while two additional plasmids containing rabbit or human kappa LC constant region to accept the VL regions. Linearized expression plasmids coding for the kappa or gamma constant region and VL/VH inserts were amplified by PCR using overlapping primers. Purified PCR products were incubated with T4 DNA-polymerase which generated single-strand overhangs. The reaction was stopped by dCTP addition. In the next step, plasmid and insert were combined and incubated with recA which induced site specific recombination. The recombined plasmids were transformed into E. coli. The next day the grown colonies were picked and tested for correct recombined plasmid by plasmid preparation, restriction analysis and DNA-sequencing. For antibody expression, the isolated HC and LC plasmids were transiently co-transfected into 2 ml (96 well plate) of FreeStyle HEK293-F cells (Invitrogen R790-07) by using 239-Free Transfection Reagent (Novagen) following procedure suggested by Reagent supplier. The supernatants were harvested after 1 week and delivered for purification.

### Example 1D: Selection of Rabbit Monoclonal Antibodies

A SET (solution equilibration titration) assay was carried out as described below.

### SET Assay

### Materials:

1. DOTAM-Biotin-Isomer Mix:
Mixture of the following components, conc.=20 ng/ml
   Pb-Dotam-Bn-biotin/TCMC-Pb-dPEG3-Biotin, isomer A
   Pb-Dotam-Bn-biotin/TCMC-Pb-dPEG3-Biotin, isomer B
   Pb-Dotam-alkyl-biotin isomer A
   Pb-Dotam-alkyl-biotin isomer B
2. PBS: DPBS, PAN, P04-36500
3. BSA: Roche, 10735086001
4. Tween 20: Polysorbat 20 (usb, #20605, 500 ml)
5. PBST: 10×, Roche, #11666789001/0,1% Tween 20
6. OSEP: PBS (10×, Roche, #11666789001)/0,5% BSA (Bovine Serum Albumin Fraction V, fatty acid free, Roche, #10735086001)/0,05% Tween 20

Preparation of assay-plate: 384-well streptavidin plates (Nunc, Microcoat #11974998001) were incubated overnight at 4° C. with 25 μl/well of a DOTAM-Biotin-Isomer Mix in PBS-buffer at a concentration of 20 ng/ml.

Equilibration of anti-DOTAM antibody samples with free DOTAM-metal chelates (Pb, Bi, Ca, Cu, Zn, Mg, Fe): 0.01 nM-1 nM of antibody were titrated with the relevant DOTAM-metal chelates in 1:3, 1:2 or 1:1.7 dilution steps starting at a concentration of 2500 nM, 500 nM or 100 nM of DOTAM-metal chelate. The samples were incubated at 4° C. overnight in sealed REMP Storage polypropylene microplates (Brooks).

After overnight incubation, streptavidin plates were washed 3× with 90 μl PBST per well. 15 μl of each sample from the equilibration plate were transferred to the assay plate and incubated for 15 min at RT, followed by 3×90 μl washing steps with PBST buffer. Detection was carried out by adding 25 μl of a goat anti-human IgG antibody-POD conjugate (Jackson, 109-036-088, 1:4000 in OSEP), followed by 6×90 μl washing steps with PBST buffer. 25 μl of TMB substrate (Roche Diagnostics GmbH, Cat. No.: 11835033001) were added to each well. Measurement took place at 370/492 nm on a Safire2 reader (Tecan).

The table below shows the properties of various monoclonal bivalent rabbit antibodies as determined using this assay. PRIT-0128 was selected as the lead candidate as it has comparable binding to chelated Pb and Bi, reduced binding to other chelated metals, and high affinity (<100 pM).

### Binding of Monoclonal Bivalent Rabbit Antibodies to Chelated Metals

| | | KD [SET-Titration] | | | |
|---|---|---|---|---|---|
| Name | Species | Pb | Bi | Ca | Zn |
| PRIT-0135 | WTRa | 0.000 | 0.000 | 0.001 | 0.285 |
| PRIT-0129 | WTRa | 0.001 | 0.024 | 0.013 | 9.251 |
| PRIT-0128 | WTRa | 0.002 | 0.003 | 0.003 | 8.152 |
| PRIT-0132 | WTRa | 0.002 | 0.046 | 0.002 | 0.217 |
| PRIT-0134 | WTRa | 0.002 | 0.059 | 0.003 | 0.443 |
| PRIT-0136 | WTRa | 0.004 | 0.029 | 0.014 | 131.926 |
| PRIT-0127 | WTRa | 0.014 | 0.092 | 0.015 | 222.339 |
| PRIT-0107 | TgRa | 1.1 | 51.0 | 48.0 | >1000 |

WTRa: Wild Type Rabbits; TgRa: transgenic rabbits

### Example 2: Humanization

### Humanization

Next, the lead candidate PRIT-0128 was subject to humanization.

For the identification of a suitable human acceptor framework during the humanization of the DOTAM binder PRIT-0128, a combination of two methodologies was used. On the one hand a classical approach was taken by searching for an acceptor framework with high sequence homology to the parental antibody and subsequent grafting of the CDR regions onto this acceptor framework. Each amino acid difference of the identified frameworks to the parental antibody was judged for impact on the structural integrity of the binder and backmutations towards the parental sequence were introduced whenever appropriate.

On the other hand, an in house developed in silica tool was used to predict the orientation of the VH and VL domains of the humanized versions towards each other (see WO2016/062734). This was carried out for the virtual grafts of the CDRs on all possible human germline combinations. The results were compared to the VH-VL domain orientation of the parental binder to select for framework combinations which are close in geometry to the starting antibody.

In each case the following CDR regions of the parental antibody were grafted onto the acceptor framework (numbering according to Kabat):

### VH_CDR1: 31-35

### VH_CDR2: 50-65

### VH_CDR3: 95-102

### VL_CDR1: 24-34

### VL_CDR2: 50-56

### VL_CDR3: 89-97

The humanization variants were produced in a format comprising a full length antibody for CEA, with the C-terminus of one of the heavy chains fused to the N-terminus of the VH domain the Dotam-binder, and the C-terminus of the other heavy chain fused to the N-terminus of the VL domain of the DOTAM-binder, forming a bispecific antibody with two binding sites for CEA and one functional binding site for DOTAM. Thus, the DOTAM binder was fused to the C-terminus of the Fc of the tumour targeting IgG as an VH/VL Fv fusion (without CH1 and Ck respectively). The parental DOTAM binder PRIT-0128 derived molecule in this bispecific format is called PRIT-0156.

Herceptin framework was included as well due to the suitability in terms of VH/VL prediction and the increased stability of the framework. For all VH humanized variants, the human J element hJH2 was used. For all VK humanized variants, the human J element hJK4 was used.

The HC4 is a grafting of PRIT-128 on the human germline IGHV3-30-02 with one backmutation Kabat A49G.

To get the variable heavy chain HC5, the CDRs were grafted on human germline hVH_2_26 with A49G as a backmutation and the deletion of the first amino acid to reflect the original rabbit N-terminus.

Grafted on Herceptin V-region (derived from human germline hVH3_66), the variant HC7 is characterized by a few modifications in the acceptor framework: deletion of N-terminus E, A49G, A71R, and S93A.

For HC10, the CDRs of PRIT-128 were grafted on the human germline IGHV4_34_01.

Here, the N-terminus was modified, starting with V2, to reflect the original rabbit antibody starting with Q2. In addition, G29F and F31L were considered as backmutation wrt Kabat nomenclature as well as V71R and F78V in framework 3.

For the light chain LC1, the CDRs were grafted on the human germline IGKV1_39_01 without any backmutation. The start was chosen as 12 to reflect the original rabbit Ab starting with A2.

The light chain variant LC3 was obtained by grafting of the CDRs on human germline hVK1_5. D1 was deleted and a I2A backmutation was considered as a new N-terminus. As additional backmutations, K42Q and A43P were taken into account.

Not all possible combinations of the humanization matrix were produced but a selection of defined combinations was chosen based on considerations like VH/VL prediction and sequence risks of the given combination.

### Selection of Candidates

The goal of the humanization was to obtain humanized binders which do not lose more than a factor of 10 in terms of affinity to DOTAM. This was achieved with several binders of comparable or even better affinity to DOTAM.

As mentioned above, PRIT-0156 is a 2:1 antibody comprising the rabbit DOTAM binder PRIT-0128 combined with the CEA binder CH1A1A. PRIT-0178 to PRIT-0204 are humanized variants in the same format with the same CEA binder. PRIT-0205 up to PRIT-0221 correspond to the PRIT-0178 to PRIT-0204 humanization variants in the DOTAM binding part, but have the CEA binder changed to T84.66.

### Solution Equilibrium Based kd Determination

To screen a larger amount of humanization candidates for their affinity to Pb-DOTAM, solution equilibrium titration (SET) was used. The table below details the SET based affinity determination of selected humanized DOTAM binders against Pb-DOTAM. All antibodies in this table are bispecific antibodies that comprise bivalent binding to CEA and monovalent binding to Pb-Dotam (2:1 format):

| | Humanized HC/LC | |
|---|---|---|
| Molecule name | combination | SET Pb-DOTAM (pM) |
| PRIT-0187-0002 | HC10 LC1 | 0.03 |
| PRIT-0193-0002 | HC5 LC3 | 0.36 |
| PRIT-0195-0004 | HC10 LC3 | 0.40 |
| PRIT-0156-0004 | Parental molecule | 0.43 |
| PRIT-0182-0002 | HC8 LC7 | 5.54 |
| PRIT-0189-0002 | HC7 LC2 | 5.81 |
| PRIT-0185-0002 | HC 2 LC1 | 5.87 |
| PRIT-0192-0002 | HC2 LC3 | 8.04 |
| PRIT-0183-0004 | HC2 LC2 | 8.11 |
| PRIT-0197-0002 | HC7 LC1 | 8.69 |
| PRIT-0198-0002 | HC7 LC3 | 9.09 |
| PRIT-0182-0004 | HC8 LC7 | 17.14 |
| PRIT-0183-0002 | HC2 LC2 | 22.45 |
| PRIT-0180-0004 | HC9 LC6 | 26.10 |
| PRIT-0190-0002 | HC9 LC2 | 33.83 |
| PRIT-0194-0002 | HC8 LC3 | 34.63 |
| PRIT-0199-0002 | HC9 LCI | 43.91 |
| PRIT-0180-0002 | HC9 LC6 | 47.70 |
| PRIT-0188-0002 | HC4 LC2 | 54.34 |
| PRIT-0178-0004 | HC1 LC6 | 60.06 |
| PRIT-0179-0004 | HC4 LC6 | 64.92 |
| PRIT-0181-0002 | HC4 LC7 | 65.11 |
| PRIT-0179-0002 | HC4 LC6 | 65.81 |
| PRIT-0178-0002 | HC1 LC6 | 66.68 |
| PRIT-0187-0004 | HC10 LC1 | 78.09 |
| PRIT-0184-0002 | HC1 LC1 | 80.56 |
| PRIT-0200-0002 | HC9 LC3 | 83.24 |
| PRIT-0191-0002 | HC1 LC3 | 111.10 |

### Kinexa Based kd Determination

For more detailed analysis and an orthogonal method for affinity determination, Kinexa was used.

### Instrumentation and Materials

A KinExA 3200 instrument from Sapidyne Instruments (Boise, Id.) with autosampler was used. Polymethylmethacrylate (PMMA) beads were purchased from Sapidyne, whereas PBS (phosphate buffered saline), BSA (bovine serum albumin fraction V) and the anti-DOTAM antibodies were prepared in-house (Roche). Dylight650®-conjugated affinity-purified goat anti-human IgG-Fc Fragment cross-adsorbed antibody was purchased from Bethyl Laboratories (Montgomery, Tex.). The biotinylated Pb-DOTAM antigens (Pb-DOTAM-alkyl-biotin isomer A and B, Pb-DOTAM-Bn-biotin/TCMC-Pb-dPEG3-Biotin, isomer A and B) and the non-biotinylated Pb-DOTAM were obtained from AREVA Med (Bethesda, Md.).

### Preparation of Antigen Coated Beads

PMMA beads were coated according to the KinExA Handbook protocol for biotinylated molecules (Sapidyne). Briefly, first, 10 μg of Biotin-BSA (Thermo Scientific) in 1 ml PBS (pH7.4) was added per vial (200 mg) of beads for adsorption coating. After rotating for 2 h at room temperature, the supernatant was removed and beads were washed 5 times with 1 ml PBS. Second, 1 ml of 100 μg of NeutrAvidin Biotin-Binding Protein (Thermo Scientific) in PBS containing 10 mg/ml BSA was added to the beads and incubated at room temperature for additional 2 h to couple NeutrAvidin to the beads and to provide additional biotin binding sites for subsequent binding of biotinylated proteins. The NeutrAvidin-coated-beads were then rinsed 5 times with 1 ml PBS. Finally, the beads were coated with 200 ng/ml biotinylated Pb-DOTAM-Isomer Mix (50 ng for each Isomer) in PBS and incubated for further 2 h at room temperature. Beads were then resuspended in 30 ml PBS and used immediately.

### KinExA Equilibrium Assays

All KinExA experiments were performed at room temperature (RT) using PBS pH 7.4 as running buffer. Samples were prepared in running buffer supplemented with 1 mg/ml BSA (“sample buffer”). A flow rate of 0.25 ml/min was used. A constant amount of anti-DOTAM antibody with 5 pM binding site concentration was titrated with Pb-DOTAM antigen by twofold serial dilution starting at 100 pM (concentration range 0.049 pM-100 pM). One sample of antibody without antigen served as 100% signal (i.e. without inhibition). Antigen-antibody complexes were incubated at RT for at least 24 h to allow equilibrium to be reached. Equilibrated mixtures were then drawn through a column of Pb-DOTAM-coupled beads in the KinExA system at a volume of 5 ml permitting unbound antibody to be captured by the beads without perturbing the equilibrium state of the solution. Captured antibody was detected using 250 ng/ml Dylight 6500-conjugated anti-human Fc-fragment specific secondary antibody in sample buffer. Each sample was measured in duplicates for all equilibrium experiments.

The KD was obtained from non-linear regression analysis of the data using a one-site homogeneous binding model contained within the KinExA software (Version 4.0.11) using the “standard analysis” method. The software calculates the KD and determines the 95% confidence interval by fitting the data points to a theroretical KD curve. The 95% confidence interval (Sapidyne TechNote TN207R0) is given as KD low and KD high.

### Thermostability Measurements for Humanized PRIT Molecules

### Method and Data Analysis

Different variants of the humanized PRIT molecules in the final format (in 20 mM Histidine, 140 mM NaCl, pH 6.0) were diluted in the same buffer to 1 mg/ml. 30 μl of each sample was transferred into a 384-well plate filter device (alongside an anti-HER3 antibody as reference). After centrifugation at 1,000 g for 1 min wells were overlaid with 10 μl of paraffin oil. The plate was centrifuged again (1,000 g for 1 min) and transferred into the DLS pklate reader (Dyna Pro PlateReader-II, Wyatt). Starting at 25° C. the temperature was increased at a speed of 0.05° C./min to 79.9° C. Scattered light was recorded using the Dynamics Software (V7.0).

Data was transferred to Excel (Microsoft), sorted by sample and temperature and a software add-in used to create melting curves. The temperature, where clear deviation from the baseline occurred, was defined as “onset of aggregation” and the inflection point of the melting curve as “melting temperature”.

### Results

| Sample | onset of aggregation (in ° C.) | melting temperature (in ° C.) |
|---|---|---|
| Her 3 | 63 ± 1 | 67.5 ± 1 |
| PRIT-0156 | 45 ± 1 | 51 ± 1 |
| (parental | | |
| molecule | | |
| with rabbit | | |
| DOTAM | | |
| binder) | | |
| PRIT 205 | 51 ± 1 | 51.5 ± 1 |
| PRIT 206 | 51 ± 1 | 58.1 ± 1 |
| PRIT 207 | 49 ± 1 | 58.1 ± 1 |
| PRIT 208 | 54 ± 1 | 57.7 ± 1 |
| PRIT 209 | 54 ± 1 | 57.7 ± 1 |
| PRIT-0213 | 54 ± 1 | 57.7 ± 1 |

### Properties of Candidates

The tables below summarize the identity of various PRIT molecules and compare their properties. Preferred compounds were PRIT-0213 and PRIT-0214.

### Summary of Candidates

| | CEA | | |
|---|---|---|---|
| Antibody | binder | Dotam binder | Format |
| PRIT 0218 | none | WT | Rabbit antibody |
| PRIT0156 | CHIA1A | PRIT-0218 | 2:1 antibody with |
| | | | PRIT 0128 dotam |
| | | | binder |
| PRIT 0186 | CH1A1A | HC5/LC1 (humanized PRIT- | 2:1 format |
| | | 0218) | |
| PRIT-0213 | T84.86 | HC5/LC1 (humanized PRIT- | 2:1 format |
| | | 0218) | |
| PRIT-0187 | CH1A1A | HC10/LC1 (humanized PRIT- | 2:1 format |
| | | 0218) | |
| PRIT-0214 | T84.86 | HC10/LC1 (humanized PRIT- | 2:1 format |
| | | 0218) | |
| PRIT-0206 | T84.86 | HC5/LC3 (humanized PRIT- | 2:1 format |
| | | 0218) | |
| PRIT-0216 | T84.86 | HC5/LC3 (humanized PRIT- | 2:1 format |
| | | 0218) | |
| PRIT-0217 | T84.86 | HC5/LC3 (humanized PRIT- | 2:1 format |
| | | 0218) | |
| PRIT-0208 | T84.86 | HC7/LC1 (humanized PRIT- | 2:1 format |
| | | 0218) | |

### Comparison of Properties

| | | | SET | Kinexa | | | | | |
|---|---|---|---|---|---|---|---|---|---|
| | CEA | DOTAM | PbDOTAM | PbDOTAM | Yield | T Agg | | | |
| | BiCEAnder | Binder | [PM] | [PM] | [mg/l] | 266 nm | Humanness | | Biodistribution |
| PRIT-0156 | CH1A1A | WT Rabbit | 0.01 | 0.25 | 25 | 45 | — | — | Ok |
| PRIT-0186 | CH1A1A | HC5/LC1 | 0.4 | 0.92 | 38 | 50 | HC | LC | Ok |
| | | | | | | | 58% | 57% | |
| PRIT-0213 | T84.66 | HC5/LC1 | 0.4 | 0.84 | 4.0 | 54 | HC | LC | Ok |
| | | | | | | | 58% | 57% | |
| PRIT-0187 | CH1A1A | HC10/ LC1 | 0.03 | 0.99 | 17.8 | 57 | HC | LC | Ok |
| | | | | | | | 40% | 57% | |
| PRIT-0214 | T84.66 | HC10/LC1 | 0.03 | 0.84 | 6.0 | 55 | HC | LC | Ok |
| | | | | | | | 40% | 57% | |
| PRIT-0206 | T84.66 | HC5/LC3 | 0.36 | 1.0 | 1.32 | 50 | HC | LC | Ok |
| | | | | | | | 58% | 55% | |
| PRIT-0216 | T84.66 | HC5/LC3 | — | 51 | 7.0 | 50 | HC | LC | Ok |
| | | | | | | | 58% | 55% | |
| PRIT-0217 | T84.66 | HC5/LC3 | — | 3 | 10.0 | 54 | HC | LC | Ok |
| | | | | | | | 58% | 55% | |
| PRIT-0208 | T84.66 | HC7/LC1 | 8.7 | 8.3 | 7.6 | 59 | HC | LC | Ok |
| | | | | | | | 51% | 57% | |

Further data for affinity values as determined by Kinexa are provided below, for PRIT-0213, (PRIT-0213 is the same molecule as PRIT-0186, except for another CEA binding VH/VL.)

### PRIT-0213

### Metal-DOTAM Chelate Affinities of CEA-DOTAM BsAb

| Antigen | KD [pM] | 95% CI[pM] |
|---|---|---|
| Pb-DOTAM | 0.84 | 0.44-1.4 |
| Ca-DOTAM | 0.95 | 0.43-1.7 |
| Bi-DOTAM | 5.7 | 4.6-6.2 |
| Cu-DOTAM | 122000 | 60000-206000 |

Additional values are as shown below:

| | | | 95% confidence |
|---|---|---|---|
| Name | Antigen | KD [pM] | internal [pM] |
| PRIT-0213 | Ca-DOTAM | 0.95 | 0.43-1.7* |
| PRIT-0214 | Ca-DOTAM | 0.52 | 0.34-0.74 |
| PRIT-0213 | Bi-DOTAM | 5.7 | 4.6-6.2* |
| PRIT-0214 | Bi-DOTAM | 6.0 | 5.5-6.4 |
| PRIT-0213 | Cu-DOTAM | 122000 | 60000-206000*° |
| PRIT-0214 | CU-DOTAM | 38000 | 19000-63000*° |

*broad confidence interval, indicating the measured K_{D} not as precise
°assay not completely optimized for nM - affinity

### Sequences

Sequences for this example are provided below. PRIT-0213 and -0214 both have a Pb-DOTAM binding site comprising CDRs of SEQ ID NO 116-121 below. The sequences of the heavy and light chain variable of the Pb-DOTAM binding site of PRIT-0213 are shown in SEQ ID NO: 122-123 and those of PRIT-0214 are shown in SEQ ID NO: 124-125.

PRIT-0214 is composed of:

i) a first heavy chain having the amino acid sequence of SEQ ID NO: 126;

ii) a second heavy chain having the amino acid sequence of SEQ ID NO: 127; and

iii) two antibody light chains having the amino acid sequence of SEQ ID NO: 128.

PRIT-0213 is composed of:

i) a first heavy chain having the amino acid sequence of SEQ ID NO: 129;

ii) a second heavy chain having the amino acid sequence of SEQ ID NO: 130; and

iii) two antibody light chains having the amino acid sequence of SEQ ID NO: 128.

| SEQ ID | | | |
|---|---|---|---|
| NO | Description | SEQUENCE | |
| 116 | heavy chain CDR1 <Pb- | gfslstysms | |
| | Dotam> PRIT-0213 | | |
| 117 | heavy chain CDR2 <Pb- | figsrgdtyyaswakg | |
| | Dotam> PRIT-0213 | | |
| 118 | heavy chain CDR3 <Pb- | erdpygggaypphl | |
| | Dotam> PRIT-0213 | | |
| 119 | light chain CDR1 <Pb- | qsshsvysdndla | |
| | Dotam> PRIT-0213 | | |
| 120 | light chain CDR2 <Pb- | qasklas | |
| | Dotam> PRIT-0213 | | |
| 121 | light chain CDR3 <Pb- | lggyddesdtyg | |
| | Dotam> PRIT-0213 | | |
| 122 | heavy chain variable | vtlkesgpvl vkptetltlt ctvsgfslst | |
| | domain 1 of <Pb-Dotam> | ysmswirqpp gkalewlgfi gsrgdtyyas wakgrltisk | |
| | PRIT-0213 | dtsksqvvlt | |
| | | mtnmdpvdta tyycarerdp ygggaypphl wgrgtlvtvs s | |
| 123 | light chain variable | iqmtqspssl sasvgdrvti tcqsshsvys | |
| | domain <Pb-Dotam> | dndlawyqqk pgkapklliy qasklasgvp srfsgsgsgt dftltissiq | |
| | PRIT-0213 | pedfatyycl ggyddesdty gfgggtkvei k | |
| 124 | heavy chain variable | vqlqqwgagl lkpsetlslt cavygfslst | |
| | domain <Pb-Dotam> | ysmswirqpp gkglewigfi gsrgdtyyas wakgrvtisr dtsknqvslk | |
| | PRIT-0214 | lssvtaadta vyycarerdp ygggaypphl wgrgtlvtvs s | |
| 125 | light chain variable | iqmtqspssl sasvgdrvti tcqsshsvys | |
| | domain <Pb-Dotam> | dndlawyqqk pgkapklliy qasklasgvp srfsgsgsgt dftltisslq | |
| | PRIT-0214 | pedfatyycl ggyddesdty gfgggtkvei k | |
| 126 | heavy chain 1 of | 1 | qvqlvqsgae vkkpgssvkv sckasgfnik dtymhwvrqa pgqglewmgr |
| | bispecific, trivalent | 51 | idpangnsky vpkfqgrvti tadtststay melsslrsed tavyycapfg |
| | <CEA/Pb-Dotam>PRIT- | 101 | yyvsdyamay wgqgtlvtvs sastkgpsvf plapssksts ggtaalgclv |
| | 0214 VH_84.66 | 151 | kdyfpepvtv swnsgaltsg vhtfpavlqs sglyslssvv tvpssslgtq |
| | | 201 | tyicnvnhkp sntkvdkkve pkscdkthtc ppcpapeaag gpsvflfppk |
| | | 251 | pkdtlmisrt pevtcvvvdv shedpevkfn wyvdgvevhn aktkpreeqy |
| | | 301 | nstyrvvsvl tvlhqdwlng keykckvsnk algapiekti skakgqprep |
| | | 351 | qvytlppcrd eltknqvslw clvkgfypsd iavewesngq pennykttpp |
| | | 401 | vldsdgsffl yskltvdksr wqqgnvfscs vmhealhnhy tqkslslspg |
| | | 451 | ggggsggggs ggggsggggs vqlqqwgagl lkpsetlslt cavygfslst |
| | | 501 | ysmswirqpp gkglewigfi gsrgdtyyas wakgrvtisr dtsknqvslk |
| | | 551 | lssvtaadta vyycarerdp ygggaypphl wgrgtlvtvs s |
| 127 | heavy chain 2 of | 1 | qvqlvqsgae vkkpgssvkv sckasgfnik dtymhwvrqa pgqglewmgr |
| | bispecific, | 51 | idpangnsky vpkfqgrvti tadtststay melsslrsed tavyycapfg |
| | trivalent <CEA/Pb-Dotam> | 101 | yyvsdyamay wgqgtlvtvs sastkgpsvf plapssksts ggtaalgclv |
| | PRIT-0214 VL_84.66 | 151 | kdyfpepvtv swnsgaltsg vhtfpavlqs sglyslssvv tvpssslgtq |
| | | 201 | tyicnvnhkp sntkvdkkve pkscdkthtc ppcpapeaag gpsvflfppk |
| | | 251 | pkdtlmisrt pevtcyvvdv shedpevkfn wyydgvevhn aktkpreeqy |
| | | 301 | nstyrvvsyl tvlhqdwlng keykckvsnk algapiekti skakgqprep |
| | | 351 | qvctlppsrd eltknqvsls cavkgfypsd iavewesngq pennykttpp |
| | | 401 | vldsdgsffl vskltvdksr wqqgnvfscs vmhealhnhy tqkslslspg |
| | | 451 | ggggsggggs ggggsggggs iqmtqspssl sasvgdrvti tcqsshsvys |
| | | 501 | dndlawyqqk pgkapklliy qasklasgvp srfsgsgsgt dftltisslq |
| | | 551 | pedfatyycl ggyddesdty gfgggtkvei k |
| 128 | light chain <CEA> | 1 | eivltqspat lslspgerat lscragesvd ifgvgflhwy qqkpgqaprl |
| | 84.66 | 51 | liyrasnrat giparfsgsg sgtdftltis slepedfavy ycqqtnedpy |
| | | 101 | tfgqgtklei krtvaapsvf ifppsdeqlk sgtasvvcll nnfypreakv |
| | | 151 | qwkvdnalqs gnsqesvteq dskdstysls stltlskady ekhkvyacev |
| | | 201 | thqglsspvt ksfnrgec |
| 129 | heavy chain 1 of | 1 | qvqlvqsgae ykkpgssvkv sckasgfnik dtymhwvrqa pgqglewmgr |
| | bispecific, trivalent | 51 | idpangnsky vpkfqgrvti tadtststay melsslrsed tavyycapfg |
| | <CEA/Pb-Dotam> PRIT- | 101 | yyvsdyamay wgqgtlvtvs sastkgpsvf plapssksts ggtaalgclv |
| | 0213 VH_84.66 → knob | 151 | kdyfpepvtv swnsgaltsg vhtfpavlqs sglyslssvv tvpssslgtq |
| | | 201 | tyicnvnhkp sntkvdkkve pkscdkthtc ppcpapeaag gpsvflfppk |
| | | 251 | pkdtlmisrt pevtcvvvdv shedpevkfn wyvdgvevhn aktkpreeqy |
| | | 301 | nstyrvvsvl tvlhqdwlng keykckvsnk algapiekti skakgqprep |
| | | 351 | qvytlppcrd eltknqvslw clvkgfypsd iavewesngq pennykttpp |
| | | 401 | vldsdgsffl yskltvdksr wqqgnvfscs vmhealhnhy tqkslslspg |
| | | 451 | ggggsggggs ggggsggggs vtlkesgpvl vkptetltlt ctvsgfslst |
| | | 501 | ysmswirqpp gkalewlgfi gsrgdtyyas wakgrltisk dtsksqvvlt |
| | | 551 | mtnmdpvdta tyycarerdp ygggaypphl wgrgtlvtvs s |
| 130 | heavy chain 2 of | 1 | qvqlvqsgae vkkpgssvkv sckasgfnik dtymhwvrqa pgqglewmgr |
| | bispecific, trivalent | 51 | idpangnsky vpkfqgrvti tadtststay melsslrsed tavyycapfg |
| | <CEA/Pb-Dotam> PRIT- | 101 | yyvsdyamay wgqgtlvtvs sastkgpsvf plapssksts ggtaalgclv |
| | 0213 VL_84.66 → hole | 151 | kdyfpepvtv swnsgaltsg vhtfpavlqs sglyslssvv tvpssslgtq |
| | | 201 | tyicnvnhkp sntkvdkkve pkscdkthtc ppcpapeaag gpsvflfppk |
| | | 251 | pkdtlmisrt pevtcvvvdv shedpevkfn wyvdgvevhn aktkpreeqy |
| | | 301 | nstyrvvsvl tvlhqdwlng keykckvsnk algapiekti skakgqprep |
| | | 351 | qvctlppsrd eltknqvsls cavkgfypsd iavewesngq pennykttpp |
| | | 401 | vldsdgsffl vskltvdksr wqqgnvfscs vmhealhnhy tqkslslspg |
| | | 451 | ggggsggggs ggggsggggs iqmtqspssl sasvgdrvti tcqsshsvys |
| | | 501 | dndlawyqqk pgkapklliy qasklasgvp srfsgsgsgt dftltisslq |
| | | 551 | pedfatyycl ggyddesdty gfgggtkvei k |
| 131 | heavy chain 1 of | 1 | qvqlvqsgae vkkpgssvkv sckasgfnik dtymhwvrqa pgqglewmgr |
| | bispecific, <CEA/Pb- | 51 | idpangnsky vpkfqgrvti tadtststay melsslrsed tavyycapfg |
| | Dotam> Rabbit Dotam_ | 101 | yyvsdyamay wgqgtlvtvs sastkgpsvf plapssksts ggtaalgclv |
| | 84.66 | 151 | kdyfpepvtv swnsgaltsg vhtfpavlqs sglyslssvv tvpssslgtq |
| | | 201 | tyicnvnhkp sntkvdkkve pkscdkthtc ppcpapeaag gpsvflfppk |
| | | 251 | pkdtlmisrt pevtcvvvdv shedpevkfn wyvdgvevhn aktkpreeqy |
| | | 301 | nstyrvvsvl tvlhqdwlng keykckvsnk algapiekti skakgqprep |
| | | 351 | qvctlppsrd eltknqvsls cavkgfypsd iavewesngq pennykttpp |
| | | 401 | vldsdgsffl vskltvdksr wqqgnvfscs vmhealhnhy tqkslslspg |
| | | 451 | ggggsggggs ggggsggggs avltqtpspv spavggtvti scqsshsvys |
| | | 501 | dndlawyqqk lgqppklliy qasklasgvs srfsgsgsgt qftltisgvq |
| | | 551 | sddaatyycl ggyddesdty gfgggtevvv k |

| | | |
|---|---|---|
| 132 | HC5 | VTLKESGPVLVKPTETLTLTCTVSGFSLSTYSMSWI |
| | | RQPPGKALEWLGFIGSRGDTYYASWAKGRLTISKD |
| | | TSKSQVVLTMTNMDPVDTATYYCARERDPYGGG |
| | | AYPPHLWGRGTLVTVSS |
| 133 | LC1 | SIQMTQSPSSLSASVGDRVTITCQSSHSVYSDNDLA |
| | | WYQQKPGKAPKLLIYQASKLASGVPSRFSGSGSGT |
| | | DFTLTISSLQPEDFATYYCLGGYDDESDTYGFGGGT |
| | | KVEIK |
| 134 | LC3 | aqmtqspstl sasvgdrvti tcqsshsvys |
| | | dndlawyqqk pgqppklliy qasklasgvp srfsgsgsgt eftltisslq |
| | | pddfatyycl ggyddesdty gfgggtkvei k |
| 135 | HC7 | vqlvesgggl vqpggslrls caasgfslst |
| | | ysmswvrqap gkglewvgfi gsrgdtyyas wakgrftisr dtskntaylq |
| | | mnslraedta vyycarerdp ygggaypphl wgrgtlvtvs s |
| 136 | HC10 | vqlqqwgagl lkpsetlslt cavygfslst |
| | | ysmswirqpp gkglewigfi gsrgdtyyas wakgrvtisr dtsknqvslk |
| | | lssvtaadta vyycarerdp ygggaypphl wgrgtlvtvs s |
| 137 | T84.66 VH 1 | qvqlvqsgae vkkpgssvkv sckasgfnik dtymhwvrqa pgqglewmgr |
| | | idpangnsky vpkfqgrvti tadtststay melsslrsed tavyycapfg |
| | | yyvsdyamay wgqgtlvtvs s |
| 138 | T84.66 VL | EIVLTQSPATLSLSPGERATLSCRAGESVDIFGVGFL |
| | | HWYQQKPGQAPRLLIYRASNRATGIPARFSGSGSG |
| | | TDFTLTISSLEPEDFAVYYCQQTNEDPYTFGQGTKL |
| | | EIK |
| 139 | CH1A1A VH | qvqlvqsgae vkkpgasvkv sckasgytft efgmnwvrqa pgqglewmgw |
| | | intktgeaty veefkgrvtf ttdtststay melrslrsdd tavyycarwd |
| | | fayyveamdy wgqgttvtvs s |
| 140 | CH1A1A VL | diqmtqspss lsasvgdrvt itckasaavg tyvawyqqkp gkapklliys |
| | | asyrkrgvps rfsgsgsgtd ftltisslqp edfatyychq yytyplftfg |
| | | qgtkleik |

### Example 3: Crystallization, Data Collection and Structure Determination of the Fab P1AA1227 Pb-DOTAM Complex

For complex formation the Fab derived from the humanized VH/VL in PRIT-0213, called P1AA1227, at 26 mg/ml was mixed with Pb-DOTAM powder in a molar ratio of 1:4.2. After 2 hour incubation at 4° C. initial crystallization trials were performed in sitting drop vapour diffusion setups at 21° C. using the JCSG+screen (Qiagen, Hilden). Crystals appeared within 5 days out of 0.2 M (NH4)2SO4, 0.1 M BIS-TRIS pH 5.5, 25% w/v PEG3350. Crystals were harvested directly from the screening plate without any further optimization step.

Data collection and structure determination. For data collection crystals were flash frozen at 100K in precipitant solution containing 10% ethylenglycol. Diffraction data were collected at a wavelength of 1.0000 Å using a PILATUS 6M detector at the beamline X10SA of the Swiss Light Source (Villigen, Switzerland). Data have been processed with XDS (Kabsch, W. Acta Cryst. D66, 133-144 (2010)) and scaled with SADABS (BRUKER). The crystals of the complex belong to space group C2 with cell axes of a=135.63 Å, b=56.42 Å, c=64.52 Å and β=108.36° and diffract to a resolution of 1.40 Å. The structure was determined by molecular replacement with PHASER (McCoy, A. J, Grosse-Kunstleve, R. W., Adams, P. D., Storoni, L. C., and Read, R. J. J. Appl. Cryst. 40, 658-674 (2007)) using the coordinates of an in house Fab structure as search model. Difference electron density was used to place the Pb-DOTAM and to change amino acids according to the sequence differences by real space refinement. Structures were refined with programs from the CCP4 suite (Collaborative Computational Project, Number 4 Acta Cryst. D50, 760-763 (1994).) and BUSTER (Bricogne, G., Blanc, E., Brandl, M., Flensburg, C., Keller, P., Paciorek, W., Roversi, P., Sharff, A., Smart, O. S., Vonrhein, C., Womack, T. O. (2011). Buster version 2.9.5 Cambridge, United Kingdom: Global Phasing Ltd.). Manual rebuilding was done with COOT (Emsley, P., Lohkamp, B., Scott, W. G. and Cowtan, K. Acta Cryst D66, 486-501 (2010)).

Data collection and refinement statistics are summarized below.

All graphical presentations were prepared with PYMOL (The Pymol Molecular Graphics System, Version 1.7.4. Schrödinger, LLC.).

### Data Collection and Refinement Statistics for Fab P1AA1227-Pb-DOTAM Complex

| | P1AA1227-Pb-DOTAM |
|---|---|
| Data collection | |
| Space group | C2 |
| Cell dimensions | |
| a, b, c (Å) | 135.63, 56.42, 64.52 |
| α, β, γ(°) | 90, 108.36, 90 |
| Resolution (Å) | 1.40 |
| R_{sym} or R_{merge} | 0.041 |
| I/δI | 10.3 (0.94) |
| Completeness (%) | 94.4 (86.1) |
| Redundancy | 3.37 (3.33) |
| Refinement | |
| Resolution (Å) | 48.9-1.40 |
| No. reflections | 81631 |
| R_{work}/R_{free} | 19.21/22.38 |
| No. atoms | |
| Protein | 3342 |
| Water | 523 |
| Pb-DOTAM | 29 |
| B-factors | |
| Protein | 14.81 |
| Water | 37.46 |
| Pb-DOTAM | 21.09 |
| R.m.s. deviations | |
| Bond lengths (Å) | 0.011 |
| Bond angles ( °) | 1.57 |

*Values in parentheses are for highest-resolution shell.

Structure of Fab P1AA1227 in Complex with Pb-DOTAM

In order to characterize the interaction details of the Pb-DOTAM with Fab P1AA1227 we determined the crystal structure of the complex at a resolution of 1.40 Å. The structure reveals Fab P1AA1227 to bind to Pb-DOTAM by main contributions of the CDR1 and CDR3 of the light chain and by CDR2 and CDR3 of the heavy chain.

Analysis of the binding interface with the program PISA reveals an interaction pattern of Fab P1AA1227 with the Pb-DOTAM via 3 hydrogen bonds, polar interactions and van der Waals contacts. The Pb-DOTAM is bound in a pocket formed by heavy and light chain. This pocket has the shape of a box which is open on one side. Side walls and bottom of the pocket contribute apolar interactions whereas at the rim of the walls polar interactions dominate. Side chain hydrogen bonds are formed between CDR3 residues of heavy chain Glu95 and Asp97 with DOTAM carbamoyl nitrogen atoms N7 and N8. An additional hydrogen bond is established via the main chain carbonyl atom of Arg96 with atom N7 of DOTAM. The complex is further stabilized through apolar interactions of heavy chain CDR2 Phe50 and Tyr58 side chains which are oriented edge to face to the azacyclododecane ring. The light chain contributes mostly the “bottom” of the pocket with CDR3 residues Gly91-Tyr96 providing apolar contacts to the tetracyclododecane ring. Asp32 entertains a hydrogen bond to the carbamoyl nitrogen atom N6 of DOTAM. (Numbering according to Kabat).

The table below shows the heavy chain paratope residues, based on analysis with the program PISA.

| Heavy Chain | | |
|---|---|---|
| Residues (Kabat | | |
| number) | Type of interaction | Pb-DOTAM |
| Phe50 | apolar | Edge to face to azacyclodo- |
| | | decane ring |
| Asp56 | polar | N8 |
| Tyr58 | apolar | Edge to face to azacyclodo- |
| | | decane ring |
| G1u95 | H-bond | N7 |
| Arg96 | H-bond (mc carbonyl) | N7 |
| Asp97 | H-bond | N8 |
| Pro98 | Edge-face | Above Pb-Dotam, 4Å distance |
| Tyr99 | polar (but distance 6A) | N5 |
| Ala100C | apolar | C12 |
| Tyr 100D | polar (mc atoms) | N7 |

The Table below shows the light chain paratope residues, based on analysis with the program PISA.

| Light chain residues (Kabat | | |
|---|---|---|
| number) | Type of interaction | Pb-DOTAM |
| Tyr28 | apolar | Edge to face to |
| | | azacyclododecane ring |
| Asp32 | polar, H-bond | N6 |
| G1y91 | apolar | below azacyclododecane |
| | | ring |
| Tyr92 | apolar | below azacyclododecane |
| | | ring |
| Asp93 | apolar | below azacyclododecane |
| | | ring |
| Thr95C | apolar | below azacyclododecane |
| | | ring |
| Tyr96 | apolar | Edge to face to |
| | | azacyclododecane ring |

Paratope residues are also underlined in the sequences below:

>P1AA1227_HC
(SEQ ID NO.: 145)
VTLKESGPVLVKPTETLTLTCTVSGFSLSTYSMSWIRQPPGKALEWLGF
IGSRGDTYYASWAKGRLTISTSKSQVVLTMTNMDPVDTATYYCARERDP
YGGGAYPPHLWGRGTLVTVSSASTKGPSVFPLAPSSKSTSGGTAALGCL
VKDYFPEPVTVSWNSGALTSGVHTFPAVLQSSGLYSLSSVVTVPSSSLG
TQTYICNVNHICPSNTKVDKKVEPKSC
>P1AA1227_LC
(SEQ ID NO.: 146)
SIQMTQSPSSLSASVGDRVTITCQSSHSVYSDNDLAWYQQKPGKAPKLL
IYQASKLASGVPSRFSGSGSGTDFTLTISSLQPEDFATYYCLGGYDDES
DTYGFGGGTKVEIKRTVAAPSVFIFPPSDEQLKSGTASVVCLLNNFYPR
EAKVQWKVDNALQSGNSQESVTEQDSISTYSLSSTLTLSKADYEKHKVY
ACEVTHQGLSSPVTKSFNRGEC

### Example 4: Generation of CEA-Split-DOTAM VD/VL Antibodies

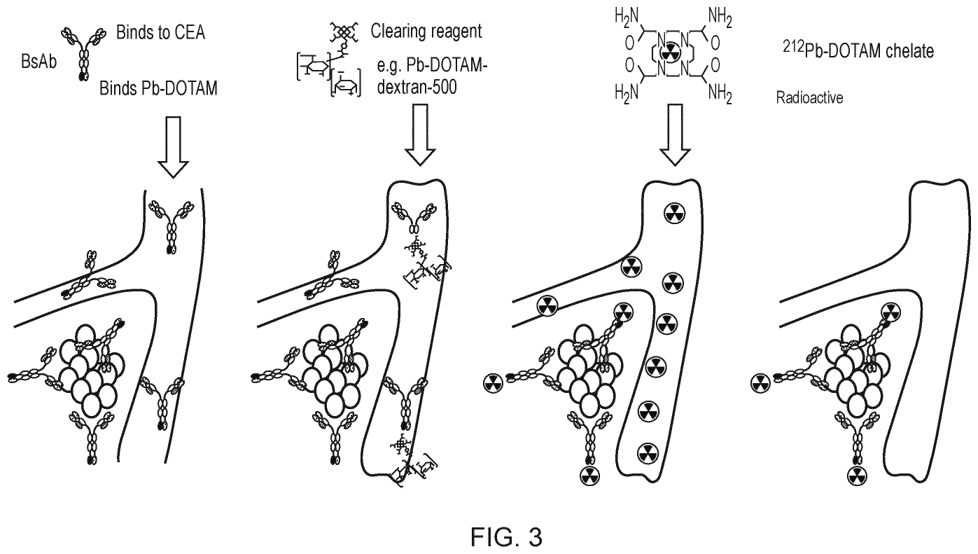

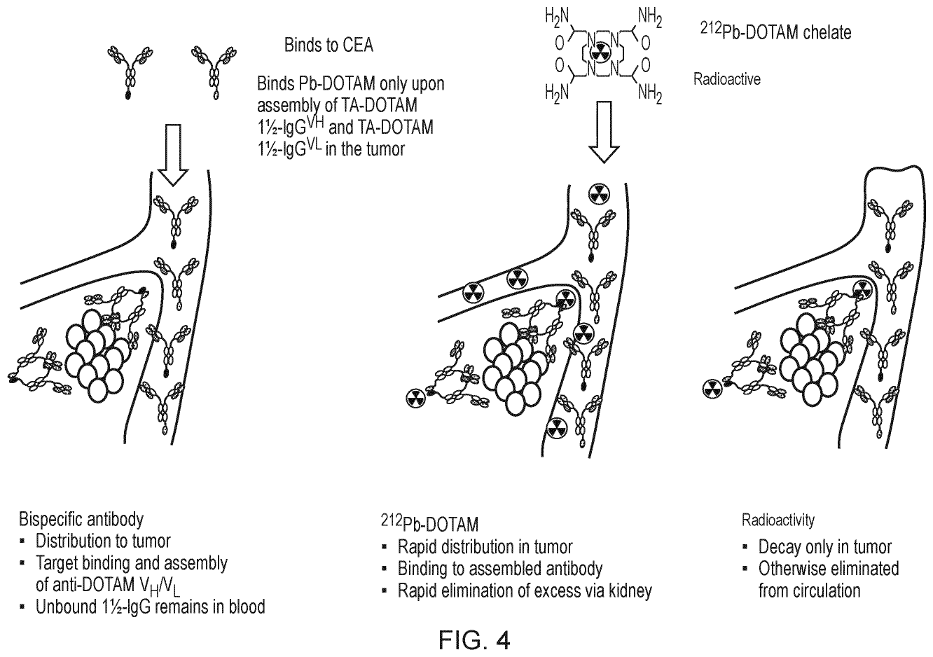

Methods of PRIT (Pretargeted radioimmunotherapy) using bispecific antibodies having a binding site for the target antigen and a binding site for the radiolabelled compound commonly use a clearing agent (CA) between the administrations of antibody and radioligand, to ensure effective targeting and high tumour-to-normal tissue absorbed dose ratios (see FIG. 3). In an example of one such method, injected BsAb is allowed sufficient time for penetrating into the tumours, generally 4-10 days, after which circulating BsAb is neutralized using a Pb-DOTAM-dextran-500 CA. The CA blocks ²¹²Pb-DOTAM binding to nontargeted BsAb without penetrating into the tumour, which would block the pretargeted sites. This pretargeting regimen allows efficient tumour accumulation of the subsequently administered radiolabelled chelate, ²¹²Pb-DOTAM.

However, in methods involving a clearing agent, the use of a CA introduces a further step to the method which is inefficient. Moreover, it can be important to choose the timing and dosing of the CA administration with care, which is a complicating factor.

To address the problems associated with use of a clearing agent, the present inventors have proposed a strategy of splitting the DOTAM VL and VH domains, such that they are found on separate antibodies.

The generation of exemplary split DOTAM VH/VL antibodies is discussed further below

### Generation of Plasmids for the Recombinant Expression of Antibody Heavy or Light Chains

Desired proteins were expressed by transient transfection of human embryonic kidney cells (HEK 293). For the expression of a desired gene/protein (e.g. full length antibody heavy chain, full length antibody light chain, or a full length antibody heavy chain containing an additional domain (e.g. an immunoglobulin heavy or light chain variable domain at its C-terminus) a transcription unit comprising the following functional elements was used:
the immediate early enhancer and promoter from the human cytomegalovirus (P-CMV) including intron A,
a human heavy chain immunoglobulin 5′-untranslated region (5′UTR),
a murine immunoglobulin heavy chain signal sequence (SS),
a gene/protein to be expressed, and
the bovine growth hormone polyadenylation sequence (BGH pA).

In addition to the expression unit/cassette including the desired gene to be expressed the basic/standard mammalian expression plasmid contained
an origin of replication from the vector pUC18 which allows replication of this plasmid in E. coli, and
a beta-lactamase gene which confers ampicillin resistance in E. coli.

### a) Expression Plasmid for Antibody Heavy Chains

Antibody heavy chain encoding genes including C-terminal fusion genes comprising a complete and functional antibody heavy chain, followed by an additional antibody V-heavy or V-light domain was assembled by fusing a DNA fragment coding for the respective sequence elements (V-heavy or V-light) separated each by a G4Sx4 linker to the C-terminus of the CH3 domain of a human IgG molecule (VH-CH1-hinge-CH2-CH3-linker-VH or VH-CH1-hinge-CH2-CH3-linker-VL). Recombinant antibody molecules bearing one VH and one VL domain at the C-termini of the two CH3 domains, respectively, were expressed using the knob-into-hole technology.

The expression plasmids for the transient expression of an antibody heavy chain with a C-terminal VH or VL domain in HEK293 cells comprised besides the antibody heavy chain fragment with C-terminal VH or VL domain expression cassette, an origin of replication from the vector pUC18, which allows replication of this plasmid in E. coli, and a beta-lactamase gene which confers ampicillin resistance in E. coli. The transcription unit of the antibody heavy chain fragment with C-terminal VH or VL domain fusion gene comprises the following functional elements:
the immediate early enhancer and promoter from the human cytomegalovirus (P-CMV) including intron A,
a human heavy chain immunoglobulin 5′-untranslated region (5′UTR),
a murine immunoglobulin heavy chain signal sequence,
an antibody heavy chain (VH-CH1-hinge-CH2-CH3-linker-VH or VH-CH1-hinge-CH2-CH3-linker-VL) encoding nucleic acid, and
the bovine growth hormone polyadenylation sequence (BGH pA).

The amino acid sequence of the mature antibody heavy chain fragment with C-terminal VH or VL domain fusion protein is shown below:

PRIT Split Antibody with DOTAM-VH-P1AD8749
>D1AC4022
(SEQ ID NO.: 28)
(SEQ ID NO.: 99)
(SEQ ID NO.: 100)
QVQLVQSGAEVICKPGASVKVSCKASGYTFTEFGMNWVRQAPGQGLEWMGW
INTKTGEATYVEEFKGRVTFTTDTSTSTAYMELRSLRSDDTAVYYCARWD
FAYYVEAMDYWGQGTTVTVSSASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKKVEPKSCDKTHTCPPCPAPEAAGGPSVFLFPPK
PICDTLMISRTPEVTCVVVDVSHEDPEVKFNWYVDGVEVHNAKTKPREEQY
NSTYRVVSVLTVLHQDWLNGKEYKCKVSNKALGAPIEKTISKAKGQPREP
QVCTLPPSRDELTKNQVSLSCAVKGFYPSDIAVEWESNGQPENNYKTTPP
VLDSDGSFFLVSKLTVDKSRWQQGNVFSCSVMHEALHNHYTQKSLSLSPG
>D1AA4507
(SEQ ID NO.: 32)
QVQLVQSGAEVKKPGASVKVSCKASGYTFTEFGMNWVRQAPGQGLEWMGW
INTKTGEATYVEEFKGRVTFTTDTSTSTAYMELRSLRSDDTAVYYCARWD
FAYYVEAMDYWGQGTTVTVSSASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKKVEPKSCDKTHTCPPCPAPEAAGGPSVFLFPPK
PKDTLMISRTPEVTCVVVDVSHEDPEVKFNWYVDGVEVHNAKTKPREEQY
NSTYRVVSVLTVLHQDWLNGKEYKCKVSNKALGAPIEKTISKAKGQPREP
QVYTLPPCRDELTKNQVSLWCLVKGFYPSDIAVEWESNGQPENNYKTTPP
VLDSDGSFFLYSKLTVDKSRWQQGNVFSCSVMHEALHNHYTQKSLSLSPG
GGGGSGGGGSGGGGSGGGGSVTLKESGPVLVKPTETLTLTCTVSGFSLST
YSMSWIRQPPGKALEWLGFIGSRGDTYYASWAKGRLTISKDTSKSQVVLT
MTNMDPVDTATYYCARERDPYGGGAYPPHLWGRGTLVTVSS
PRIT split antibody with DOTAM-VL-P1AD8592
>:D1AA4506
(SEQ ID NO.: 33)
QVQLVQSGAEVKKPGASVKVSCKASGYTFTEFGMNWVRQAPGQGLEWMGW
INTKTGEATYVEEFKGRVTFTTDTSTSTAYMELRSLRSDDTAVYYCARWD
FAYYVEAMDYWGQGTTVTVSSASTKGPSVFPLAPSSKSTSGGTAALGCLV
YFPEPVTVSWNSGALTSGVHTFPAVLQSSGLYSLSSVVTVPSSSLGTQ
TYICNVNHKPSNTKVDKKVEPKSCDKTHTCPPCPAPEAAGGPSVFLFPPK
PKDTLMISRTPEVTCVVVDVSHEDPEVKFNWYVDGVEVHNAKTKPREEQY
NSTYRVVSVLTVLHQDWLNGKEYKCKVSNKALGAPIEKTISKAKGQPREP
QVCTLPPSRDELTKNQVSLSCAVKGFYPSDIAVEWESNGQPENNYKTTPP
VLDSDGSFFLVSKLTVDKSRWQQGNVFSCSVMHEALHNHYTQKSLSLSPG
GGGGSGGGGSGGGGSGGGGSIQMTQSPSSLSASVGDRVTITCQSSHSVYS
DNDLAWYQQKPGKAPKLLIYQASKLASGVPSRFSGSGSGTDFTLTISSLQ
PEDFATYYCLGGYDDESDTYGFGGGTKVEIK
>:D1AC4023
(SEQ ID NO.: 30, 97)
QVQLVQSGAEVKKPGASVKVSCKASGYTFTEFGMNWVRQAPGQGLEWMGW
INTKTGEATYVEEFKGRVTFTTDTSTSTAYMELRSLRSDDTAVYYCARWD
FAYYVEAMDYWGQGTTVTVSSASTKGPSVFPLAPSSKSTSGGTAALGCLV
YFPEPVTVSWNSGALTSGVHTFPAVLQSSGLYSLSSVVTVPSSSLGTQ
TYICNVNHKPSNTKVDKKVEPKSCDKTHTCPPCPAPEAAGGPSVFLFPPK
PKDTLMISRTPEVTCVVVDVSHEDPEVKFNWYVDGVEVHNAKTKPREEQY
NSTYRVVSVLTVLHQDWLNGKEYKCKVSNKALGAPIEKTISKAKGQPREP
QVYTLPPCRDELTKNQVSLWCLVKGFYPSDIAVEWESNGQPENNYKTTPP
VLDSDGSFFLYSKLTVDKSRWQQGNVFSCSVMHEALHNHYTQKSLSLSPG

### b) Expression Plasmid for Antibody Light Chains

Antibody light chain encoding genes comprising a complete and functional antibody light chain was assembled by fusing a DNA fragment coding for the respective sequence elements.

The expression plasmid for the transient expression of an antibody light chain comprised besides the antibody light chain fragment an origin of replication from the vector pUC18, which allows replication of this plasmid in E. coli, and a beta-lactamase gene which confers ampicillin resistance in E. coli. The transcription unit of the antibody light chain fragment comprises the following functional elements:
the immediate early enhancer and promoter from the human cytomegalovirus (P-CMV) including intron A,
a human heavy chain immunoglobulin 5′-untranslated region (5′UTR),
a murine immunoglobulin heavy chain signal sequence,
an antibody light chain (VL-CL) encoding nucleic acid, and
the bovine growth hormone polyadenylation sequence (BGH pA).

The amino acid sequence of the mature antibody light chain fragment is the same for both P1AD8592 and P1AD8749.

>D1AA3384
(SEQ ID NO: 34)
(SEQ ID NO.: 103)
(SEQ ID NO.: 115)
DIQMTQSPSSLSASVGDRVTITCKASAAVGTYVAWYQQKPGKAPKWYS
ASYRKRGVPSRFSGSGSGTDFTLTISSLQPEDFATYYCHQYYTYPLFTFG
QGTKLEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC,,

### Transient Expression of the Antibody Molecules

The antibody molecules were generated in transiently transfected HEK293 cells (human embryonic kidney cell line 293-derived) cultivated in F17 Medium (Invitrogen Corp.). For transfection “293-Free” Transfection Reagent (Novagen) was used. The respective antibody heavy- and light chain molecules as described above were expressed from individual expression plasmids. Transfections were performed as specified in the manufacturer's instructions. Immunoglobulin-containing cell culture supernatants were harvested three to seven (3-7) days after transfection. Supernatants were stored at reduced temperature (e.g. −80° C.) until purification.

General information regarding the recombinant expression of human immunoglobulins in e.g. HEK293 cells is given in: Meissner, P. et al., Biotechnol. Bioeng. 75 (2001) 197-203.

The PRIT Hemibodies (split antibodies) were purified by a MabSelect Sure (Affinity Chromatography) and followed by Superdex 200 (Size Exclusion Chromatography). For the PRIT Split Antibody with DOTAM-VL-P1AD8592 5 mg with a concentration of 1.372 mg/mL and a purity >96% based on analytical SEC and CE-SDS were produced. For the PRIT Split Antibody with DOTAM-VH-P1AD8749 14 mg with a concentration of 2.03 mg/mL and a purity >91% based on analytical SEC and CE-SDS were produced.

Antibodies P1AE4956 and P1AE4957 were also generated and their sequences are provided herein. (P1AE4956 has heavy chains of SEQ ID NO: 51 and 52 and the light chain of SEQ ID NO: 54; P1AE4957 has heavy chains of SEQ ID NO 55 and 56 and the light chain of SEQ ID NO: 58). For the PRIT Split Antibody with DOTAM-VL-P1AE4957 19 mg with a concentration of 2.6 mg/mL and a purity >81.6% based on analytical SEC and CE-SDS were produced. For the PRIT Split Antibody with DOTAM-VH-P1AE4956 6.9 mg with a concentration of 1.5 mg/mL and a purity >90% based on analytical SEC and CE-SDS were produced. ESI-MS was used too confirm the identity of the PRIT hemibodies.

### Example 5: FACS Analysis of Split Antibody Functionality

To assess the functionality of the split antibodies or hemibodies, MKN-45 cells were detached from the culture vessel using accutase at 37° C. for 10 minutes. Subsequently, the cells were washed twice in PBS, and seeded into 96 well v-bottom plates to a final density of 4×10⁶ cells/well.

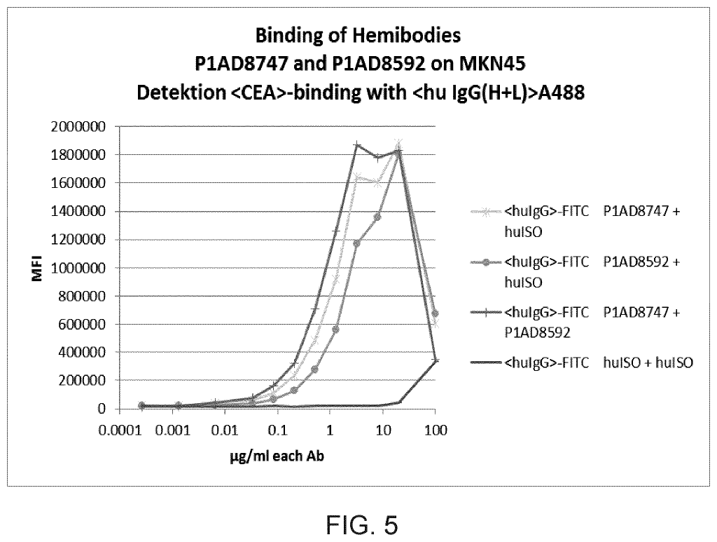

The hemibodies P1AD8749 and P1AD8592 and a human ISO control were mixed 1:1 added to the cells in concentrations as indicated in FIG. 5. Subsequently, the cells were incubated for 1 h on ice and washed twice in PBS. The cell pellet was resuspended and 40 μl/well of detection reagent was added, either <human IgG(H+L)>FITC, (10 μg/ml) or Pb_Dotam_FITC 1:100=>(10 μg/ml) in PBS/5% FCS. After 60 min incubation on ice, the cells were washed twice in PBS and resuspended in 200 μl PBS/5% FCS for measurement of FITC fluorescence using a FACS canto.

To assess the binding capability of the hemibodies to CEA on MKN-45 cells, they were detected using of antibodies using human IgG specific secondary antibodies (FIG. 5). As expected, no significant binding of the human ISO control is observed on these cells. When adjusted to the same IgG concentration, both hemibodies as well as the combination of both shows a dose dependent binding to MKN-45 cells, with a pronounced hook effect at very high concentrations as expected. This experiment demonstrates that the CEA binding is functional in the hemibodies.

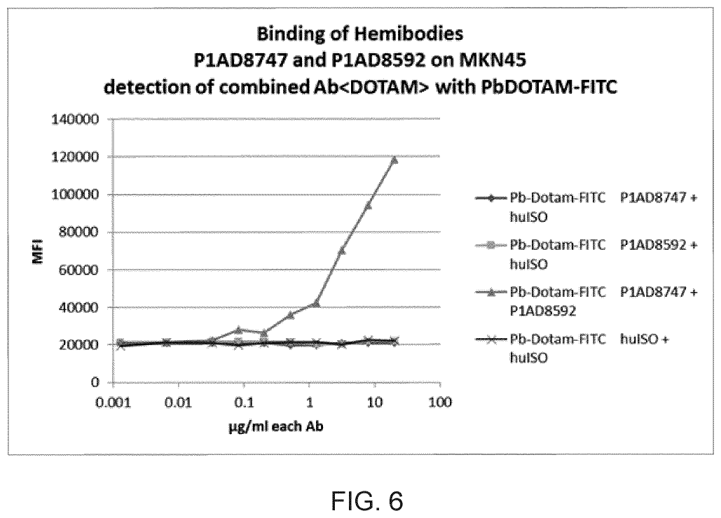

To assess the binding capability of the hemibodies to DOTAM, they were bound to the cells either in the presence of a human ISO control or their respective split antibody partner in a 1:1 ratio. After their binding to MKN-45 cells, the cells were washed to remove unbound antibody. Subsequently, Pb-DOTAM-FITC (fluorescently labelled Pb-DOTAM) was added to detect DOTAM binding competent cell bound antibodies (FIG. 6). As expected, no significant FITC is observed on these cells when one of the split antibody partners is combined with the of the human ISO control. Only a combination of both hemibodies in a 1:1 ratio shows a dose dependent FITC signal. This experiment shows that the DOTAM binding site becomes functional when both hemibodies come together on one cell.

### Example 6: In Vivo Studies

### Example 6a: Materials and Methods—General

All experimental protocols were reviewed and approved by the local authorities (Comité Régional d'Ethique de l'Expérimentation Animale du Limousin [CREEAL], Laboratoire Départemental d'Analyses et de Recherches de la Haute-Vienne). Female severe combined immunodeficiency (SCID) mice (Charles River) were maintained under specific and opportunistic pathogen free (SOPF) conditions with daily cycles of light and darkness (12 h/12 h), in line with ethical guidelines. No manipulations were performed during the first 5 days after arrival, to allow the animals to acclimatize to the new environment. Animals were controlled daily for clinical symptoms and detection of adverse events.

Solid xenografts were established by subcutaneous (SC) injection of CEA-expressing tumor cells in cell culture media mixed 1:1 with Corning® Matrigel® basement membrane matrix (growth factor reduced; cat No. 354230). Tumor volumes were estimated through manual calipering 3 times per week, calculated according to the formula: volume=0.5×length×width². Additional tumor measurements were made as needed depending on the tumor growth rate.

Mice were euthanized before the scheduled endpoint if they showed signs of unamenable distress or pain due to tumor burden, side effects of the injections, or other causes. Indications of pain, distress, or discomfort include, but are not limited to, acute body weight (BW) loss, scruffy fur, diarrhea, hunched posture, and lethargy. The BW of treated animals was measured 3 times per week, with additional measurements as needed depending on the health status. Wet food was provided to all mice starting the day after the radioactive injection, for 7 days or until all individuals had recovered sufficiently from any acute BW loss. Mice whose BW loss exceeded 20% of their initial BW or whose tumor volume reached 3000 mm³ were euthanized immediately. Other factors taken into account for euthanasia for ethical reasons were tumor status (e.g. necrotic areas, blood/liquid leaking out, signs of automutilation) and general appearance of the animal (e.g. fur, posture, movement).

To minimize re-ingestion of radioactive urine/feces, all efficacy study mice were placed in cages with grilled floors for 4 hours after ²¹²Pb-DOTAM administration, before being transferred to new cages with standard bedding. All cages were then changed at 24 hours post injection (p.i.). This procedure was not performed for mice sacrificed for biodistribution purposes within 24 hours after the radioactive injection.

Blood was collected at the time of euthanasia from the venous sinus using retro-orbital bleeding on anesthetized mice, before termination through cervical dislocation followed by additional tissue harvest for radioactive measurements and/or histological analysis, as mandated by the protocols. Unexpected or abnormal conditions were documented. Tissues collected for formalin fixation were immediately put in 10% neutral buffered formalin (4° C.) and then transferred to phosphate-buffered saline (PBS; 4° C.) after 5 days. Organs and tissues collected for biodistribution purposes were weighed and measured for radioactivity using a 2470 WIZARD² automatic gamma counter (PerkinElmer), and the percent injected dose per gram of tissue (% ID/g) subsequently calculated, including corrections for decay and background.

Statistical analysis was performed using GraphPad Prism 7 (GraphPad Software, Inc.) and JMP 12 (SAS Institute Inc.). Curve analysis of tumor growth inhibition (TGI) was performed based on mean tumor volumes using the formula:

$$T G I = 100 - \frac{\overline{{v}_{treatment , d} - {v}_{treatmet , 0}}}{\overline{{v}_{ref , d} - {v}_{ref , 0}}} \times 100$$

where d indicates study day and 0 the baseline value. Vehicle was selected as the reference group. Tumor regression (TR) was calculated according to:

$$T R = \frac{\overline{{v}_{treatment , 0} - {v}_{treatment , d}}}{{v}_{treatment , 0}}$$

where positive values indicated tumor regression, and values below −1 growth beyond the double baseline value.

### Test Compounds

The compounds utilized in the described studies are presented in the tables below, respectively for bispecific antibodies, clearing agents, and radiolabeled chelates.

CEA-DOTAM (R07198427, PRIT-0213) is a fully humanized BsAb targeting the T84.66 epitope of CEA, whereas DIG-DOTAM (R07204012) is a non-CEA-binding BsAb used as a negative control. P1 AD8749, P1 AD8592, P1AE4956, and P1AE4957 are CEA-split-DOTAM-VH/VL antibodies targeting the CH1A1A or A5B7 epitopes of CEA. All antibody constructs were stored at −80° C. until the day of injection when they were thawed and diluted in standard vehicle buffer (20 mM Histidine, 140 mM NaCl; pH 6.0) or 0.9% NaCl to their final respective concentrations for intravenous (IV) or intraperitoneal (IP) administration.

The Pb-DOTAM-dextran-500 CA (R07201869) was stored at −20° C. until the day of injection when it was thawed and diluted in PBS for IV or IP administration.

The DOTAM chelate for radiolabeling was provided by Macrocyclics and maintained at −20° C. before radiolabeling, performed by Orano Med (Razes, France). ²¹²Pb-DOTAM (R07205834) was generated by elution with DOTAM from a thorium generator, and subsequently quenched with Ca after labeling. The ²¹²Pb-DOTAM solution was diluted with 0.9% NaCl to obtain the desired ²¹²Pb activity concentration for IV injection. Mice in vehicle control groups received multiple injections of vehicle buffer instead of BsAb, CA, and ²¹²Pb-DOTAM.

### Bispecific Antibodies

| Compound | Target | Protocols |
|---|---|---|
| CEA-DOTAM | T84.66 | 144, 158, 160 |
| (RO7198427, PRIT- | | |
| 0213 ) | | |
| DIG-DOTAM | Digoxigenin | 160 |
| (RO7204012) | | |
| CEA-split-DOTAM-VH | CH1A1A | 144, 158 |
| P1AD8749 | | |
| CEA-split-DOTAM- | CH1A1A | 175, 185, 189 |
| VH-AST | | |
| PlAF0171 | | |
| CEA-split-D-OTAM-VL | CH1A1A | 144, 158 |
| P1AD8592 | | 175, 185, 189 |
| CEA-split-DOTAM-VH | A5B7 | 158 |
| PlAE4956 | | |
| CEA-split-DOTAM-VL | A5B7 | 158 |
| PlAE4957 | | |
| CEA-split-DOTAM- | T84.66 | 185, 189 |
| VH-AST | | |
| PlAF0298 | | |
| CEA-split-DOTAM-VL | T84.66 | 185, 189 |
| PlAF0709 | | |

### Clearing Agents

| Compound | Protocols |
|---|---|
| Ca-DOTAM-dextran-500 | 144, 158, 160 |
| (RO7201869) | |

### Radiolabeled Chelates

| Compound | Quenching | Protocols |
|---|---|---|
| ²¹²Pb-DOTAM | Ca | 144, 158, |
| (RO7205834) | | 160, 175, |
| | | 185, 189 |
| ²¹²Pb-DOTAM-CEA- | Ca | 160 |
| DOTAM | | |

### Tumor Models

The tumor cell line used and the injected amount for inoculation in mice is described in the table below. BxPC3 is a human primary pancreatic adenocarcinoma cell line, naturally expressing CEA. Cells were cultured in RPMI 1640 Medium, GlutaMAX™ Supplement, HEPES (Gibco, ref. No. 72400-021) enriched with 10% fetal bovine serum (GE Healthcare Hyclone SH30088.03). Solid xenografts were established in each SCID mouse on study day 0 by subcutaneous injection of cells in RPMI media mixed 1:1 with Corning® Matrigel® basement membrane matrix (growth factor reduced; cat No. 354230), into the right flank.

### Tumor Cell Lines

| Cell line | Cells per mouse | Injected volume | Protocols | Supplier |
|---|---|---|---|---|
| BxPC3 | 5×10⁶ | 100 μL | 144, 158, 160, | ECACC* |
| | | | 175, 185, 189 | |

*European Collection of Authenticated Cell Cultures (Salisbury, UK)

### Example 6b: Protocol 144

The aim of protocol 144 was to provide PK and in vivo distribution data of pretargeted ²¹²Pb-DOTAM in SCID mice carrying SC BxPC3 tumors after 2-step PRIT using CEA-split-DOTAM-VH/VL BsAbs.

Two-step PRIT was performed by injection of the CEA-split-DOTAM-VH and CEA-split-DOTAM-VL (P1AD8749 and P1AD8592), separately or together, followed 7 days later by ²¹²Pb-DOTAM. Mice were sacrificed 6 hours after the radioactive injection, and blood and organs harvested for radioactive measurement. The 2-step scheme was compared with 3-step PRIT using the standard CEA-DOTAM bispecific antibody, followed 7 days later by Ca-DOTAM-dextran-500 CA, and ²¹²Pb-DOTAM 24 hours after the CA.

PK data of CEA-split-DOTAM-VH/VL clearance was collected by repeated blood sampling from 1 hour to 7 days after the antibody injection, and subsequently analyzed by an ELISA.

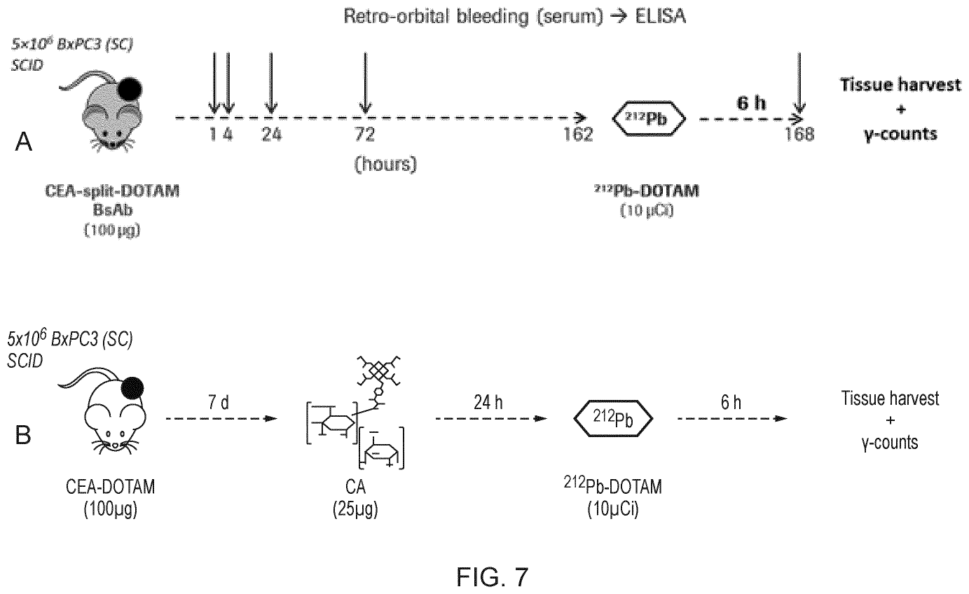

The study outline is shown in FIG. 7. FIG. 7a shows the outline of the 2-step PRIT regimen, including blood sampling for CEA-split-DOTAM-VH/VL PK, in SCID mice carrying SC BxPC3 tumors. FIG. 7b shows the outline of the 3-step PRIT regimen, performed in SCID mice carrying SC BxPC3 tumors (h=hours, d=days).

### Study Design

The time course and design of protocol 144 is shown in the tables below.

### Time Course of Protocol 144

| Study day | Date | Experimental procedure |
|---|---|---|
| 0 | 2018 Apr. 2 | Preparation of BxPC3 cells and filling of syringes |
| 0 | 2018 Apr. 2 | SC injection of BxPC3 cells |
| 14 | 2018 Apr. 16 | IV injection of CEA-DOTAM BsAb (group D) |
| 15 | 2018 Apr. 17 | IV injection of CEA-split-DOTAM-VH/VL |
| | | BsAbs (groups Aa, Ab, Ba, Bb, Ca, Cb) |
| 15 | 2018 Apr. 17 | Retro-orbital bleeding (1 and 4 h p.i.; groups |
| | | Aa, Ba, Ca, and Ab, Bb, Cb, respectively) |
| 16 | 2018 Apr. 18 | Retro-orbital bleeding |
| | | (24 h p.i.; groups Aa, Ba, Ca) |
| 18 | 2018 Apr. 20 | Retro-orbital bleeding |
| | | (72 h p.i.; groups Ab, Bb, Cb) |
| 21 | 2018 Apr. 23 | IV injection of CA (group D) |
| 21 | 2018 Apr. 23 | Elution of ²¹²Pb-DOTAM and filling of syringes |
| 22 | 2018 Apr. 24 | IV injection of²¹²Pb-DOTAM ) |
| | | (groups Aa, Ba, Ca, D |
| 22 | 2018 Apr. 24 | Retro-orbital bleeding (168 h p.i.) and euthanasia |
| | | (groups Ab, Bb, Cb) |
| 22 | 2018 Apr. 24 | Euthanasia and tissue harvest, incl. |
| | | retro-orbital bleeding (6 h p.i.) + gamma |
| | | counting (groups Aa, Ba, Ca, D) |

### Study Groups in Protocol 144

| | P1AD8749 | P1AD8592 | | | | | | |
|---|---|---|---|---|---|---|---|---|
| | (VH) | (VL) | CEA-DOTAM | | | | | |
| | CH1A1A | CH1A1A | BsAb | PK | CA | ²¹²Pb | BD | n |
| Group | (μg) | (μg) | (μg) | (h p.i.) | (μg) | (μCi) | (h p.i.) | (mice) |
| Aa | 100 | 0 | 0 | 1, 24, 168 | 0 | 10 | 6 | 4 |
| Ab | 100 | 0 | 0 | 4, 72, 168 | 0 | 0 | — | 4 |
| Ba | 0 | 100 | 0 | 1, 24, 168 | 0 | 10 | 6 | 4 |
| Bb | 0 | 100 | 0 | 4, 72, 168 | 0 | 0 | — | 4 |
| Ca | 100 | 100 | 0 | 1, 24, 168 | 0 | 10 | 6 | 4 |
| Cb | 100 | 100 | 0 | 4, 72, 168 | 0 | 0 | — | 4 |
| D | 0 | 0 | 100 | — | 25 | 10 | 6 | 4 |

Solid xenografts were established in each SCID mouse on study day 0 by SC injection of 5×10⁶ cells (passage 26) in RPMI/Matrigel into the right flank. Fourteen days after tumor cell injection, mice were sorted into experimental groups with an average tumor volume of 116 mm³. The ²¹²Pb-DOTAM was injected on day 22 after inoculation; the average tumor volume was 140 mm³ on day 21.

Blood from mice in groups Aa, Ba, and Ca was collected through retro-orbital bleeding under anesthesia 1 h (right eye), 24 h (left eye), and 168 h (right eye, at termination) after CEA-split-DOTAM-VH/VL injection. Similarly, samples were taken from mice in groups Ab, Bb, and Cb 4 h (right eye), 72 h (left eye), and 168 h (right eye, at termination) after CEA-split-DOTAM-VH/VL injection.

Mice in groups Aa, Ba, Ca, and D were sacrificed and necropsied 6 hours after injection of ²¹²Pb-DOTAM, and the following organs and tissues harvested for measurement of radioactive content: blood, skin, bladder, stomach, small intestine, colon, spleen, pancreas, kidneys, liver, lung, heart, femoral bone, muscle, brain, tail, ears, and tumor.

### Results

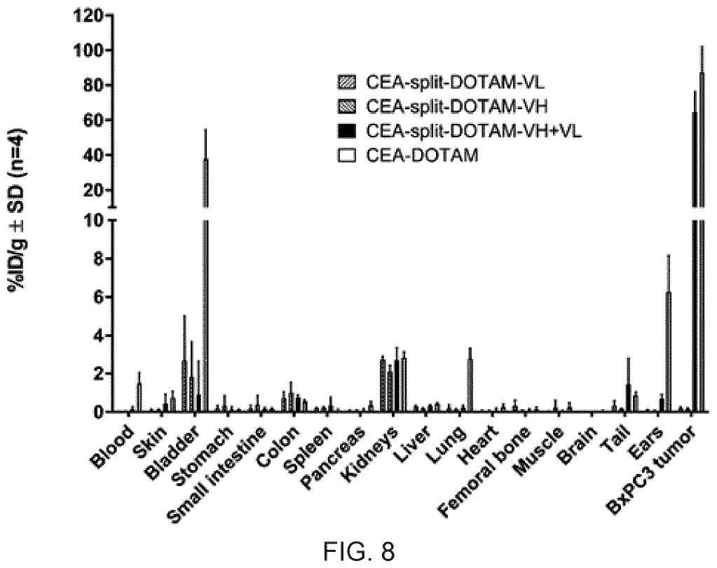

The average ²¹²Pb accumulation and clearance in all collected tissues 6 hours after injection is displayed in FIG. 8. Pretargeting with either CEA-split-DOTAM-VH or CEA-split-DOTAM-VL alone resulted in no accumulation of radioactivity in tumors. Combined, the two complimentary antibodies resulted in a tumor uptake after 2-step PRIT of 65±12% ID/g, to be compared with 87±15% ID/g for the standard 3-step PRIT regimen. Two-way analysis of variance (ANOVA) with Tukey's multiple comparisons test showed that the difference in tumor uptake between the two PRIT treatments was significant, as was the difference in bladder (1±2% ID/g and 38±17% ID/g for 2- and 3-step PRIT, respectively); no other differences in tissue accumulation were statistically significant using this test (p=0.05).

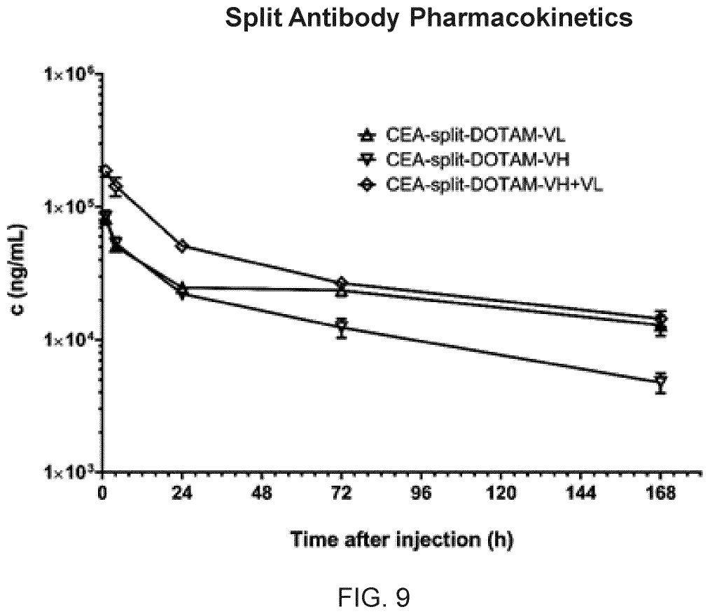

The clearance of IV injected CEA-split-DOTAM-VH/VL constructs as analyzed by an enzyme-linked immunosorbent assay (ELISA) is shown in FIG. 9.

### Adverse Events and Toxicity

There were no adverse events or toxicity associated with this study.

### Conclusion

The results of the study demonstrated proof-of-concept of CA-independent 2-step pretargeting using complimentary CEA-split-DOTAM-VH/VL antibodies. High and specific tumor uptake of ²¹²Pb-DOTAM was achieved using 2-step PRIT and standard 3-step PRIT, with very little accumulation of radioactivity in normal tissues using the complimentary CEA-split-DOTAM-VH/VL antibodies.

### Example 6c: Protocol 158

The aim of protocol 158 was to assess the association of ²¹²Pb-DOTAM to subcutaneous BxPC3 tumors in mice pretargeted by bi-paratopic (CH1A1A and A5B7) pairs of CEA-split-DOTAM-VH/VL antibodies for clearing agent-independent 2-step CEA-PRIT. The tumor uptake was compared with that of standard 3-step CEA-PRIT.

Mice carrying subcutaneous BxPC3 tumors were injected with either
CEA-split-DOTAM-VH/VL antibodies followed 7 days later by the radiolabeled ²¹²Pb-DOTAM (2-step PRIT), or
CEA-DOTAM BsAb followed 7 days later by the CA, and finally the radiolabeled ²¹²Pb-DOTAM 24 hours later (3-step PRIT).

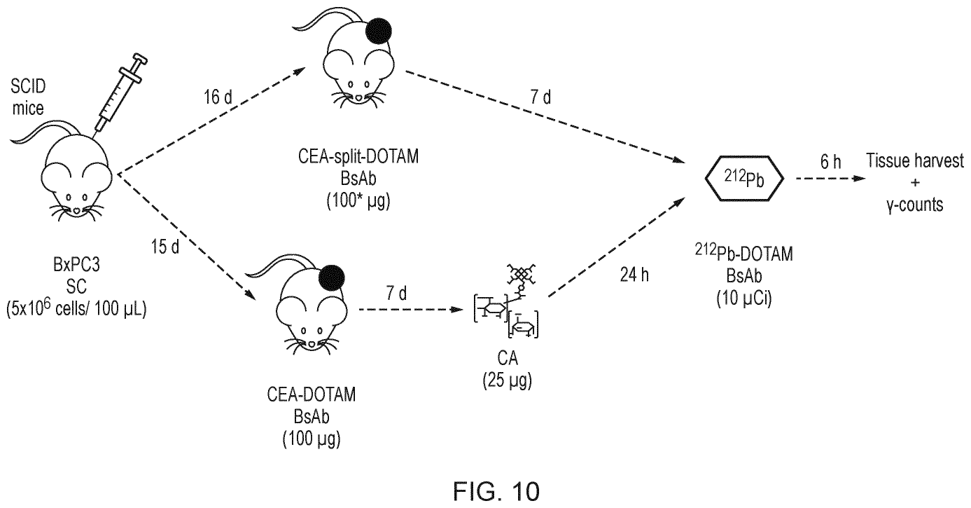

The in vivo distribution of ²¹²Pb-DOTAM was assessed 6 hours after the radioactive injection. The study outline is shown in FIG. 10.

### Study Design

The time course and design of protocol 158 is shown in the tables below.

### Time Course of Protocol 158

| Study day | Date | Experimental procedure |
|---|---|---|
| 0 | 2018 Nov. 26 | Preparation of BxPC3 cells and filling of syringes |
| 0 | 2018 Nov. 26 | SC injection of BxPC3 cells |
| 15 | 2018 Dec. 11 | IV injection of CEA-DOTAM BsAb (group C) |
| 16 | 2018 Dec. 12 | IV injection of CEA-split-DOTAM-VH/VL |
| | | BsAbs (groups A, B) |
| 22 | 2018 Dec.18 | IV injection of CA (group C) |
| 22 | 2018 Dec.18 | Elution of ²¹²Pb-DOTAM and filling of syringes |
| 23 | 2018 Dec.19 | IV injection of ²¹²Pb-DOTAM (all) |
| 23 | 2018 Dec.19 | Euthanasia and tissue harvest, incl retro- |
| | | orbital bleeding (6 h p.i.) + gamma counting (all) |

### Study Groups in Protocol 158

| | P1AD8749 | P1AD8592 | P1AE4956 | P1AE4957 | | | | |
|---|---|---|---|---|---|---|---|---|
| | (VH) | (VL) | (VH) | (VL) | CEA-DOTAM | | | |
| | CH1A1A | CH1A1A | A5B7 | A5B7 | BsAb | CA | ²¹²Pb | n |
| Group | (μg) | (μg) | (μg) | (μg) | (μg) | (μg) | (μCi) | (mice) |
| A | 154* | 0 | 0 | 100 | 0 | 0 | 10 | 4 |
| B | 0 | 100 | 167** | 0 | 0 | 0 | 10 | 4 |
| C | 0 | 0 | 0 | 0 | 100 | 25 | 10 | 4 |

*P1AD8749 dose adjusted to 154 μg to compensate for a 35% hole/hole impurity;**P1AD8592 dose adjusted to 167 μg to compensate for a 40% hole/hole impurity.

Solid xenografts were established in each SCID mouse on study day 0 by SC injection of 5×10⁶ cells (passage 27) in RPMI/Matrigel into the right flank. Fourteen days after tumor cell injection, mice were sorted into experimental groups with an average tumor volume of 177 mm³. The ²¹²Pb-DOTAM was injected on day 20 after inoculation; the average tumor volume was 243 mm³ on day 21.

Mice in all groups were sacrificed and necropsied 6 hours after injection of ²¹²Pb-DOTAM, and the following organs and tissues harvested for measurement of radioactive content: blood, skin, bladder, stomach, small intestine, colon, spleen, pancreas, kidneys, liver, lung, heart, femoral bone, muscle, brain, tail, and tumor.

### Results

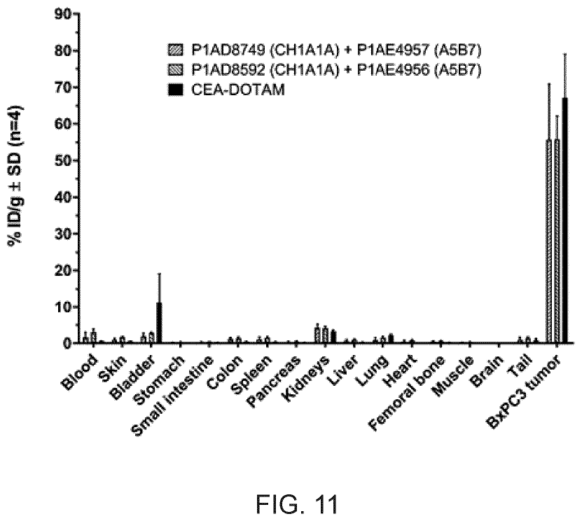

The average ²¹²Pb distribution in all collected tissues 6 hours after injection is shown in FIG. 11. Two-way ANOVA with Tukey's multiple comparisons test showed that there was no significant difference in normal tissue uptake of ²¹²Pb between the three treatments, except for bladder, where both bi-paratopic CEA-split-DOTAM-VH/VL pairs yielded lower accumulation than the standard 3-step PRIT. The kidney uptake was 3-4% ID/g for all three treatments. Either bi-paratopic combination resulted in tumor accumulation of approximately 56% ID/g, compared with 67% ID/g for 3-step PRIT; the difference between 2- and 3-step PRIT was statistically significant (p<0.0001).

### Adverse Events and Toxicity

There were no adverse events or toxicity associated with this study.

### Conclusion

This study assessed the association of ²¹²Pb-DOTAM to SC BxPC3 tumors in mice pretargeted by bi-paratopic pairs of CEA-split-DOTAM-VH/VL antibodies for CA-independent 2-step CEA-PRIT, compared with standard 3-step PRIT. The distribution of ²¹²Pb 6 hours after injection was comparable for 2- and 3-step PRIT, with high accumulation in tumor and very little radioactivity in healthy tissues. This demonstrated proof of concept of bi-paratopic pretargeting of CEA-expressing tumors for 2-step CEA-PRIT using CEA-split-DOTAM-VH/VL antibodies.

### Example 6d: Protocol 160

The aim of protocol 160 was to compare the therapeutic efficacy after 3 cycles of CA-independent 2-step CEA-PRIT using complimentary CEA-split-DOTAM-VH/VL antibodies, with that of standard 3-step CEA-PRIT in mice bearing SC BxPC3 tumors. A comparison was also made with 1-step CEA-RIT, using BsAbs that were pre-incubated with ²¹²Pb-DOTAM before injection.

Mice carrying SC BxPC3 tumors were injected with either
CEA-DOTAM BsAb followed 7 days later by the CA, and finally the radiolabeled ²¹²Pb-DOTAM 24 hours later (3-step PRIT),
CEA-split-DOTAM-VH/VL antibodies followed 7 days later by the radiolabeled ²¹²Pb-DOTAM (2-step PRIT), or
²¹²Pb-DOTAM-CEA-DOTAM BsAb (pre-incubated; 1-step RIT).

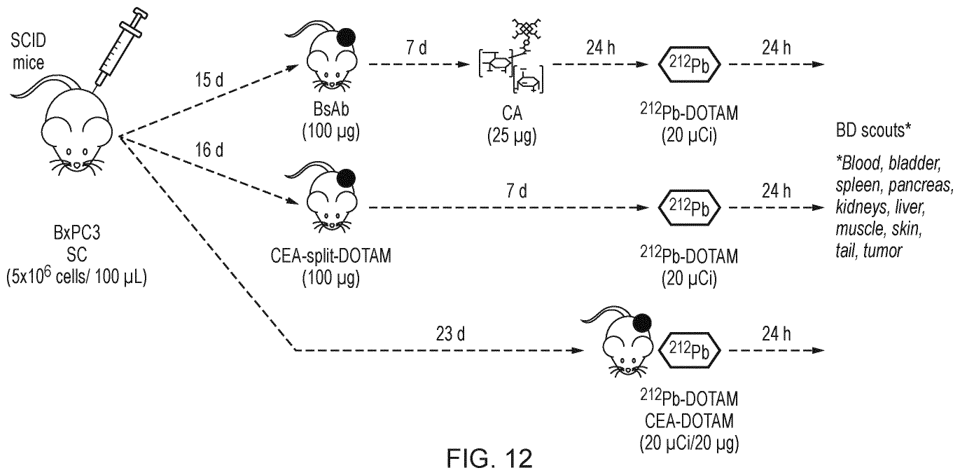

The therapy was administered in 3 repeated cycles of 20 μCi of ²¹²Pb-DOTAM, also including comparison with a non-CEA binding control antibody (DIG-DOTAM), and no treatment (vehicle). Dedicated mice were sacrificed for biodistribution purposes to confirm ²¹²Pb-DOTAM targeting and clearance at each treatment cycle. The treatment efficacy was assessed in terms of TGI and TR, and the mice were carefully monitored for the duration of the study to assess the tolerability of the treatment. The study outline is shown in FIG. 12.

The time course and design of protocol 160 are shown in the tables below.

### Time Course of Protocol 160

| Study day | Date | Experimental procedure |
|---|---|---|
| 0 | 2019 Sep. 29 | Preparation of BxPC3 cells and filling of syringes |
| 0 | 2019 Sep. 29 | SC injection of BxPC3 cells |
| 15 | 2019 Sep. 13 | IP injection of BsAb or histidine buffer (groups |
| | | A, B, C, F, G, H, I) |
| 16 | 2019 Feb.14 | IP injection of CEA-split-DOTAM-VH/VL |
| | | or histidine buffer (groups D, J, K, L) |
| 22 | 2019 Feb.20 | IP injection of CA or PBS ) |
| | | (groups A, B, C, F, G, H, I |
| 23 | 2019 Feb.21 | Elution of ²¹²Pb-DOTAM and filling of syringes |
| 23 | 2019 Feb.21 | IV injection of ²¹²Pb-DOTAM-CEA- |
| | | DOTAM or histidine buffer (groups E, M) |
| 23 | 2019 Feb.21 | IV injection of ²¹²Pb-DOTAM or 0.9% NaCl |
| | | (groups B, C, D, F, G, H, I, J, K, L) |
| 24 | 2019 Feb.22 | Euthanasia and tissue harvest (24 h p.i.) + gamma |
| | | counting (groups F, G, J, M) |
| 29 | 2019 Feb.27 | IP injection of PRIT BsAb or PBS |
| | | (groups A, B, C, H, I) |
| 30 | 2019 Feb.28 | IP injection of CEA-split-DOTAM-VH/VL |
| | | or histidine buffer (groups D, K, L) |
| 36 | 2019 Mar. 06 | IP injection of CA or PBS (groups A, B, C, H, I) |
| 37 | 2019 Mar. 07 | Elution of ²¹²Pb-DOTAM and filling of syringes |
| 37 | 2019 Mar. 07 | IV injection of ²¹²Pb-DOTAM or 0.9% NaC1 |
| | | (groups B, C, D, H, I, K, L) |
| 38 | 2019 Mar. 08 | Euthanasia and tissue harvest (24 h p.i.) + gamma |
| | | counting (groups H, K) |
| 43 | 2019 Mar. 13 | IP injection of PRIT BsAb or PBS |
| | | (groups A, B, C, I) |
| 44 | 2019 Mar. 14 | IP injection of CEA-split-DOTAM-VH/VL |
| | | or histidine buffer (groups D, L) |
| 50 | 2019 Mar. 20 | IP injection of CA or PBS (groups A, B, C, I) |
| 51 | 2019 Mar. 21 | Elution of Pb-DOTAM and filling of syringes |
| 51 | 2019 Mar. 21 | IV injection of ²¹²Pb-DOTAM or 0.9% NaC1 |
| | | (groups B, C, D, I, L) |
| 52 | 2019 Mar. 22 | Euthanasia and tissue harvest (24 h p.i.) + gamma |
| | | counting (groups I, L) |

### Study Groups in Protocol 160

| | | BsAb | CA | ²¹²Pb- | | |
|---|---|---|---|---|---|---|
| | | per | per | DOTAM | | |
| | | cycle | cycle | per cycle | Cycles | n |
| Group | BsAb | (μg) | (μg) | (μCi) | (#) | (mice) |
| A | — | 0 | 0 | 0 | 3 | 10 |
| B | DIG-DOTAM | 100 | 25 | 20 | 3 | 10 |
| C | CEA-DOTAM | 100 | 25 | 20 | 3 | 10 |
| D | CEA-split- | 154* + | 0 | 20 | 3 | 10 |
| | DOTAM | 100** | | | | |
| E | ²¹²Pb-DOTAM- | 100 | 0 | 20 | 1** | 10 |
| | CEA-DOTAM | | | | | |
| F | DIG-DOTAM | 100 | 25 | 20 | 1 | 3 |
| G | CEA-DOTAM | 100 | 25 | 20 | 1 | 3 |
| H | CEA-DOTAM | 100 | 25 | 20 | 2 | 3 |
| I | CEA-DOTAM | 100 | 25 | 20 | 3 | 3 |
| J | CEA-split- | 154* + | 0 | 20 | 1 | 3 |
| | DOTAM | 100** | | | | |
| K | CEA-split- | 154* + | 0 | 20 | 2 | 3 |
| | DOTAM | 100** | | | | |
| L | CEA-split- | 154* + | 0 | 20 | 3 | 3 |
| | DOTAM | 100** | | | | |
| M | ²¹²Pb-DOTAM- | 100 | 0 | 20 | 1 | 3 |
| | CEA-DOTAM | | | | | |

*P1AD8749: dose adjusted to 154 μg to compensate for a 35% hole/hole impurity in the stock solution;
**P1AD8592;
***Adjusted from 3 cycles to 1 cycle due to acute radiation-induced toxicity at the first treatment cycle.

Solid xenografts were established in SCID mice on study day 0 by SC injection of 5×10⁶ cells (passage 24) in RPMI/Matrigel into the right flank. Fifteen days after tumor cell injection, mice were sorted into experimental groups with an average tumor volume of 122 mm³. The ²¹²Pb-DOTAM was injected on day 23 after inoculation; the average tumor volume was 155 mm³ on day 22.

The CEA-DOTAM and DIG-DOTAM antibodies were diluted in vehicle buffer to a final concentration of 100 μg per 200 μL for IP administration according to the table above (Study groups in protocol 160). The CEA-split-DOTAM-VH/VL antibodies were mixed together into one single injection solution for IP administration, containing 100 μg of each construct per 200 μL. For P1AD8749, the dosing was adjusted to 154 μg to compensate for a 35% hole/hole impurity in the stock solution (the side of the molecule that does not carry the VH/VL). The Ca-DOTAM-dextran-500 CA was administered IP (25 μg per 200 μL of PBS) 7 days after the BsAb injection, followed 24 hours later by ²¹²Pb DOTAM (R07205834) according to the experimental schedule in FIG. 12. PRIT-treated mice (2- and 3-step) were injected IV with 100 μL of the Ca-quenched ²¹²Pb-DOTAM solution (20 μCi in 100 μL 0.9% NaCl).

Mice treated with 1-step RIT received only one injection: pre-bound ²¹²Pb-DOTAM-CEA-DOTAM (20 μC₁/20 μg BsAb in 100 μL 0.9% NaCl for IV injection). The direct-labeled antibody was prepared by incubating the ²¹²Pb-DOTAM with the CEA-DOTAM BsAb for 10 minutes at 37° C.

The following organs and tissues were harvested from mice in groups A-E at the time of euthanasia: serum, liver, spleen, kidneys, pancreas, and tumor. Before euthanasia, the live mouse was anesthetized for retro-orbital blood collection. The collected blood samples were centrifuged at 10 000 ref during 5 minutes and the resulting serum fractions isolated, frozen, and stored at −20° C. The excised tissues were immediately put in 10% neutral buffered formalin (4° C.) and then transferred to 1×PBS (4° C.) after 24 hours. The formalin-fixed samples were shipped to Roche Pharma Research and Early Development, Roche Innovation Center Basel, for further processing and analysis.

Mice in groups F, G, J, and M were sacrificed and necropsied 24 hours after their first and only injection of ²¹²Pb-DOTAM or ²¹²Pb-DOTAM-BsAb; groups H and K were sacrificed and necropsied 24 hours after their second ²¹²Pb-DOTAM injection; groups I and L were sacrificed and necropsied 24 hours after their third ²¹²Pb-DOTAM injection. Blood was collected at the time of euthanasia from the venous sinus using retro-orbital bleeding on anesthetized mice, before termination through cervical dislocation. The following organs and tissues were also harvested for biodistribution purposes: bladder, spleen, kidneys, liver, lung, muscle, tail, skin, and tumor.

### Results

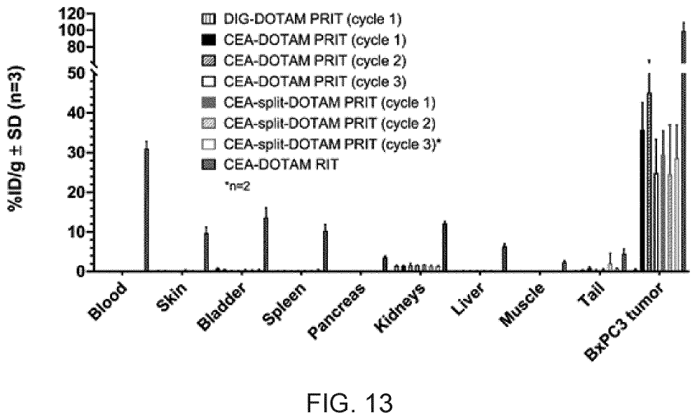

The average ²¹²Pb accumulation and clearance in all collected tissues 24 hours after injection is shown for each therapy and treatment cycle in FIG. 13. The negative control resulted in no uptake (0.4% ID/g) in tumor. Two-way analysis of variance (ANOVA) with Tukey's multiple comparisons test showed that the distributions were not significantly different at any cycle for the 2-step and 3-step PRIT; however, the differences were at all cycles statistically significant compared with the negative control and the 1-step RIT (p<0.05). The tumor uptake was 25-45% ID/g for 3-step PRIT and 25-30% ID/g for 2-step PRIT, without any statistically significant difference between either treatment or cycle. For 1-step RIT, the tumor uptake was 99% at the one and only treatment cycle. The uptake in normal tissues was very low for both PRIT regimens, but significantly higher in all organs and tissues after 1-step RIT, due to the much longer circulating time of the pre-incubated antibody compared with the small, radiolabeled DOTAM chelate.

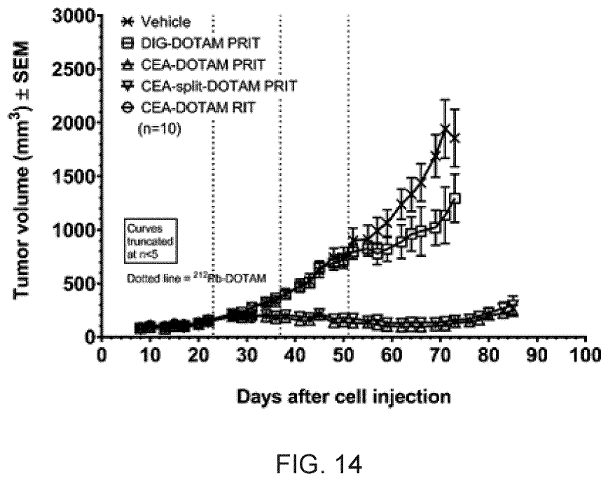

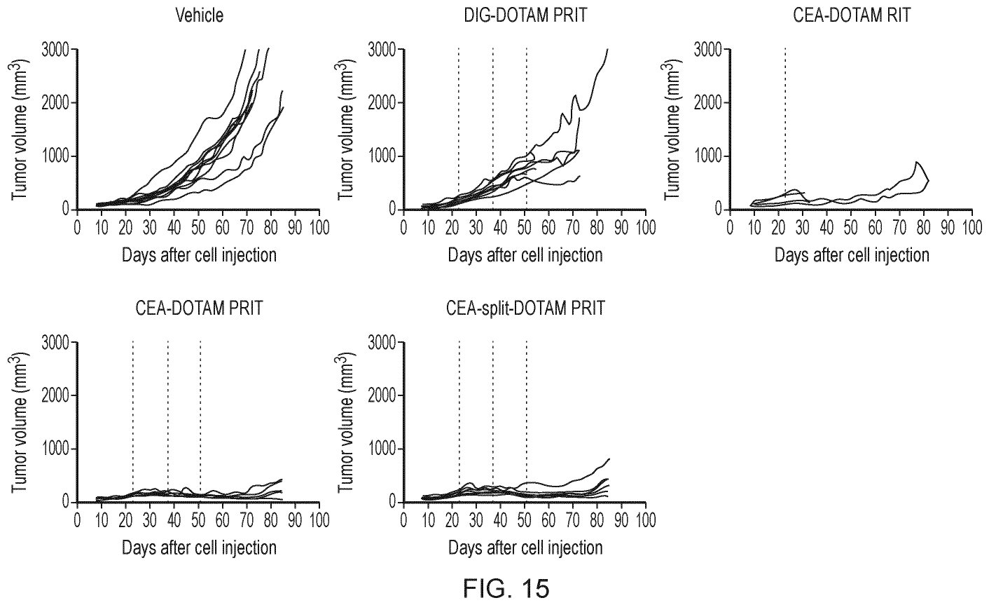

The average tumor development and the individual tumor growth curves are shown in FIG. 14 and FIG. 15, respectively. Tumors in the non-treated vehicle group and the DIG-DOTAM group grew steadily, albeit with slightly lower doubling rate in the latter after the third treatment. In contrast, tumors in the PRIT and RIT groups decreased in size after the first treatment cycle, and maintained tumor control until approximately 10 weeks after inoculation, when the tumors started to increase in size. The 2-step and 3-step PRIT treatments resulted in near identical tumor control. No tumors regressed completely.

On study day 83, the last day on which all treatment groups could be analyzed based on means, the TGI was 91.7% and 88.4% for PRIT using CEA-DOTAM (3-step) and CEA-split-DOTAM-VH/VL (2-step), respectively, compared with the vehicle control. The corresponding number for 1-step RIT was 72.6%, whereas the TGI was −59.7% for the non-specific DIG-DOTAM control. On the same day, the TR based on means was −1.9 for 3-step CEA-DOTAM PRIT, −2.9 for 2-step CEA-split-DOTAM-VH/VL PRIT, −4.7 for 1-step RIT, −28.8 for DIG-DOTAM PRIT, and −39.3 for the vehicle control.

Due to the adverse events described below, survival analysis was not considered statistically relevant.

### Adverse Events and Toxicity

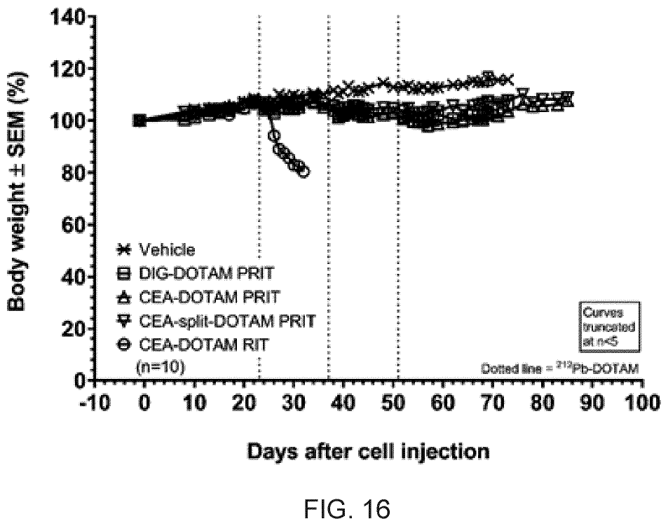

The BW development in all therapy groups is shown in FIG. 16. The multiple cycles of 2- and 3-step PRIT with 20 μCi of ²¹²Pb-DOTAM were well tolerated, but acute BW loss occurred in mice receiving 1-step RIT, with 8/10 mice in group E euthanized after the first RIT cycle (6-11 days after ²¹²Pb irradiation) due to a drop in BW of 20% or more. The remaining 2 RIT mice were not given any further ²¹²Pb-DOTAM-CEA-DOTAM injections but were continuously followed up for tumor growth assessment.

In addition, a number of mice were sacrificed for ethical reasons due to declining tumor status, i.e. tumors opening up or leaking. In the DIG-DOTAM group, 9/10 mice were euthanized before reaching a tumor volume of 3000 mm³ for this reason; for the non-treated vehicle control, the corresponding number was 5/10. The problem was less pronounced in the PRIT and RIT groups, with 1/10, 2/10, and 2/10 mice euthanized for this reason in the 3-step PRIT, 2-step PRIT, and 1-step RIT groups, respectively. This is reflected in the individual tumor growth curves in FIG. 15.

Finally, 1 mouse in group C was euthanized due to a degrading wound under the anus. All adverse events are listed in the table below.

### Adverse Events in Protocol 160

| | Mice | | |
|---|---|---|---|
| | sacrificed | | Reason for |
| Group | (n per group) | Study day | termination |
| A: Vehicle | 5(10) | 53, 71, 71, 73, 75 | Declining |
| | | | tumor status |
| B : DIG-DOTAM | 9(10) | 55, 55, 55, 55, 61, | Declining |
| | | 73, 73, 73, 74 | tumor status |
| C : CEA -DOTAM | 1(10) | 83 | Wound under |
| | | | the anus |
| C : CEA -DOTAM | 1(10) | 85 | Declining |
| | | | tumor status |
| D : CEA-split-DOTAM | 2(10) | 83, 83 | Declining |
| | | | tumor status |
| E : ²¹²Pb-DOTAM-CEA- | 8(10) | 29, 29, 30, 31, 31, | BW loss 20% |
| DOTAM | | 32, 32, 34 | |
| E :²¹²Pb-DOTAM-CEA- | 2(10) | 83, 83 | Declining |
| DOTAM | | | tumor status |

²¹²Pb irradiation was performed on study day 23 (cycle 1), 37 (cycle 2), and 51 (cycle 3).

### Conclusion

No difference was seen between CEA-PRIT using the 3-step scheme (CEA-DOTAM BsAb, CA, and ²¹²Pb-DOTAM) and the 2-step scheme (CEA-split-DOTAM-VH/VL antibodies and ²¹²Pb-DOTAM); the TGI was significant and near identical for the two treatments, and 3 cycles of 20 μC₁ could be safely administered in both cases. Contrastingly, 20 μC₁ of ²¹²Pb-DOTAM pre-bound to CEA-DOTAM before injection (1-step RIT) was not tolerated by a large majority of the treated mice.

The study thus demonstrated tolerability and therapeutic efficacy of CA-independent 2-step PRIT using the developed CEA-split-DOTAM-VH/VL constructs.

### Example 7: Protocol 175

The aim of protocol 175 was to assess the impact of increased injected pretargeting antibody amount on the subsequent ²¹²Pb accumulation in tumor and healthy tissues. Two different doses of CEA-split-DOTAM-VH/VL antibodies were compared: the standard amount (100 μg) and 2.5 times higher dose (250 μg). Moreover, a modification was made to the CEA-split-DOTAM-VH construct to extend its VH to avoid anti-drug antibody (ADA) formation (this was used together with a previously tested CEA-split-DOTAM-VL construct). The VH was extended to comprise the first three amino acids from the antibody CH1 domain: alanine, serine, and threonine (AST), and the construct hereafter referred to as CEA-split-DOTAM-VH-AST.

Antibody P1AD8592 has already been described above, in example 4. P1AF0171 is the same as P1AD8749 except that the fusion HC is extended by the residues AST—thus, antibody P1AD0171 consists of the light chain D1AA3384 as described above (SEQ ID NO: 34), the first heavy chain D1AC4022 as described above (SEQ ID NO: 28), and a second heavy chain D1AE3669 as shown below:

D1AE3669 (HC1cnob <CEA> CH1A1A Dotam-VH-AST)
(SEQ ID NO.: 147)
QVQLVQSGAEVKKPGASVKVSCKASGYTFTEFGMNWVRQAPGQGLEWMG
WINTKTGEATYVEEFKGRVTFTTDTSTSTAYMELRSLRSDDTAVYYCAR
WDFAYYVEAMDYWGQGTTVTVSSASTKGPSVFPLAPSSKSTSGGTAALG
CLVKDYFPEPVTVSWNSGALTSGVHTFPAVLQSSGLYSLSSVVTVPSSS
LGTQTYICNVNHKPSNTKVDKKVEPKSCDKTHTCPPCPAPEAAGGPSVF
LFPPKPKDTLMISRTPEVTCVVVDVSHEDPEVKFNWYVDGVEVHNAKTK
PREEQYNSTYRVVSVLTVLHQDWLNGKEYKCKVSNICALGAPIEKTISK
AKGQPREPQVYTLPPCRDELTKNQVSLWCLVKGFYPSDIAVEWESNGQP
ENNYKTTPPVLDSDGSFFLYSKLTVDKSRWQQGNVFSCSVMHEALHNHY
TQKSLSLSPGGGGGSGGGGSGGGGSGGGGSVTLKESGPVLVKPTETLTL
TCTVSGFSLSTYSMSWIRQPPGICALEWLGFIGSRGDTYYASWAKGRLT
ISKDTSKSQVVLTMTNMDPVDTATYYCARERDPYGGGAYPPHLWGRGTL
VTVSSAST

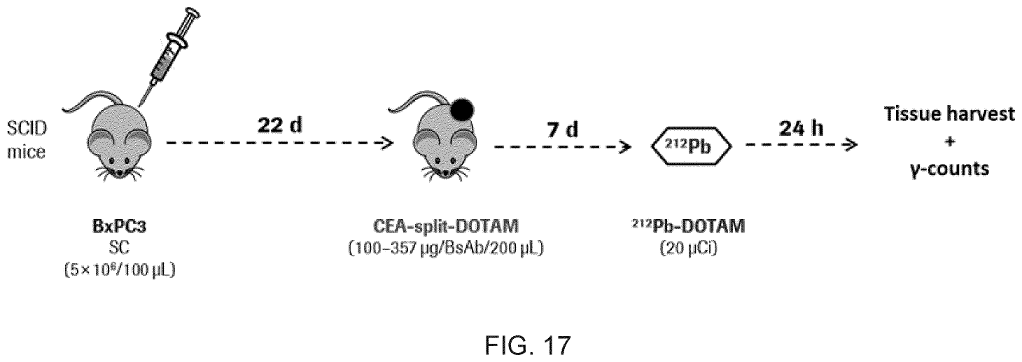

Mice carrying SC BxPC3 tumors were injected with either
1× the standard dose of CEA-split-DOTAM-VH/VL BsAb followed 7 days later by the radiolabeled ²¹²Pb-DOTAM, or
2.5× the standard dose of CEA-split-DOTAM-VH/VL BsAb followed 7 days later by the radiolabeled ²¹²Pb-DOTAM.
   The in vivo distribution of ²¹²Pb-DOTAM was assessed 24 hours after the radioactive injection. The study outline is shown in FIG. 17.

### Study Design

The time course and design of protocol 175 are shown below.

### Time Course of Protocol 175

| Study day | Experimental procedure |
|---|---|
| 0 | Preparation of BxPC3 cells and filling of syringes |
| 0 | SC injection of BxPC3 cells |
| 22 | IP* injection of CEA-split-DOTAM-VH/VL BsAbs |
| 29 | Elution of ²¹²Pb-DOTAM and filling of syringes |
| 29 | IV injection of ²¹²Pb-DOTAM |
| 30 | Euthanasia and tissue harvest (24 h p.i.) + gamma counting |

*IP injection required due to low compound concentration (200 μL., per construct = 400 μL in total)

### Study Groups in Protocol 175

| | P1AF0171 | P1AD85 | | | |
|---|---|---|---|---|---|
| | (VH-AST) | 92 (VL) | ²¹²Pb | BD | n |
| Group | (μg) | (μg) | (μCi) | (h p.i.) | (mice) |
| A | 143* | 100 | 10 | 24 | 4 |
| B | 357* | 250 | 10 | 24 | 4 |

*P1AF0171 dose adjusted and 143 to 357 μg to compensate for a ~30% hole/hole impurity.

Solid xenografts were established in each SCID mouse on study day 0 by SC injection of 5×10⁶ cells (passage 24) in RPMI/Matrigel into the right flank. Twenty-one days after tumor cell injection, mice were sorted into experimental groups with an average tumor volume of 310 mm³. The ²¹²Pb-DOTAM was injected on day 29 after inoculation; the average tumor volume was 462 mm³ on day 30.
All mice were sacrificed and necropsied 24 hours after injection of ²¹²Pb-DOTAM, and the following organs and tissues harvested for measurement of radioactive content: blood, skin, spleen, pancreas, kidneys, liver, muscle, tail, and tumor.

### Results

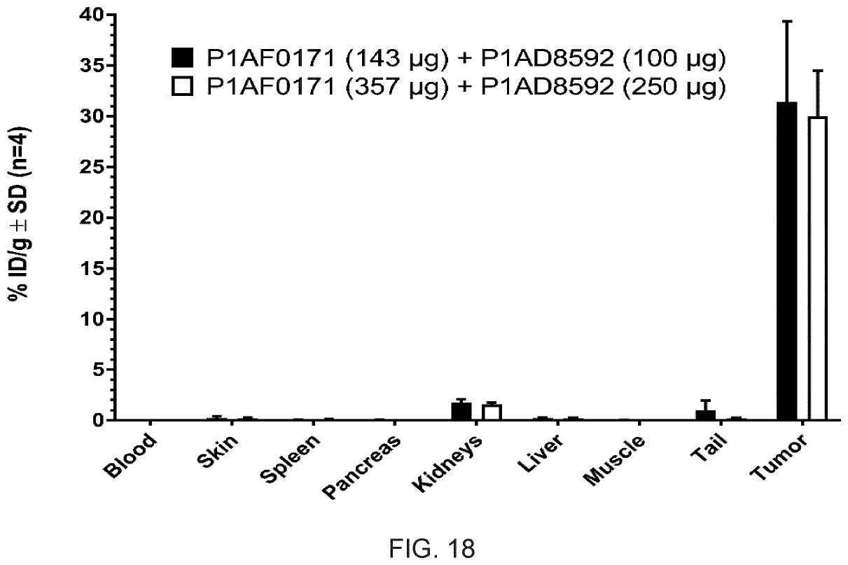

The average ²¹²Pb distribution in all collected tissues 24 hours after injection is shown in FIG. 18. There was no significant difference in tumor or normal tissue uptake of ²¹²Pb between the two dose levels. The tumor accumulation was 30-31% ID/g for both treatment groups, with a kidney uptake of <2% ID/g at this time point. One mouse had ˜1% ID/g in the tail due to ²¹²Pb-DOTAM injection issues, but no other collected healthy tissues showed any appreciable ²¹²Pb accumulation.

### Adverse Events and Toxicity

There were no adverse events or toxicity associated with this study.

### Conclusion

Increasing the dose of the pretargeting CEA-split-DOTAM-VH/VL antibodies by 2.5-fold did not improve the tumor accumulation of subsequently administered ²¹²Pb-DOTAM in this in vivo model. However, it also did not increase the accumulation of radioactivity in normal tissues, highlighting the strong specificity achieved using this 2-step pretargeting regimen. Finally, the results verified the function of the extended-VH CEA-split-DOTAM-VH-AST construct.

### Example 8: Protocol 185

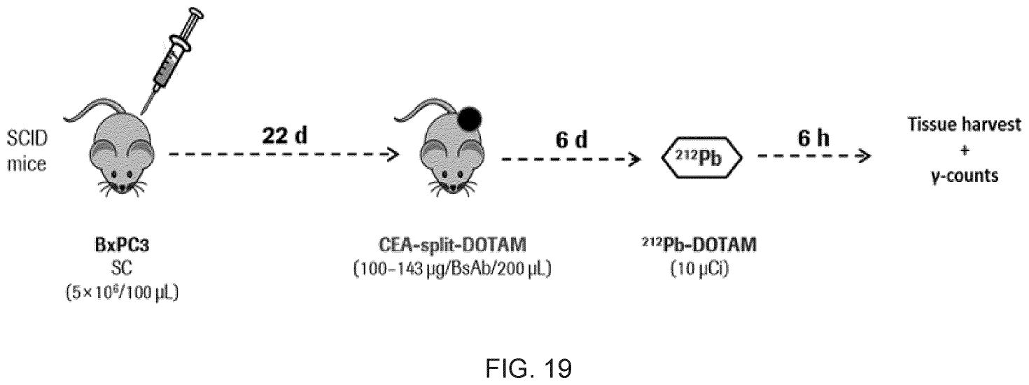

The aim of protocol 185 was to assess a CEA-split-DOTAM-VH/VL targeting the T84.66 epitope. Sequences of P1AF0709 and P1AF0298 are provided herein. P1AF0709 has a first heavy chain of D1AE4688 (SEQ ID NO: 83) and a second heavy chain of D1AA4920 (SEQ ID NO: 84). P1AF0298 has a first heavy chain of D1AE4687 (SEQ ID NO: 86) and a second heavy chain of D1AE3668 (SEQ ID NO: 87). Both have the light chain of D1AA4120 (SEQ ID NO: 89).
Mice carrying SC BxPC3 tumors were injected with the standard dose of CEA-split-DOTAM-VH/VL BsAb (100 μg per antibody) followed 6 days later by the radiolabeled ²¹²Pb-DOTAM. The in vivo distribution of ²¹²Pb-DOTAM was assessed 6 hours after the radioactive injection. The study outline is shown in FIG. 19.

### Study Design

The time course and design of protocol 185 is shown below.

### Time Course of Protocol 185

| Study day | Date | Experimental procedure |
|---|---|---|
| 0 | 2020 Mar. 04 | Preparation of BxPC3 cells and filling |
| | | of syringes |
| 0 | 2020 Mar. 04 | SC injection of BxPC3 cells |
| 22 | 2020 Mar. 26 | IV injection of CEA-split-DOTAM-VH/VL BsAbs |
| 27 | 2020 Mar. 31 | Elution of ²¹²Pb-DOTAM and filling of syringes |
| 28 | 2020 Apr. 01 | IV injection of ²¹²Pb-DOTAM |
| 28 | 2020 Apr. 01 | Euthanasia and tissue harvest (6 h p.i.) + |
| | | gamma counting |

### Study Groups in Protocol 185

| | P1AF0298 | P1AF0709 | P1AF0171 | P1AD8592 | | | |
|---|---|---|---|---|---|---|---|
| | T84.66 | T84.66 | CH1A1A | CH1A1A | | | |
| | (VH-AST) | (VL) | (VH-AST) | (VL) | ²¹²Pb | BD | n |
| Group | (μg) | (μg) | (μg) | (μg) | (μCi) | (h p.i.) | (mice) |
| A | 100 | 100 | 0 | 0 | 10 | 6 | 5 |
| B | 0 | 0 | 143* | 100 | 10 | 6 | 5 |

*P1AF0171 dose adjusted to 143 μg to compensate for a ~30% hole/hole impurity.

Solid xenografts were established in each SCID mouse on study day 0 by SC injection of 5×10⁶ cells (passage 27) in RPMI/Matrigel into the right flank. Twenty-two days after tumor cell injection, mice were sorted into experimental groups with an average tumor volume of 224 mm³. The ²¹²Pb-DOTAM was injected on day 28 after inoculation, at which point the average tumor volume had reached 385 mm³.
All mice were sacrificed and necropsied 6 hours after injection of ²¹²Pb-DOTAM, and the following organs and tissues harvested for measurement of radioactive content: blood, skin, spleen, pancreas, kidneys, liver, muscle, tail, and tumor. Collected tumors were split in two pieces: one was measured for radioactive content, and the other put in a cryomold containing Tissue-Tek® optimum cutting temperature (OCT) embedding medium, and put on dry ice for rapid freezing. Frozen samples in OCT were maintained at −80° C. before cryosectioning, immunofluorescence staining, and analysis using a Zeiss Axio Scope.A1 modular microscope.

### Results

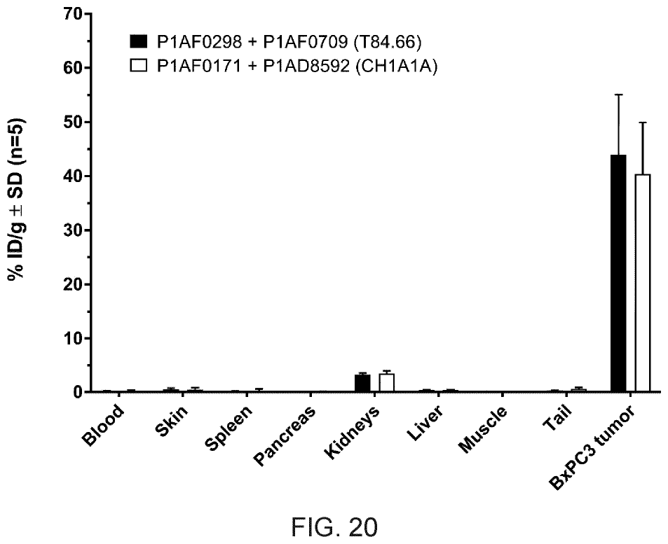

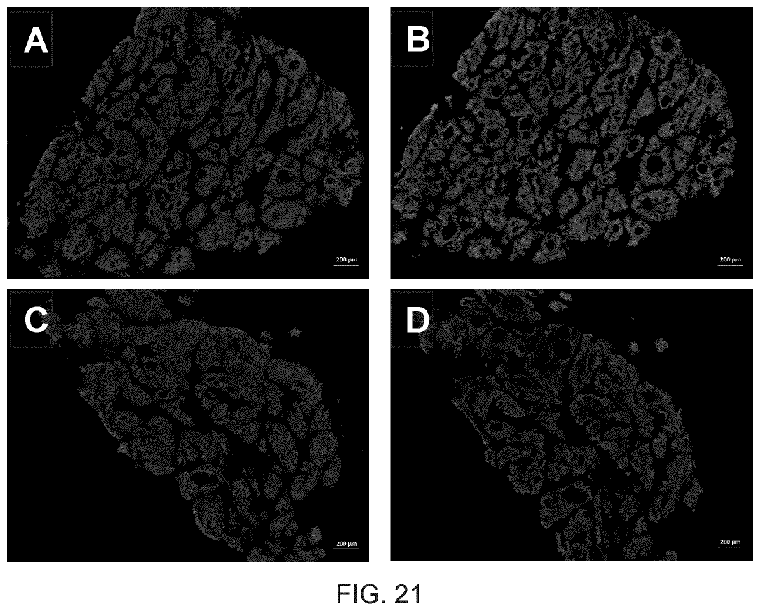

The average ²¹²Pb distribution in all collected tissues 6 hours after injection is shown in FIG. 20. The tumor accumulation was 40% ID/g (CH1A1A) or 44% ID/g (T84.66). The only other appreciable accumulation of radioactivity was found in kidneys: 3-5% ID/g at 6 h p.i. for the two groups.
Examples of the intratumoral distribution of CEA-split-DOTAM-VH/VL pairs targeting either T84.66 (group A) or CH1A1A (group B) are shown in FIG. 21. Panels A and C show that the CEA expression is high and homogeneous in BxPC3 tumors, and panels B and D demonstrate that the antibody distribution 7 days after injection is distributed similarly. However, the samples from group A displayed a stronger signal overall, compared with tumor samples from group B, providing evidence that T84.66 is a stronger binder than CH1A1A.

### Adverse Events and Toxicity

There were no adverse events or toxicity associated with this study.

### Conclusion

The results verified the function of CEA-split-DOTAM-VH/VL constructs targeting the T84.66 epitope of CEA. The resulting accumulation of 212Pb in pretargeted CEA-expressing tumors was high and specific, and CEA-split-DOTAM-VH/VL pairs targeting either the CH1A1A or T84.66 epitope were homogeneously distributed inside the CEA-expressing tumors.

### Example 9: Protocol 189

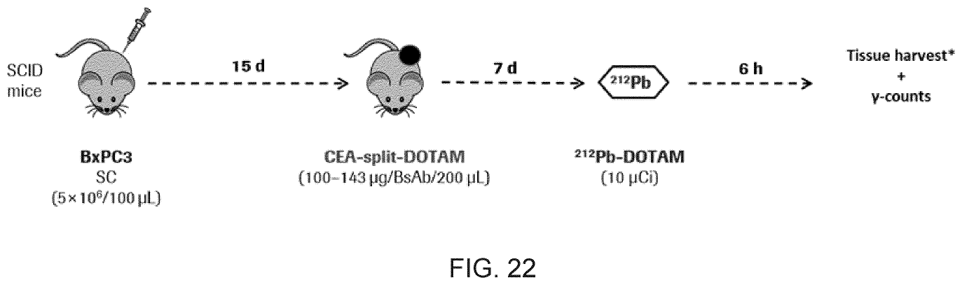

The aim of protocol 189 was to assess bi-paratopic CEA-split-DOTAM-VH/VL antibody pairs targeting T84.66 VH-AST/CH1A1A VL and T84.66 VL/CH1A1 VH-AST, compared with the positive control pair targeting CH1A1A VH-AST/VL. This bi-paratopic combination precludes formation of the full Pb-DOTAM binder on soluble CEA that only expresses one of the two epitopes (e.g. T84.66), thereby mitigating potential adverse effects thereof, such as increased circulating radioactivity and associated radiation-induced toxicity, and decreased efficacy from competition with off-tumor targets.
Mice carrying SC BxPC3 tumors were injected with the standard dose of CEA-split-DOTAM-VH/VL BsAb (100 μs per antibody) followed 7 days later by the radiolabeled ²¹²Pb-DOTAM. The in vivo distribution of ²¹²Pb-DOTAM was assessed 6 hours after the radioactive injection. The study outline is shown in FIG. 22.

### Study Design

The time course and design of protocol 189 is shown below.

### Time Course of Protocol 189

| Study day | Experimental procedure |
|---|---|
| 0 | Preparation of BxPC3 cells and filling of syringes |
| 0 | SC injection of BxPC3 cells |
| 15 | IV injection of CEA-split-DOTAM-VH/VL BsAbs |
| 21 | Elution of ²¹²Pb-DOTAM and filling of syringes |
| 22 | IV injection of ^{2I2}Pb-DOTAM _ |
| 22 | Euthanasia and tissue harvest (6 h p.i.) + gamma counting |

### Study Groups in Protocol 189

| | P1AF0298 | P1AF0709 | P1AF0171 | P1AD8592 | | | |
|---|---|---|---|---|---|---|---|
| | T84.66 | T84.66 | CH1A1A | CH1A1A | | | |
| | (VH-AST) | (VL) | (VH-AST) | (VL) | ²¹²Pb | BD | n |
| Group | (μg) | (μg) | (μg) | (μg) | (μCi) | (h p.i.) | (mice) |
| A | 100 | 0 | 0 | 100 | 10 | 6 | 5 |
| B | 0 | 100 | 143* | 0 | 10 | 6 | 5 |
| C | 0 | 0 | 143* | 100 | 10 | 6 | 3 |

*P1AF0171 dose adjusted to 143 μg to compensate for a ~30% hole/hole impurity.

Solid xenografts were established in each SCID mouse on study day 0 by SC injection of 5×10⁶ cells (passage 31) in RPMI/Matrigel into the right flank. Fourteen days after tumor cell injection, mice were sorted into experimental groups with an average tumor volume of 343 mm³. The ²¹²Pb-DOTAM was injected on day 22 after inoculation; the average tumor volume had reached 557 mm³ on day 21.
All mice were sacrificed and necropsied 6 hours after injection of ²¹²Pb-DOTAM, and the following organs and tissues harvested for measurement of radioactive content: blood, skin, spleen, pancreas, kidneys, liver, muscle, tail, and tumor.

### Results

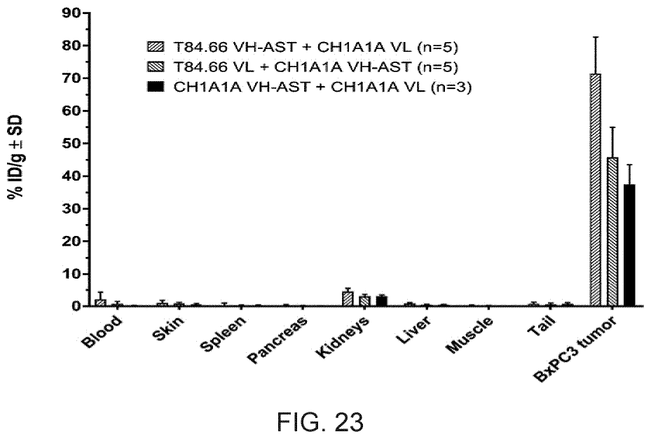

The average ²¹²Pb distribution in all collected tissues 6 hours after injection is shown in FIG. 23. The tumor accumulation of the bi-paratopic variations was 71% ID/g and 46% ID/g for T84.66 VH-AST+CH VL and T84.66 VL+CH VH-AST, respectively. The positive CH control resulted in 37% ID/g. Two-way ANOVA with Tukey's multiple comparisons test showed that all three groups were significantly different from each other in terms of tumor uptake (p<0.0001 for T84.66 VH-AST+CH1A1A VL versus the two other groups; p=0.0020 for T84.66 VL+CH VH-AST versus CH1A1A only). No other organs showed statistically significant differences between groups, although a slightly higher retention in blood was indicated for the T84.66 VH-AST+CH1A1A VL combination compared with the two other groups: 2% ID/g compared with <1% ID/g. The kidney uptake was similarly slightly higher, although not statistically significantly so: 4.5% ID/g for T84.66 VH-AST+CH1A1A compared with 3% ID/g for the other two.

### Adverse Events and Toxicity

There were no adverse events or toxicity associated with this study. However, the BxPC3 tumor growth was significantly faster, and with greater variability, in this study compared with the standard growth rate. On necropsy, it was concluded that the big tumors (a majority) were filled with liquid, which was emptied when tumors were cut in half before radioactive measurement; this liquid likely caused the accelerated growth rate, but did not affect the % IA/g to any great extent as the tumors were weighed and measured after being opened.

### Conclusion

The results verified the function of bi-paratopic targeting of the T84.66 and CH1A1A epitopes of CEA using the tested CEA-split-DOTAM-VH/VL constructs and demonstrated surprisingly high efficacy for this combination as compared to the positive CH1A1A control. The resulting accumulation of ²¹²Pb in pretargeted CEA-expressing tumors was high and specific, with indications of a particular advantage for the T84.66 VH-AST+CH1A1A VL pair.

### Example 10

These examples investigate recruitment of Pb-DOTA to cells by split antibodies as described herein.
P1AF0712 has a first heavy chain of SEQ ID NO:97, a second heavy chain of SEQ ID NO: 98 and a light chain of SEQ ID NO: 103. P1AF0713 has a first heavy chain of SEQ ID NO: 100, a second heavy chain of SEQ ID NO: 101 and a light chain of SEQ ID NO: 103.
MKN-45 cells were detached from the culture bottle using Trypsin and were counted using a Casy cell counter. After pelleting at 4° C., 300 g the cells were resuspended in FACS Buffer (2.5% FCS in PBS), adjusted to 2.0E+06 cells/mL dispensed to 96-well PP V-bottom-Platte (25 μL/well=5.0E+04 Zellen/well).

### FACS Staining Using DOTA-FITC

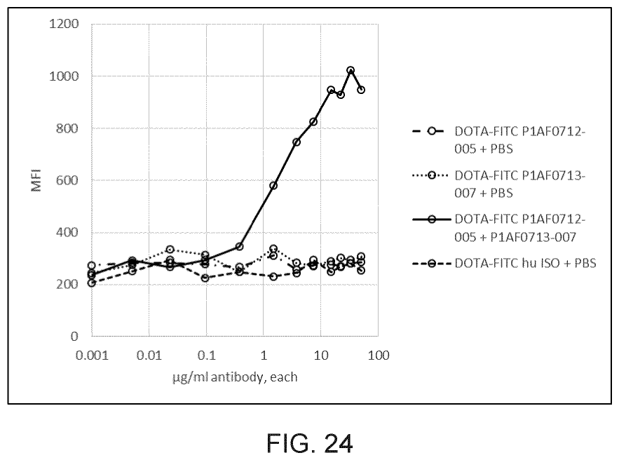

The CEA specific SPLIT antibodies (P1AF0712 or P1AF0713 respectively) were adjusted to 40 μg/mL in FACS buffer, resulting in a final concentration of 10 μg/mL. Both antibodies were added to the cells either combined or separated and followed by buffer and incubated at 4° C. for 1 h. Subsequently, Pb-DOTA labeled with FITC was added to the cells in equimolar ratio to the antibodies and incubated for 1 h at 4° C. The cells were then washed twice in FACS buffer and resuspended in 70 μl/well FACS buffer for measurement using a FACS Canto (BD, Pharmingen). It was shown (FIG. 24) that neither of the SPLIT halves was giving rise to a fluorescence signal, indicating a lack of Pb-DOTA binding capability. Only a combination of both SPLIT halves was able to recruit Pb-DOTAM-FITC to the target cells (FIG. 24).
FACS Staining Using <huIgG(H+L)A488>
The CEA specific SPLIT antibodies (P1AF0712 or P1AF0713 respectively) were adjusted to 40 μg/mL in FACS buffer, resulting in a final concentration of 10 μg/mL. Both antibodies were added to the cells either separated followed by buffer or combined and incubated at 4° C. for 1 h. The cells were then washed twice in FACS buffer. After washing, the cells were resuspended in 50 μL FACS-buffer containing secondary antibody (<huIgG(H+L)>-Alexa488, c=10 μg/mL) and incubated 1 h at 4° C. The cells were then washed twice in FACS buffer and resuspended in 70 μl/well FACS buffer for measurement using a FACS Canto (BD, Phaimingen). EC50 for both SPLIT antibodies was comparable, indicating CEA specific cell binding of both SPLIT antibodies. Due to the higher amount of antibody in the mixture, a lower EC50 was obtained under these circumstances, as shown in the table below.

### EC50 Determination of SPLIT Antibodies

| | | EC50 |
|---|---|---|
| | | μg/ml |
| | | absolute |
| <hu>A488 | P1AF0712+PBS | 2.7 |
| | P1AF0713+PBS | 2.3 |
| | P1AF0712+P1AF0713 | 0.9 |
| | hu ISO +PBS | |
| DOTA-FITC | P1AF0712+PBS | na |
| | P1AF0713+PBS | na |
| | P1AF0712+P1AF0713 | 2.4 |
| | hu ISO +PBS | |

EC50 was determined for the SPLIT antibodies using either secondary antibody based detection (<hu>488, top panel) or Pb-DOTA-FITC (DOTA-FITC, bottom panel)

### Example 11: Biacore Binding Experiments

This example tests binding of TA-split-DOTAM-VH and TA-split-DOTAM-VL individually to DOTAM, as compared to the reference antibody CEA-DOTAM (R07198427, PRIT-0213). It further tests binding of DOTAM to the TA-split-DOTAM-VH/VL pairs, as compared to the reference antibody.
The correspondence between the coding used in these examples and the protein numbers used elsewhere in this application is shown below. Sequences are also provided. The reference antibody is coded as “PRIT_RS” in this example.

| | SPR Code | | | | LC | HC | Fusion HC |
|---|---|---|---|---|---|---|---|
| | (Prodrug | SPR | | | (SEQ | (SEQ | (SEQ |
| Target binder | A + B) | Code | DOTAM | Protein | ID NO) | ID NO) | ID NO) |
| <CEA> CH1A1A | P1_AB | P1_A | VL | P1AD8592 | 34 | 30 | 33 |
| | | P1 _B | VH | P1AD8749 | 34 | 28 | 32 |
| <CEA> CH1A1A | P2_AB | P2_A | VL | P1AD8592 | 34 | 30 | 33 |
| | | P2_B | VH | P 1AF0171 | 34 | 28 | 147 |
| <CEA> T84.66 | P3_AB | P3_A | VL | P1AF0709 | 89 | 83 | 84 |
| | | P3_B | VH | P1AF0298 | 89 | 86 | 89 |
| <CEA> 28A9 | P4_AB | P4_A | VL | P1AF0710 | 96 | 90 | 91 |
| | | P4 _B | VH | P1AF0711 | 96 | 93 | 94 |
| <CEA> | P5_AB | P5_A | VL | P1AE4957 | 58 | 55 | 56 |
| A5H1EL1(G54A) | | P5_B | VH | P1AE4956 | 54 | 51 | 52 |
| <CEA> CH1A1A | P6_AB | P6_A | VL | P1AF0712 | 103 | 97 | 98 |
| | | P6_B | VH | P1AF0713 | 103 | 100 | 101 |
| <GPRC5D> | P7_AB | P7_A | VL | P1AF8284 | 107 | 104 | 105 |
| | | P7_B | VH | P1AF8285 | 107 | 104 | 106 |

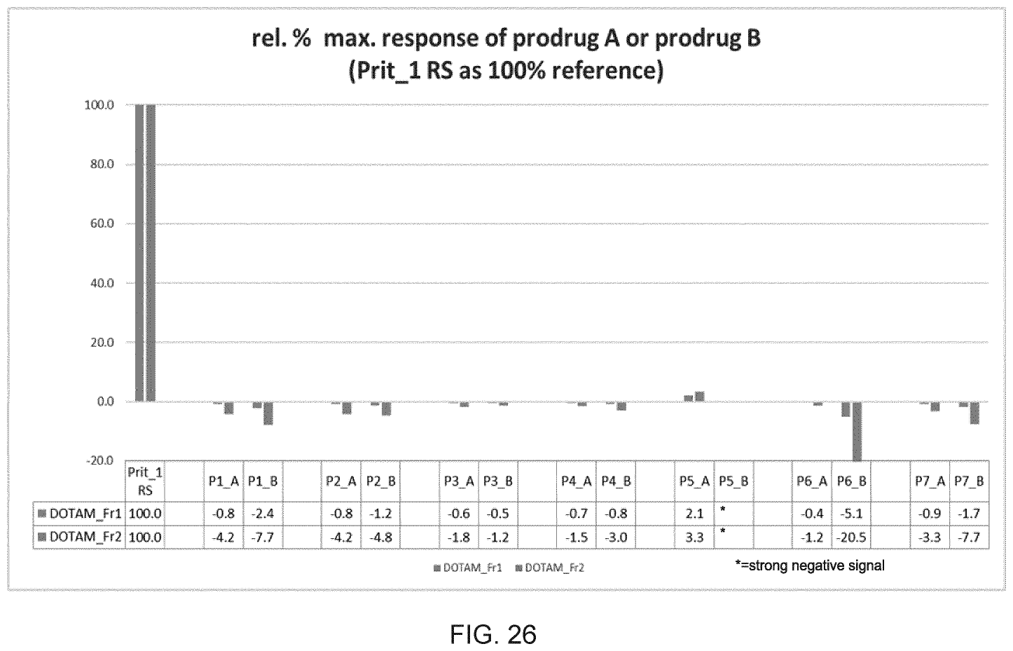

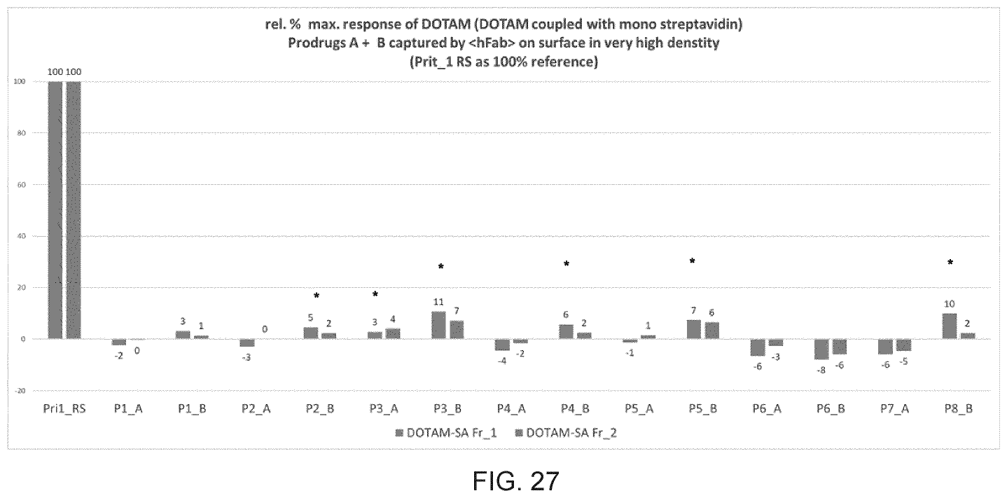

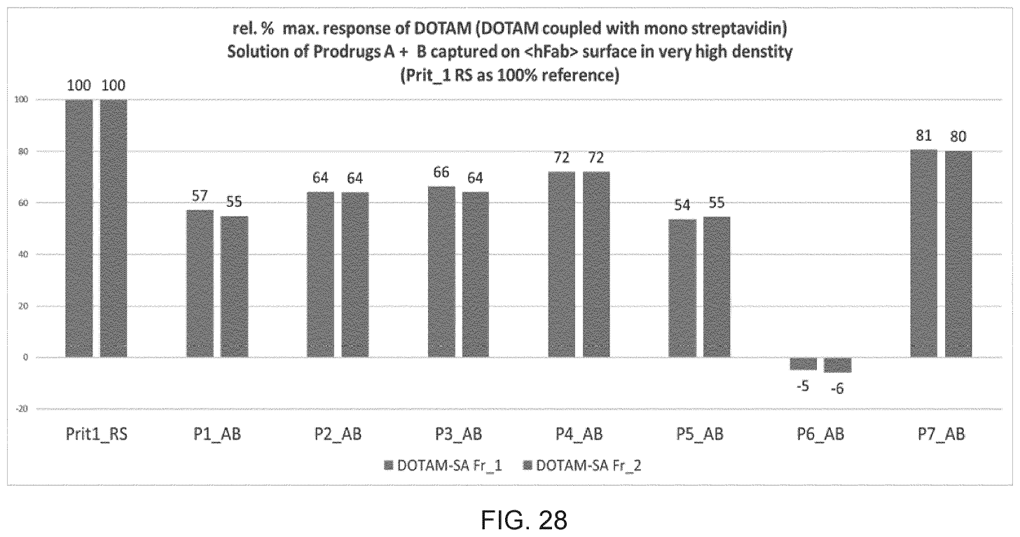

For these experiments, the PRIT SPLIT antibodies were purified by a first step of MabSelect Sure (Affinity Chromatography) and a second step of ion exchange chromatography (e.g. POROS XS), and then polished by Superdex 200 (Size Exclusion Chromatography).
The experiments were performed with Biacore T200 at 25° C. measuring temperature. All Biacore T200 experiments were carried out in HBS-P+(GE Healthcare, Br-1008-27) pH 7,4 running buffer. Two experiments were performed for each test antibody/antibody pair, using different DOTAM fractions.
1. In a first experiment, the binding of individual TA-split-DOTAM-VH and TA-split-DOTAM-VL antibodies to biotinylated DOTAM captured on a chip was assessed, relative to the reference antibody.
DOTAM (120 nM solution in HBS-P+) was captured in high density on CAP Chip Surface (10 μl/min, 60 Sec). Then the 600 nM solutions in HBS-P+ of Prodrug_A or Prodrug_B were injected over the DOTAM surface (10 μl/min, 90 sec). The dissociation was monitored for 240 sec at a flow rate of 10 μl/min. The relative maximum response determination was evaluated using T200 evaluation software.
The results are shown in FIG. 26. None of the individual antibodies showed binding to the captured DOTAM.
2. In a second experiment, individual TA-split-DOTAM-VH and TA-split-DOTAM-VL antibodies were first captured in a chip using an immobilized anti-hFab, and then binding of a DOTAM-monoStreptavidin complex (DOTAM+monoSteptavidin coupling 600 nM, 1:1 mol, 1 h at RT) was assessed.
The 600 nM solution in HBS-P+ of Prodrug_A or Prodrug B was injected over the anti hFab (GE Healthcare, BR-1008-27) CM5 Chip surface (10 μl/min, 120 sec). After the high density capturing of Prodrug A or B solution the DOTAM-monoStreptavidin complex was injected (20 μl/min, 90 sec). The dissociation was monitored for 180 sec at a flow rate of 20 μl/min. For new cycle the surface was regenerated by using of Glycin 2.1 and 75 sec regeneration time with 10 μl/min. The relative maximum response determination was evaluated using T200 evaluation software.
The results are shown in FIG. 27. Low percentage max. responses (as marked with * in the figure) are believed to be “traces” or unspecific interactions with DOTAM-SA, and reflect a need to optimize the assay.
3. In a third experiment, binding of the TA-split-DOTAM-VH/VL pairs to DOTAM is assessed, as compared to the reference antibody. Antibodies were first captured in a chip using an immobilized anti-hFab, and then binding of a DOTAM-monoStreptavidin complex (DOTAM+monoSteptavidin coupling 600 nM, 1:1 mol, 1 h at RT) was assessed.
The 300 nM solution in HBS-P+ of Prodrug_A and Prodrug B was injected over the anti hFab (GE Healthcare, BR-1008-27) CM5 Chip surface (10 μl/min, 120 sec). After the high density capturing of Prodrug A and B solution the DOTAM-monoStreptavidin complex was injected (20 μl/min, 90 sec). The dissociation was monitored for 180 sec at a flow rate of 20 μl/min. For new cycle the surface was regenerated by using of Glycin 2.1 and 75 sec regeneration time with 10 μl/min. The relative maximum response determination was evaluated using T200 evaluation software.
The results are shown in FIG. 28. All TA-split-DOTAM-VH/VL pairs showed a significant amount of binding for DOTAM, except for the P6_AB (P1AF0712/P1AF0713) pair, which are DOTA binders.
Similar results showing a significant amount of DOTAM binding for the TA-split-DOTAM-VH/VL pair but not for the individual members of the pair have also been obtained for the FAP-binders P1AF8286 and P1AF8287. P1AF8286 is composed of a first heavy chain of SEQ ID NO: 108, a second heavy chain of SEQ ID NO: 109 and a light chain of SEQ ID NO: 111, and P1AF8287 is composed of a first heavy chain of SEQ ID NO: 108, a second heavy chain of SEQ ID NO: 110 and a light chain of SEQ ID NO: 111. However, this assay still needs to be optimised.
Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

What is claimed is:

1. A set of antibodies comprising:
i) a first antibody that binds to an antigen expressed on the surface of a target cell, and which further comprises a VH domain of an antigen binding site for a radiolabelled compound, but which does not comprise a VL domain of an antigen binding site for the radiolabelled compound; and
ii) a second antibody that binds to said antigen expressed on the surface of the target cell, and which further comprises a VL domain of an antigen binding site for the radiolabelled compound, but which does not comprise a VH domain of the antigen binding site for the radiolabelled compound,
wherein said VH domain of the first antibody and said VL domain of the second antibody are together capable of forming a functional antigen binding site for the radiolabelled compound.

2. The set of antibodies of claim 1, wherein the first and second antibody each comprise i) an antibody fragment comprising an antigen binding site specific for said antigen expressed on the surface of a target cell and ii) either a VL domain or a VH domain of the antigen binding site for the radiolabelled compound.

3. The set of antibodies of claim 2, wherein the antibody fragment is selected from at least one Fv, scFv, or Fab or cross-Fab fragment.

4. The set of antibodies of claim 3, wherein the first antibody comprises or consists of:
a) a Fab fragment binding said antigen, and
b) a polypeptide comprising or consisting of
i) an antibody heavy chain variable domain (VH) of an antigen binding site for a radiolabelled compound, or
ii) an antibody heavy chain variable domain (VH) of an antigen binding site for a radiolabelled compound and an antibody heavy chain constant domain, wherein the C-terminus of VH domain is fused to the N-terminus of the constant domain;
wherein the polypeptide is fused by the N-terminus of the VH domain to the C terminus of the CL or CH1 domain of the Fab fragment;
and wherein the second antibody comprises or consists of:
c) a Fab fragment binding said antigen, and
d) a polypeptide comprising or consisting of
iii) an antibody light chain variable domain (VL) of an antigen binding site for a radiolabelled compound, or
iv) an antibody light chain variable domain (VL) of an antigen binding site for a radiolabelled compound and an antibody light chain constant domain,
wherein the C-terminus of the VL domain is fused to the N-terminus of the constant domain;
wherein the polypeptide is fused by the N-terminus of the VL domain to the C-terminus of the CL or CH1 domain of the Fab fragment;
and wherein antibody heavy chain variable domain (VH) of the polypeptide of (b) and antibody light chain variable domain (VL) of polypeptide of (d) are together capable of forming a functional antigen binding site for the radiolabelled compound.

5. The set of antibodies according to claim 4, wherein the polypeptide of (b) is fused by the N-terminus of the VH domain to the C terminus of the CL or CH1 domain of the Fab fragment of (a) via a peptide linker; and wherein the polypeptide of (d) is fused by the N-terminus of the VL domain to the C-terminus of the CL or CH1 domain of the Fab fragment of (c) via a peptide linker.

6. The set of antibodies according to claim 3, wherein the first antibody comprises
a) a tandem Fab comprising two Fab fragments, wherein the first and the second Fab fragment each bind the same epitope of said antigen and wherein the first and the second Fab fragment are connected via a peptide tether, wherein the first Fab is connected via its C-terminus to the N-terminus of the second Fab; and
b) a polypeptide comprising or consisting of
i) an antibody heavy chain variable domain (VH); or
ii) an antibody heavy chain variable domain (VH) and an antibody constant domain (CH1), wherein the C-terminus of VH domain is fused to the N-terminus of the CH1 domain;
wherein said polypeptide is fused by the N-terminus of the VH domain to the C-terminus of the CL or CH1 domain of the second Fab fragment;
and the second antibody comprises
c) a tandem Fab comprising two Fab fragments, wherein the first and the second Fab fragment each bind the same epitope of said antigen and wherein the first and the second Fab fragment are connected via a peptide tether, wherein the first Fab is connected via its C-terminus to the N-terminus of the second Fab; and
d) a polypeptide comprising or consisting of
i) an antibody light chain variable domain (VL); or
ii) an antibody light chain variable domain (VL) and an antibody light chain constant domain (CL), wherein the C-terminus of VH domain is fused to the N-terminus of the constant domain;
wherein said polypeptide is fused by the N-terminus of the VL domain to the C-terminus of the CL or CH1 domain of the second Fab fragment;
and wherein antibody heavy chain variable domain (VH) of the polypeptide of (b) and antibody light chain variable domain (VL) of polypeptide of (d) are together capable of forming a functional antigen binding site for the radiolabelled compound.

7. The set of antibodies according to claim 6, wherein the polypeptide of (b) is fused by the N-terminus of the VH domain to the C-terminus of the CL or CH1 domain of the second Fab fragment of (a) via a peptide linker; and wherein the polypeptide of (d) is fused by the N-terminus of the VL domain to the C-terminus of the CL or CH1 domain of the second Fab fragment of (c) via a peptide linker.

8. The set of antibodies according to claim 3, wherein the first antibody comprises
a) a tandem Fab comprising a first fragment and a second fragment, wherein the first fragment is connected by its C-terminus via a peptide tether to the N-terminus of the second fragment, wherein the first fragment binds a first epitope of said antigen and the second fragment binds a second epitope of said antigen, and wherein one of the fragments selected from the first and second fragments is a Fab and the other is a cross-Fab,
b) a polypeptide comprising or consisting of
i) an antibody heavy chain variable domain (VH); or
ii) an antibody heavy chain variable domain (VH) and an antibody heavy chain constant domain (CH1), wherein the C-terminus of VH domain is fused to the N-terminus of the CH1 domain;
wherein said polypeptide is fused by the N-terminus of the VH domain to the C-terminus of one of the chains of the second fragment; and
wherein the second antibody comprises
c) a tandem Fab comprising a first fragment and a second fragment, wherein the first fragment is connected by its C-terminus to the N-terminus of the second fragment, wherein the first fragment binds the a first epitope of said antigen and the second fragment binds a second epitope of said antigen, and wherein one of the fragments selected from the first and second fragments is a Fab and the other is a cross-Fab; and
d) a polypeptide comprising or consisting of
i) an antibody light chain variable domain (VL); or
ii) an antibody light chain variable domain (VL) and an antibody light chain constant domain (CL), wherein the C-terminus of VL domain is fused to the N-terminus of the light chain constant domain
wherein said polypeptide is fused by the N-terminus of the VL domain to the C-terminus of one of the chains of the second fragment;
and wherein antibody heavy chain variable domain (VH) of the polypeptide of (b) and antibody light chain variable domain (VL) of polypeptide of (d) are together capable of forming a functional antigen binding site for the radiolabelled compound.

9. The set of antibodies according to claim 8, wherein the polypeptide of (b) is fused by the N-terminus of the VL domain to the C-terminus of one of the chains of the second fragment of (a) via a peptide linker; and wherein the polypeptide of (d) is fused by the N-terminus of the VL domain to the C-terminus of one of the chains of the second fragment of (c) via a peptide linker.

10. The set of antibodies according to any one of claims 1-3, wherein the first and second antibody each further comprise an Fc domain.

11. The set of antibodies of claim 10, wherein the first and second antibody each comprise i) an antibody fragment comprising an antigen binding site specific for said antigen expressed on the surface of a target cell, ii) and Fc region and iii) either a VL domain or a VH domain of the antigen binding site for the radiolabelled compound fused to the Fc region.

12. The set of antibodies of claim 10 or 11, wherein the Fc domain is modified to reduce or eliminate effector function.

13. The set of antibodies according to any one of claims 10 to 12, wherein the first antibody comprises or consists of:
i) a complete light chain fragment;
ii) a complete heavy chain;
iii) an additional Fc chain lacking Fd; and
iv) a polypeptide comprising or consisting of the VH domain of the antigen binding site for the radiolabeled compound;
wherein the light chain of (i) and the heavy chain of (ii) together provide an antigen binding site for said antigen; and wherein the polypeptide comprising or consisting of the VH domain of the antigen binding site for the radiolabeled compound is fused by its N-terminus to the C-terminus of either (ii) or (iii); and
wherein the second antibody comprises or consists of
v) a complete light chain fragment;
vi) a complete heavy chain;
vii) an additional Fc chain lacking Fd; and
viii) a polypeptide comprising or consisting of the VL domain of the antigen binding site for the radiolabeled compound;
wherein the light chain of (v) and the heavy chain of (vi) together provide an antigen binding site for said antigen; and wherein the polypeptide comprising or consisting of the VL domain of the antigen binding site for the radiolabeled compound is fused by its N-terminus to the C-terminus of either (vi) or (vii);
wherein the antibody heavy chain variable domain (VH) of the polypeptide of (iv) and the antibody light chain variable domain (VL) of the polypeptide of (viii) are together capable of forming a functional antigen binding site for the radiolabelled compound.

14. The set of antibodies according to claim 13, wherein the polypeptide of (iv) is fused by its N-terminus to the C-terminus of either (ii) or (iii) via a linker; and the polypeptide of (viii) is fused by its N-terminus to the C-terminus of either (vi) or (vii) via a linker.

15. The set of antibodies according to any one of claims 10-12, wherein each of the first and second antibody comprise a) an Fc domain b) at least one antibody fragment, such as an scFv, Fv, Fab or cross-Fab fragment, comprising an antigen binding site for the target antigen and c) a polypeptide comprising either a VL domain or a VH domain of the antigen binding site for the radiolabelled compound, wherein the C-terminus of the antibody fragment of (b) is fused to the N-terminus of one chain of the Fc domain, and the C-terminus of the polypeptide of (c) is fused to the N-terminus of the other chain of the Fc domain.

16. The set of antibodies of claim 15, wherein the first antibody comprises:
i) a complete light chain;
ii) a complete heavy chain;
iii) an additional Fc chain; and
iv) a polypeptide comprising or consisting of the VH domain of the antigen binding site for the radiolabeled compound;
wherein the light chain of (i) and the heavy chain of (ii) together provide an antigen binding site for the target antigen; and wherein the polypeptide comprising or consisting of the VH domain of the antigen binding site for the radiolabeled compound is fused by its C-terminus via a linker to the N-terminus of (iii); and
the second antibody comprises:
v) a complete light chain;
vi) a complete heavy chain;
vii) an additional Fc chain; and
viii) a polypeptide comprising or consisting of the VL domain of the antigen binding site for the radiolabeled compound;
wherein the light chain of (v) and the heavy chain of (vi) together provide an antigen binding site for the target antigen; and wherein the polypeptide comprising or consisting of the VL domain of the antigen binding site for the radiolabeled compound is fused by its C-terminus via a linker to the N-terminus of (vii).

17. The set of antibodies according to claim 16, wherein:
i) the first antibody comprises a first heavy chain of SEQ ID NO:112, a second heavy chain of SEQ ID NO: 114 and a light chain of SEQ ID NO: 115; and
ii) the second antibody comprises a first heavy chain of SEQ ID NO:112, a second heavy chain of SEQ ID NO: 113 and a light chain of SEQ ID NO: 115.

18. The set of antibodies of any of claims 10 to 12, wherein the first antibody comprises
a) a first full length antibody and consisting of two antibody heavy chains and two antibody light chains, wherein at least one arm of the full length antibody binds said antigen; and
b) a polypeptide consisting of
i) an antibody heavy chain variable domain (VH); or
ii) an antibody heavy chain variable domain (VH) and an antibody constant domain (CH1),
wherein said polypeptide is fused by the N-terminus of the VH domain to the C-terminus of one of the two heavy chains of said first full-length antibody and
wherein the second antibody comprises:
c) a second full length antibody and consisting of two antibody heavy chains and two antibody light chains, wherein at least one arm of the antibody binds to said antigen; and
d) a polypeptide consisting of
i) an antibody light chain variable domain (VL); or
ii) an antibody light chain variable domain (VL) and an antibody light chain constant domain (CL),
wherein said polypeptide is fused by the N-terminus of the VL domain to the C-terminus of one of the two heavy chains of said second full-length antibody,
wherein the polypeptide of (b) and the polypeptide of (d) are together capable of forming a functional antigen-binding site for the radiolabelled compound.

19. The set of antibodies according to claim 18, wherein the polypeptide (b) is fused by the N-terminus of the VH domain to the C-terminus of one of the two heavy chains of said first full-length antibody via a peptide linker; and the polypeptide of (d) is fused by the N-terminus of the VL domain to the C-terminus of one of the two heavy chains of said second full-length antibody via a peptide linker.

20. The set of antibodies of claim 18 or 19, wherein each of the first and second antibodies is bivalent for said antigen.

21. The set of antibodies of claim 20, wherein both arms of the full length antibody bind to the same epitope of said antigen.

22. The set of antibodies of claim 18 or 19, wherein each of the first and second antibodies is biparatopic for said antigen.

23. The set of antibodies of claim 22, wherein the two arms of the first antibody each bind to an epitope on said antigen and bind to different epitopes from each other; and wherein the two arms of the second antibody each bind to an epitope on said antigen and bind to different epitopes from each other.

24. The set of antibodies of any one of the preceding claims, wherein the radiolabelled compound comprises radiolabelled DOTA or a salt or functional variant thereof.

25. The set of antibodies of any one of the preceding claims, wherein the radiolabelled compound is DOTA or a salt or functional variant thereof radiolabelled with a Lu or Y radioisotope.

26. The set of antibodies according to claim 24 or 25, wherein the VH domain of the antigen binding site for the radiolabelled compound comprises (a) CDR-H1 comprising the amino acid sequence of 35; (b) CDR-H2 comprising the amino acid sequence of 36; (c) CDR-H3 comprising the amino acid sequence of 37.

27. The set of antibodies according to any one of claims 24 to 26, wherein the VH domain of the antigen binding site for the radiolabelled compound comprises the amino acid sequence of SEQ ID NO: 41, or a variant thereof comprising an amino acid sequence having at least 90, 91, 92, 93, 94, 95, 96, 97, 98, or 99% identity to SEQ ID NO: 41.

28. The set of antibodies of claim 27, wherein one or more alanine residues are added to the C-terminus of the VH domain of the antigen binding site for the radiolabelled compound, or wherein one or more residues from the N-terminus of the CH1 domain are added to the C-terminus of the VH domain of the antigen binding site for the radiolabelled compound.

29. The set of antibodies of claim 28, wherein residues AST are added to the C-terminus of the VH domain of the antigen binding site for the radiolabelled compound.

30. The set of antibodies of any one of claims 24 to 29, wherein the VL domain of the antigen binding site for the radiolabelled compound comprises (d) CDR-L1 comprising the amino acid sequence of 38; (e) CDR-L2 comprising the amino acid sequence of 39; and (f) CDR-L3 comprising the amino acid sequence of 40.

31. The set of antibodies of any one of claims 24 to 30, wherein the VL domain of the antigen binding site for the radiolabelled compound comprises an amino acid sequence of SEQ ID NO: 42 or a variant thereof comprising an amino acid sequence having at least 90, 91, 92, 93, 94, 95, 96, 97, 98, or 99% identity to SEQ ID NO: 42.

32. The set of antibodies of any one of claims 1 to 23, wherein the radiolabelled compound comprises Pb-DOTAM.

33. The set of antibodies according to claim 32, wherein the functional binding site for Pb-DOTAM binds with a Kd value of the binding affinity of 100 pM, 50 pM, 20 pM, 10 pM, 5 pM, 1 pM or less, e.g, 0.9 pM or less, 0.8 pM or less, 0.7 pM or less, 0.6 pM or less or 0.5 pM or less.

34. The set of antibodies according to claim 32 or claim 33, wherein the functional binding site for Pb-DOTAM binds to Pb-DOTAM and to Bi-DOTAM.

35. The set of antibodies of any one of claims 32 to 34, wherein the VH domain of the antigen binding site for the radiolabelled compound comprises:
a) heavy chain CDR2 comprising the amino acid sequence FIGSRGDTYYASWAKG (SEQ ID NO:2), or a variant thereof having up to 1, 2, or 3 substitutions in SEQ ID NO: 2, wherein these substitutions do not include Phe50, Asp56 and/or Tyr58, and optionally also do not include Gly52 and/or Arg54;
b) heavy chain CDR3 comprising the amino acid sequence ERDPYGGGAYPPHL (SEQ ID NO:3), or a variant thereof having up to 1, 2, or 3 substitutions in SEQ ID NO: 3, wherein these substitutions do not include Glu95, Arg96, Asp97, Pro98, and optionally also do not include Ala100C, Tyr100D, and/or Pro100E and/or optionally also do not include Tyr99;
and a heavy chain CDR1 which is optionally:
c) a heavy chain CDR1 comprising the amino acid sequence GFSLSTYSMS (SEQ ID NO:1) or a variant thereof having up to 1, 2, or 3 substitutions in SEQ ID NO: 1.

36. The set of antibodies of claim 35, wherein the VH domain of the antigen binding site for the radiolabelled compound comprises (a) CDR-H1 comprising the amino acid sequence of GFSLSTYSMS (SEQ ID NO:1); (b) CDR-H2 comprising the amino acid sequence of FIGSRGDTYYASWAKG (SEQ ID NO:2) and (c) CDR-H3 comprising the amino acid sequence of ERDPYGGGAYPPHL (SEQ ID NO:3).

37. The set of antibodies of any one of claims 32 to 36, wherein the VH domain of the antigen binding site for the radiolabelled compound comprises an amino acid sequence selected from the group consisting of SEQ ID NO: 7 and SEQ ID NO 9, or a variant thereof comprising an amino acid sequence having at least 90, 91, 92, 93, 94, 95, 96, 97, 98, or 99% identity to SEQ ID NO: 7 or SEQ ID NO: 9.

38. The set of antibodies of claim 37, wherein one or more alanine residues are added to the C-terminus of the VH domain of the antigen binding site for the radiolabelled compound, or wherein one or more residues from the N-terminus of the CH1 domain are added to the C-terminus of the VH domain of the antigen binding site for the radiolabelled compound.

39. The set of antibodies of claim 38, wherein residues AST are added to the C-terminus of the VH domain of the antigen binding site for the radiolabelled compound.

40. The set of antibodies of any one of claims 32 to 39, wherein the VL domain of the antigen binding site for the radiolabelled compound comprises
d) light chain CDR1 comprising the amino acid sequence QSSHSVYSDNDLA (SEQ ID NO:4) or a variant thereof having up to 1, 2, or 3 substitutions in SEQ ID NO: 4, wherein these substitutions do not include Tyr28 and Asp32;
e) light chain CDR3 comprising the amino acid sequence LGGYDDESDTYG (SEQ ID NO:6) or a variant thereof having up to 1, 2, or 3 substitutions in SEQ ID NO: 6, wherein these substitutions do not include Gly91, Tyr92, Asp93, Thr95c and Tyr96, and a light chain CDR2 which is optionally
f) light chain CDR 2 comprising the amino acid sequence QASKLAS (SEQ ID NO: 5) or a variant thereof having at least 1, 2 or 3 substitutions in SEQ ID NO: 5, optionally not including Gln50.

41. The set of antibodies of claim 40, wherein the VL domain of the antigen binding site for the radiolabelled compound comprises (d) CDR-L1 comprising the amino acid sequence of QSSHSVYSDNDLA (SEQ ID NO:4); (e) CDR-L2 comprising the amino acid sequence of QASKLAS (SEQ ID NO:5); and (f) CDR-L3 comprising the amino acid sequence of LGGYDDESDTYG (SEQ ID NO:6).

42. The set of antibodies of any one of claims 32 to 41, wherein the VL domain of the antigen binding site for the radiolabelled compound comprises an amino acid sequence of SEQ ID NO: 8, or a variant thereof comprising an amino acid sequence having at least 90, 91, 92, 93, 94, 95, 96, 97, 98, or 99% identity to SEQ ID NO: 8.

43. The set of antibodies of any one of the preceding claims, wherein the first and second antibody bind to the same epitope of the antigen expressed on the surface of a target cell.

44. The set of antibodies of any one of claims 1 to 42, wherein the first antibody binds to a different epitope of the antigen from the second antibody.

45. The set of antibodies of any one of the preceding claims, wherein the antigen expressed on the surface of a target cell is a tumour-associated antigen.

46. The set of antibodies of any one of the preceding claims, wherein the antigen expressed on the surface of a target cell is selected from the group consisting of carcinoembryonic antigen (CEA), CD20, HER2, EGP-1 (epithelial glycoprotein-1, also known as trophoblast-2), colon-specific antigen-p (CSAp), a pancreatic mucin MUC1, GPRC5D and FAP.

47. The set of antibodies of any one of the preceding claims, wherein the antigen expressed on the surface of a target cell is selected from the group consisting of CEA, GPRC5D and FAP.

48. The set of antibodies according to claim 47, wherein:
i) the first antibody comprises a first heavy chain of SEQ ID NO: 104, a second heavy chain of SEQ ID NO:106, wherein the C-terminal alanine of SEQ ID NO: 106 is optional and may be absent or replaced by an alternative C-terminal extension, and a light chain of SEQ ID NO: 107; and
ii) the second antibody comprises a first heavy chain of SEQ ID NO: 104, a second heavy chain of SEQ ID NO: 105 and a light chain of SEQ ID NO: 107.

49. The set of antibodies according to claim 47, wherein
i) the first antibody comprises a first heavy chain of SEQ ID NO: 108, a second heavy chain of SEQ ID NO:110, wherein the C-terminal alanine of SEQ ID NO: 110 is optional and may be absent or replaced by an alternative C-terminal extension, and a light chain of SEQ ID NO: 111; and
ii) the second antibody comprises a first heavy chain of SEQ ID NO: 108, a second heavy chain of SEQ ID NO:109 and a light chain of SEQ ID NO: 111.

50. The set of antibodies of any one of claims 1 to 47, wherein the antigen expressed on the surface of a target cell is CEA

51. The set of antibodies according to claim 50, wherein the first antibody comprises an antigen-binding site which binds to CEA, comprising
a heavy chain variable region comprising (a) CDR-H1 comprising the amino acid sequence of SEQ ID NO:19; (b) CDR-H2 comprising the amino acid sequence of SEQ ID NO:20; and (c) CDR-H3 comprising the amino acid sequence of SEQ ID NO:21; and
a light chain variable region comprising (d) CDR-L1 comprising the amino acid sequence of SEQ ID NO:22; (e) CDR-L2 comprising the amino acid sequence of SEQ ID NO:23; and (f) CDR-L3 comprising the amino acid sequence of SEQ ID NO:24.

52. The set of antibodies according to any one of claims 50-51, wherein the first antibody comprises an antigen-binding site for CEA comprising a VH sequence comprising an amino acid sequence selected from the group consisting of SEQ ID NO: 25, or a variant thereof comprising an amino acid sequence having at least 90, 91, 92, 93, 94, 95, 96, 97, 98, or 99% identity to SEQ ID NO:25.

53. The set of antibodies according to any one of claims 50-52, wherein the first antibody comprises an antigen-binding site for CEA comprising a VL sequence comprising an amino acid sequence selected from the group consisting of SEQ ID NO: 26, or a variant thereof comprising an amino acid sequence having at least 90, 91, 92, 93, 94, 95, 96, 97, 98, or 99% identity to SEQ ID NO:26.

54. The set of antibodies according to claim 50, wherein the first antibody comprises an antigen-binding site which binds to CEA, comprising a heavy chain variable region comprising (a) CDR-H1 comprising the amino acid sequence of SEQ ID NO:43; (b) CDR-H2 comprising the amino acid sequence of SEQ ID NO:44; and (c) CDR-H3 comprising the amino acid sequence of SEQ ID NO:45; and a light chain variable region comprising (d) CDR-L1 comprising the amino acid sequence of SEQ ID NO:46; (e) CDR-L2 comprising the amino acid sequence of SEQ ID NO:47; and (0 CDR-L3 comprising the amino acid sequence of SEQ ID NO:48.

55. The set of antibodies according to claim 50 or 54, wherein the first antibody comprises an antigen-binding site for CEA comprising a VH sequence comprising an amino acid sequence selected from the group consisting of SEQ ID NO: 49, or a variant thereof comprising an amino acid sequence having at least 90, 91, 92, 93, 94, 95, 96, 97, 98, or 99% identity to SEQ ID NO:49.

56. The set of antibodies according to any one of claim 50, 54 or 55, wherein the first antibody comprises an antigen-binding site for CEA comprising a VL sequence comprising an amino acid sequence selected from the group consisting of SEQ ID NO: 50, or a variant thereof comprising an amino acid sequence having at least 90, 91, 92, 93, 94, 95, 96, 97, 98, or 99% identity to SEQ ID NO:50.

57. The set of antibodies according to claim 50, wherein the first antibody comprises an antigen-binding site which binds to CEA, comprising
a heavy chain variable region comprising (a) CDR-H1 comprising the amino acid sequence of SEQ ID NO:11; (b) CDR-H2 comprising the amino acid sequence of SEQ ID NO:12; and (c) CDR-H3 comprising the amino acid sequence of SEQ ID NO:13; and
a light chain variable region comprising (d) CDR-L1 comprising the amino acid sequence of SEQ ID NO:14; (e) CDR-L2 comprising the amino acid sequence of SEQ ID NO:15; and (f) CDR-L3 comprising the amino acid sequence of SEQ ID NO:16.

58. The set of antibodies according to claim 50 or 57, wherein the first antibody comprises an antigen-binding site for CEA comprising a VH sequence comprising an amino acid sequence selected from the group consisting of SEQ ID NO: 17, or a variant thereof comprising an amino acid sequence having at least 90, 91, 92, 93, 94, 95, 96, 97, 98, or 99% identity to SEQ ID NO:17.

59. The set of antibodies according to claim 50, 57 or 58, wherein the first antibody comprises an antigen-binding site for CEA comprising a VL sequence comprising an amino acid sequence selected from the group consisting of SEQ ID NO: 18, or a variant thereof comprising an amino acid sequence having at least 90, 91, 92, 93, 94, 95, 96, 97, 98, or 99% identity to SEQ ID NO:18.

60. The set of antibodies according to claim 50, wherein the first antibody comprises an antigen-binding site which binds to CEA, comprising
a heavy chain variable region comprising (a) CDR-H1 comprising the amino acid sequence of SEQ ID NO:59; (b) CDR-H2 comprising the amino acid sequence of SEQ ID NO:60; and (c) CDR-H3 comprising the amino acid sequence of SEQ ID NO:61; and
a light chain variable region comprising (d) CDR-L1 comprising the amino acid sequence of SEQ ID NO:62; (e) CDR-L2 comprising the amino acid sequence of SEQ ID NO:63; and (0 CDR-L3 comprising the amino acid sequence of SEQ ID NO:64.

61. The set of antibodies according to claim 50 or claim 60, wherein the first antibody comprises an antigen-binding site for CEA comprising a VH sequence comprising an amino acid sequence selected from the group consisting of SEQ ID NO: 65, or a variant thereof comprising an amino acid sequence having at least 90, 91, 92, 93, 94, 95, 96, 97, 98, or 99% identity to SEQ ID NO:65.

62. The set of antibodies according to claim 50, 60 or 61, wherein the first antibody comprises an antigen-binding site for CEA comprising a VL sequence comprising an amino acid sequence selected from the group consisting of SEQ ID NO: 66, or a variant thereof comprising an amino acid sequence having at least 90, 91, 92, 93, 94, 95, 96, 97, 98, or 99% identity to SEQ ID NO:66.

63. The set of antibodies according to claim 50, wherein the first antibody comprises an antigen-binding site which binds to CEA, comprising a heavy chain variable region comprising (a) CDR-H1 comprising the amino acid sequence of SEQ ID NO:156; (b) CDR-H2 comprising the amino acid sequence of SEQ ID NO:157 or 158; (c) CDR-H3 comprising the amino acid sequence of SEQ ID NO:159; and a light chain variable region comprising (d) CDR-L1 comprising the amino acid sequence of SEQ ID NO:160, 161 or 162; (e) CDR-L2 comprising the amino acid sequence of SEQ ID NO:163, 164 or 165; and (f) CDR-L3 comprising the amino acid sequence of SEQ ID NO:166.

64. The set of antibodies according to claim 50 or 63, wherein the first antibody comprises an antigen-binding site which binds to CEA, comprising a heavy chain variable region (VH) comprising an amino acid sequence selected from SEQ ID NO: 169, 170, 171, 172, 173 or 174, or a sequence with 90, 91, 92, 93, 94, 95, 96, 97, 98, or 99% identity thereto; and a light chain variable region (VL) comprising an amino acid sequence selected from SEQ ID NO: 175, 176, 177, 178, 179 or 180, or a sequence with 90, 91, 92, 93, 94, 95, 96, 97, 98, or 99% identity thereto.

65. The set of antibodies according to claim 50, 63 or 64, wherein the first antibody comprises an antigen-binding site which binds to CEA, comprising:
(a) a VH domain comprising an amino acid sequence of SEQ ID NO:169 and a VL domain comprising an amino acid sequence of SEQ ID NO:179, or
(b) a VH domain comprising an amino acid sequence of SEQ ID NO:173 and a VL domain comprising an amino acid sequence of SEQ ID NO:179, or
(c) a VH domain comprising an amino acid sequence of SEQ ID NO:170 and a VL domain comprising an amino acid sequence of SEQ ID NO:179, or
(d) a VH domain comprising an amino acid sequence of SEQ ID NO:174 and a VL domain comprising an amino acid sequence of SEQ ID NO:178, or
(e) a VH domain comprising an amino acid sequence of SEQ ID NO:173 and a VL domain comprising an amino acid sequence of SEQ ID NO:178, or
(f) a VH domain comprising an amino acid sequence of SEQ ID NO:171 and a VL domain comprising an amino acid sequence of SEQ ID NO:178, or
(g) a VH domain comprising an amino acid sequence of SEQ ID NO:169 and a VL domain comprising an amino acid sequence of SEQ ID NO:178.

66. The set of antibodies according to any one of claims 50 to 65, wherein the second antibody comprises an antigen-binding site which binds to CEA, comprising
a heavy chain variable region comprising (a) CDR-H1 comprising the amino acid sequence of SEQ ID NO:19; (b) CDR-H2 comprising the amino acid sequence of SEQ ID NO:20; and (c) CDR-H3 comprising the amino acid sequence of SEQ ID NO:21; and
a light chain variable region comprising (d) CDR-L1 comprising the amino acid sequence of SEQ ID NO:22; (e) CDR-L2 comprising the amino acid sequence of SEQ ID NO:23; and (f) CDR-L3 comprising the amino acid sequence of SEQ ID NO:24.

67. The set of antibodies according to any one of claims 50-66, wherein the second antibody comprises an antigen-binding site for CEA comprising a VH sequence comprising an amino acid sequence selected from the group consisting of SEQ ID NO: 25, or a variant thereof comprising an amino acid sequence having at least 90, 91, 92, 93, 94, 95, 96, 97, 98, or 99% identity to SEQ ID NO:25.

68. The set of antibodies according to any one of claims 50-67, wherein the second antibody comprises an antigen-binding site for CEA comprising a VL sequence comprising an amino acid sequence selected from the group consisting of SEQ ID NO: 26, or a variant thereof comprising an amino acid sequence having at least 90, 91, 92, 93, 94, 95, 96, 97, 98, or 99% identity to SEQ ID NO:26.

69. The set of antibodies according to any one of claims 50 to 65, wherein the second antibody comprises an antigen-binding site which binds to CEA, comprising a heavy chain variable region comprising (a) CDR-H1 comprising the amino acid sequence of SEQ ID NO:43; (b) CDR-H2 comprising the amino acid sequence of SEQ ID NO:44; and (c) CDR-H3 comprising the amino acid sequence of SEQ ID NO:45; and a light chain variable region comprising (d) CDR-L1 comprising the amino acid sequence of SEQ ID NO:46; (e) CDR-L2 comprising the amino acid sequence of SEQ ID NO:47; and (f) CDR-L3 comprising the amino acid sequence of SEQ ID NO:48.

70. The set of antibodies according to any one of claim 50 to 65 or 69, wherein the second antibody comprises an antigen-binding site for CEA comprising a VH sequence comprising an amino acid sequence selected from the group consisting of SEQ ID NO: 49, or a variant thereof comprising an amino acid sequence having at least 90, 91, 92, 93, 94, 95, 96, 97, 98, or 99% identity to SEQ ID NO:49.

71. The set of antibodies according to any one of claim 50 to 65, 69 or 70, wherein the second antibody comprises an antigen-binding site for CEA comprising a VL sequence comprising an amino acid sequence selected from the group consisting of SEQ ID NO: 50, or a variant thereof comprising an amino acid sequence having at least 90, 91, 92, 93, 94, 95, 96, 97, 98, or 99% identity to SEQ ID NO:50.

72. The set of antibodies according to any one of claims 50 to 65, wherein the second antibody comprises an antigen-binding site which binds to CEA, comprising
a heavy chain variable region comprising (a) CDR-H1 comprising the amino acid sequence of SEQ ID NO:11; (b) CDR-H2 comprising the amino acid sequence of SEQ ID NO:12; and (c) CDR-H3 comprising the amino acid sequence of SEQ ID NO:13; and
a light chain variable region comprising (d) CDR-L1 comprising the amino acid sequence of SEQ ID NO:14; (e) CDR-L2 comprising the amino acid sequence of SEQ ID NO:15; and (f) CDR-L3 comprising the amino acid sequence of SEQ ID NO:16.

73. The set of antibodies according to any one of claim 50 to 65 or 72, wherein the second antibody comprises an antigen-binding site for CEA comprising a VH sequence comprising an amino acid sequence selected from the group consisting of SEQ ID NO: 17, or a variant thereof comprising an amino acid sequence having at least 90, 91, 92, 93, 94, 95, 96, 97, 98, or 99% identity to SEQ ID NO:17.

74. The set of antibodies according to any one of claim 50 to 65, 72 or 73, wherein the second antibody comprises an antigen-binding site for CEA comprising a VL sequence comprising an amino acid sequence selected from the group consisting of SEQ ID NO: 18, or a variant thereof comprising an amino acid sequence having at least 90, 91, 92, 93, 94, 95, 96, 97, 98, or 99% identity to SEQ ID NO:18.

75. The set of antibodies according to any one of claims 50 to 65, wherein the second antibody comprises an antigen-binding site which binds to CEA, comprising
a heavy chain variable region comprising (a) CDR-H1 comprising the amino acid sequence of SEQ ID NO:59; (b) CDR-H2 comprising the amino acid sequence of SEQ ID NO:60; and (c) CDR-H3 comprising the amino acid sequence of SEQ ID NO:61; and
a light chain variable region comprising (d) CDR-L1 comprising the amino acid sequence of SEQ ID NO:62; (e) CDR-L2 comprising the amino acid sequence of SEQ ID NO:63; and (f) CDR-L3 comprising the amino acid sequence of SEQ ID NO:64.

76. The set of antibodies according to any one of claim 50 to 65 or 75, wherein the second antibody comprises an antigen-binding site for CEA comprising a VH sequence comprising an amino acid sequence selected from the group consisting of SEQ ID NO: 65, or a variant thereof comprising an amino acid sequence having at least 90, 91, 92, 93, 94, 95, 96, 97, 98, or 99% identity to SEQ ID NO:65.

77. The set of antibodies according to claim 50 to 65, 75 or 76, wherein the second antibody comprises an antigen-binding site for CEA comprising a VL sequence comprising an amino acid sequence selected from the group consisting of SEQ ID NO: 66, or a variant thereof comprising an amino acid sequence having at least 90, 91, 92, 93, 94, 95, 96, 97, 98, or 99% identity to SEQ ID NO:66.

78. The set of antibodies according to claims 50 to 65, wherein the second antibody comprises an antigen-binding site which binds to CEA, comprising a heavy chain variable region comprising (a) CDR-H1 comprising the amino acid sequence of SEQ ID NO:156; (b) CDR-H2 comprising the amino acid sequence of SEQ ID NO:157 or 158; (c) CDR-H3 comprising the amino acid sequence of SEQ ID NO:159; and a light chain variable region comprising (d) CDR-L1 comprising the amino acid sequence of SEQ ID NO:160, 161 or 162; (e) CDR-L2 comprising the amino acid sequence of SEQ ID NO:163, 164 or 165; and (0 CDR-L3 comprising the amino acid sequence of SEQ ID NO:166.

79. The set of antibodies according to claim 50 to 65 or 78, wherein the second antibody comprises an antigen-binding site which binds to CEA, comprising a heavy chain variable region (VH) comprising an amino acid sequence selected from SEQ ID NO: 169, 170, 171, 172, 173 or 174, or a sequence with 90, 91, 92, 93, 94, 95, 96, 97, 98, or 99% identity thereto; and a light chain variable region (VL) comprising an amino acid sequence selected from SEQ ID NO: 175, 176, 177, 178, 179 or 180, or a sequence with 90, 91, 92, 93, 94, 95, 96, 97, 98, or 99% identity thereto.

80. The set of antibodies according to claim 50 to 65, 78 or 79, wherein the second antibody comprises an antigen-binding site which binds to CEA, comprising:
(a) a VH domain comprising an amino acid sequence of SEQ ID NO:169 and a VL domain comprising an amino acid sequence of SEQ ID NO:179, or
(b) a VH domain comprising an amino acid sequence of SEQ ID NO:173 and a VL domain comprising an amino acid sequence of SEQ ID NO:179, or
(c) a VH domain comprising an amino acid sequence of SEQ ID NO:170 and a VL domain comprising an amino acid sequence of SEQ ID NO:179, or
(d) a VH domain comprising an amino acid sequence of SEQ ID NO:174 and a VL domain comprising an amino acid sequence of SEQ ID NO:178, or
(e) a VH domain comprising an amino acid sequence of SEQ ID NO:173 and a VL domain comprising an amino acid sequence of SEQ ID NO:178, or
(f) a VH domain comprising an amino acid sequence of SEQ ID NO:171 and a VL domain comprising an amino acid sequence of SEQ ID NO:178, or
(g) a VH domain comprising an amino acid sequence of SEQ ID NO:169 and a VL domain comprising an amino acid sequence of SEQ ID NO:178.

81. The set of antibodies according to claim 50, wherein the first antibody is an antibody according to any one of claims 57 to 59 and the second antibody is an antibody according to any one of claims 66 to 68.

82. The set of antibodies according claim 1, wherein the first antibody comprises a first heavy chain of SEQ ID NO: 28, a second heavy chain of SEQ ID NO: 32 and a light chain of SEQ ID NO: 34.

83. The set of antibodies according to claim 82, wherein one or more alanine residues is added to the C-terminus of SEQ ID NO: 32, or wherein the sequence AST is added to the C-terminus of SEQ ID NO: 32.

84. The set of antibodies according to claim 1, wherein the first antibody comprises a first heavy chain of SEQ ID NO: 51, and second heavy chain of SEQ ID NO: 52 and a light chain of SEQ ID NO: 54.

85. The set of antibodies according to claim 84, wherein one or more alanine residues is added to the C-terminus of SEQ ID NO:52 or wherein the sequence AST is added to the C-terminus of SEQ ID NO: 32.

86. The set of antibodies according to claim 1, wherein the first antibody comprises a first heavy chain of SEQ ID NO: 86, a second heavy chain of SEQ ID NO: 87 wherein the C-terminal AST residues are optional and may be absent or replaced by a different C-terminal extension, and a light chain of SEQ ID NO: 89.

87. The set of antibodies according to claim 1, wherein the first antibody comprises a first heavy chain of SEQ ID NO: 93, a second heavy chain of SEQ ID NO: 94 wherein the C-terminal AST residues are optional and may be absent or replaced by a different C-terminal extension, and a light chain of SEQ ID NO: 96.

88. The set of antibodies according to claim 1, or 82-87 wherein the second antibody comprises a first heavy chain of SEQ ID NO: 30, a second heavy chain of SEQ ID NO: 33 and a light chain of SEQ ID NO: 34.

89. The set of antibodies according to claim 1, or 82-87, wherein the second antibody comprises a first heavy chain of SEQ ID NO: 55, and second heavy chain of SEQ ID NO: 56 and a light chain of SEQ ID NO: 58.

90. The set of antibodies according to claim 1 or 82-87, wherein the second antibody comprises a first heavy chain of SEQ ID NO: 83, and second heavy chain of SEQ ID NO: 84 and a light chain of SEQ ID NO: 89.

91. The set of antibodies according to claim 1 or 82-87, wherein the second antibody comprises a first heavy chain of SEQ ID NO:90, and second heavy chain of SEQ ID NO: 91 and a light chain of SEQ ID NO: 96.

92. A set of nucleic acids expressing the set of antibodies of any one of the preceding claims.

93. An expression vector or set of expression vectors comprising the set of nucleic acids according to claim 92.

94. A host cell or set of host cells comprising the expression vector or set of expression vectors of claim 93.

95. A method of pretargeting radioimmunotherapy, comprising
i) administering to a subject the set of antibodies according to any one of claims 1 to 91, wherein the first and second antibodies are administered simultaneously or sequentially, in either order, wherein the antibodies bind to the target antigen and localise to the surface of a cell expressing the target antigen; and wherein association of the first and second antibody forms a functional binding site for the radiolabelled compound; and
ii) subsequently administering a radiolabelled compound, wherein the radiolabelled compound binds to functional binding site for the radiolabelled compound.

96. The method of claim 95, wherein the method does not comprise a step of administering a clearing or blocking agent.

97. The method of claim 95 or 96, wherein the subject is human.

98. The method of any one of claims 95 to 97, wherein the target antigen is a cancer- or tumour-associated antigen and the method is a method of radioimmunotherapy of a tumour or cancer.

99. The set of antibodies according to any one of claims 1 to 91, for use in a method of pretargeting radioimmunotherapy according to any one of claims 95 to 98.

100. A method of targeting a radioisotope to a tissue or organ for radioimaging comprising:
i) administering to a subject a set of antibodies according to any one of claims 1 to 91, wherein the first and second antibodies are administered simultaneously or sequentially, in either order, wherein the antibodies bind to a target antigen and localise to the surface of a cell expressing the target antigen, wherein association of the first and second antibody forms a functional binding site for the radiolabelled compound;
and
ii) subsequently administering a radiolabelled compound, wherein the radiolabelled compound binds to the functional binding site for the radiolabelled compound.

101. The method of claim 100, wherein the method does not comprise a step of administering a clearing or blocking agent.

102. The method of claim 100 or 101, wherein the method further comprises a step of imaging.

103. The method of claim 102, wherein the target antigen is a cancer or tumour-associated antigen and the method is a method of imaging tumours or cancer.
